(19) United States
(12) Patent Application Publication
(10) Pub. No.: US 2020/0353088 A1
(43) Pub. Date: Nov. 12, 2020

(54) PEPTIDE CONJUGATE CGRP RECEPTOR ANTAGONISTS AND METHODS OF PREPARATION AND USES THEREOF

(71) Applicant: Auckland UniServices Limited, Auckland (NZ)

(72) Inventors: Paul William Richard Harris, Auckland (NZ); Kerry Martin Loomes, Auckland (NZ); Deborah Lucy Hay, Auckland (NZ); Aqfan Jamaluddin, Auckland (NZ); Christopher Stuart Walker, Auckland (NZ); Elyse Thomasine Williams, Auckland (NZ); Margaret Anne Brimble, Auckland (NZ)

(21) Appl. No.: 16/762,098

(22) PCT Filed: Nov. 6, 2018
(86) PCT No.: PCT/IB2018/058684

§ 371 Date: May 6, 2020

(30) Foreign Application Priority Data
Nov. 6, 2017 (NZ) 736960

(51) Int. Cl. A61K 47/54; C07K 14/575; A61P 9/00; A61P 25/06

(52) U.S. Cl. CPC A61K 47/543; C07K 14/57527; A61K 38/00; A61P 25/06; A61P 9/00

(57) ABSTRACT

Disclosed are peptide conjugates that are calcitonin gene-related peptide (CGRP) receptor antagonists comprising a CGRP peptide, wherein at least one amino acid of the peptide is covalently conjugated to a lipid-containing moiety. Also disclosed are pharmaceutical compositions and kits comprising such conjugates, methods of preparing such conjugates, and uses of such antagonists.

### FIELD OF THE INVENTION

The present invention generally relates to peptide conjugates that are antagonists of calcitonin gene-related peptide (CGRP) receptors, pharmaceutical compositions and kits comprising such conjugates, methods of preparing such conjugates, and uses of such antagonists.

### BACKGROUND OF THE INVENTION

CGRP is a sensory neuropeptide that exists in two forms in humans (α- and β-CGRP). The two forms of CGRP both comprise a 37 amino acid sequence but are encoded by different genes and differ from each other by three amino acids.

CGRP and its associated receptors are found in both the central and the peripheral nervous system and are expressed in cell types that play a role in inflammation and/or nociception.

As such, CGRP is found in a wide range of cells throughout the body, for example in blood vessels, in sensory ganglia and in the gastrointestinal tract, as well as in organs such as, for example, the skin, lungs, kidney and heart.

CGRP is stored in sensory nerves and is released from neurons in response to neuronal depolarisation. CGRP exerts its effects by binding and activating associated receptors.

Activation of a CGRP receptor has been associated with migraines. CGRP receptor antagonists represent promising targets for the treatment of migraines and various other diseases and conditions associated with CGRP receptor and various other disease and conditions, such as metabolic disorders or syndromes.

Known CGRP receptor antagonists include peptide antagonists such as the CGRP fragment CGRP₈₋₃₇ and non-peptide antagonists such as the “gepant” class of antagonists, for example olcegepant (BIBN4096BS) and telcagepant (MK0974), both of which have been investigated for the treatment of migraines.

There is an ongoing need for further CGRP antagonists. It is an object of the present invention to go some way to meeting this need and/or to at least provide the public with a useful choice.

Other objects of the invention may become apparent from the following description which is given by way of example only.

In this specification where reference has been made to patent specifications, other external documents, or other sources of information, this is generally for the purpose of providing a context for discussing the features of the invention. Unless specifically stated otherwise, reference to such external documents is not to be construed as an admission that such documents, or such sources of information, in any jurisdiction, are prior art, or form part of the common general knowledge in the art.

### SUMMARY OF THE INVENTION

In a first aspect, the present invention broadly consists in a peptide conjugate comprising a calcitonin gene-related peptide (CGRP) peptide, wherein at least one amino acid of the peptide is covalently conjugated to a lipid-containing moiety, wherein the peptide conjugate is a CGRP receptor antagonist.

The following embodiments and preferences may relate alone or in any combination of any two or more to any of the aspects herein.

In various embodiments, the at least one amino acid is covalently conjugated to the lipid containing moiety via heteroatom of the amino acid.

In various embodiments, the heteroatom is of a side chain of the amino acid.

In various embodiments, the at least one amino acid is covalently conjugated to the lipid containing moiety via a sulfur atom of a sulfide group.

In another aspect, the present invention broadly consists in a peptide conjugate comprising a calcitonin gene-related peptide (CGRP) peptide, wherein at least one amino acid of the peptide is covalently conjugated to a lipid-containing moiety via a sulfur atom of a sulfide group, wherein the peptide conjugate is a CGRP receptor antagonist.

In some embodiments, the peptide conjugate has an antagonist potency value (pA₂) more than a value about 10-fold less than, 5-fold less than, 3-fold less than, 2-fold less than, 1-fold less than the antagonist potency (pA₂) of α-CGRP8-37 (SEQ ID No:96) at a CGRP receptor or has an antagonist potency value (pA₂) more than a value equal to the antagonist potency (pA₂) of α-CGRP8-37 (SEQ ID No:96) at a CGRP receptor, for example as measured by a cAMP assay as described in the Examples herein.

In some embodiments, the CGRP receptor is a CLR/RAMP1 CGRP receptor or a CTR/RAMP1 AMY1 CGRP receptor.

In some embodiments, the peptide conjugate has a half life at least 2-, 3-, 4-, 5-, 10-, 20-, 30-, 40-, or -50-fold longer than the half life of α-CGRP8-37 (SEQ ID No:96), for example as measured in a suitable rodent model, for example a rat model.

In some embodiments, the peptide conjugate and α-CGRP8-37 (SEQ ID No:96) each independently have a first antagonist potency value (pA₂) at a CGRP receptor and a second antagonist potency value (pA₂) at a CGRP receptor;
wherein the first antagonist potency value (pA₂) at a CGRP receptor is after incubating the receptor and peptide conjugate or α-CGRP8-37 (SEQ ID No:96) and not washing the receptor prior to determining the antagonist potency value;
wherein the second antagonist potency value (pA₂) at a CGRP receptor is after incubating the receptor and peptide conjugate or α-CGRP8-37 (SEQ ID No:96) and then washing the receptor prior to determining the antagonist potency value;
wherein the second antagonist potency value (pA₂) is less than the first antagonist potency value (pA₂); and
the fold change reduction in antagonist potency between the first antagonist potency value (pA₂) of the peptide conjugate and the second antagonist potency value (pA₂) of the peptide conjugate is less than the fold change reduction in antagonist potency between the the first antagonist potency value (pA₂) of α-CGRP8-37 (SEQ ID No:96) and the second antagonist potency value (pA₂) of α-CGRP8-37 (SEQ ID No:96).

In various embodiments, the antagonist potency value (pA₂) at a CGRP receptor is measured by a cAMP assay, for example as described in the Examples herein, optionally wherein the CGRP receptor is a CLR/RAMP1 CGRP receptor or a CTR/RAMP1 AMY1 CGRP receptor.

In various embodiments, the fold change reduction in antagonist potency between the first antagonist potency value (pA₂) of the peptide conjugate and the second antagonist potency value (pA₂) of the peptide conjutage is less than about 50, 45, 40, 35, 30, 25, 20, 15, 10, 9, 8, 7, 6, 5, 4, 3, or 2, wherein the antagonist potency value (pA₂) at a CGRP receptor is measured by a cAMP assay, wherein the CGRP receptor is a CLR/RAMP1 CGRP receptor, for example as described in the Examples herein.

In various embodiments, the fold change reduction in antagonist potency between the first antagonist potency value (pA₂) of the peptide conjugate and the second antagonist potency value (pA₂) of the peptide conjutage is less than about 20, 15, 14, 13, 12, 11, 10, 9, 8, 7, 6, 5, 4, 3, or 2, wherein the antagonist potency value (pA₂) at a CGRP receptor is measured by a cAMP assay, wherein the CGRP receptor is a CTR/RAMP1 AMY1 CGRP receptor, for example as described in the Examples herein.

In various embodiments, the fold change reduction in antagonist potency of the peptide conjugate is at least about 2-, 3-, 4-, 5-, 6-, 7-, 8-, 9-, 10-, 11-, 12-, 13-, 14-, 15-, 20-, 25-, 50-, 100-, 250-, or 500-fold less than the fold change reduction in antagonist potency of α-CGRP8-37 (SEQ ID No:96).

In certain embodiments, the at least one amino acid is cysteine or homocysteine. In exemplary embodiments, the at least one amino acid is cysteine.

In exemplary embodiments, the peptide conjugate comprises only one amino acid conjugated to a lipid-containing moiety. In other embodiments, the peptide conjugate comprises two or more amino acids each conjugated to a lipid-containing moiety.

In some embodiments, the lipid-containing moiety comprises one or more straight or branched aliphatic or heteroaliphatic chains each containing at least 4 or at least 6 chain-linked atoms.

In certain embodiments, the lipid-containing moiety comprises one or more saturated or unsaturated fatty acid esters.

In various embodiments, the fatty acid is saturated.

In some embodiments, the lipid-containing moiety is of the formula (A):

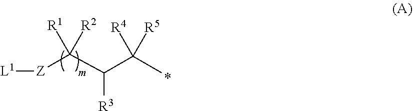

wherein
   * represents a bond to the sulfur atom of the sulfide group of the amino acid to which the lipid-containing moiety is conjugated;
   Z and Z¹ are each independently selected from the group consisting of —O—, —NR—, —S—, —S(O)—, —SO₂—, —C(O)O—, —OC(O)—, —C(O)NR—, —NRC(O)—, —C(O)S—, —SC(O)—, —OC(O)O—, —NRC(O)O—, —OC(O)NR—, and —NRC(O)NR—;
   R is hydrogen or C₁₋₆aliphatic;
   m is an integer from 0 to 4;
   n is 1 or 2;
   R¹ and R² at each instance of m are each independently hydrogen, C₁₋₆aliphatic; or
R¹ is L²-Z¹—C₁₋₆alkyl;
   R³, R⁴, and R⁵ are each independently hydrogen or C₁₋₆aliphatic; or R³ is L²-Z¹—C₁₋₆ alkyl;
   L¹ and L² are each independently C₅₋₂₁aliphatic or C₄₋₂₀heteroaliphatic;
   provided that:
      when R³ is L²-Z¹—C₁₋₆alkyl, R¹ is not L²-Z¹—C₁₋₆alkyl; and
      when m is an integer from 2 to 4, no more than one R¹ is L²-Z¹—C₁₋₆alkyl; and
   wherein any aliphatic, alkyl, or heteroaliphatic present in any of R, R¹, R², R³, R⁴, R⁵, L¹, and L² is optionally substituted with one or more independently selected optional substituents.

In some embodiments
R is hydrogen, C₁₋₆alkyl, or C₃₋₆cycloalkyl;
m is an integer from 0 to 4;
n is 1 or 2;
R¹ and R² at each instance of m are each independently hydrogen, C₁₋₆alkyl, or C₃₋₆cycloalkyl; or R¹ is L²-Z¹—C₁₋₆alkyl;
R³, R⁴, and R⁵ are each independently hydrogen, C₁₋₆alkyl, or C₃₋₆cycloalkyl; or R³ is L²-Z¹—C₁₋₆alkyl;
L¹ and L² are each independently C₅₋₂₁alkyl, C₅₋₂₁alkenyl, or C₄₋₂₀heteroalkyl;
wherein any alkyl, alkenyl, cycloalkyl, or heteroalkyl present in any of R, R¹, R², R³, R⁴, R⁵, L¹, and L² is optionally substituted with one or more independently selected optional substituents.

In some embodiments
R is hydrogen or C₁₋₆alkyl;
m is an integer from 0 to 4;
n is 1 or 2;
R¹ and R² at each instance of m are each independently hydrogen or C₁₋₆alkyl; or R¹ is L²-Z¹—C₁₋₆alkyl;
R³, R⁴, and R⁵ are each independently hydrogen or C₁₋₆alkyl; or R³ is L²-Z¹—C₁₋₆alkyl;
L¹ and L² are each independently C₅₋₂₁alkyl, C₅₋₂₁alkenyl, or C₄₋₂₀heteroalkyl;
wherein any alkyl, alkenyl, or heteroalkyl present in any of R, R¹, R², R³, R⁴, R⁵, L¹, and L² is optionally substituted with one or more independently selected optional substituents.

In some embodiments, Z and Z¹ are each independently selected from —C(O)O—, —C(O)NR—, and —C(O)S—, preferably —C(O)O—.

In certain embodiments, the lipid-containing moiety is of the formula (I)

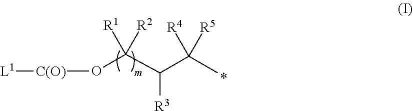

wherein
m, L¹, R¹, R², R³, R⁴, and R⁵ are as defined in any of the embodiments herein; and
Z¹ when present is —C(O)O—.

In some embodiments, m is an integer from 0 to 2. In certain embodiments, m is 0 or 1. In exemplary embodiments, m is 0.

In certain embodiments, R¹ and R² at each instance of m are each independently hydrogen.

In certain embodiments, R⁴ and R⁵ are each hydrogen.

In some embodiments, R³ is hydrogen or C₁₋₆alkyl.

In some embodiments, the lipid-containing moiety is of the formula (IV):

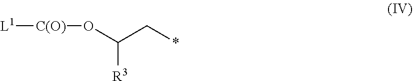

wherein
   R³ is hydrogen, L²-C(O)—OCH₂, or L²-C(O)—OCH₂CH₂; and
   L¹ and L² are each independently C₅₋₂₁alkyl, C₅₋₂₁alkenyl, or C₄₋₂₀heteroalkyl.

In certain embodiments, L¹ and L² are each independently C₅₋₂₁aliphatic, for example C₉₋₂₁alihpatic, C₁₁₋₂₁aliphatic, or C₉-, C₁₁-, C₁₃-, C₁₅-, C₁₇-, or C₁₉-aliphatic.

In certain embodiments, L¹ and L² are each independently C₅₋₂₁alkyl. In various embodiments, L¹ and L² are each independently C₉₋₂₁alkyl. In some embodiments, L¹ and L² are each independently is C₁₁₋₂₁alkyl.

In various exemplary embodiments, L¹ and L² are each independently C₉, C₁₁, C₁₃, C₁₅, C₁₇, or C₁₉alkyl, preferably n-alkyl.

In various specifically contemplated embodiments, L¹ and L² are each independently C₁₅alkyl. In certain embodiments, L¹ and L² are each independently linear C₅alkyl.

In various embodiments, L¹ and L² each independently comprise a linear chain of 9-21 carbon atoms.

In some embodiments, R³ is L²-C(O)—OCH₂CH₂. In some embodiments, R³ is L²-C(O)—OCH₂. In exemplary embodiments, R³ is hydrogen.

In one embodiment, L¹ is C₅₋₂₁alkyl; m is 0; R³ is hydrogen, L²-C(O)—OCH₂, or L²-C(O)—OCH₂CH₂; L² is C₁₁₋₂₁alkyl; and R⁴ and R⁵ are each hydrogen.

In one embodiment, L¹ is C₅₋₂₁alkyl; m is 0; R³ is hydrogen; L² is C₁₋₂₁alkyl; and R⁴ and R⁵ are each hydrogen.

In one embodiment, L¹ is C₅₋₂₁alkyl; m is 0; R³ is L²-C(O)—OCH₂; L² is C₁₋₂₁alkyl; and R⁴ and R⁵ are each hydrogen.

In one embodiment, L is C₅₋₂₁alkyl; m is 0; R³ is L²-C(O)—OCH₂CH₂; L² is C₁₋₂₁alkyl; and R⁴ and R⁵ are each hydrogen.

Those skilled in the art will appreciate that, in certain embodiments, the moieties L¹-Z¹- and L²-Z²— may be fatty acid groups, for example fatty acid esters.

In various embodiments, the moieties L¹-Z¹— and L²-Z²— may be saturated or unsaturated fatty acid esters. In some embodiments, the fatty acid is saturated.

In various embodiments, the fatty acid is a C₄₋₂₂ fatty acid. In some embodiments, the fatty acid is a C₆₋₂₂ fatty acid. In certain embodiments, the fatty acid is a C₁₀₋₂₂ fatty acid. In certain specifically contemplated embodiments, the fatty acid is a C₁₂₋₂₂ fatty acid. In various exemplary embodiments, the fatty acid is a C₁₀, C₁₂, C₁₄, C₁₆, C₁₈, or C₂₀ fatty acid.

In some embodiments, the fatty acid is decanoic acid, lauric acid, myristic acid, palmitic acid, stearic acid, arachic acid, palmitoleic acid, oleic acid, elaidic acid, linoleic acid, α-linolenic acid, and arachidonic acid. In various embodiments, the fatty acid is decanoic acid, lauric acid, myristic acid, palmitic acid, or stearic acid.

In certain exemplary embodiments, the fatty acid is palmitic acid (and the moieties L¹-Z¹- and L²-Z²— are each palmitoyl groups).

In various embodiments, the one or more independently selected optional substituents are selected from halo, CN, NO₂, OH, NH₂, NHR^{x}, NR^{x}R^{y}, C₁₋₆haloalkyl, C₁₋₆haloalkoxy, C(O)NH₂, C(O)NHR^{x}, C(O)NR^{x}R^{y}, SO₂R^{x}, OR^{y}, SR^{x}, S(O)R^{x}, C(O)R^{x}, and C₁₋₆aliphatic; wherein R^{x} and R^{y} are each independently C₁₋₆aliphatic, for example C₁₋₆alkyl.

In some embodiments, said optionally substituted groups are unsubstituted.

In various embodiments, the N-terminal group of the peptide is —NR^{a}R^{b}, wherein R^{a} and R^{b} are each independently hydrogen, alkyl, cycloalkyl, acyl, aryl, or arylalkyl; and/or the C-terminal group of the peptide is —CH₂OR^{c}, —C(O)OR^{c} or —C(O)NR^{c}R^{d}, wherein R^{c} and R^{d} are each independently hydrogen, alkyl, cycloalkyl, aryl, or arylalkyl.

In various embodiments, the N-terminal group of the peptide is —NR^{a}R^{b}, wherein R^{a} and R^{b} are each independently hydrogen, alkyl, cycloalkyl, acyl, aryl, or arylalkyl; and/or the C-terminal group of the peptide is —C(O)OR or —C(O)NR^{c}R^{d}, wherein R^{c} and R^{d} are each independently hydrogen, alkyl, cycloalkyl, aryl, or arylalkyl.

In certain embodiments, the N-terminal group of the peptide is —NH₂ or —NH(acyl), for example —NHAc; and/or the C-terminal group of the peptide is —C(O)NH₂.

In exemplary embodiments, the N-terminal group of the peptide is —NH₂.

In exemplary embodiments, the C-terminal group of the peptide is —C(O)NR^{c}R^{d}.

In exemplary embodiments, the C-terminal group of the peptide is —C(O)NH₂.

In various embodiments, the peptide conjugate is a lipopeptide.

In some embodiments, the peptide comprises or consists of an amino acid sequence of the formula:

[SEQ ID No. 1]
Z-Xaa⁸Xaa⁹Xaa¹⁰Xaa¹¹Leu¹²Xaa¹³Xaa¹⁴Xaa¹⁵Leu¹⁶Xaa¹⁷
Xaa¹⁸Xaa¹⁹Xaa²⁰Xaa²¹Xaa²²Xaa²³Xaa²⁴Xaa²⁵Xaa²⁶Phe²⁷
Xaa²⁸Xaa²⁹Thr³⁰Xaa³¹Val³²Gly³³Xaa³⁴Xaa³⁵Xaa³⁶Phe³⁷

wherein:
   Z is absent or is Xaa¹Xaa²Xaa³Xaa⁴Xaa⁵Xaa⁶Xaa⁷, Xaa²Xaa³Xaa⁴Xaa⁵Xaa⁶Xaa⁷, Xaa³Xaa⁴Xaa⁵Xaa⁶Xaa⁷, Xaa⁴Xaa⁵Xaa⁶Xaa⁷, Xaa⁵Xaa⁶Xaa⁷, Xaa⁶Xaa⁷ or Xaa⁷
   wherein:
      Xaa¹ is alanine, valine, leucine, isoleucine, proline, phenylalanine, methionine, tryptophan, serine, glycine, asparagine, glutamine, threonine, tyrosine or cysteine;
      Xaa² is cysteine, serine, alanine, glycine, asparagine, glutamine, threonine, tyrosine;
      Xaa³ is aspartate, glutamate, asparagine, glutamine, glycine, serine, threonine, tyrosine or cysteine;
      Xaa⁴ is threonine, glycine, asparagine, glutamine, serine, phenylalanine, tyrosine, valine, isoleucine or cysteine;
      Xaa⁵ is alanine, valine, leucine, isoleucine, proline, phenylalanine, tyrosine methionine or tryptophan;
      Xaa⁶ is threonine, glycine, asparagine, glutamine, serine, tyrosine, phenylalanine, valine, isoleucine or cysteine;
      Xaa⁷ is cysteine, serine, alanine, glycine, asparagine, glutamine, threonine, phenylalanine or tyrosine;
   Xaa⁸ is valine, alanine, leucine, isoleucine, proline, phenylalanine, tyrosine methionine, tryptophan or threonine;
   Xaa⁹ is threonine, glycine, asparagine, glutamine, serine, tyrosine, valine, isoleucine or cysteine;
   Xaa¹⁰ is histidine, lysine, arginine, asparagine, glutamine, serine, alanine, glycine, valine, leucine or isoleucine;
   Xaa¹¹ is arginine, lysine, histidine, glutamine or asparagine;
   Xaa¹³ is alanine, valine, leucine, isoleucine, proline, phenylalanine, methionine, tryptophan, serine, glycine, asparagine, glutamine, threonine, tyrosine or cysteine;
   Xaa¹⁴ is glycine, proline, alanine, asparagine, glutamine, serine, threonine, phenylalanine, tyrosine, cysteine, glutamate or aspartate;
   Xaa¹⁵ is leucine, isoleucine, valine, alanine, methionine, phenylalanine, tyrosine, proline or tryptophan;
   Xaa¹⁷ is serine, threonine, alanine, valine, leucine, isoleucine, proline, phenylalanine, tyrosine, methionine, tryptophan, arginine, lysine, histidine, glutamine, asparagine or cysteine;
   Xaa¹⁸ is arginine, lysine, histidine, glutamine or asparagine;
   Xaa¹⁹ is serine, threonine, alanine, valine, leucine, isoleucine, proline, phenylalanine, tyrosine, methionine, tryptophan or cysteine;
   Xaa²⁰ is glycine, proline, alanine, beta alanine, asparagine, glutamine, serine, threonine, phenylalanine or tyrosine;
   Xaa²¹ is glycine, proline, alanine, beta alanine, asparagine, glutamine, serine, threonine, phenylalanine or tyrosine;
   Xaa²² is valine, alanine, leucine, isoleucine, proline, phenylalanine, tyrosine, methionine or tryptophan or threonine;
   Xaa²³ is valine, alanine, leucine, isoleucine, proline, phenylalanine, tyrosine, methionine, tryptophan or threonine;
   Xaa²⁴ is lysine, arginine, glutamine, asparagine or histidine;
   Xaa²⁵ is asparagine, glutamine, glycine, serine, threonine, tyrosine, phenylalanine, alanine, glutamate, aspartate or cysteine;
   Xaa²⁶ is asparagine, glutamine, glycine, serine, threonine, phenylalanine, tyrosine or cysteine;
   Xaa²⁸ is valine, alanine, leucine, isoleucine, proline, phenylalanine, tyrosine, methionine, tryptophan or threonine;
   Xaa²⁹ is proline, alanine, valine, leucine, isoleucine, glycine, phenylalanine, tyrosine, methionine or tryptophan;
   Xaa³¹ is asparagine, glutamine, glycine, serine, threonine, phenylalanine, tyrosine, glutamate, aspartate or cysteine;
   Xaa³⁴ is serine, threonine, alanine, valine, leucine, isoleucine, proline, phenylalanine, tyrosine, methionine, tryptophan or cysteine;
   Xaa³⁵ is lysine, arginine, glutamine, asparagine, histidine, aspartate or glutamate; and
   Xaa³⁶ is alanine, valine, leucine, isoleucine, proline, phenylalanine, tyrosine, methionine or tryptophan;
wherein one or more of Xaa¹-Xaa¹¹, Xaa¹³-Xaa¹⁵, Xaa¹⁷-Xaa²⁶, Xaa²⁸, Xaa²⁹, Xaa³¹ and Xaa³⁴-Xaa³⁶ is or is substituted with an amino acid that is covalently conjugated to a lipid-containing moiety.

In some embodiments, Z is absent, or is Xaa¹Xaa²Xaa³Xaa³Xaa⁴Xaa⁵Xaa⁶Xaa⁷ or Xaa⁷.

In some embodiments
a) Xaa¹ is alanine, valine, leucine, isoleucine, serine, glycine, or threonine;
b) Xaa² is cysteine, serine or alanine;
c) Xaa³ is aspartate, glutamate, asparagine or glutamine;
d) Xaa⁴ is threonine, glycine, asparagine, glutamine or serine;
e) Xaa⁵ is alanine, valine, leucine or isoleucine;
f) Xaa⁶ is threonine, glycine, asparagine, glutamine or serine;
g) Xaa⁷ is cysteine, serine, or alanine;
h) Xaa⁸ is valine, alanine, leucine, isoleucine, phenylalanine or methionine;
i) Xaa⁹ is threonine, glycine, asparagine, glutamine or serine;
j) Xaa¹⁰ is histidine, lysine or arginine;
k) Xaa¹¹ is arginine, lysine or histidine;
l) Xaa¹³ is alanine, valine, leucine, isoleucine, serine, glycine, or threonine;
m) Xaa¹⁴ is glycine, proline, alanine, aspartate or glutamate;
n) Xaa¹⁵ is leucine, isoleucine, valine, alanine, methionine or phenylalanine;
o) Xaa¹⁷ is serine, threonine, alanine, arginine, lysine or histidine;
p) Xaa¹⁸ is arginine, lysine or histidine;
q) Xaa¹⁹ is serine, threonine or alanine;
r) Xaa²⁰ is glycine, proline or alanine;
s) Xaa²¹ is glycine, proline or alanine;
t) Xaa²² is valine, alanine, leucine, isoleucine, phenylalanine or methionine;
u) Xaa²³ is valine, alanine, leucine, isoleucine, proline, phenylalanine, methionine, tryptophan or threonine;
v) Xaa²⁴ is lysine, arginine or histidine;
w) Xaa²⁵ is asparagine, glutamine, serine, threonine, alanine;
x) Xaa²⁶ is asparagine, serine, glutamate or glutamine;
y) Xaa²⁸ is valine, alanine, leucine, isoleucine, proline, phenylalanine, methionine, tryptophan or threonine;
z) Xaa²⁹ is proline, alanine or glycine;
aa) Xaa³¹ is asparagine, glutamine, glutamate or aspartate;
bb) Xaa³⁴ is serine, threonine or alanine;
cc) Xaa⁵ is lysine, arginine, histidine, aspartate or glutamate;
dd) Xaa³⁶ is alanine, valine, leucine or isoleucine; or
ee) any combination of any two or more of a) to dd);

wherein one or more of Xaa¹-Xaa¹¹, Xaa¹³-Xaa¹⁵, Xaa¹⁷-Xaa²⁶, Xaa²⁸, Xaa²⁹, Xaa³¹ and Xaa³⁴-Xaa³⁶ is or is substituted with an amino acid that is covalently conjugated to a lipid-containing moiety.

In some embodiments
a) Xaa¹ is alanine or serine;
b) Xaa² is cysteine;
c) Xaa³ is aspartate or glutamate;
d) Xaa⁴ is threonine;
e) Xaa⁵ is alanine;
f) Xaa⁶ is threonine;
g) Xaa⁷ is cysteine;
h) Xaa⁸ is valine;
i) Xaa⁹ is threonine;
j) Xaa¹⁰ is histidine;
k) Xaa¹¹ is arginine;
l) Xaa³ is alanine;
m) Xaa¹⁴ is glycine or aspartate;
n) Xaa¹⁵ is leucine;
o) Xaa¹⁷ is serine or arginine;
p) Xaa¹⁸ is arginine;
q) Xaa¹⁹ is serine;
r) Xaa²⁰ is glycine;
s) Xaa²¹ is glycine;
t) Xaa²² is valine or methionine;
u) Xaa²³ is valine or leucine;
v) Xaa²⁴ is lysine;
w) Xaa²⁵ is asparagine or serine;
x) Xaa²⁶ is asparagine, serine or glutamate;
y) Xaa²⁸ is valine;
z) Xaa²⁹ is proline;
aa) Xaa³¹ is asparagine or aspartate;
bb) Xaa³⁴ is serine;
cc) Xaa³⁵ is lysine or glutamate;
dd) Xaa³⁶ is alanine; or ee) any combination of any two or more of a) to dd);

wherein one or more of Xaa¹-Xaa¹¹, Xaa¹³-Xaa¹⁵, Xaa¹⁷-Xaa²⁶, Xaa²⁸, Xaa²⁹, Xaa³¹ and Xaa³⁴-Xaa³⁶ is or is substituted with an amino acid that is covalently conjugated to a lipid-containing moiety.

In some embodiments, the peptide comprises or consists of an amino acid sequence of the formula:

[SEQ ID No. 2]
Z-Xaa⁸Thr⁹Xaa¹⁰Xaa¹¹Leu¹²Ala¹³Xaa¹⁴Leu¹⁵Leu¹⁶Xaa¹⁷
Xaa¹⁸Xaa¹⁹Gly²⁰Xaa²¹Xaa²²Xaa²³Xaa²⁴Xaa²⁵Asn²⁶Phe²⁷
Val²⁸Pro²⁹Thr³⁰Xaa³¹Val³²Gly³³Ser³⁴Xaa³⁵Ala³⁶Phe³⁷

wherein:
   Z is absent or is Xaa¹Xaa²Xaa³Thr⁴Ala⁵Xaa⁶Xaa⁷, Xaa²Xaa³Thr⁴Ala⁵Xaa⁶Xaa⁷, Xaa³Thr⁴Ala⁵Xaa⁶Xaa⁷, Thr⁴Ala⁵Xaa⁶Xaa⁷, Ala⁵Xaa⁶Xaa⁷, Xaa⁶Xaa⁷ or Xaa⁷
   wherein:
   a) Xaa¹ is alanine or serine;
   b) Xaa² is cysteine or homocysteine;
   c) Xaa³ is aspartate or asparagine;
   d) Xaa⁶ is threonine, cysteine or homocysteine;
   e) Xaa⁷ is cysteine or homocysteine;
   f) Xaa⁸ is valine, cysteine or homocysteine;
   g) Xaa¹⁰ is histidine, cysteine or homocysteine,
   h) Xaa¹¹ is arginine, cysteine or homocysteine;
   i) Xaa¹⁴ is glycine or aspartate;
   j) Xaa¹⁷ is serine, arginine, cysteine or homocysteine,
   k) Xaa¹⁸ is arginine, cysteine or homocysteine;
   l) Xaa¹⁹ is a serine, cysteine or homocysteine;
   m) Xaa²¹ is glycine, cysteine or homocysteine;
   n) Xaa²² is valine or methionine;
   o) Xaa²³ is valine or leucine;
   p) Xaa²⁴ is lysine, cysteine or homocysteine;
   q) Xaa²⁵ is asparagine, serine or aspartate;
   r) Xaa³¹ is asparagine or aspartate; and
   s) Xaa³⁵ is lysine, glutamate, cysteine or homocysteine;

wherein at least one cysteine or homocysteine in the peptide is covalently conjugated to a lipid-containing moiety.

In some embodiments, one or more of Xaa6-Xaa8, Xaa10, Xaa11, Xaa17-Xaa19, Xaa21, Xaa24 and Xaa35 is or is substituted with an amino acid that is covalently conjugated to a lipid-containing moiety.

In some embodiments, one or more of Xaa7, Xaa8, Xaa11, Xaa24 and Xaa35 is or is substituted with an amino acid that is covalently conjugated to a lipid-containing moiety.

In some embodiments, one or more of Xaa7, Xaa8, Xaa24 and Xaa35 is or is substituted with an amino acid that is covalently conjugated to a lipid-containing moiety.

In some embodiments, 1 or 2 of Xaa6-Xaa8, Xaa10, Xaa11, Xaa17-Xaa19, Xaa21, Xaa24 and Xaa35 is or is substituted with an amino acid that is covalently conjugated to a lipid-containing moiety.

In some embodiments, 1 or 2 of Xaa7, Xaa8, Xaa11, Xaa24 and Xaa35 is or is substituted with an amino acid that is covalently conjugated to a lipid-containing moiety.

In some embodiments, two or more of Xaa6-Xaa8, Xaa10, Xaa11, Xaa17-Xaa19, Xaa21, Xaa24 and Xaa35 is or is substituted with an amino acid that is covalently conjugated to a lipid-containing moiety.

In some embodiments two or more of Xaa7, Xaa8, Xaa11, Xaa24 and Xaa35 is or is substituted with an amino acid that is covalently conjugated to a lipid-containing moiety.

In some embodiments, the peptide comprises or consists of
a) the amino acid sequence of SEQ ID NO:3;
b) 25 or more contiguous amino acids of SEQ ID NO:3;
c) amino acids 7-37 of SEQ ID No:3;
d) amino acids 8-37 of SEQ ID NO:3;
e) the amino acid sequence of SEQ ID NO:4;
f) 25 or more contiguous amino acids of SEQ ID NO:4;
g) amino acids 7-37 of SEQ ID No:4;
h) amino acids 8-37 of SEQ ID NO:4; or
i) a functional variant of any one of a) to h) comprising or consisting of an amino acid sequence having at least about 60% amino acid sequence identity to the sequence defined in any one of a) to h);
wherein one or more amino acids in the sequence is or is substituted with an amino acid covalently conjugated to a lipid-containing moiety.

In some embodiments, the amino acid sequence has at least about 90% sequence identity to the sequence defined in a)-h) of the embodiment above.

In some embodiments, the peptide comprises or consists of an amino acid sequence selected from
a) amino acids 2-37 of SEQ ID No:3 or SEQ ID No:4;
b) amino acids 3-37 of SEQ ID No:3 or SEQ ID No:4;
c) amino acids 4-37 of SEQ ID No:3 or SEQ ID No:4;
d) amino acids 5-37 of SEQ ID No:3 or SEQ ID No:4;
e) amino acids 6-37 of SEQ ID No:3 or SEQ ID No:4; or
f) a functional variant of any one of a) to e) comprising or consisting of an amino acid sequence having at least about 60% amino acid sequence identity to the sequence defined in any one of a) to e);
wherein one or more amino acids in the sequence is or is substituted with an amino acid covalently conjugated to a lipid-containing moiety.

In some embodiments, the amino acid sequence has at least about 90% sequence identity to the sequence defined in a)-e) of the embodiment above.

In some embodiments the peptide comprises or consists of a functional variant of any CGRP peptide amino acid sequence of the embodiments above wherein the amino acid sequence of the functional variant has at least about 60%, 65%, 70%, 75%, 80%, 85%, 90%, 95% or at least about 99% sequence identity to the CGRP peptide amino sequence of the embodiments above.

In various embodiments the peptide comprises or consists of
a) the amino acid sequence of SEQ ID NO:3;
b) the amino acid sequence of SEQ ID NO:95;
c) the amino acid sequence of SEQ ID NO:96;
d) the amino acid sequence of SEQ ID NO:4;
e) the amino acid sequence of SEQ ID NO:97;
f) the amino acid sequence of SEQ ID NO:31;
wherein at least one cysteine in the sequence is covalently conjugated to a lipid-containing moiety.

In some embodiments, the peptide comprises an amino acid covalently conjugated to a lipid-containing moiety at one or more amino acid positions corresponding to positions 1-11, 13-15, 17-26, 28, 29, 31 and 34-36 of SEQ ID No 3 or SEQ ID No:4.

In some embodiments, the peptide comprises an amino acid covalently conjugated to a lipid-containing moiety at one or more amino acid positions corresponding to positions 6-8, 10, 11, 17-19, 21, 24 and 35 of SEQ ID No 3 or SEQ ID NO:4.

In some embodiments, the peptide comprises an amino acid covalently conjugated to a lipid-containing moiety at one or more amino acid positions corresponding to positions 6-8, 10, 11, 21, 24 and 35 of SEQ ID No 3 or SEQ ID NO: 4.

In some embodiments, the peptide comprises an amino acid covalently conjugated to a lipid-containing moiety at 1, 2, 3, 4 or 5 amino acid positions corresponding to positions 6-8, 10, 11, 21, 24 and 35 of SEQ ID No 3 or SEQ ID NO: 4.

In some embodiments, the peptide comprises an amino acid covalently conjugated to a lipid-containing moiety at one or more amino acid positions corresponding to positions 7, 8, 24 and 35 of SEQ ID No 3 or SEQ ID NO: 4.

In some embodiments, the peptide comprises an amino acid covalently conjugated to a lipid-containing moiety at one or more amino acid positions corresponding to positions 7, 8, 11, 24 and 35 of SEQ ID No 3 or SEQ ID NO: 4.

In some embodiments, the peptide comprises an amino acid covalently conjugated to a lipid-containing moiety at 1, 2, 3, or 4 amino acid positions corresponding to positions 7, 8, 24 and 35 of SEQ ID No 3 or SEQ ID NO: 4.

In some embodiments, the peptide comprises an amino acid covalently conjugated to a lipid-containing moiety at one or more amino acid positions corresponding to positions 7, 8, 11, 24 and 35 of SEQ ID No 3 or SEQ ID NO: 4.

In some embodiments, the N-terminal amino acid of the peptide is covalently conjugated to a lipid-containing moiety.

In some embodiments, the peptide comprises one or more amino acids covalently conjugated to a lipid-containing moiety in
a) a region of the peptide comprising amino acids Xaa1-Xaa7 or a region of the peptide corresponding to amino acids 1-7 of SEQ ID No:3 or SEQ ID No:4;
b) a region of the peptide comprising amino acids Xaa8-Xaa18 or a region of the peptide corresponding to amino acids 8-18 of SEQ ID No:3 or SEQ ID No:4;
c) a region of the peptide comprising amino acids Xaa19-Xaa26 or a region of the peptide corresponding to amino acids 19-26 of SEQ ID No:3 or SEQ ID No:4;
d) a region of the peptide comprising Xaa27-Xaa37 or a region of the peptide corresponding to amino acids 27-37 of SEQ ID No:3 or SEQ ID No:4; or
e) any combination of any two or more of a) to d).

In some embodiments, the peptide comprises from about 1 to about 5 amino acids covalently conjugated to a lipid-containing moiety.

In some embodiments, the peptide comprises from about 1 to about 3 amino acids covalently conjugated to a lipid-containing moiety.

In some embodiments, the peptide comprises 1 or 2 amino acids covalently conjugated to a lipid-containing moiety.

In some embodiments, the amino acid covalently conjugated to a lipid-containing moiety is cysteine or homocysteine.

In some embodiments, the cysteine or homocysteine is covalently conjugated to the lipid-containing moiety via a sulfur atom of a sulfide group of the cysteine or homocysteine.

In some embodiments, the amino acid covalently conjugated to a lipid-containing moiety is cysteine or homocysteine, and the the lipid-containing moiety is covalently attached via the sulfur atom of the sulfide group of the cysteine or homocysteine.

In some embodiments, the peptide comprises a C-terminal amide (that is, the C-terminal amino acid is amidated). In some embodiments, the peptide comprises an N-terminal acyl group, for example an acetyl group (that is, the N-terminal amino acid is acetylated).

In some embodiments, the peptide comprises or consists of an amino acid sequence selected from

a)
[SEQ ID No: 5]
AXDTATXVTHRLAGLLSRSGGVVKNNFVPTNVGSKAF;
b)
[SEQ ID No: 6]
XXDTATXVTHRLAGLLSRSGGVVKNNFVPTNVGSKAF;
c)
[SEQ ID No: 7]
AXXTATXVTHRLAGLLSRSGGVVKNNFVPTNVGSKAF;
d)
[SEQ ID No: 8]
AXDXATXVTHRLAGLLSRSGGVVKNNFVPTNVGSKAF;
e)
[SEQ ID No: 9]
AXDTXTXVTHRLAGLLSRSGGVVKNNFVPTNVGSKAF;
f)
[SEQ ID No: 10]
AXDTAXXVTHRLAGLLSRSGGVVKNNFVPTNVGSKAF;
g)
[SEQ ID No: 11]
XDTATXVTHRLAGLLSRSGGVVKNNFVPTNVGSKAF;
h)
[SEQ ID No: 12]
DTATXVTHRLAGLLSRSGGVVKNNFVPTNVGSKAF;
i)
[SEQ ID No: 13]
XTATXVTHRLAGLLSRSGGVVKNNFVPTNVGSKAF;
j)
[SEQ ID No: 14]
TATXVTHRLAGLLSRSGGVVKNNFVPTNVGSKAF;
k)
[SEQ ID No: 15]
ATXVTHRLAGLLSRSGGVVKNNFVPTNVGSKAF;
l)
[SEQ ID No: 16]
TXVTHRLAGLLSRSGGVVKNNFVPTNVGSKAF;
m)
[SEQ ID No: 17]
XVTHRLAGLLSRSGGVVKNNFVPTNVGSKAF;
n)
[SEQ ID No: 18]
XTHRLAGLLSRSGGVVKNNFVPTNVGSKAF;
o)
[SEQ ID No: 19]
VXHRLAGLLSRSGGVVKNNFVPTNVGSKAF;
p)
[SEQ ID No: 20]
VTXRLAGLLSRSGGVVKNNFVPTNVGSKAF;
q)
[SEQ ID No: 21]
VTHXLAGLLSRSGGVVKNNFVPTNVGSKAF;
r)
[SEQ ID No: 22]
VTHRLXGLLSRSGGVVKNNFVPTNVGSKAF;
s)
[SEQ ID No: 23]
VTHRLAXLLSRSGGVVKNNFVPTNVGSKAF;
t)
[SEQ ID No: 24]
VTHRLAGXLSRSGGVVKNNFVPTNVGSKAF;
u)
[SEQ ID No: 25]
VTHRLAGLLXRSGGVVKNNFVPTNVGSKAF;
v)
[SEQ ID No: 26]
VTHRLAGLLSXSGGVVKNNFVPTNVGSKAF;
w)
[SEQ ID No: 27]
VTHRLAGLLSRXGGVVKNNFVPTNVGSKAF;
x)
[SEQ ID No: 28]
VTHRLAGLLSRSXGVVKNNFVPTNVGSKAF;
y)
[SEQ ID No: 29]
VTHRLAGLLSRSGXVVKNNFVPTNVGSKAF;
z)
[SEQ ID No: 30]
VTHRLAGLLSRSGGXVKNNFVPTNVGSKAF;
aa)
[SEQ ID No: 32]
VTHRLAGLLSRSGGVXKNNFVPTNVGSKAF;
bb)
[SEQ ID No: 33]
VTHRLAGLLSRSGGVVXNNFVPTNVGSKAF;
cc)
[SEQ ID No: 34]
VTHRLAGLLSRSGGVVIONFVPTNVGSKAF;
dd)
[SEQ ID No: 35]
VTHRLAGLLSRSGGVVKNXFVPTNVGSKAF;
ee)
[SEQ ID No: 36]
VTHRLAGLLSRSGGVVKNNFXPTNVGSKAF;
ff)
[SEQ ID No: 37]
VTHRLAGLLSRSGGVVKNNFVXTNVGSKAF;
gg)
[SEQ ID No: 38]
VTHRLAGLLSRSGGVVKNNFVPTXVGSKAF;
hh)
[SEQ ID No: 39]
VTHRLAGLLSRSGGVVKNNFVPTNVGXKAF;
ii)
[SEQ ID No: 40
VTHRLAGLLSRSGGVVKNNFVPTNVGSXAF;
jj)
[SEQ ID No: 41]
VTHRLAGLLSRSGGVVKNNFVPTNVGSKXF;
kk)
[SEQ ID No: 42]
AXNTATXVTHRLAGLLSRSGGMVKSNFVPTNVGSKAF,
ll)
[SEQ ID No: 43]
XXNTATXVTHRLAGLLSRSGGMVKSNFVPTNVGSKAF;
mm)
[SEQ ID No: 44]
AXXTATXVTHRLAGLLSRSGGMVKSNFVPTNVGSKAF;
nn)
[SEQ ID No: 45]
AXNXATXVTHRLAGLLSRSGGMVKSNFVPTNVGSKAF;
oo)
[SEQ ID No: 46]
AXNTXTXVTHRLAGLLSRSGGMVKSNFVPTNVGSKAF;
pp)
[SEQ ID No: 47]
AXNTAXXVTHRLAGLLSRSGGMVKSNFVPTNVGSKAF;
qq)
[SEQ ID No: 48]
XNTATXVTHRLAGLLSRSGGMVKSNFVPTNVGSKAF;
rr)
[SEQ ID No: 49]
NTATXVTHRLAGLLSRSGGMVKSNFVPTNVGSKAF;
ss)
[SEQ ID No: 50]
AXNXTATXVTHRLAGLLSRSGGMVKSNFVPTNVGSKAF;
tt)
[SEQ ID No: 51]
XTATXVTHRLAGLLSRSGGMVKSNFVPTNVGSKAF;
uu)
[SEQ ID No: 52]
TATXVTHRLAGLLSRSGGMVKSNFVPTNVGSKAF;
vv)
[SEQ ID No: 53]
ATXVTHRLAGLLSRSGGMVKSNFVPTNVGSKAF;
ww)
[SEQ ID No: 54]
TXVTHRLAGLLSRSGGMVKSNFVPTNVGSKAF;
xx)
[SEQ ID No: 55]
XVTHRLAGLLSRSGGMVKSNFVPTNVGSKAF;
yy)
[SEQ ID No: 56]
XTHRLAGLLSRSGGMVKSNFVPTNVGSKAF;
zz)
[SEQ ID No: 57]
VXHRLAGLLSRSGGMVKSNFVPTNVGSKAF;
aaa)
[SEQ ID No: 58]
VTXRLAGLLSRSGGMVKSNFVPTNVGSKAF;
bbb)
[SEQ ID No: 59]
VTHXLAGLLSRSGGMVKSNFVPTNVGSKAF;
ccc)
[SEQ ID No: 60]
VTHRLXGLLSRSGGMVKSNFVPTNVGSKAF;
ddd)
[SEQ ID No: 61]
VTHRLAXLLSRSGGMVKSNFVPTNVGSKAF;
eee)
[SEQ ID No: 62]
VTHRLAGXLSRSGGMVKSNFVPTNVGSKAF;
fff)
[SEQ ID No: 63]
VTHRLAGLLXRSGGMVKSNFVPTNVGSKAF;
ggg)
[SEQ ID No: 64]
VTHRLAGLLSXSGGMVKSNFVPTNVGSKAF;
hhh)
[SEQ ID No: 65]
VTHRLAGLLSRXGGMVKSNFVPTNVGSKAF;
iii)
[SEQ ID No: 66]
VTHRLAGLLSRSXGMVKSNFVPTNVGSKAF;
jjj)
[SEQ ID No: 67]
VTHRLAGLLSRSGXMVKSNFVPTNVGSKAF;
kkk)
[SEQ ID No: 68]
VTHRLAGLLSRSGGXVKSNFVPTNVGSKAF;
lll)
[SEQ ID No: 69]
VTHRLAGLLSRSGGMXKSNFVPTNVGSKAF;
mmm)
[SEQ ID No: 70]
VTHRLAGLLSRSGGMVXSNFVPTNVGSKAF;
nnn)
[SEQ ID No: 71]
VTHRLAGLLSRSGGMVIONFVPTNVGSKAF;
ooo)
[SEQ ID No: 72]
VTHRLAGLLSRSGGMVKSXFVPTNVGSKAF;
ppp)
[SEQ ID No: 73]
VTHRLAGLLSRSGGMVKSNFXPTNVGSKAF;
qqq)
[SEQ ID No: 74]
VTHRLAGLLSRSGGMVKSNFVXTNVGSKAF;
rrr)
[SEQ ID No: 75]
VTHRLAGLLSRSGGMVKSNFVPTXVGSKAF;
sss)
[SEQ ID No: 76]
VTHRLAGLLSRSGGMVKSNFVPTNVGXKAF;
ttt)
[SEQ ID No: 77]
VTHRLAGLLSRSGGMVKSNFVPTNVGSXAF;
or
uuu)
[SEQ ID No: 78]
VTHRLAGLLSRSGGMVKSNFVPTNVGSKXF;

wherein X is cysteine or homocysteine and wherein at least one X in the peptide is covalently conjugated to a lipid-containing moiety.

In some embodiments, the peptide comprises or consists of an amino acid sequence selected from

a)
[SEQ ID No: 17]
XVTHRLAGLLSRSGGVVKNNFVPTNVGSKAF;
b)
[SEQ ID No: 18]
XTHRLAGLLSRSGGVVKNNFVPTNVGSKAF;
c)
[SEQ ID No: 33]
VTHRLAGLLSRSGGVVXNNFVPTNVGSKAF;
d)
[SEQ ID No: 40]
VTHRLAGLLSRSGGVVKNNFVPTNVGSXAF;
e)
[SEQ ID No: 10]
AXDTAXXVTHRLAGLLSRSGGVVKNNFVPTNVGSKAF;
f)
[SEQ ID No: 20]
VTXRLAGLLSRSGGVVKNNFVPTNVGSKAF;
g)
[SEQ ID No: 21]
VTHXLAGLLSRSGGVVKNNFVPTNVGSKAF;
h)
[SEQ ID No: 29]
VTHRLAGLLSRSGXVVKNNFVPTNVGSKAF;
i)
[SEQ ID No: 55]
XVTHRLAGLLSRSGGMVKSNFVPTNVGSKAF;
j)
[SEQ ID No: 56]
XTHRLAGLLSRSGGMVKSNFVPTNVGSKAF;
k)
[SEQ ID No: 70]
VTHRLAGLLSRSGGMVXSNFVPTNVGSKAF;
l)
[SEQ ID No: 77]
VTHRLAGLLSRSGGMVKSNFVPTNVGSXAF;
m)
[SEQ ID No: 47]
AXNTAXXVTHRLAGLLSRSGGMVKSNFVPTNVGSKAF;
n)
[SEQ ID No: 58]
VTXRLAGLLSRSGGMVKSNFVPTNVGSKAF;
o)
[SEQ ID No: 59]
VTHXLAGLLSRSGGMVKSNFVPTNVGSKAF;
or
p)
[SEQ ID No: 67]
VTHRLAGLLSRSGXMVKSNFVPTNVGSKAF;

wherein X is cysteine or homocysteine,

and wherein at least one X in the peptide is covalently conjugated to a lipid-containing moiety.

In some embodiments, wherein the peptide comprises or consists of an amino acid sequence selected from

a)
[SEQ ID No: 79]
CVTHRLAGLLSRSGGVVKNNFVPTNVGSKAF;
b)
[SEQ ID No: 80]
CTHRLAGLLSRSGGVVKNNFVPTNVGSKAF;
c)
[SEQ ID No: 81]
VTHRLAGLLSRSGGVVCNNFVPTNVGSKAF;
d)
[SEQ ID No: 82]
VTHRLAGLLSRSGGVVKNNFVPTNVGSCAF;
e)
[SEQ ID No: 83]
ACDTACCVTHRLAGLLSRSGGVVKNNFVPTNVGSKAF;
f)
[SEQ ID No: 84]
VTCRLAGLLSRSGGVVKNNFVPTNVGSKAF;
g)
[SEQ ID No: 85]
VTHCLAGLLSRSGGVVKNNFVPTNVGSKAF;
h)
[SEQ ID No: 86]
VTHRLAGLLSRSGCVVKNNFVPTNVGSKAF;
i)
[SEQ ID No: 87]
CVTHRLAGLLSRSGGMVKSNFVPTNVGSKAF;
j)
[SEQ ID No: 88]
CTHRLAGLLSRSGGMVKSNFVPTNVGSKAF;
k)
[SEQ ID No: 89]
VTHRLAGLLSRSGGMVCSNFVPTNVGSKAF;
l)
[SEQ ID No: 90]
VTHRLAGLLSRSGGMVKSNFVPTNVGSCAF;
m)
[SEQ ID No: 91]
ACNTACCVTHRLAGLLSRSGGMVKSNFVPTNVGSKAF;
n)
[SEQ ID No: 92]
VTCRLAGLLSRSGGMVKSNFVPTNVGSKAF;
o)
[SEQ ID No: 93]
VTHCLAGLLSRSGGMVKSNFVPTNVGSKAF;
or
p)
[SEQ ID No: 94]
VTHRLAGLLSRSGCMVKSNFVPTNVGSKAF;

wherein at least one C in the peptide is covalently conjugated to a lipid-containing moiety.

In some embodiments, the peptide comprises or consists of an amino acid sequence selected from

a)
[SEQ ID No: 100]
XXTHRLAGLLSRSGGVVKNNFVPTNVGSKAF;
b)
[SEQ ID No: 101]
XVTHXLAGLLSRSGGVVKNNFVPTNVGSKAF;
c)
[SEQ ID No: 102]
XVTHRLAGLLSRSGGVVXNNFVPTNVGSKAF;
d)
[SEQ ID No: 103]
XVTHRLAGLLSRSGGVVKNNFVPTNVGSXAF;
e)
[SEQ ID No: 104]
XTHXLAGLLSRSGGVVKNNFVPTNVGSKAF;
f)
[SEQ ID No: 105]
XTHRLAGLLSRSGGVVXNNFVPTNVGSKAF;
g)
[SEQ ID No: 106]
XTHRLAGLLSRSGGVVKNNFVPTNVGSXAF;
h)
[SEQ ID No: 107]
VTHXLAGLLSRSGGVVXNNFVPTNVGSKAF;
i)
[SEQ ID No: 108]
VTHXLAGLLSRSGGVVKNNFVPTNVGSXAF;
j)
[SEQ ID No: 109]
VTHRLAGLLSRSGGVVXNNFVPTNVGSXAF;
k)
[SEQ ID No: 110]
XXTHRLAGLLSRSGGMVKSNFVPTNVGSKAF;
l)
[SEQ ID No: 111]
XVTHXLAGLLSRSGGMVKSNFVPTNVGSKAF;
m)
[SEQ ID No: 112]
XVTHRLAGLLSRSGGMVXSNFVPTNVGSKAF;
n)
[SEQ ID No: 113]
XVTHRLAGLLSRSGGMVKSNFVPTNVGSXAF;
o)
[SEQ ID No: 114]
XTHXLAGLLSRSGGMVKSNFVPTNVGSKAF;
p)
[SEQ ID No: 115]
XTHRLAGLLSRSGGMVXSNFVPTNVGSKAF;
q)
[SEQ ID No: 116]
XTHRLAGLLSRSGGMVKSNFVPTNVGSXAF;
r)
[SEQ ID No: 117]
VTHXLAGLLSRSGGMVXSNFVPTNVGSKAF;
s)
[SEQ ID No: 118]
VTHXLAGLLSRSGGMVKSNFVPTNVGSXAF;
or
t)
[SEQ ID No: 119]
VTHRLAGLLSRSGGMVXSNFVPTNVGSXAF;

wherein X is cysteine or homocysteine,
and wherein at least two X in the peptide are covalently conjugated to a lipid-containing moiety.

In some embodiments, the peptide comprises or consists of an amino acid sequence selected from

a)
[SEQ ID No: 120]
CCTHRLAGLLSRSGGVVKNNFVPTNVGSKAF;
b)
[SEQ ID No: 121]
CVTHCLAGLLSRSGGVVKNNFVPTNVGSKAF;
c)
[SEQ ID No: 122]
CVTHRLAGLLSRSGGVVCNNFVPTNVGSKAF;
d)
[SEQ ID No: 123]
CVTHRLAGLLSRSGGVVKNNFVPTNVGSCAF;
e)
[SEQ ID No: 124]
CTHCLAGLLSRSGGVVKNNFVPTNVGSKAF;
f)
[SEQ ID No: 125]
CTHRLAGLLSRSGGVVCNNFVPTNVGSKAF;
g)
[SEQ ID No: 126]
CTHRLAGLLSRSGGVVKNNFVPTNVGSCAF;
h)
[SEQ ID No: 99]
VTHCLAGLLSRSGGVVCNNFVPTNVGSKAF;
i)
[SEQ ID No: 127]
VTHCLAGLLSRSGGVVKNNFVPTNVGSCAF;
j)
[SEQ ID No: 128]
VTHRLAGLLSRSGGVVCNNFVPTNVGSCAF;
k)
[SEQ ID No: 129]
CCTHRLAGLLSRSGGMVKSNFVPTNVGSKAF;
l)
[SEQ ID No: 130]
CVTHCLAGLLSRSGGMVKSNFVPTNVGSKAF;
m)
[SEQ ID No: 131]
CVTHRLAGLLSRSGGMVCSNFVPTNVGSKAF;
n)
[SEQ ID No: 132]
CVTHRLAGLLSRSGGMVKSNFVPTNVGSCAF;
o)
[SEQ ID No: 133]
CTHCLAGLLSRSGGMVKSNFVPTNVGSKAF;
p)
[SEQ ID No: 134]
CTHRLAGLLSRSGGMVCSNFVPTNVGSKAF;
q)
[SEQ ID No: 135]
CTHRLAGLLSRSGGMVKSNFVPTNVGSCAF;
r)
[SEQ ID No: 136]
VTHCLAGLLSRSGGMVCSNFVPTNVGSKAF;
s)
[SEQ ID No: 137]
VTHCLAGLLSRSGGMVKSNFVPTNVGSCAF;
or
t)
[SEQ ID No: 138]
VTHRLAGLLSRSGGMVCSNFVPTNVGSCAF;

wherein at least two C in the peptide are covalently conjugated to a lipid-containing moiety.

In another aspect, the present invention broadly consists in a pharmaceutical composition comprising a peptide conjugate of the present invention; and a pharmaceutically acceptable carrier.

In another aspect, the present invention broadly consists in a kit comprising a peptide/conjugate of the present invention; and instructions for use.

In another aspect, the present invention broadly consists in a method of antagonising a CGRP receptor in a subject in need thereof, comprising administering to the subject an effective amount of a peptide conjugate of the invention.

In another aspect, the present invention broadly consists in a method of treating a disease or condition mediated by or modulated by a CGRP receptor or characterised by excessive CGRP receptor activation in a subject in need thereof, comprising administering to the subject a therapeutically effective amount of a peptide conjugate of the invention.

In another aspect, the present invention relates to a method of treating a disease or condition associated with or characterised by increased vasodilation in a subject in need thereof, comprising administering to the subject a therapeutically effective amount of a peptide conjugate according to the present invention.

In another aspect, the present invention broadly consists in a method of treating a disease or condition selected from the group consisting of thermal injury, circulatory shock, menopausal hot flushes, asthma, sepsis, neurogenic inflammation, inflammatory skin conditions (for example psoriasis and contact dermatitis), allergic rhinitis, joint disorders (for example arthritis and temporomandibular joint disorder, preferably arthritis), cachexia (for example cancer-induced cachexia), pain, for example craniofacial pain disorders (for example migraine, headache, trigeminal neuralgia and dental pain, preferably migraine), and metabolic disorders or syndromes (for example obesity, type II diabetes, insulin resistance, dyslipidemia, hypertension, atherosclerosis and thrombosis) in a subject in need thereof, comprising administering to the subject a therapeutically effective amount of a peptide conjugate of the invention.

In another aspect, the present invention broadly consists in a peptide conjugate of the invention for use in antagonising a CGRP receptor.

In another aspect, the present invention broadly consists in a peptide conjugate of the invention for use in treating a disease or condition mediated by or modulated by a CGRP receptor or characterised by excessive CGRP receptor activation.

In another aspect, the present invention relates to a peptide conjugate of the invention for use in treating a disease or condition associated with or characterised by increased vasodilation.

In another aspect, the present invention broadly consists in a peptide conjugate of the invention for use in treating a disease or condition selected from the group consisting of thermal injury, circulatory shock, menopausal hot flushes, asthma, sepsis, neurogenic inflammation, inflammatory skin conditions (for example psoriasis and contact dermatitis), allergic rhinitis, joint disorders (for example arthritis and temporomandibular joint disorder, preferably arthritis), cachexia (for example cancer-induced cachexia), pain, for example craniofacial pain disorders (for example migraine, headache, trigeminal neuralgia and dental pain, preferably migraine), and metabolic disorders or syndromes (for example obesity, type II diabetes, insulin resistance, dyslipidemia, hypertension, atherosclerosis and thrombosis).

In another aspect, the present invention broadly consists in use of a peptide conjugate of the invention in the manufacture of a medicament for antagonising a CGRP receptor.

In another aspect, the present invention broadly consists in use of a peptide conjugate of the invention in the manufacture of a medicament for treating a disease or condition mediated by or modulated by a CGRP receptor or characterised by excessive CGRP receptor activation.

In another aspect, the present invention relates to use of a peptide conjugate of the present invention in the manufacture of a medicament for treating a disease or condition associated with or characterised by increased vasodilation.

In another aspect, the present invention broadly consists in use of a peptide conjugate of the invention in the manufacture of a medicament for treating a disease or condition selected from the group consisting of thermal injury, circulatory shock, menopausal hot flushes, asthma, sepsis, neurogenic inflammation, inflammatory skin conditions (for example psoriasis and contact dermatitis), allergic rhinitis, joint disorders (for example arthritis and temporomandibular joint disorder, preferably arthritis), cachexia (for example cancer-induced cachexia), pain, for example craniofacial pain disorders (for example migraine, headache, trigeminal neuralgia and dental pain, preferably migraine), and metabolic disorders or syndromes (for example obesity, type II diabetes, insulin resistance, dyslipidemia, hypertension, atherosclerosis and thrombosis).

In another aspect, the present invention broadly consists in a method of antagonising a CGRP receptor comprising contacting a cell and a peptide conjugate of the invention in an amount effective to antagonise the CGRP receptor.

In various embodiments, antagonising the CGRP receptor comprises treating a disease or condition mediated by or modulated by the CGRP receptor or characterised by excessive CGRP receptor activation.

In various embodiments, antagonising the CGRP receptor comprises contacting a cell and a peptide conjugate of the invention in an amount effective to antagonise the CGRP receptor.

In various embodiments, the disease or condition is selected from the group consisting of thermal injury, circulatory shock, menopausal hot flushes, asthma, sepsis, neurogenic inflammation, inflammatory skin conditions (for example psoriasis and contact dermatitis), allergic rhinitis, joint disorders (for example arthritis and temporomandibular joint disorder, preferably arthritis), cachexia (for example cancer-induced cachexia), pain, for example craniofacial pain disorders (for example migraine, headache, trigeminal neuralgia and dental pain, preferably migraine), and metabolic disorders or syndromes (for example obesity, type II diabetes, insulin resistance, dyslipidemia, hypertension, atherosclerosis and thrombosis).

In various embodiments, the disease or condition is selected from the group consisting of thermal injury, circulatory shock, menopausal hot flushes, asthma, sepsis, neurogenic inflammation, inflammatory skin conditions (for example psoriasis and contact dermatitis), allergic rhinitis, joint disorders (for example arthritis and temporomandibular joint disorder, preferably arthritis), pain, for example craniofacial pain disorders (for example migraine, headache, trigeminal neuralgia and dental pain, preferably migraine), and metabolic disorders or syndromes (for example obesity, type II diabetes, insulin resistance, dyslipidemia, hypertension, atherosclerosis and thrombosis) In various embodiments the disease or condition is selected from pain or metabolic disorders.

In various embodiments the disease or condition is pain.

In various embodiments the disease or condition is migraine or headache (for example cluster headaches and post-traumatic headache).

In various embodiments the disease or condition is migraine.

In various embodiments, the inflammatory skin condition is rosacea, psoriasis, and contact dermatitis. In various embodiments, the inflammatory skin condition is rosacea.

In another aspect, the present invention broadly consists in a method for preparing a peptide conjugate of the invention, the method comprising
(A) providing an amino acid conjugate comprising an amino acid of a calcitonin gene-related peptide (CGRP) peptide, wherein the amino acid is covalently conjugated to a lipid-containing moiety via a sulfur atom of a sulfide group; and
coupling the amino acid of the amino acid conjugate to one or more amino acids and/or one or more peptides to provide the peptide conjugate of the invention; or
(B) providing a peptide-conjugate comprising a peptide fragment of a calcitonin gene-related peptide (CGRP) peptide, wherein at least one amino acid of the peptide fragment is covalently conjugated to a lipid-containing moiety via a sulfur atom of a sulfide group; and
coupling an amino acid of the peptide conjugate to one or more amino acids and/or one or more peptides to provide the peptide conjugate of the invention.

In various embodiments, the amino acid conjugate or the peptide conjugate comprising the peptide fragment is bound to a solid phase support; or the amino acid conjugate or the peptide conjugate is coupled to an amino acid or peptide bound to a solid phase.

In various embodiments, the amino acid conjugate or the peptide conjugate comprising the peptide fragment is bound to a solid phase support.

In another aspect, the present invention broadly consists in a method for preparing a peptide conjugate of the invention, the method comprising reacting
a lipid-containing conjugation partner comprising a carbon carbon double bond, and
an amino acid-comprising conjugation partner comprising at least one amino acid comprising a thiol
under conditions effective to conjugate the lipid-containing conjugation partner to the amino acid-comprising conjugation partner.

In various embodiments, the conditions are effective to conjugate the lipid-containing conjugation partner to the amino acid-comprising conjugation partner by the hydrothiolation of the carbon-carbon double bond with the thiol.

In some embodiments, the at least one amino acid comprising the thiol is cysteine or homocysteine. In exemplary embodiments, the at least one amino acid comprising the thiol is cysteine.

In one embodiment, the lipid-containing conjugation partner comprises one or more straight or branched aliphatic or heteroaliphatic chains each containing at least 4 or at least 6 chain-linked atoms.

In one specifically contemplated embodiment, the one or more chains are aliphatic. In one specifically contemplated embodiment, the one or more chains are saturated. In some embodiments, the one or more chains are substituted with one or more aryl groups.

In some embodiments, the one or more chains comprise at least 4, 6, 8, 10, 12, or 14 chain-linked atoms. In some embodiments, the one or more chains comprise from 4-22, 6-22, 8-22, 10-22, 12-22, or 14-22 chain-linked atoms.

In one embodiment, the one or more chains are covalently bound to a moiety comprising the carbon-carbon double bond by a heteroatom containing functional group. Examples of heteroatom containing functional groups include but are not limited to ether, amine, sulfide, sulfoxide, sulfone, ester, amide, carbonate, carbamate, and urea groups.

In exemplary embodiments, the one or more chains are covalently bound to the moiety comprising the carbon-carbon double bond by an ester functional group.

In one embodiment, the lipid-containing conjugation partner comprises one or more saturated or unsaturated fatty acid esters. In one embodiment, one or more fatty acid ester is bound to the moiety comprising to carbon-carbon double bond. In one embodiment, the ester is an ester of the carboxyl group of the fatty acid and an alcohol of the moiety comprising the carbon-carbon double bond.

In one exemplary embodiment, the lipid-containing conjugation partner comprises one or two fatty acid esters. In a specifically contemplated embodiment, the lipid-containing conjugation partner comprises one fatty acid ester.

In certain embodiments, the fatty acid ester is an ester of an alcohol comprising the carbon-carbon double bond. In one embodiment, the alcohol is a monohydric, dihydric, or trihydric C₂₋₆ aliphatic alcohol. In another embodiment, the alcohol is a monohydric or dihydric C₂₋₄ aliphatic alcohol. In one exemplary embodiment, the alcohol is a monohydric C₂ aliphatic or monohydric or dihydric C₃ aliphatic alcohol. In a specifically contemplated embodiment, the alcohol is a monohydric C₂ alcohol.

In a specifically contemplated embodiment, the alcohol is vinyl alcohol.

In various embodiments, the lipid-containing conjugation partner is a compound of the formula (A-1):

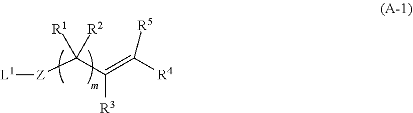

wherein
   Z and Z¹ are each independently selected from the group consisting of —O—, —NR—, —S—, —S(O)—, —SO₂—, —C(O)O—, —OC(O)—, —C(O)NR—, —NRC(O)—, —C(O)S—, —SC(O)—, —OC(O)O—, —NRC(O)O—, —OC(O)NR—, and —NRC(O)NR—;
   R is hydrogen or C₁₋₆aliphatic;
   m is an integer from 0 to 4;
   n is 1 or 2;
   R¹ and R² at each instance of m are each independently hydrogen, C₁₋₆aliphatic; or R¹ is L²-Z¹—C₁₋₆alkyl;
   R³, R⁴, and R⁵ are each independently hydrogen or C₁₋₆aliphatic; or R³ is L²-Z¹—C₁₋₆ alkyl;
   L¹ and L² are each independently C₅₋₂₁aliphatic or C₄₋₂₀heteroaliphatic; provided that:
      when R³ is L²-Z¹—C₁₋₆alkyl, R¹ is not L²-Z¹—C₁₋₆alkyl; and
      when m is an integer from 2 to 4, no more than one R¹ is L²-Z¹—C₁₋₆alkyl; and
   wherein any aliphatic, alkyl, or heteroaliphatic present in any of R, R¹, R², R³, R⁴, R⁵, L¹, and L² is optionally substituted with one or more independently selected optional substituents.

In various embodiments, the lipid-containing conjugation partner is a compound of the formula (II):

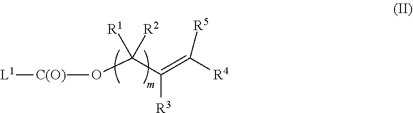

wherein
m, L¹, R, R², R³, R⁴, and R are as defined in any of the embodiments herein; and
Z¹ when present is —C(O)O—.

In various embodiments, Z, Z, R, m, n, R, R², R³, R⁴, R⁵, L¹, and/or L² are as defined in any of the embodiments herein for the moiety of formula (A) or (I).

In various embodiments, the lipid-containing conjugation partner is a vinyl ester of a fatty acid, for example vinyl palmitate.

In various embodiments, the method comprises
reacting the lipid-containing conjugation partner and an amino acid-comprising conjugation partner comprising a calcitonin gene-related peptide (CGRP) peptide, wherein at least one amino acid of the peptide comprises a thiol, to provide the peptide conjugate of the invention.

In various embodiments, the method comprises
reacting the lipid-containing conjugation partner and an amino acid-comprising conjugation partner comprising a peptide fragment of a calcitonin gene-related peptide (CGRP) peptide, wherein at least one amino acid of the peptide fragment comprises a thiol, to provide a peptide conjugate; and
coupling an amino acid of the peptide conjugate to one or more amino acids and/or one or more peptides to provide the peptide conjugate of the invention.

In various embodiments, the method comprises
reacting the lipid-containing conjugation partner and an amino acid-comprising conjugation partner comprising an amino acid of a calcitonin gene-related peptide (CGRP) peptide, wherein the amino acid comprises a thiol, to provide an amino acid conjugate; and
coupling the amino acid of the amino acid conjugate to one or more amino acids and/or one or more peptides to provide the peptide conjugate of the invention.

In various embodiments, the method comprises reacting the lipid containing conjugation partner and an amino acid-comprising conjugation partner bound to a solid phase support to provide a solid phase bound amino acid conjugate or peptide conjugate.

In various embodiments, the method comprises coupling one or more amino acids and/or one or more peptides to the solid phase bound amino acid conjugate or peptide conjugate to provide a solid phase bound peptide conjugate.

In various embodiments, the solid phase bound peptide conjugate has the amino acid sequence of the peptide conjugate of the invention.

In various embodiments, the method further comprises cleaving the peptide conjugate from the solid phase.

In various embodiments, the one or more amino acids and/or one or more peptides are coupled by SPPS. That is, in some embodiments, the method comprises coupling one or more amino acids and/or one or more peptides by SPPS.

In various embodiments, the method comprises
synthesising the amino acid sequence of the peptide of the amino acid-comprising conjugation partner by solid phase peptide synthesis (SPPS);
reacting the lipid-containing conjugation partner and the solid phase bound amino-acid comprising conjugation partner to provide a solid phase bound peptide conjugate; and
cleaving the peptide conjugate from the solid phase to provide the peptide conjugate of the invention.

In various embodiments, the method comprises
synthesising the amino acid sequence of the peptide of the amino acid-comprising conjugation partner by SPPS;
cleaving the amino acid-comprising conjugation partner from the solid phase; and
reacting the lipid-containing conjugation partner and the amino-acid comprising conjugation partner to provide the peptide conjugate of the invention.

In various embodiments, the method comprises
synthesising the amino acid sequence of the peptide fragment of the amino acid-comprising conjugation partner by SPPS;
reacting the lipid-containing conjugation partner and the solid phase bound amino-acid comprising conjugation partner to provide a solid phase bound peptide conjugate;
coupling an amino acid of the solid phase bound peptide conjugate to one or more amino acids and/or one or more peptides by SPPS to provide a solid phase bound peptide conjugate having the amino acid sequence of the peptide conjugate of the invention; and
cleaving the peptide conjugate from the solid phase to provide the peptide conjugate of the invention.

In various embodiments, the method comprises
synthesising the amino acid sequence of the peptide fragment of the amino acid-comprising conjugation partner by SPPS;
cleaving the amino acid-comprising conjugation partner from the solid phase;
reacting the lipid-containing conjugation partner and the amino acid-comprising conjugation partner to provide a peptide conjugate; and
coupling an amino acid of the peptide conjugate to one or more amino acids and/or one or more peptides to provide the peptide conjugate of the invention.

In various embodiments, the method comprises
coupling an amino acid of the peptide conjugate comprising the peptide fragment and optionally one or more amino acids and/or one or more peptides to a solid phase bound amino acid or peptide by SPPS to provide a solid phase bound peptide conjugate having
the amino acid sequence of the peptide conjugate of the invention; and cleaving the peptide conjugate from the solid phase to provide the peptide conjugate of the invention.

In various embodiments, the method comprises
coupling the amino acid of the amino acid conjugate and optionally one or more amino acids and/or one or more peptides to a solid phase bound amino acid or peptide by SPPS to provide a solid phase bound peptide conjugate having the amino acid sequence of the peptide conjugate of the invention; and
cleaving the peptide conjugate from the solid phase to provide the peptide conjugate of the invention.

In various embodiments, the method comprises acylating, for example acetylating, the Nα-amino group of the N-terminal amino acid of the peptide or peptide conjugate.

In various embodiments, the method comprises coupling coupling one or more amino acid and/or one or more peptide that reduces peptide aggregation during SPPS, for example a pseudoproline dipeptide such as Fmoc-Leu-Ser[Ψ(Me,Me)Pro]-OH.

In various embodiments, the method comprises
providing a protected amino acid-comprising conjugation partner comprising at least one amino acid comprising a thiol protected with a protecting group; and
removing the protecting group from the thiol to provide the amino acid comprising conjugation partner.

In various embodiments, the protected amino acid-comprising conjugation partner comprises one or more additional amino acids protected with one or more protecting groups.

In various embodiments, the protected amino acid-comprising conjugation partner comprises one or more additional amino acids protected with one or more protecting groups different to the protecting group of the at least one amino acid comprising the thiol; and the method comprises selectively removing the protecting group from the thiol of the at least one amino acid comprising a thiol to provide the amino acid-comprising conjugation partner.

In various embodiments, one or more or all protecting groups are removed on cleaving the peptide from the solid phase support.

In various embodiments, the SPPS is Fmoc-SPPS.

In one embodiment, the conditions effective to conjugate the lipid-containing conjugation partner to the amino acid-comprising conjugation partner comprises the generation of one or more free radicals.

In some embodiments, the generation of one or more free radicals is initiated thermally and/or photochemically. In certain embodiments, the generation of one or more free radicals is initiated by the thermal and/or photochemical degradation of a free radical initiator. In exemplary embodiments, the generation of one or more free radicals is initiated by the thermal degradation of a thermal initiator or the photochemical degradation of a photochemical initiator.

In some embodiments, thermal degradation of the free radical initiator comprises heating the reaction mixture at a suitable temperature. In some embodiments, the reaction mixture is heated at a temperature is from about 40° C. to about 200° C., from about 50° C. to about 180° C., from about 60° C. to about 150° C., from about 65° C. to about 120° C., from about 70° C. to about 115° C., from about 75° C. to about 110° C., or from about 80° C. to about 100° C. In other embodiments, the reaction mixture is heated at a temperature of at least about 40° C., at least about 50° C., at least about 60° C., or at least about 65° C. In one specifically contemplated embodiment, the reaction mixture is heated at a temperature of about 90° C.

In some embodiments, photochemical degradation of the free radical initiator comprises irradiation with ultraviolet light, preferably having a frequency compatiable with the side chains of naturally occurring amino acids. In a specifically contemplated embodiment, the ultraviolet light has a wavelength of about 365 nm. In exemplary embodiments, photochemical degradation of the free radical initiator is carried out at about ambient temperature.

In one specifically contemplated embodiment, the thermal initiator is 2,2′-azobisisobutyronitrile (AIBN) and/or the photoinitiator is 2,2-dimethoxy-2-phenylacetophenone (DMPA).

In certain embodiments, the reaction is carried out in a liquid medium. In one embodiment, the liquid medium comprises a solvent. In one embodiment, the solvent is selected from the group consisting of N-methylpyrrolidone (NMP), dimethylsulfoxide (DMSO), N,N-dimethylformamide (DMF), dichloromethane (DCM), 1,2-dichloroethane, and mixtures thereof. In one specifically contemplated embodiment, the solvent comprises NMP, DMF, DMSO, or a mixture thereof.

In one specifically contemplated embodiment, the solvent comprises DMSO or NMP. In exemplary embodiments, the solvent comprises NMP. In some embodiments, the solvent comprises DMF.

In some embodiments, the reaction is carried out in the presence of one or more additives that inhibit the formation of by-products and/or that improve the yield of or conversion to the desired conjugate.

In various embodiments, the one or more additive is an extraneous thiol, an acid, an organosilane, or a combination of any two or more thereof.

In some exemplary embodiments, the extraneous or exogenous thiol is selected from the group consisting of reduced glutathione (GSH), 2,2′-(ethylenedioxy)diethanethiol (DODT), 1,4-dithiothreitol (DTT), protein, and sterically hindered thiols. In a specifically contemplated embodiment, the extraneous or exogenous thiol is DTT. In some embodiments, the extraneous or exogenous thiol is a sterically hindered thiol, for example tert-butyl mercaptan.

In various embodiments, the acid additive is a strong inorganic or organic acid. In various embodiments, the acid is a strong organic acid. In various embodiments, the acid is TFA.

In various embodiments, the organosilane is a trialkylsilane, for example TIPS.

In some embodiments, the one or more additive is selected from the group consisting of TFA, tert-butyl mercaptan, TIPS, and combinations of any two or more thereof.

In certain embodiments, the one or more additive is a combination of an acid and an extraneous thiol, for example TFA and tert-butyl mercaptan.

In other embodiments, the one or more additive is a combination of an acid and an organosilane, for example TFA and TIPS.

In other embodiments, the one or more additive is a combination of an extraneous thiol and an organosilane, and optionally an acid, for example a combination of t-BuSH and TIPS, and TFA.

In some embodiments, the reaction is carried out for a period of time from about 5 minutes to about 48 h, 5 minutes to about 24 h, from about 5 minutes to about 12 hours, from about 5 minutes to about 6 hours, from about 5 minutes to about 3 hours, 5 minutes to 2 hours, or form about 5 minutes to about 1 hour. In exemplary embodiments, the reaction is carried out for a period of time from about 5 minutes to about 1 h. In some embodiments, the reaction is carried out until one of the conjugation partner is at least about 70%, 80%, 90%, 95%, 97%, 99%, or 100% consumed.

In certain embodiments, the reaction is carried out under substantially oxygen free conditions.

In various embodiments, the lipid containing conjugation partner is in stoichiometric excess to the amino acid-comprising conjugation partner. In various embodiments, the mole ratio of the lipid containing conjugation partner to amino acid-comprising conjugation partner is at least 7:1.

In one embodiment, the amino acid-comprising conjugation partner is a peptide-containing conjugation partner, and the lipid-containing conjugation partner is coupled to the peptide of the peptide-containing conjugation partner. In some embodiments, the lipid-containing conjugation partner is conjugated to the or an amino acid of the amino acid-comprising conjugation partner or the peptide of the peptide-containing conjugation partner. In certain embodiments, the lipid-containing conjugation partner is conjugated to the or an amino acid of the amino acid-comprising conjugation partner.

In various embodiments, the peptide conjugate is a lipopeptide, such that the method is for making a lipopeptide.

In various embodiments, the amino acid-comprising conjugation partner is a peptide-containing conjugation partner. In one embodiment, the amino acid-comprising conjugation partner consists of a peptide. In one embodiment, the peptide-containing conjugation partner consists of a peptide.

In various embodiments, the amino acid of the amino acid- or peptide conjugate to which the lipid containing moiety is conjugated is a cysteine residue.

In various embodiments, the amino-acid comprising conjugation partner is cysteine, a protected cysteine (including Nα-amine and/or carboxyl protected cysteine), or a peptide comprising a cysteine residue (including an Nα-amine or carboxyl protected cysteine residue), for example, an N-terminal cysteine residue (including an Nα-amine protected cysteine residue).

In various embodiments, the amino acid-comprising conjugation partner consists of an amino acid, for example cysteine (including Nα-amino and/or C-terminus protected cysteines).

In various embodiments, the C-terminus of the amino acid comprising conjugation partner is protected with a protecting group and/or the Nα-amino group of the amino acid comprising conjugation partner is protected with a protecting group.

In various embodiments, the carboxyl group of the C-terminus of the amino acid is protected with a carboxyl protecting group or a carboxamide protecting group and/or the Nα-amino group of the amino acid is protected with an amino protecting group.

In various embodiments, the carboxyl group of the C-terminus of the amino acid is protected with a carboxyl protecting group and/or the Nα-amino group of the amino acid is protected with an amino protecting group.

In some embodiments, the carboxyl group of the C-terminus of the peptide is protected with a carboxyl protecting group and/or the Nα-amino group of the peptide is protected with an amino protecting group.

In some embodiments, the amino protecting group is Boc, Fmoc, Cbz (carboxybenzyl), Nosyl (o- or p-nitrophenylsulfonyl), Bpoc (2-(4-biphenyl)isopropoxycarbonyl) and Dde (1-(4,4-dimethyl-2,6-dioxohexylidene)ethyl).

In various embodiments, the amino protecting group is Boc or Fmoc. In some embodiments, the amino protecting group is Fmoc.

In some embodiments, the carboxyl protecting group is tert-butyl, benzyl, or allyl.

In various embodiments, the carboxamide protecting group is Dmcp or Trityl.

In certain embodiments, one or more reactive functional groups of one or more amino acids of the amino acid-comprising conjugation partner are unprotected.

In certain embodiments, the amino acid-comprising conjugation partner comprises a peptide, wherein the reactive functional groups of the side chains of the amino acids of the peptide are unprotected, with the exception of any thiols other than the thiol to be reacted.

In certain embodiments, one or more reactive functional groups of the amino acid of the amino acid conjugate are unprotected. In certain embodiments, one or more reactive functional groups of one or more amino acids of the peptide conjugate are unprotected.

In one embodiment, the amino acid-comprising conjugation partner and/or peptide conjugate comprises a synthetic peptide. In some embodiments, the synthetic peptide is a peptide prepared by a method comprising solid phase peptide synthesis (SPPS).

In various embodiments, the method comprises coupling the amino acid of the amino acid conjugate or an amino acid of the peptide conjugate to an amino acid or an amino acid of a peptide to provide a peptide conjugate.

In various embodiments, the method comprises coupling the amino acid of the amino acid conjugate to an amino acid or an amino acid of a peptide to provide a peptide conjugate.

In some embodiments, coupling a peptide comprises individually coupling one or more amino acids and/or one or more peptides.

In some embodiments, the coupling comprises SPPS.

In some embodiments, the method comprises
synthesising the amino acid sequence of a peptide by solid phase peptide synthesis (SPPS);
coupling the amino acid of an amino acid conjugate or an amino acid of a peptide conjugate to the solid phase bound peptide by SPPS so as to provide a peptide conjugate, for example a peptide conjugate of the invention.

In some embodiments, the method comprises
reacting the lipid-containing conjugation partner and an amino acid-comprising conjugation partner to provide an amino acid or peptide conjugate;
synthesising the amino acid sequence of a peptide by solid phase peptide synthesis (SPPS);
coupling the amino acid of the amino acid conjugate or an amino acid of the peptide conjugate to the solid phase bound peptide by SPPS so as to provide a peptide conjugate, for example a peptide conjugate of the invention.

In some embodiments, the method comprises
synthesising the amino acid sequence of the peptide of the peptide-containing conjugation partner by solid phase peptide synthesis (SPPS); and
reacting the lipid-containing conjugation partner and peptide-containing conjugation partner prior to or after cleaving from the solid phase support in accordance with any of the embodiments described herein.

In some embodiments, the method comprises
synthesising the amino acid sequence of the peptide of the peptide-containing conjugation partner by solid phase peptide synthesis (SPPS); and
reacting the lipid-containing conjugation partner and solid phase bound peptide-containing conjugation partner in accordance with any of the embodiments described herein.

In exemplary embodiments, the method comprises
synthesising the amino acid sequence of the peptide of the peptide-containing conjugation partner by SPPS,
cleaving the peptide from the solid phase support; and
reacting the lipid-containing conjugation partner and peptide-containing conjugation partner in accordance with any of the embodiments described herein.

In one embodiment, the peptide-containing conjugation partner is not purified prior to reaction with the lipid-containing conjugation partner.

In one embodiment, the method further comprises separating the peptide conjugate from the reaction medium and optionally purifying the peptide conjugate.

In various embodiments, the amino acid of the amino acid conjugate is coupled under conditions that reduce epimerisation at the α-carbon of the amino acid. In various embodiments, the conditions are such that less than about 35, 30, 25, 20, 15, 10, 5, 3, 2, or 1% by mol of the amino acid is epimerised.

In various embodiments, the conditions that reduce epimerisation comprise the use of PyBOP as the coupling reagent. In various embodiments, the conditions comprise the use of PyBOP and 2,4,6-trimethylpyridine.

In certain embodiments, the peptide conjugate and/or amino acid-comprising conjugation partner comprises only naturally occurring amino acids. In other embodiments, 75% or more, 80% or more, 85% or more, 90% or more, 95% or more, 97% or more, or 99% or more of the amino acid residues in the peptide conjugate and/or amino acid-comprising conjugation partner are naturally occurring amino acids.

Those skilled in the art will appreciate that the peptide of the peptide conjugate and/or peptide-containing conjugation partner may, as described herein, be optionally substituted, modified, or bound to various other moieties as described herein.

In another aspect, the present invention broadly consists in a peptide conjugate of the invention made by a method of the invention.

It is intended that reference to a range of numbers disclosed herein (for example, 1 to 10) also incorporates reference to all rational numbers within that range (for example, 1, 1.1, 2, 3, 3.9, 4, 5, 6, 6.5, 7, 8, 9 and 10) and also any range of rational numbers within that range (for example, 2 to 8, 1.5 to 5.5 and 3.1 to 4.7) and, therefore, all sub-ranges of all ranges expressly disclosed herein are hereby expressly disclosed. These are only examples of what is specifically intended and all possible combinations of numerical values between the lowest value and the highest value enumerated are to be considered to be expressly stated in this application in a similar manner.

This invention may also be said broadly to consist in the parts, elements and features referred to or indicated in the specification of the application, individually or collectively, and any or all combinations of any two or more said parts, elements or features, and where specific integers are mentioned herein which have known equivalents in the art to which this invention relates, such known equivalents are deemed to be incorporated herein as if individually set forth.

Although the present invention is broadly as defined above, those persons skilled in the art will appreciate that the invention is not limited thereto and that the invention also includes embodiments of which the following description gives examples.

### BRIEF DESCRIPTION OF THE FIGURES

The invention will be described with reference to the accompanying figures in which:

FIGS. 1 and 2 show concentration-response curves for CGRP-stimulated cyclic adenosine monophosphate (cAMP) production in the absence or presence of increasing concentrations of antagonist A4 at CGRP and AMY₁ receptors respectively. Mean data from three independent experiments are shown. For each of FIGS. 1 and 2, the black circles ( [symbol] ) represent the data points corresponding to cAMP production in the absence of an antagonist, the squares ( [symbol] ) represented data points corresponding to cAMP production in the presence of 10 nM of antagonist A4, the triangles pointing up ( [symbol] ) represent data points corresponding to cAMP production in the presence of 30 nM of antagonist A4 present, and the triangles pointing down ( [symbol] ) represent data points corresponding to cAMP production in the presence of 100 nM of anatagonist A4.

FIGS. 3 and 4 show concentration-response curves for CGRP-stimulated cAMP production in the absence or presence of antagonist A or antagonist A4 at the CGRP receptor respectively with or without a wash step as described in the methods of the examples section. Mean data of three replicates from a single experiment is shown. For FIG. 3, the black circles ( [symbol] ) represent the data points corresponding cAMP production in the absence of an antagonist, the triangles pointing down ( [symbol] ) represent data points corresponding to cAMP production in the presence of 300 nM of antagonist A with a wash step and the squares ( [symbol] ) represent data points corresponding to cAMP production in the presence of 300 nM of antagonist A without a wash step. For FIG. 4, the black circles ( [symbol] ) represent the data points corresponding cAMP production in the absence of an antagonist, the triangles pointing down ( [symbol] ) represent data points corresponding to cAMP production in the presence of 300 nM of antagonist A4 with a wash step and the squares ( [symbol] ) represent data points corresponding to cAMP production in the presence of 300 nM of antagonist A4 without a wash step.

FIG. 5 shows capsaicin-evoked vasodilation in the ears of mice following prior administration of vehicle (saline+0.1% BSA+3.2% DMSO) or 960 nmol/kg of either antagonist A or antagonist A4 as described in the methods of the examples section. The combined mean±s.e.m from 4 independent experiments for saline, antagonist A and antagonist A4 are shown. Timepoints at −3, −2, and −1 minutes denote the baseline laser doppler signal prior to capsaicin application to the ear. Timepoints 1-15 minutes show the vasodilatory flux signal following application of capsaicin to the ear. All data is normalised to the mean of the baseline data at −3, −2, and −1 minutes and therefore shows percentage increase over the normalised baseline at 100%. Black circles ( [symbol] ) represent the data points corresponding to the vehicle group in the absence of antagonist, the open circles (∘) represent the data points corresponding to treatment with antagonist A, and the squares ( [symbol] ) represent data points corresponding to treatment with antagonist A4.

FIG. 6 shows area under curve analyses corresponding to FIG. 5. Each data point represents an independent experiment with the mean±s.e.m combined from the 4 independent experiments.

### DETAILED DESCRIPTION OF THE INVENTION

The term “comprising” as used in this specification and claims means “consisting at least in part of”. When interpreting each statement in this specification and claims that includes the term “comprising”, features other than that or those prefaced by the term may also be present. Related terms such as “comprise” and “comprises” are to be interpreted in the same manner.

As used herein the term “and/or” means “and” or “or”, or both.

As used herein “(s)” following a noun means the plural and/or singular forms of the noun.

Asymmetric centers may exist in the compounds described herein. The asymmetric centers may be designated as (R) or (S), depending on the configuration of substituents in three dimensional space at the chiral carbon atom. All stereochemical isomeric forms of the compounds, including diastereomeric, enantiomeric, and epimeric forms, as well as d-isomers and 1-isomers, and mixtures thereof, including enantiomerically enriched and diastereomerically enriched mixtures of stereochemical isomers, are within the scope of the invention.

Individual enantiomers can be prepared synthetically from commercially available enantiopure starting materials or by preparing enantiomeric mixtures and resolving the mixture into individual enantiomers. Resolution methods include conversion of the enantiomeric mixture into a mixture of diastereomers and separation of the diastereomers by, for example, recrystallization or chromatography, and any other appropriate methods known in the art. Starting materials of defined stereochemistry may be commercially available or made and, if necessary, resolved by techniques well known in the art. The compounds described herein may also exist as conformational or geometric isomers, including cis, trans, syn, anti, entgegen (E), and zusammen (Z) isomers. All such isomers and any mixtures thereof are within the scope of the invention.

Also within the scope of the invention are any tautomeric isomers or mixtures thereof of the compounds described. As would be appreciated by those skilled in the art, a wide variety of functional groups and other structures may exhibit tautomerism. Examples include, but are not limited to, keto/enol, imine/enamine, and thioketone/enethiol tautomerism.

The compounds described herein may also exist as isotopologues and isotopomers, wherein one or more atoms in the compounds are replaced with different isotopes. Suitable isotopes include, for example, ¹H, ²H (D), ³H (T), ¹²C, ¹³C, ¹⁴C, ¹⁶O, and ¹⁸O. Procedures for incorporating such isotopes into the compounds described herein will be apparent to those skilled in the art. Isotopologues and isotopomers of the compounds described herein are also within the scope of the invention.

Also within the scope of the invention are salts of the compounds described herein, including pharmaceutically acceptable salts. Such salts include, acid addition salts, base addition salts, and quaternary salts of basic nitrogen-containing groups.

Acid addition salts can be prepared by reacting compounds, in free base form, with inorganic or organic acids. Examples of inorganic acids include, but are not limited to, hydrochloric, hydrobromic, nitric, sulfuric, and phosphoric acid. Examples of organic acids include, but are not limited to, acetic, trifluoroacetic, propionic, succinic, glycolic, lactic, malic, tartaric, citric, ascorbic, maleic, fumaric, pyruvic, aspartic, glutamic, stearic, salicylic, methanesulfonic, benzenesulfonic, isethionic, sulfanilic, adipic, butyric, and pivalic.

Base addition salts can be prepared by reacting compounds, in free acid form, with inorganic or organic bases. Examples of inorganic base addition salts include alkali metal salts, alkaline earth metal salts, and other physiologically acceptable metal salts, for example, aluminium, calcium, lithium, magnesium, potassium, sodium, or zinc salts. Examples of organic base addition salts include amine salts, for example, salts of trimethylamine, diethylamine, ethanolamine, diethanolamine, and ethylenediamine. Quaternary salts of basic nitrogen-containing groups in the compounds may be may be prepared by, for example, reacting the compounds with alkyl halides such as methyl, ethyl, propyl, and butyl chlorides, bromides, and iodides, dialkyl sulfates such as dimethyl, diethyl, dibutyl, and diamyl sulfates, and the like.

The compounds described herein may form or exist as solvates with various solvents. If the solvent is water, the solvate may be referred to as a hydrate, for example, a mono-hydrate, a di-hydrate, or a tri-hydrate. All solvated forms and unsolvated forms of the compounds described herein are within the scope of the invention.

The general chemical terms used herein have their usual meanings.

The term “aliphatic” is intended to include saturated and unsaturated, nonaromatic, straight chain, branched, acyclic, and cyclic hydrocarbons. Those skilled in the art will appreciate that aliphatic groups include, for example, alkyl, alkenyl, alkynyl, cycloalkyl, and cycloalkenyl groups, and hybrids thereof such as (cycloalkyl)alkyl, (cycloalkenyl)alkyl and (cycloalkyl)alkenyl groups. In various embodiments, aliphatic groups comprise from 1-12, 1-8, 1-6, or 1-4 carbon atoms. In some embodiments, aliphatic groups comprise 5-21, from 9-21, or from 11-21 carbon atoms, such as from 11, 13, 15, 17, or 19 carbon atoms. In some embodiments, the aliphatic group is saturated.

The term “heteroaliphatic” is intended to include aliphatic groups, wherein one or more chain and/or ring carbon atoms are independently replaced with a heteroatom, preferably a heteroatom selected from oxygen, nitrogen and sulfur. In some embodiments, the heteroaliphatic is saturated. Examples of heteroaliphatic groups include, but are not limited to, linear or branched, heteroalkyl groups.

The term “alkyl” is intended to include saturated straight chain and branched chain hydrocarbon groups. In some embodiments, alkyl groups have from 1 to 12, 1 to 10, 1 to 8, 1 to 6, or from 1 to 4 carbon atoms. In some embodiments, alkyl groups have from 5-21, from 9-21, or from 11-21 carbon atoms, such as from 11, 13, 15, 17, or 19 carbon atoms. Examples of straight chain alkyl groups include, but are not limited to, methyl, ethyl, n-propyl, n-butyl, n-pentyl, n-hexyl, n-heptyl, and n-octyl. Examples of branched alkyl groups include, but are not limited to, isopropyl, iso-butyl, sec-butyl, tert-butyl, neopentyl, isopentyl, and 2,2-dimethylpropyl.

The term “alkenyl” is intended to include straight and branched chain alkyl groups having at least one double bond between two carbon atoms. In some embodiments, alkenyl groups have from 2 to 12, from 2 to 10, from 2 to 8, from 2 to 6, or from 2 to 4 carbon atoms. In some embodiments, alkenyl groups have from 5-21, from 9-21, or from 11-21 carbon atoms, such as from 11, 13, 15, 17, or 19 carbon atoms. In some embodiments, alkenyl groups have one, two, or three carbon-carbon double bonds. Examples of alkenyl groups include, but are not limited to, vinyl, allyl, —CH═CH(CH₃), —CH═C(CH₃)₂, —C(CH₃)═CH₂, and —C(CH₃)═CH(CH₃).

The term “alkynyl” is intended to include straight and branched chain alkyl groups having at least one triple bond between two carbon atoms. In some embodiments, the alkynyl group have from 2 to 12, from 2 to 10, from 2 to 8, from 2 to 6, or from 2 to 4 carbon atoms. In some embodiments, alkynyl groups have one, two, or three carbon-carbon triple bonds. Examples include, but are not limited to, —C≡CH, —C≡CH₃, —CH₂C≡CH₃, and —C≡CH₂CH(CH₂CH₃)₂.

The term “heteroalkyl” is intended to include alkyl groups, wherein one or more chain carbon atoms are replaced with a heteroatom, preferably a heteroatom selected from the group consisting of oxygen, nitrogen, and sulfur. Heteroalkyl groups include, for example, polyethylene glycol groups and polyethylene glycol ether groups, and the like.

The term “cycloalkyl” is intended to include mono-, bi- or tricyclic alkyl groups. In some embodiments, cycloalkyl groups have from 3 to 12, from 3 to 10, from 3 to 8, from 3 to 6, from 3 to 5 carbon atoms in the ring(s). In some embodiments, cycloalkyl groups have 5 or 6 ring carbon atoms. Examples of monocyclic cycloalkyl groups include, but are not limited to, cyclopropyl, cyclobutyl, cyclopentyl, cyclohexyl, cycloheptyl, and cyclooctyl. In some embodiments, the cycloalkyl group has from 3 to 8, from 3 to 7, from 3 to 6, from 4 to 6, from 3 to 5, or from 4 to 5 ring carbon atoms. Bi- and tricyclic ring systems include bridged, spiro, and fused cycloalkyl ring systems. Examples of bi- and tricyclic ring cycloalkyl systems include, but are not limited to, bicyclo[2.1.1]hexanyl, bicyclo[2.2.1]heptanyl, adamantyl, and decalinyl.

The term “cycloalkenyl” is intended to include non-aromatic cycloalkyl groups having at least one double bond between two carbon atoms. In some embodiments, cycloalkenyl groups have one, two or three double bonds. In some embodiments, cycloalkenyl groups have from 4 to 14, from 5 to 14, from 5 to 10, from 5 to 8, or from 5 to 6 carbon atoms in the ring(s). In some embodiments, cycloalkenyl groups have 5, 6, 7, or 8 ring carbon atoms. Examples of cycloalkenyl groups include cyclohexenyl, cyclopentenyl, cyclohexadienyl, butadienyl, pentadienyl, and hexadienyl.

The term “aryl” is intended to include cyclic aromatic hydrocarbon groups that do not contain any ring heteroatoms. Aryl groups include monocyclic, bicyclic and tricyclic ring systems. Examples of aryl groups include, but are not limited to, phenyl, azulenyl, heptalenyl, biphenyl, fluorenyl, phenanthrenyl, anthracenyl, indenyl, indanyl, pentalenyl, and naphthyl. In some embodiments, aryl groups have from 6 to 14, from 6 to 12, or from 6 to 10 carbon atoms in the ring(s). In some embodiments, the aryl groups are phenyl or naphthyl. Aryl groups include aromatic-aliphatic fused ring systems. Examples include, but are not limited to, indanyl and tetrahydronaphthyl.

The term “arylalkyl” refers to an alkyl group, as defined herein, substituted with an aryl group, as defined herein. Arylalkyl groups are attached to the parent molecular moiety via the alkyl group. Examples of arylalkyl groups include, but are not limited to, benzyl, 2-phenylethyl, 3-phenylpropyl, 2-naphth-2-ylethyl, and the like.

The term “acyl” is intended to include Rⁿ—C(O)— groups, wherein Rⁿ is an aliphatic, alkyl, alkenyl, cycloalkyl, cycloalkenyl, aryl, arylalkyl group as defined herein. In certain embodiments, Rⁿ is an alkyl group as defined herein, for example an acetyl group.

The term “halo” or “halogen” is intended to include F, Cl, Br, and I.

The term “heteroatom” is intended to include oxygen, nitrogen, sulfur, selenium, or phosphorus. In some embodiments, the heteroatom is selected from the group consisting of oxygen, nitrogen, and sulfur.

As used herein, the term “substituted” is intended to mean that one or more hydrogen atoms in the group indicated is replaced with one or more independently selected suitable substituents, provided that the normal valency of each atom to which the substituent/s are attached is not exceeded, and that the substitution results in a stable compound. In various embodiments, optional substituents in the compounds described herein include but are not limited to halo, CN, NO₂, OH, NH₂, NHR^{x}, NR^{x}R^{y}, C₁₋₆haloalkyl, C₁₋₆haloalkoxy, C(O)NH₂, C(O)NHR^{x}, C(O)NR^{x}R^{y}, SO₂R^{x}, OR^{y}, SR^{x}, S(O)R^{x}, C(O)R^{x}, and C₁₋₆aliphatic; wherein R^{x} and R^{y} are each independently C₁₋₆aliphatic, for example C₁₋₆alkyl.

The term “carboxyl protecting group” as used herein is means a group that is capable of readily removed to provide the OH group of a carboxyl group and protects the carboxyl group against undesirable reaction during synthetic procedures. Such protecting groups are described in Protective Groups in Organic Synthesis edited by T. W. Greene et al. (John Wiley & Sons, 1999) and ‘Amino Acid-Protecting Groups’ by Fernando Albericio (with Albert Isidro-Llobet and Mercedes Alvarez) Chemical Reviews 2009 (109) 2455-2504. Examples include, but are not limited to, alkyl and silyl groups, for example methyl, ethyl, tert-butyl, methoxymethyl, 2,2,2-trichloroethyl, benzyl, diphenylmethyl, trimethylsilyl, and tert-butyldimethylsilyl, and the like.

The term “amine protecting group” as used herein means a group that is capable of being readily removed to provide the NH₂ group of an amine group and protects the amine group against undesirable reaction during synthetic procedures. Such protecting groups are described in Protective Groups in Organic Synthesis edited by T. W. Greene et al. (John Wiley & Sons, 1999) and ‘Amino Acid-Protecting Groups’ by Fernando Albericio (with Albert Isidro-Llobet and Mercedes Alvarez) Chemical Reviews 2009 (109) 2455-2504. Examples include, but are not limited to, acyl and acyloxy groups, for example acetyl, chloroacetyl, trichloroacetyl, o-nitrophenylacetyl, o-nitrophenoxy-acetyl, trifluoroacetyl, acetoacetyl, 4-chlorobutyryl, isobutyryl, picolinoyl, aminocaproyl, benzoyl, methoxy-carbonyl, 9-fluorenylmethoxycarbonyl, 2,2,2-trifluoroethoxycarbonyl, 2-trimethylsilylethoxy-carbonyl, tert-butyloxycarbonyl, benzyloxycarbonyl, p-nitrobenzyloxycarbonyl, 2,4-dichloro-benzyloxycarbonyl, and the like. Further examples include Cbz (carboxybenzyl), Nosyl (o- or p-nitrophenylsulfonyl), Bpoc (2-(4-biphenyl)isopropoxycarbonyl) and Dde (1-(4,4-dimethyl-2,6-dioxohexylidene)ethyl).

The term “carboxamide protecting group” as used herein means a group that is capable of being readily removed to provide the NH₂ group of a carboxamide group and protects the carboxamide group against undesirable reaction during synthetic procedures. Such protecting groups are described in Protective Groups in Organic Synthesis edited by T. W. Greene et al. (John Wiley & Sons, 1999) and ‘Amino Acid-Protecting Groups’ by Fernando Albericio (with Albert Isidro-Llobet and Mercedes Alvarez) Chemical Reviews 2009 (109) 2455-2504. Examples include, but are not limited to, 9-xanthenyl (Xan), trityl (Trt), methyltrityl (Mtt), cyclopropyldimethylcarbinyl (Cpd), and dimethylcyclopropylmethyl (Dmcp).

### Peptide Conjugates

The present invention relates to a peptide conjugate comprising a calcitonin gene-related peptide (CGRP) peptide, wherein at least one amino acid of the peptide is covalently conjugated to a lipid-containing moiety via a sulfur atom of a sulfide group, wherein the peptide is a CGRP receptor antagonist.

The inventors have surprisingly found, as shown in the Examples, that certain peptide conjugates of the present invention have useful activity as antagonists of CGRP receptors.

α-CGRP and β-CGRP each comprise 37 amino acids and differ by three amino acids. The sequences of human wild type α-CGRP and β-CGRP are as follows:

α-CGRP:
[SEQ ID NO: 3]
ACDTATCVTHRLAGLLSRSGGVVKNNFVPTNVGSKAF
β-CGRP:
[SEQ ID NO: 4]
ACNTATCVTHRLAGLLSRSGGMVKSNFVPTNVGSKAF

α-CGRP and β-CGRP comprise the following regions: a disulfide-bonded N-terminal region comprising amino acids 1-7 wherein an intramolecular disulfide bond is formed between the cysteines at positions 2 and 7, an alpha-helical region comprising amino acids 8-18, a hinge region comprising amino acids 19-26 and a receptor binding region comprising amino acids 27-37. The peptide conjugates of the invention may comprise one or more amino acids covalently conjugated to a lipid-containing moiety in any one or more of these four regions.

CGRP binds to a receptor known as the CGRP receptor that comprises a G protein-coupled receptor—the calcitonin receptor-like receptor (CLR), associated with receptor activity-modifying proteins (RAMP1). CLR/RAMP1 receptor complexes are located on a number of cell types such as, for example, on vascular smooth muscle and/or endothelial cells. Additionally, CLR/RAMP1 receptor complexes are located on a number of cell types with limited sensory and motor connections, and as a result it is thought that CGRP may also play a hormonal role. A second high affinity CGRP receptor is formed by a related G protein-coupled receptor, the calcitonin receptor (CTR), associated with RAMP1. This receptor is the AMY₁ receptor. This receptor is also found in sensory neurons.

Antagonists of CGRP receptors compete with the natural ligand, CGRP for binding to the receptor binding site. Unlike the binding of CGRP or CGRP receptor agonists, the interaction of an antagonist with a CGRP receptor does not lead to receptor activation.

Once bound, a CGRP receptor antagonist blocks the binding of CGRP to a CGRP receptor, by directly occluding the CGRP binding site or in an allosteric manner to prevent CGRP binding and thereby preventing the downstream intra-cellular signalling pathways that would normally be triggered on CGRP-receptor binding.

A “CGRP peptide” as used herein refers to a peptide that preferentially binds a CGRP receptor under physiological conditions of temperature, pH, and ionic strength. CGRP receptors include the CLR/RAMP1 receptor and the CTR/RAMP1 receptor (the AMY₁ receptor) described above. CGRP peptides may bind the CLR/RAMP1 receptor; the AMY₁ receptor; or both the CLR/RAMP1 receptor and the AMY₁ receptor. For purposes of the present invention, CGRP peptides include those having a full native CGRP peptide sequence and non-native CGRP peptide analogs containing modifications of a native CGRP sequence (e.g., amino acid substitutions, insertions, deletions, and/or amino terminal end truncations as further described herein) relative to a native CGRP sequence of interest, which can be, e.g., any known CGRP sequence, such as but not limited to, the native human αCGRP sequence or human βCGRP sequence. In various embodiments, the CGRP receptors are human CGRP receptors.

A “CGRP receptor antagonist” as used herein refers to a peptide, including a CGRP peptide, a peptide conjugate of the invention, a non-CGRP peptide or a non-peptide molecule that antagonizes, blocks, decreases, reduces, impedes, or inhibits CGRP receptor activation by full length native αCGRP or βCGRP under physiological conditions of temperature, pH, and ionic strength. CGRP receptor antagonists include full and partial antagonists. The present invention does not depend on any particular mechanism of antagonism. For example, CGRP receptor antagonists can act as competitive antagonists or noncompetitive antagonists. Such antagonist activity can be detected by methods described herein, including in the Examples, and by known in vitro methods or in vivo functional assay methods. (See, e.g., Smith et al., Modifications to the N-terminus but not the C-terminus of calcitonin gene-related peptide (8-37) produce antagonists with increased affinity, J. Med. Chem., 46:2427-2435 (2003)).

The antagonist activity of the peptide conjugates of the invention at a CGRP receptor may be defined in terms of an antagonist potency value (pA₂). The antagonist activity of the peptide conjugates of the invention at a CGRP receptor may be compared with the antagonist activity of human wild type α-CGRP8-37, a known CGRP receptor antagonist. The peptide conjugates of the invention in some embodiments may have antagonist potency value (pA₂) more than a value 10-fold (i.e. 10-times) less than the antagonist potency (pA₂) of CGRP8-37. Preferably, the peptide conjugates of the invention have an antagonist potency (pA₂) value similar to or more than the antagonist potency (pA₂) value of CGRP8-37. The antagonist potency may be against a CLR/RAMP1 CGRP receptor or a CTR/RAMP1 AMY1 CGRP receptor. Antagonist potency (pA₂) values for peptide conjugates of the invention and human wild type α-CGRP8-37 at a CGRP receptor can be obtained by, for example, using cAMP based assays, such as those described in the Examples herein. Other suitable assays will be apparent to those skilled in the art.

Without wishing to be bound by theory, the inventors believe that certain peptide conjugates of the present invention may have increased half lives compared to CGRP peptides that are CGRP receptor antagonists lacking the covalently conjugated lipid-containing moiety or moieties present in the peptide conjugates of the invention, for example CGRP8-37. The peptide conjugate of the invention in some embodiments may have a half life that is at least 2-fold (i.e. 2-times) longer than the half life of human wild type α-CGRP8-37. The half life may be measured by any suitable method known in the art, for example in a suitable rodent model, preferably a rat model.

Additionally, the inventors have found that certain peptide conjugates of the invention exhibit prolonged or persistent antagonist activity at certain CGRP receptors, continuing to exhibit antagonist activity even after the receptors have been washed, which is not observed with human wild type α-CGRP8-37, as described in the Examples herein. Without wishing to be bound by theory, the inventors believe that this prolonged or persistent antagonist activity is due the formation of a stable interaction, such as a stable binding interaction, with the receptor. The peptide conjugate may have a first antagonist potency value (pA₂) at a CGRP receptor after incubating the receptor and peptide conjugate and not washing the receptor prior to determining the antagonist potency value and a second antagonist potency value (pA₂) at a CGRP receptor after incubating the receptor and peptide conjugate and then washing the receptor prior to determining the antagonist potency value. The second antagonist potency value is lower than the first antagonist potency value, but the fold change reduction in antagonist potency is less than the fold change reduction in antagonist potency for α-CGRP8-37 (SEQ ID No:96) in the same assays. The fold change reduction in antagonist potency between the first and second antagonist potency values of the peptide conjugate may be less than about 50, 45, 40, 35, 30, 25, 20, 15, 10, 9, 8, 7, 6, 5, 4, 3, or 2, when measured by a cAMP assay for a CLR/RAMP1 CGRP receptor. Alternatively or additionally, the fold change reduction in antagonist potency between the first and second antagonist potency values of the peptide conjugate may be less than about 20, 15, 14, 13, 12, 11, 10, 9, 8, 7, 6, 5, 4, 3, or 2 when measured by a cAMP assay for a CTR/RAMP1 AMY1 CGRP receptor.

Various CGRP peptides, including CGRP peptides that are CGRP receptor antagonists, have been characterised and are suitable for use in the present invention. All CGRP peptides and CGRP peptides that are CGRP receptor antagonists, whether or not presently characterized, are contemplated herein.

Known antagonists of CGRP receptors include peptide antagonists such as the CGRP fragment CGRP8-37. The sequences of human wild type α-CGRP8-37 and β-CGRP8-37 are as follows:

α-CGRP8-37:
[SEQ ID NO: 96]
VTHRLAGLLSRSGGVVKNNFVPTNVGSKAF
β-CGRP8-37:
[SEQ ID NO: 31]
VTHRLAGLLSRSGGMVKSNFVPTNVGSKAF

In some embodiments the CGRP peptides comprise or consist of CGRP8-37 or a functional variant thereof wherein at least one amino acid is or is substituted with an amino acid covalently conjugated to a lipid-containing moiety. Examples include peptides comprising or consisting of an amino acid sequence of any one of SEQ ID Nos:1, 2, 19-41, 56-78, 81, 82, 85, 86, 8, 90, 96 and 31.

In other embodiments the CGRP peptides comprise full length CGRP or a functional variant thereof wherein at least one amino acid is or is substituted with an amino acid coavalently conjugated to a lipid-containing moiety. In some embodiments the full length CGRP peptide or functional variant thereof has been modified to inhibit, either partially or fully, binding to or agonism of one or more CGRP receptors by α-CGRP or β-CGRP, for example by substitution of one or more amino acids in the disulfide-bonded N-terminal region, including substitution with one or more amino acids covalently conjugated to a lipid-containing moiety. In other embodiments the CGRP peptides comprise an N-terminally truncated CGRP peptide or functional variant thereof, for example, CGRP 7-37 wherein at least one amino acid is or is substituted with an amino acid coavalently conjugated to a lipid-containing moiety. Examples include peptides comprising or consisting of an amino acid sequence of any one of SEQ ID Nos:1, 2, 3-18, 42-55, 79, 80, 83, 84, 87, 88, 91-95 and 97.

Also contemplated are CGRP peptides, including CGRP peptides that are CGRP receptor antagonists, comprising one or more amino acid substitutions, such as one or more conservative amino acid substitutions.

A “conservative amino acid substitution” is one in which an amino acid residue is replaced with another residue having a chemically similar or derivatised side chain. Families of amino acid residues having similar side chains, for example, have been defined in the art. These families include, for example, amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Amino acid analogs (e.g., phosphorylated or glycosylated amino acids) are also contemplated in the present invention, as are peptides substituted with non-naturally occurring amino acids, including but not limited to N-alkylated amino acids (e.g. N-methyl amino acids), D-amino acids, β-amino acids, and γ-amino acids.

Fragments and variants of CGRP peptides, including CGRP peptides that are CGRP antagonists, are also specifically contemplated.

A “fragment” of a peptide, is a subsequence of the peptide. In the context of the peptide conjugates of the invention, a “fragment” of a peptide is typically a subsequence of the peptide that performs a function that is required for the enzymatic or binding activity and/or provides three dimensional structure of the peptide, such as the three dimensional structure of a polypeptide. In the context of the methods of synthesis described herein, a “fragment” of a peptide (or “peptide fragment”) refers to any subsequence of the peptide, whether or not that subsequence performs a biological function.

The term “variant” as used herein refers to peptide sequences, including for example peptide sequences different from the specifically identified sequences, wherein one or more amino acid residues is deleted, substituted, or added. Variants are naturally-occurring variants, or non-naturally occurring variants. Variants are from the same or from other species and may encompass homologues, paralogues and orthologues. The term “functional variant” refers to variants of peptides that possess biological activities that are the same or similar to those of the wild type peptides. For example, a functional variant of a CGRP peptide, for example CGRP8-37, is a variant of the CGRP peptide that exhibits similar CGRP receptor antagonism, which may be determined using methods described herein. The term “variant” with reference to peptides encompasses all forms of peptides as defined herein. The degree of sequence identity between a variant and the sequence of a peptide described herein can be determined by comparing a candidate amino acid sequence to a sequence described herein, such as full length CGRP or CGRP8-37 thereof using the BLAST suite of programs (version 2.2.12; 28 Aug. 2005) that is publicly available from NCBI (ftp://ftp.ncbi.nih.gov/blast/).

The term “α-amino acid” or “amino acid” refers to a molecule containing both an amino group and a carboxyl group bound to a carbon which is designated the α-carbon. Suitable amino acids include, without limitation, both the D- and L-isomers of the naturally-occurring amino acids, as well as non-naturally occurring amino acids prepared by organic synthesis or other metabolic routes. Unless the context specifically indicates otherwise, the term amino acid, as used herein, is intended to include amino acid analogs. In certain embodiments the peptide-conjugates of the present invention comprise only naturally occurring amino acids.

The term “naturally occurring amino acid” refers to any one of the twenty amino acids commonly found in peptides synthesized in nature, and known by the one letter abbreviations A, R, N, C, D, Q, E, G, H, I, L, K, M, F, P, S, T, W, Y and V.

Also contemplated are CGRP peptides, including CGRP peptides that are CGRP receptor antagonists, comprising one or more amino acid substitutions with a non-canonical amino acid.

The term “non-canonical amino acid” as used herein includes naturally rare (in peptides or proteins) amino acids or non-naturally occurring amino acid residues. Non-canonical amino acid includes amino acids in D- or L-form that are not among the 20 naturally occurring amino acids. “Non-canonical amino acids” include molecules which are structurally similar to an amino acid and which can be substituted for an amino acid, including without limitation, compounds which are structurally identical to an amino acid, as defined herein, except for the inclusion of one or more additional methylene groups between the amino and carboxyl group (e.g., α-amino β-carboxy acids), or for the substitution of the amino or carboxy group by a similarly reactive group (e.g., substitution of the primary amine with a secondary or tertiary amine (e.g. N-methyl-amino acids), or substitution or the carboxy group with an ester or carboxamide); peptoids, wherein the amino acid side chain is appended to the nitrogen atom of the Nα-amino group, rather than the α-carbon; α,α-disubstituted amino acids, for example α-alkyl amino acids, wherein the α-carbon is substituted with an alkyl group in addition to the side chain of the amino acid; and α,α-diamino acids, wherein the α-carbon is substituted with two amino groups.

Other examples of non-canonical amino acids may include, for example conformationally constrained amino acids.

Examples of non-canonical amino acids include, without limitation (in the L-form or D-form; abbreviated as in parentheses): citrulline (Cit), homocitrulline (hCit), Nα-methylcitrulline (NMeCit), Nα-methylhomocitrulline (Nα-MeHoCit), ornithine (Orn), Nα-Methylornithine (Nα-MeOrn or NMeOrn), sarcosine (Sar), homolysine (hLys or hK), homoarginine (hArg or hR), homoglutamine (hQ), Nα-methylarginine (NMeR), Nα-methylleucine (Nα-MeL or NMeL), N-methylhomolysine (NMeHoK), Nα-methylglutamine (NMeQ), norleucine (Nle), norvaline (Nva), 1,2,3,4-tetrahydroisoquinoline (Tic), Octahydroindole-2-carboxylic acid (Oic), 3-(1-naphthyl)alanine (1-Nal), 3-(2-naphthyl)alanine (2-Nal), 1,2,3,4-tetrahydroisoquinoline (Tic), 2-indanylglycine (Igl), para-iodophenylalanine (pI-Phe), para-aminophenylalanine (4AmP or 4-Amino-Phe), 4-guanidino phenylalanine (Guf), nitrophenylalanine (nitrophe), aminophenylalanine (aminophe or Amino-Phe), benzylphenylalanine (benzylphe), γ-carboxyglutamic acid (γ-carboxyglu), hydroxyproline (hydroxypro), p-carboxyl-phenylalanine (Cpa), α-aminoadipic acid (Aad), Nα-methyl valine (NMeVal), N-α-methyl leucine (NMeLeu), Nα-methylnorleucine (NMeNle), cyclopentylglycine (Cpg), cyclohexylglycine (Chg), acetylarginine (acetylarg), α,β-diaminopropionoic acid (Dpr), α,γ-diaminobutyric acid (Dab), diaminopropionic acid (Dap), cyclohexylalanine (Cha), 4-methyl-phenylalanine (MePhe), β,β-diphenyl-alanine (BiPhA), aminobutyric acid (Abu), 4-phenyl-phenylalanine (or biphenylalanine; 4Bip), α-amino-isobutyric acid (Aib), beta-alanine, beta-aminopropionic acid, piperidinic acid, aminocaprioic acid, aminoheptanoic acid, aminopimelic acid, desmosine, diaminopimelic acid, N-ethylglycine, N-ethylaspargine, hydroxylysine, allo-hydroxylysine, isodesmosine, allo-isoleucine, N-methylglycine, N-methylisoleucine, N-methylvaline, 4-hydroxyproline (Hyp), γ-carboxyglutamate, ε-N,N,N-trimethyllysine, ε-N-acetyllysine, O-phosphoserine, N-acetylserine, N-formylmethionine, 3-methylhistidine, 5-hydroxylysine, ω-methylarginine, and other similar amino acids, and derivatized forms of any of these.

In some embodiments the one or more amino acid substitutions with a non-naturally occurring amino acid is made to reduce the susceptibility of the CGRP peptide to enzymatic proteolysis. This reduced susceptibility may be due to an effect on a protease binding site or cleavage site for an exopeptidase or endopeptidase. Examples of such substitutions include the substitution of one or more arginines or one or more lysines for a D-arginine, N-methylarginine, citrulline, dimethylarginine, homoarginine, N-methyl-citrulline, homocitrulline, 4-guanidino phenylalanine, D-lysine, N-methyl lysine, homolysine, 4-amino phenylalanine or ornithine.

The terms “polypeptide” and “peptide” and the like are used herein interchangeably to refer to any polymer of amino acid residues of any length. The polymer can be linear or non-linear (e.g., branched), it can comprise modified amino acids or amino acid analogs. The term also encompasses amino acid polymers that have been modified naturally or by intervention, for example, by disulfide bond formation, glycosylation, lipidation, acetylation, phosphorylation, or any other modification or manipulation, for example conjugation with labeling or bioactive components.

Unless otherwise indicated, conventional techniques of molecular biology, microbiology, cell biology, biochemistry and immunology, which are within the skill of the art may be employed in practicing the methods described herein. Such techniques are explained fully in the literature, such as, Molecular Cloning: A Laboratory Manual, second edition (Sambrook et al., 1989); Oligonucleotide Synthesis (M. J. Gait, ed., 1984); Animal Cell Culture (R. I. Freshney, ed., 1987); Handbook of Experimental Immunology (D. M. Weir & C. C. Blackwell, eds.); Gene Transfer Vectors for Mammalian Cells (J. M. Miller & M. P. Calos, eds., 1987); Current Protocols in Molecular Biology (F. M. Ausubel et al., eds., 1987); PCR: The Polymerase Chain Reaction, (Mullis et al., eds., 1994); Current Protocols in Immunology (J. E. Coligan et al., eds., 1991); The Immunoassay Handbook (David Wild, ed., Stockton Press NY, 1994); Antibodies: A Laboratory Manual (Harlow et al., eds., 1987); and Methods of Immunological Analysis (R. Masseyeff, W. H. Albert, and N. A. Staines, eds., Weinheim: VCH Verlags gesellschaft mbH, 1993).

A lipid containing moiety is covalently conjugated (that is, covalenly bound) to at least one amino acid of the CGRP peptide of the peptide conjugate of the invention, for example via a heteroatom of a side chain of the amino acid, such as a sulfur atom of a sulfide group. Lipid containing moieties of various structures are contemplated for use herein. A lipid containing moiety comprises a lipid and may comprise one or more other moiety, for example through which the lipid is attached to the amino acid. The term “lipid” as used herein unless indicated otherwise refers to substances that are soluble in organic solvents, including, but not limited to, oils, fats, fatty acids and esters thereof, and the like. In various embodiments, the lipid or lipid containing moiety is lipophilic and/or hydrophobic.

### Methods of Preparation

The peptide conjugates of the invention may be prepared using the methods and procedures described herein. Other suitable methods for preparing compounds of the invention will be apparent to those skilled in the art.

The peptide conjugates of the invention may be prepared from readily available starting materials using the methods and procedures described herein. It will be appreciated that where typical or preferred process conditions (for example, reaction temperatures, times, mole ratios of reactants, solvents, pressures, etc.) are indicated, other process conditions can also be used unless otherwise stated. Optimum reaction conditions may vary with the particular reactants used.

Conventional protecting groups may be necessary to prevent certain functional groups from undergoing undesired reactions. The need for protection and deprotection and the selection of appropriate protecting groups can be readily determined by a person skilled in the art. Suitable protecting groups for various functional groups as well as suitable conditions for protecting and deprotecting particular functional groups are well known in the art (see, for example, T. W. Greene and G. M. Wuts, Protecting Groups in Organic Synthesis, Third Edition, Wiley, New York, 1999).

The starting materials useful in the methods and reactions are commercially available or can be prepared by known procedures or modifications thereof, for example those described in in standard reference texts such as Fieser and Fieser's Reagents for Organic Synthesis, Volumes 1-15 (John Wiley and Sons, 1991), Organic Reactions, Volumes 1-40 (John Wiley and Sons, 1991), March's Advanced Organic Chemistry, (John Wiley and Sons, 4^{th} Edition), and Larock's Comprehensive Organic Transformations (VCH Publishers Inc., 1989).

The various starting materials, intermediates, and compounds may be isolated and purified where appropriate using conventional techniques such as precipitation, filtration, crystallization, evaporation, distillation, and chromatography. Characterization of the compounds may be performed using conventional methods such as by melting point, mass spectrum, nuclear magnetic resonance, and various other spectroscopic analyses.

The present invention relates to a method for preparing a peptide conjugate of the invention comprising
(A) providing an amino acid conjugate comprising an amino acid of a calcitonin gene-related peptide (CGRP) peptide, wherein the amino acid is covalently conjugated to a lipid-containing moiety via a sulfur atom of a sulfide group; and
coupling the amino acid of the amino acid conjugate to one or more amino acids and/or one or more peptides to provide the peptide conjugate of the invention; or
(B) providing a peptide-conjugate comprising a peptide fragment of a calcitonin gene-related peptide (CGRP) peptide, wherein at least one amino acid of the peptide fragment is covalently conjugated to a lipid-containing moiety via a sulfur atom of a sulfide group; and
coupling an amino acid of the peptide conjugate to one or more amino acids and/or one or more peptides to provide the peptide conjugate of the invention.

The present invention also relates to a method for preparing a peptide conjugate of the invention, the method comprising reacting
a lipid-containing conjugation partner comprising a carbon carbon double bond, and
an amino acid-comprising conjugation partner comprising at least one amino acid comprising a thiol
under conditions effective to conjugate the lipid-containing conjugation partner to the amino acid-comprising conjugation partner.

The present invention also relates to a peptide conjugate of the present invention made by a method of the present invention.

The amino acid acid conjugate comprising an amino acid of a calcitonin gene-related peptide (CGRP) peptide of (A) or peptide-conjugate comprising a peptide fragment of a calcitonin gene-related peptide (CGRP) peptide of (B) may be provided by methods known in the art or analogous thereto. Such methods include the conjugation methods described in WO 2014/207708 A2, WO 2016/103192 A1, and WO 2017/145097 A2, each of which are incorporated herein by reference in their entirety.

Such methods also include the thiol-ene reaction based conjugation method of the present invenion.

The thiol-ene reaction involves the addition of a thiol across a non-aromatic carbon-carbon double bond (i.e. hydrothiolation of the carbon-carbon double bond). In the method the present invention, the amino acid-comprising conjugation partner comprises the thiol and the lipid-containing conjugation partner comprises the carbon carbon double bond. The lipid-containing conjugation partner and amino acid-comprising conjugation partner in the reaction are as defined in any of the embodiments described herein.

The reaction proceeds via a free radical mechanism. There are three distinct phases in the reaction: initiation, polymerisation or coupling, and termination. Radical generation gives rise to an electrophilic thiyl radical which propagates across the ene group, forming a carbon-centred radical.

Typically, the carbon-centred radical may be quenched by chain transfer from additional thiol molecule to give the final hydrothiolation product. However, in some embodiments, depending on the reaction conditions used, the carbon-centred radical may react with a carbon carbon double bond of a second molecule of a lipid-containing conjugation partner to provide a bis-addition product (or bis-adduct) in which the sulfur atom from the thiol is conjugated to a carbon atom from the carbon-carbon double bond of a first lipid-containing conjugation partner, and a carbon atom from the carbon-carbon double bond of the first lipid-containing conjugation partner is conjugated to a carbon atom from the carbon-carbon double bond of a second lipid-containing conjugation partner. Preferably, the first lipid-containing conjugation partner and second lipid-containing conjugation partner are identical. The two pathways are believed to be competitive. The method of the present invention encompasses the preparation of both such mono- and bis-lipid containing conjugation partner addition products.

In some embodiments, the amino acid comprising conjuation parter is a peptide-containing conjugation partner. In other embodiments, the amino acid comprising conjugation partner comprises, consists essentially of, or consists of an amino acid (as opposed to a peptide).

In some embodiments, the amino acid comprising conjugation partner comprises a peptide of a CGRP peptide, wherein at least one amino acid of the peptide comprises a thiol. In some of such embodiments, reaction with the lipid-containing conjugation partner provides a peptide conjugate of the invention.

In other embodiments, the amino acid comprising conjugation partner comprises a peptide fragment of a CGRP peptide, wherein at least one amino acid of the peptide fragment comprises a thiol. In other embodiments, the amino acid comprising conjugation partner comprises an amino acid of a CGRP peptide, wherein the amino acid comprises a thiol. In some of such embodiments, reaction with the lipid containing conjugation partner provides an amino acid conjugate or peptide conjugate, which may be coupled to one or more amino acids and/or one or more peptides to provide a peptide conjugate of the invention.

One or more free radicals may be generated in the reaction by any method known in the art. The free radicals may be generated thermally and/or photochemically. One or more free radical initiators may be used to initiate the generation of free radicals. Suitable free radical initiators include thermal initiators and photoinitiators.

Free radicals are generated from thermal initiators by heating. The rate of degradation of the thermal initiator and resulting free radical formation depends on the initiator and the temperature at which the initiator is heated. Higher temperatures generally result in faster decomposition. A person skilled in the art will be able to select an appropriate temperature for heating the initiator without undue experimentation. Numerous thermal initiators are commercially available. Examples of thermal initiators include but are not limited to tert-amyl peroxybenzoate, 1,1′-azobis(cyclohexanecarbonitrile), 2,2′-azobisisobutyronitrile (AIBN), benzoyl peroxide, tert-butyl hydroperoxide, tert-butyl peracetate, tert-butyl peroxide, tert-butyl peroxybenzoate, tert-butylperoxy isopropyl carbonate, lauroyl peroxide, peracetic acid, and potassium persulfate.

Free radicals may be generated from photoinitiators by irradiation with light. The frequency of light necessary to induce degradation of the photoinitiators and free radical formation depends on the initiator. Many photoinitiators can be initiated with ultraviolet light. Light of a specific wavelength or wavelength range may be used to selectively irradiate the initiator, where the lipid-containing conjugation partner or amino acid-comprising conjugation partner, for example a peptide-containing conjugation partner, comprises photosensitive groups. In certain embodiments of the method of the present invention, a frequency of about 365 nm is used. Light of this frequency is generally compatible with the side chains of naturally occurring amino acids.

A wide range of photoinitiators are commercially available. Examples of photoinitiators include but are not limited to acetophenone, anisoin, anthraquinone, anthraquinone-2-sulfonic acid, benzil, benzoin, benzoin ethyl ether, benzoin isobutyl ether, benzoin methyl ether, benzophenone, 3,3′,4,4′-benzophenonetetracarboxylic dianhydride, 4-benzoylbiphenyl, 2-benzyl-2-(dimethylamino)-4′-morpholinobutyrophenone, 4′-bis(diethylamino)benzophenone, 4,4′-bis(dimethylamino)benzophenone, camphorquinone, 2-chlorothioxanthen-9-one, dibenzosuberenone, 2,2-diethoxyacetophenone, 4,4′-dihydroxybenzophenone, 2,2-dimethoxy-2-phenylacetophenone (DMPA), 4-(dimethylamino)benzophenone, 4,4′-dimethylbenzil, 2,5-dimethylbenzophenone, 3,4-dimethylbenzophenone, 4′-ethoxyacetophenone, 2-ethylanthraquinone, 3′-hydroxyacetophenone, 4′-hydroxyacetophenone, 3-hydroxybenzophenone, 4-hydroxybenzophenone, 1-hydroxycyclohexyl phenyl ketone, 2-hydroxy-2-methylpropiophenone, 2-methylbenzophenone, 3-methylbenzophenone, methybenzoylformate, 2-methyl-4′-(methylthio)-2-morpholinopropiophenone, phenanthrenequinone, 4′-phenoxyacetophenone, and thioxanthen-9-one.

A person skilled in the art will be able to select appropriate free radical initiators for use in the method having regard to, for example, the nature of the lipid-containing conjugation partner, amino acid-comprising conjugation partner, and any other components present in the reaction mixture. In some embodiments, the initiator is present in the reaction in a stoichiometric ratio relative to the starting material comprising the thiol of from about 20:1 to about 0.05:1, from about 10:1 to about 0.05:1, from about 5:1 to about 0.05:1, from about 3:1 to about 0.5:1.

The lipid-containing conjugation partner and amino acid-comprising conjugation partner may be prepared using known synthetic chemistry techniques or modifications thereof (for example, the methods generally described in Louis F Fieser and Mary F, Reagents for Organic Synthesis v. 1-19, Wiley, New York (1967-1999 ed.) or Beilsteins Handbuch der organischen Chemie, 4, Aufl. Ed. Springer-Verlag Berlin, including supplements (also available via the Beilstein online database)) or, in some embodiments, may be commercially available.

For example, lipid-containing conjugation partner compounds of the formula (II)

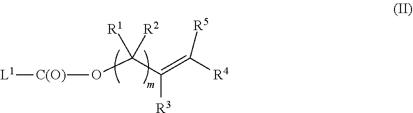

wherein m, L¹, R¹, R², R³, R⁴, and R are as defined in any of the embodiments described herein; and Z¹ when present is —C(O)O— may be prepared by reacting a compound of the formula (VI)

L¹-C(O)—X (VI)

wherein X is OH or a suitable leaving group with a compound of the formula (VII):

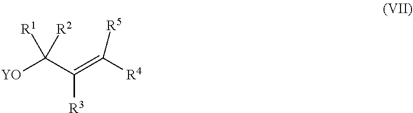

wherein Y is H, a metal or metalloid, or acyl (for example, alkylcarbonyl) under conditions effective for esterification (or transesterification where Y is an acyl group).

Methods for esterification are well known in the art. For example, when X is chloro, the reaction may be carried out in the presence of a base, such as pyridine or triethylamine, in a suitable solvent. The acid chloride may be converted in situ to a more reactive species (e.g. to the corresponding iodide, using sodium iodide). The temperature at which the reaction is carried out depends on the reactivity of the acid species and the solvent used.

For example, vinyl esters of the formula (II) may be produced by transesterification with vinyl acetate (itself produced industrially by the reaction of acetic acid and acetylene or acetic acid and ethylene over a suitable catalyst) using an acid or metal catalyst. See, for example, EP 0376075 A2 and S. K. Karmee, J. Oil Palm Res., 2012, 1518-1523. Vinyl esters of the formula (II) may also be prepared by the addition a carboxylic acid to a terminal acetylene in the presence of a catalyst (usually a palladium or ruthenium complex). See, for example, V. Cadierno, J. Francos, J. Gimeno Organometallics, 2011, 30, 852-862; S. Wei, J. Pedroni, A. Meissner, A. Lumbroso, H.-J. Drexler, D. Heller, B. Breit, Chem. Eur. J., 2013, 19, 12067-12076. Non-terminal acetylenes may also be reacted. See, for example, N. Tsukada, A. Takahashi, Y. Inoue, Tetrahedron Lett., 2011, 52, 248-250 and M. Rotem, Y. Shvo, J. Organometallic Chem. 1993, 448, 159-204.

Further examples of methods for preparing vinyl esters of formula (II) include: reaction of divinylmercury with aromatic and aliphatic acids [see, for example, D. J. Foster, E. Tobler, J. Am. Chem. Soc. 1961, 83, 851]; Cu(II)-catalyzed esterification of arene carboxylic acids with trimethoxy(vinyl)silane in the presence of AgF [see, for example, F. Luo, C. Pan, P. Qian, J. Cheng, Synthesis 2010, 2005]; vinyl transfer reactions from vinyl acetate to primary and secondary alcohols, and also to carboxylic acids with a catalyst system consisting of 2 mol-% of [AuCl(PPh₃)] and 2 mol-% of AgOAc [see, for example, A. Nakamura, M. Tokunaga, Tetrahedron Lett. 2008, 49, 3729]; and Ir complex ([Ir(cod)Cl]₂/P(OMe)₃)-catalyzed transvinylation [see, for example, H. Nakagawa, Y. Okimoto, S. Sakaguchi, Y. Ishii, Tetrahedron Lett. 2003, 44, 103]. Other suitable methods for preparing compounds of formula (II) will be apparent to those skilled in the art.

Numerous compounds of formula (VI) are commercially available. Others may be prepared using standard synthetic chemistry techniques from commercially available precursors. For example, compounds of formula (VI) wherein X is chloro may be prepared treating the corresponding carboxylic acid with thionyl chloride in a suitable solvent or mixture of solvents. Similarly, compounds of formula (VII) are also commercially available or may be prepared from commercially available precursors using standard synthetic chemistry techniques.

The order in which the lipid-containing conjugation partner and amino acid-comprising conjugation partner and any other components present in the reaction mixture are introduced into the reaction vessel may vary. The reaction may be carried out as a one-pot procedure.

The ratio of the lipid-containing conjugation partner to amino acid-comprising conjugation partner in the reaction may also vary. In some embodiments, the mole ratio of the first lipid-containing conjugation partner and second lipid-containing conjugation partner combined (i.e. in total) to the amino acid-comprising conjugation partner is at least 7:1, for example 8:1, 9:1, 10:1, 20:1, 30:1, 35:1, 40:1, 50:1, 60:1, or 70:1.

The reaction may be carried out at any suitable temperature. In some embodiments, the reaction is carried out at a temperature from about −25° C. to about 200° C., from about −10° C. to about 150° C., from about 0° C. to about 125° C., from about ambient temperature to about 100° C. In some embodiments, the reaction is carried out at ambient temperature. In some embodiments, the reaction is carried out at a temperature above ambient temperature.

In one embodiment, the reaction is carried out at a temperature from 40 to 200° C., from 50 to 150° C., from 60 to 100° C., from 65 to 90° C., or from 70 to 80° C.

The temperature at which the reaction is carried out may depend on how free radicals are generated in the reaction. The temperature used may be selected to control the rate of the reaction and may be adjusted during the course of the reaction to control the rate of the reaction. If free radicals are generated thermally (e.g. using a thermal initiator), the reaction will generally be carried out at a temperature above ambient temperature. The temperature will depend on the reactivity of the species from which free radicals are generated. If free radicals are generated photochemically the reaction may be carried out, advantageously, at ambient temperature. In certain embodiments, it may be desirable to cool the reaction mixture to slow the rate of reaction or conversely heat the reaction mixture to increase the rate of reaction. A person skilled in the art will be able to select appropriate temperatures for carrying out the method having regard to the reactivity of the starting materials and other reactants present. The temperature at which the reaction is carried out may be controlled by heating or cooling the reaction mixture by suitable method known in the art. Heat may be applied to the reaction mixture, for example, using a heat exchanger within the reaction vessel, a heating jacket surrounding the reaction vessel, or by immersing the reaction vessel in a heated liquid (e.g. an oil or sand bath). In certain exemplary embodiments, the reaction mixture is heated by microwave irradiation.

The progress of the reaction may be monitored by any suitable means, for example, by thin layer chromatography (TLC) or high performance liquid chromatorgraphy (HPLC). The reaction may be allowed to proceed to substantial completion, as monitored by the consumption of at least one of the starting materials. In some embodiments, the reaction is carried out until at least about 50%, at least about 60%, at least about 70%, at least about 75%, at least about 80%, at least about 85%, at least about 90%, at least about 95%, at least about 97%, at least about 99% of the amino acid-comprising conjugation partner has been consumed. The consumption of starting materials may be monitored by any suitable method, for example, HPLC. In some embodiments, the reaction is allowed to proceed for a period of time from 1 minute to 7 days, 5 minutes to 72 hours, 10 minutes to 48 hours, 10 minutes to 24 hours. In other embodiments, the reaction is allowed to proceed for a period of time less than 72 h, less than 48 h, less than 24 h, less than 12 h, less than 6 h, less than 4 h, less than 2 h, or less than 1 h.

The reaction mixture may be mixed by any suitable method known in the art, for example, using a magnetic or mechanical stirrer. The method used may depend on the scale on which the reaction is carried out.

The reaction is generally carried out in a liquid reaction medium. The liquid reaction medium may comprise a solvent. Examples of suitable solvents include N-methylpyrrolidone (NMP), dimethylformamide, dichloromethane, 1,2-dichloroethane, chloroform, carbon tetrachloride, water, methanol, ethanol, dimethylsulfoxide, trifluoroacetic acid, acetic acid, acetonitrile, and mixtures thereof. The solvent may be selected based on the solubility of the starting materials and other reactants present, for example the free radical initiator. In some embodiments, the lipid-containing conjugation partner is hydrophobic. The hydrophobicity or hydrophilicity of an amino acid-comprising conjugation partner may vary depending on, for example, the amino acid sequence of the peptide of a peptide-containing conjugation partner. A person skilled in the art will be able to select an appropriate solvent or mixture of solvents without undue experimentation.

The reaction may be carried out under substantially oxygen-free conditions. Oxygen may quench free radicals formed in the reaction. The reaction mixture may be degassed with an inert gas (e.g. nitrogen or argon) that is substantially oxygen-free to remove any dissolved oxygen before free radicals are generated. Alternatively, individual components of the reaction mixture may be degassed with inert gas that is substantially oxygen-free prior to being combined in the reaction vessel. The reaction may be carried out under an atmosphere of inert gas that is substantially oxygen-free. The method may be carried out at ambient pressure.

One or more additive that inhibits the formation of undesireable by-products and/or that improves the yield of or conversion to the desired product may be included in the reaction mixture in the thiolene method of the present invention. The additive is generally used in an amount sufficient to minimise the formation of undesirable by products without adversely affecting the reaction or any, optional, subsequent steps in the method. The one or more additive may be an extraneous thiol, an acid, an organosilane, or a combination of any two or more thereof.

The inventors have found that in some embodiments the inclusion of an extraneous or exogenous thiol as an additive in the reaction mixture reduces the formation of undesirable by products. The extraneous thiol may, in some embodiments, increase the efficiency or conversion of the desired thiolene reaction. Examples of suitable extraneous thiols include but are not limited to reduced glutathione, DODT, DTT, protein, sterically hindered thiols, and the like. In some embodiments, the extraneous thiol is DTT. In other embodiments, the extraneous thiol is a sterically hindered thiol. Non-limiting examples of a suitable sterically hindered extraneous thiol include tert-butyl mercaptan and 1-methylpropyl mercaptan.

In various embodiments, the extraneous thiol is present in the reaction in a stoichiometric ratio relative to the amino acid comprising conjugation partner of from about 200:1 to about 0.05:1, 100:1 to 0.05:1, 80:1 to 0.05:1, 60:1 to 0.05:1, 40:1 to 0.05:1, 20:1 to about 0.05:1, 10:1 to about 0.5:1, 5:1 to about 1:1, or 3:1 to about 1:1. In certain embodiments, a sterically hindered thiol such as t-BuSH is present in the reaction in a stoichiometric ratio relative to the amino acid comprising conjugation partner of from about 100:1 to 0.05:1, for example about 80:1, about 40:1, or about 3:1. Without wishing to be bound by theory, the inventors believe that in certain embodiments an extraneous thiol such as tert-butylmercaptan can provide a proton to quench carbon centred radical intermediates formed by propagation during the reaction and that the resulting tert-butyl-thiyl radical can propagate the reaction by generating another mole of thiyl radical from the amino acid comprising conjugation partner.

The inclusion of an acid in some embodiments may also reduce the formation of undesireable by-products. The acid may be a strong inorganic acid, for example HCl, or organic acid, for example TFA. In certain embodiments, the additive is TFA. Without wishing to be bound by theory, the inventors believe that decreasing the pH of the reaction mixture may result in the protonation of electron rich side chains of residues such as lysine, etc. which could otherwise participate in single electron transfers and form radical species in the reaction. In various embodiments, the reaction mixture comprises from about 0.01 to 25, 0.01 to 15, 0.01 to 10, or 1 to 10% v/v acid additive. In certain embodiments, the reaction mixture comprises from 1-10% v/v TFA, for example 5% v/v TFA.

The inventors have found that in some embodiments including both tert-butyl mercaptan and TFA as additives in the reaction mixture can reduce the the formation of undesirable by products and increase the conversion of starting material to the desired product. Accordingly, in certain exemplary embodiments, the reaction mixture comprises a combination of an acid and an exogenous thiol, such as a combination of a strong organic acid and a sterically hindered thiol, for example a combination of TFA and tert-butyl mercaptan.

An organosilane may also be included as an additive in the thiolene reaction. Organosilanes are radical-based reducing agents, the activity of which can be modulated by varying the substituents on the silicon atom. In various embodiments, the organosilane is a compound of the formula (R^{q})₃SiH, wherein R^{q} at each instance is independently hydrogen or an organic group, for example alkyl or aryl, provided that at least one R^{q} is not hydrogen. Examples of organosilanes include but are not limited to triethylsilane (TES), triphenylsilane, diphenylsilane, triisopropylsilane (TIPS), and the like. In various embodiments, the organosilane is a trialkylsilane, for example TIPS or TES. Without wishing to be bound by theory, the inventors believe that, as with an extraneous thiol, in certain embodiments an organosilane such as TIPS can act as a hydrogen donor to provide the desired conjugate and promote propagation of the reaction. In various embodiments, the organosilane is present in the reaction in a stoichiometric ratio relative to the amino acid comprising conjugation partner of from about 200:1 to about 0.05:1, 100:1 to 0.05:1, 80:1 to 0.05:1, 60:1 to 0.05:1, 40:1 to 0.05:1, 20:1 to 0.05:1, 10:1 to 0.5:1, 5:1 to about 1:1, or 3:1 to about 1:1. In certain embodiments, a trialkylsilane such as TIPS is present in the reaction in a stoichiometric ratio relative to the amino acid comprising conjugation partner of from about 100:1 to 0.05:1, for example about 80:1 or about 40:1.

The organosilane may be used as an additive in combination with an extraneous thiol. Alternatively, the organosilane may be used instead of an extraneous thiol. An acid, such as TFA, may also be present.

The products formed in the reaction and conversion to the desired product may be determined by, for example, HPLC.

The concentration of the lipid-containing conjugation partner and amino acid-comprising conjugation partner, respectively, in the reaction mixture may also affect the reaction. Those skilled in the art will be able to vary the concentration of the lipid-containing conjugation partner and peptide-containing conjugation partner in the reaction mixture to e.g. optimise yield and purity without undue experimentation. In some embodiments, the starting material comprising the thiol is present in a concentration from about 0.05 mM to about 1 M, from about 0.5 mM to about 1 M, from about 1 mM to about 1 M. In some embodiments, the concentration is at least about 0.05 mM, 0.5 mM, or 1 mM. In some embodiments, the concentration of the starting materials comprising the alkenes is at least about 0.05 mM, 0.5 mM, or 1 mM.

In some embodiments, the amino acid conjugate or peptide conjugate is separated from the reaction medium after the conjugation reaction and optionally purified. The conjugate may be separated from the reaction medium using any suitable method known in the art, for example, by precipitation. In some embodiments, the amino acid or peptide conjugate is purified after separating it from the reaction medium. For example, the conjugate may be purified by HPLC using one or more suitable solvents.

The peptide conjugates of the present invention produced by, the peptide conjugates used in, the amino acid-comprising conjugation partners used in, and/or the peptides coupled in the methods of the present invention may comprise a synthetic peptide. Synthetic peptides may be prepared using solid phase peptide synthesis (SPPS).

The coupling of one or more amino acids and/or one or more peptides to provide peptides or peptide conjugates, for example a peptide conjugate of the invention, in the methods of the invention, thus, may also be carried out by SPPS.

Synthetic peptides may also be prepared by liquid phase peptide synthesis.

### Solid Phase Peptide Synthesis (SPPS)

The basic principle for SPPS is a stepwise addition of amino acids to a growing polypeptide chain anchored via a linker molecule to a solid phase support, typically a resin particle, which allows for cleavage and purification once the polypeptide chain is complete. Briefly, a solid phase resin support and a starting amino acid are attached to one another via a linker molecule. Such resin-linker-acid matrices are commercially available.

The amino acid to be coupled to the resin is protected at its Nα-terminus by a chemical protecting group. The amino acid may also have a side-chain protecting group. Such protecting groups prevent undesired or deleterious reactions from taking place during the process of forming the new peptide bond between the carboxyl group of the amino acid to be coupled and the unprotected Nα-amino group of the peptide chain attached to the resin.

The amino acid to be coupled is reacted with the unprotected Nα-amino group of the N-terminal amino acid of the peptide chain, increasing the chain length of the peptide chain by one amino acid. The carboxyl group of the amino acid to be coupled may be activated with a suitable chemical activating agent to promote reaction with the Nα-amino group of the peptide chain. The Nα-protecting group of N-terminal amino acid of the peptide chain is then removed in preparation for coupling with the next amino acid residue. This technique consists of many repetitive steps making automation attractive whenever possible. Those skilled in the art will appreciate that peptides may be coupled to the Nα-amino group of the solid phase bound amino acid or peptide instead of an individual amino acid, for example where a convergent peptide synthesis is desired.

When the desired sequence of amino acids is achieved, the peptide is cleaved from the solid phase support at the linker molecule.

SPPS may be carried out using a continuous flow method or a batch flow method. Continuous flow permits real-time monitoring of reaction progress via a spectrophotometer, but has two distinct disadvantages—the reagents in contact with the peptide on the resin are diluted, and scale is more limited due to physical size constraints of the solid phase resin. Batch flow occurs in a filter reaction vessel and is useful because reactants are accessible and can be added manually or automatically.

Two types of protecting groups are commonly used for protecting the N-alpha-amino terminus: “Boc” (tert-butyloxycarbonyl) and “Fmoc” (9-fluorenylmethyloxycarbonyl). Reagents for the Boc method are relatively inexpensive, but they are highly corrosive and require expensive equipment and more rigorous precautions to be taken. The Fmoc method, which uses less corrosive, although more expensive, reagents is typically preferred.

For SPPS, a wide variety of solid support phases are available. The solid phase support used for synthesis can be a synthetic resin, a synthetic polymer film or a silicon or silicate surface (e.g. controlled pore glass) suitable for synthesis purposes. Generally, a resin is used, commonly polystyrene suspensions, or polystyrene-polyethyleneglycol, or polymer supports for example polyamide.

Examples of resins functionalized with linkers suitable for Boc-chemistry include PAM resin, oxime resin SS, phenol resin, brominated Wang resin and brominated PPOA resin. Examples of resins suitable for Fmoc chemistry include amino-methyl polystyrene resins, AMPB-BHA resin, Sieber amide resin, Rink acid resin, Tentagel S AC resin, 2-chlorotrityl chloride resin, 2-chlorotrityl alcohol resin, TentaGel S Trt-OH resin, Knorr-2-chlorotrityl resin, hydrazine-2-chlorotrityl resin, ANP resin, Fmoc photolable resin, HMBA-MBHA resin, TentaGel S HMB resin, Aromatic Safety Catch resinBAl resin and Fmoc-hydroxylamine 2 chlorotrityl resin. Other resins include PL C₁-Trt resin, PL-Oxime resin and PL-HMBA Resin. Generally resins are interchangeable.

For each resin appropriate coupling conditions are known in the literature for the attachment of the starting monomer or sub-unit.

Preparation of the solid phase support includes solvating the support in an appropriate solvent (e.g. dimethylformamide). The solid phase typically increases in volume during solvation, which in turn increases the surface area available to carry out peptide synthesis.

A linker molecule is then attached to the support for connecting the peptide chain to the solid phase support. Linker molecules are generally designed such that eventual cleavage provides either a free acid or amide at the C-terminus. Linkers are generally not resin-specific. Examples of linkers include peptide acids for example 4-hydroxymethylphenoxyacetyl-4′-methylbenzyhydrylamine (HMP), or peptide amides for example benzhydrylamine derivatives.

The first amino acid of the peptide sequence may be attached to the linker after the linker is attached to the solid phase support or attached to the solid phase support using a linker that includes the first amino acid of the peptide sequence. Linkers that include amino acids are commercially available.

The next step is to deprotect the Nα-amino group of the first amino acid. For Fmoc SPPS, deprotection of the Nα-amino group may be carried out with a mild base treatment (piperazine or piperidine, for example). Side-chain protecting groups may be removed by moderate acidolysis (trifluoroacetic acid (TFA), for example). For Boc SPPS, deprotection of the Nα-amino group may be carried out using for example TFA.

Following deprotection, the amino acid chain extension, or coupling, proceeds by the formation of peptide bonds. This process requires activation of the C-α-carboxyl group of the amino acid to be coupled. This may be accomplished using, for example, in situ reagents, preformed symmetrical anhydrides, active esters, acid halides, or urethane-protected N-carboxyanhydrides. The in situ method allows concurrent activation and coupling. Coupling reagents include carbodiimide derivatives, for example N,N′-dicyclohexylcarbodiimide or N,N-diisopropylcarbodiimide. Coupling reagents also include uronium or phosphonium salt derivatives of benzotriazol. Examples of such uronium and phosphonium salts include HBTU (O-1H-benzotriazole-1-yl)-N,N,N′,N′-tetramethyluronium hexafluorophosphate), BOP (benzotriazole-1-yl-oxy-tris-(dimethylamino)-phosphonium hexafluorophosphate), PyBOP (Benzotriazole-1-yl-oxy-tripyrrolidinophosphonium hexafluorophosphate), PyAOP, HCTU (O-(1H-6-chloro-benzotriazole-1-yl)-1,1,3,3-tetramethyluronium hexafluorophosphate), TCTU (O-1H-6-chlorobenzotriazole-1-yl)-1,1,3,3-tetramethyluronium tetrafluoroborate), HATU (0-(7-azabenzotriazol-1-yl)-1,1,3,3-tetramethyluronium hexafluorophosphate), TATU (0-(7-azabenzotriazol-1-yl)-1,1,3,3-tetramethyluronium tetrafluoroborate), TOTU (O-[cyano(ethoxycarbonyl)methyleneamino]-N,N,N′,N″-tetramethyluronium tetrafluoroborate), and HAPyU (O-(benzotriazol-1-yl)oxybis-(pyrrolidino)-uronium hexafluorophosphate. In some embodiments, the coupling reagent is HBTU, HATU, BOP, or PyBOP.

After the desired amino acid sequence has been synthesized, the peptide is cleaved from the resin. The conditions used in this process depend on the sensitivity of the amino acid composition of the peptide and the side-chain protecting groups. Generally, cleavage is carried out in an environment containing a plurality of scavenging agents to quench the reactive carbonium ions that originate from the protective groups and linkers. Common cleaving agents include, for example, TFA and hydrogen fluoride (HF). In some embodiments, where the peptide is bound to the solid phase support via a linker, the peptide chain is cleaved from the solid phase support by cleaving the peptide from the linker.

The conditions used for cleaving the peptide from the resin may concomitantly remove one or more side-chain protecting groups. In some embodiments, one or more or all protecting groups are removed on cleaving the peptide from the solid phase support.

The use of protective groups in SPPS is well established. Examples of common protective groups include but are not limited to acetamidomethyl (Acm), acetyl (Ac), adamantyloxy (AdaO), benzoyl (Bz), benzyl (Bzl), 2-bromobenzyl, benzyloxy (BzlO), benzyloxycarbonyl (Z), benzyloxymethyl (Bom), 2-bromobenzyloxycarbonyl (2-Br—Z), tert-butoxy (tBuO), tert-butoxycarbonyl (Boc), tert-butoxymethyl (Bum), tert-butyl (tBu), tert-buthylthio (tButhio), 2-chlorobenzyloxycarbonyl (2-Cl—Z), cyclohexyloxy (cHxO), 2,6-dichlorobenzyl (2,6-DiCl-Bzl), 4,4′-dimethoxybenzhydryl (Mbh), 1-(4,4-dimethyl-2,6-dioxo-cyclohexylidene)3-methyl-butyl (ivDde), 4-{N-[1-(4,4-dimethyl-2,6-dioxo-cyclohexylidene)3-methylbutyl]-amino) benzyloxy (ODmab), 2,4-dinitrophenyl (Dnp), fluorenylmethoxycarbonyl (Fmoc), formyl (For), mesitylene-2-sulfonyl (Mts), 4-methoxybenzyl (MeOBzl), 4-methoxy-2,3,6-trimethyl-benzenesulfonyl (Mtr), 4-methoxytrityl (Mmt), 4-methylbenzyl (MeBzl), 4-methyltrityl (Mtt), 3-nitro-2-pyridinesulfenyl (Npys), 2,2,4,6,7-pentamethyldihydrobenzofurane-5-sulfonyl (Pbf), 2,2,5,7,8-pentamethyl-chromane-6-sulfonyl (Pmc), tosyl (Tos), trifluoroacetyl (Tfa), trimethylacetamidomethyl (Tacm), trityl (Trt) and xanthyl (Xan).

Where one or more of the side chains of the amino acids of the peptide contains functional groups, such as for example additional carboxylic, amino, hydroxy or thiol groups, additional protective groups may be necessary. For example, if the Fmoc strategy is used, Mtr, Pme, Pbf may be used for the protection of Arg; Trt, Tmob may be used for the protection of Asn and Gln; Boc may be used for the protection of Trp and Lys; tBu may be used for the protection of Asp, Glu, Ser, Thr and Tyr; and Acm, tBu, tButhio, Trt and Mmt may be used for the protection of Cys. A person skilled in the art will appreciate that there are numerous other suitable combinations.

The methods for SPPS outlined above are well known in the art. See, for example, Atherton and Sheppard, “Solid Phase Peptide Synthesis: A Practical Approach,” New York: IRL Press, 1989; Stewart and Young: “Solid-Phase Peptide Synthesis 2nd Ed.,” Rockford, Ill.: Pierce Chemical Co., 1984; Jones, “The Chemical Synthesis of Peptides,” Oxford: Clarendon Press, 1994; Merrifield, J. Am. Soc. 85:2146-2149 (1963); Marglin, A. and Merrifield, R. B. Annu. Rev. Biochem. 39:841-66 (1970); and Merrifield R. B. JAMA. 210(7):1247-54 (1969); and “Solid Phase Peptide Synthesis—A Practical Approach” (W. C. Chan and P. D. White, eds. Oxford University Press, 2000). Equipment for automated synthesis of peptides or polypeptides is readily commercially available from suppliers such as Perkin Elmer/Applied Biosystems (Foster City, Calif.) and may be operated according to the manufacturer's instructions.

Following cleavage from the resin, the peptide may be separated from the reaction medium, e.g. by centrifugation or filtration. The peptide may then be subsequently purified, e.g. by HPLC using one or more suitable solvents.

In some embodiments the peptide-containing conjugation partner may be used in the methods of the present invention without purification following cleavage of the peptide from the resin.

In some embodiments the method of the present invention can be carried out using a peptide-containing conjugation partner, wherein the peptide does not contain an Nα-amino group protecting group or any side chain protecting groups. The reaction is generally selective for reaction of a thiol and a non-aromatic carbon-carbon double bond.

In some embodiments, the method comprises providing a protected amino acid comprising conjugation partner comprising at least one amino acid comprising a thiol protected with a protecting group; and removing the protecting group from the thiol to provide the amino acid comprising conjugation partner. A protected amino acid comprising conjugation partner may comprise one or more additional amino acids protected with protecting groups. The protecting groups of the one or more additional protected amino acids may be different to the thiol protecting group, such that the thiol protecting group may be selectively removed to facilitate reaction with the lipid containing conjugation partner.

It may be necessary to protect thiol groups other than the thiol group to be reacted with lipid-containing conjugation partner in the peptide-containing conjugation partner (e.g. in other cysteine residues of the peptide) with a protective group to prevent undesirable competing reactions in the methods of the present invention. Such thiol groups may be protected with a protective group that is not removable under the conditions used to remove one or more other protecting groups present in the peptide or to cleave the peptide from the resin. Typically, the peptide will be synthesised using amino acids bearing the appropriate protecting groups. A person skilled in the art will be able to select appropriate protecting groups without undue experimentation.

The amino acid-comprising conjugation partner and/or lipid-containing conjugation partner may comprise one or more unsaturated carbon-carbon bonds in addition to the carbon-carbon double bonds of the lipid containing conjugation partner to be reacted. Those skilled in the art will appreciate that the selectivity of the thiol for the carbon-carbon double bond to be reacted in such embodiments may depend on, for example, the steric and/or electronic environment of the carbon-carbon double bond relative to the one or more additional unsaturated carbon-carbon bonds. In certain embodiments, the carbon-carbon double bonds to be reacted are activated relative to any other unsaturated carbon-carbon bonds in the amino acid-comprising conjugation partner and lipid-containing conjugation partner. In certain embodiments, the carbon-carbon double bonds to be reacted are activated relative to any other unsaturated carbon-carbon bonds in the peptide-containing conjugation partner and lipid-containing conjugation partner.

In some embodiments, the Nα-amino group of the N-terminal amino acid of the peptide conjugate of the invention is acylated, for example acetylated. Thus, in some embodiments, the methods of the present invention may comprise acylating, for example acetylating, the Nα-amino group of the N-terminal amino acid of the peptide or peptide conjugate.

Where a peptide-containing conjugation partner and/or peptide conjugate has been synthesised by SPPS, acylation may be carried out prior to or after cleavage from the resin. In some embodiments, the method comprises acylating the N-terminal amino group prior to cleavage from the resin. In some embodiments, the method comprises acylating, for example acetylating, the Nα-amino group of the N-terminal amino acid of the amino acid conjugate or the amino acid residue of the peptide conjugate to which the lipid containing moiety is conjugated.

Acylation of the Nα-amino group of an amino acid may be carried out by reacting an amino acid or peptide with an acylating agent in the presence of base in a suitable solvent, for example DMF. Non-limiting examples of acylating agents include acid halides, for example acid chlorides such as acetyl chloride, and acid anhydrides, for example acetic anhydride. Such agents maybe commercially available or may be prepared by methods well known in the art. Non-limiting examples of suitable bases include triethylamine, diisopropylethylamine, 4-methylmorpholine, and the like.

One or more amino acid and/or one or more peptide that reduces peptide aggregation during SPPS may be coupled during the synthesis of the peptides described herein. Such agents are well known in the art. Examples include, but are not limited to, pseudoproline dipeptides, such as Fmoc-Leu-Ser[Ψ(Me,Me)Pro]-OH, and the like. Pseudoproline dipeptides include serine- or threonine-derived oxazolidines and cysteine-derived thiazolidines. The dipeitdes are introduced into the peptide sequence using standard coupling methods, substituting any amino acid-Ser, amino acid-Thr, amino acid-Cys dipeptide motif. The native sequence is regenerated on deprotection and cleavage of the peptide from the solid phase support. In some embodiments, one or more amino acid and/or one or more peptide are coupled so as to form a cleaveable solubising group that reduces aggregation.

The methods of the invention may comprising coupling one or more amino acid and/or one or more peptide. The one or more amino acid and/or one or more peptide may be coupled by SPPS. In some embodiments, all of the one or more amino acid and/or one or more peptides are coupled by SPPS.

In some embodiments, the methods comprise coupling the amino acid of the amino acid conjugate to one or more amino acid and/or one or more peptide to provide the peptide conjugate of the invention. In some embodiments, the method comprises coupling the amino acid of the amino acid conjugate to an amino acid or peptide bound to a solid phase support by SPPS. In some embodiments, the method comprises coupling the amino acid of the amino acid conjugate to a peptide bound to a solid phase support by SPPS. The method may comprise synthesising the peptide bound to the solid phase support by SPPS.

In some embodiments, the method comprises coupling the amino acid of the amino acid conjugate or an amino acid of the peptide conjugate to one or more amino acid and/or one or more peptide to provide the peptide conjugate of the invention. The coupling may be carried out by SPPS as described herein.

In one embodiment, the peptide of the peptide conjugate to be coupled is bound to a solid phase support, and the method comprises coupling an amino acid of the peptide conjugate to be coupled to one or more amino acid and/or one or more peptide to provide a solid phase bound peptide conjugate. The coupling may be carried out by SPPS as described herein.

In an alternate embodiment, the method comprises coupling an amino acid of the peptide conjugate to an amino acid or peptide bound to a solid phase support by SPPS to provide a solid phase bound peptide conjugate.

It will be appreciated by those skilled in the art that coupling an amino acid or a peptide to another amino acid or peptide as described herein typically comprises forming a peptide bond between the Nα-terminus of the amino acid or an amino acid of the peptide of one coupling partner and the C-terminus of the amino acid or an amino acid of the peptide of the other coupling partner.

In some embodiments, the method of the present invention comprises synthesising the amino acid sequence of the peptide of the peptide-containing conjugation partner by SPPS; and reacting the peptide-containing conjugation partner with the lipid containing conjugation partner.

In some embodiments, synthesising the amino acid sequence of the peptide of the peptide-containing conjugation partner by SPPS comprises coupling one or more amino acid and/or one or more peptide to an amino acid or peptide bound to a solid phase support to provide the amino acid sequence of the peptide or a portion thereof. In certain embodiments, the amino acid sequence of the entire peptide of the peptide-containing conjugation partner is synthesised by SPPS.

The amino acid comprising conjugation partner, for example the peptide containing conjugation partner, may be reacted with the lipid-containing conjugation partner while bound to a solid phase support. Alternatively, the peptide containing conjugation partner may be cleaved from the solid phase support, and optionally purified, prior to reaction, for example with the lipid-containing conjugation partner.

Confirmation of the identity of the peptides synthesized may be conveniently achieved by, for example, amino acid analysis, mass spectrometry, Edman degradation, and the like.

The method of the present invention may further comprise separating the peptide conjugate of the invention from the liquid reaction medium. Any suitable separation methods known in the art may be used, for example, precipitation and filtration. The conjugate may be subsequently purified, for example, by HPLC using one or more suitable solvents.

The peptide conjugates, thus, may be pure or purified, or substantially pure or purified.

As used herein “purified” does not require absolute purity; rather, it is intended as a relative term where the material in question is more pure than in the environment it was in previously. In practice the material has typically, for example, been subjected to fractionation to remove various other components, and the resultant material has substantially retained its desired biological activity or activities. The term “substantially purified” refers to materials that are at least about 60% free, preferably at least about 75% free, and most preferably at least about 90% free, at least about 95% free, at least about 98% free, or more, from other components with which they may be associated during manufacture.

### Uses

The inventors have found that peptide conjugates of the present invention have useful CGRP receptor antagonist activity.

Accordingly, the present invention relates to a method of antagonising a CGRP receptor in a subject in need thereof, comprising administering to the subject an effective amount of a peptide conjugate of the present invention.

The present invention also relates to a method of treating a disease or condition mediated by or modulated by a CGRP receptor or characterised by excessive CGRP receptor activation, in a subject in need thereof, comprising administering to the subject a therapeutically effective amount of a peptide conjugate of the invention.

The present invention also relates to a method of treating a disease or condition associated with or characterised by increased vasodilation in a subject in need thereof, comprising administering to the subject a therapeutically effective amount of a peptide conjugate according to the present invention. The present invention also relates to the use of a peptide congute of the invention for the same and a peptide conjugate of the invention for use in the same. The diseases and conditions associated with or characterised by increased vasodilation are preferably those mediated by or modulated by a CGRP receptor or characterised by excessive CGRP receptor activation. In some embodiments, the disease or condition may include, for example, any form of hypotension that involves CGRP or CGRP receptor activation, for example, in microvascular events. In some embodiments, the disease or condition is nasal congestion. In some of such embodiments, the peptide conjugate acts as a decongestant and may be applied topically, for example as a nasal spray.

The present invention also relates to a method of treating a disease or condition selected from the group consisting of thermal injury, circulatory shock, menopausal hot flushes, asthma, sepsis, neurogenic inflammation, inflammatory skin conditions (for example psoriasis and contact dermatitis), allergic rhinitis, joint disorders (for example arthritis and temporomandibular joint disorder, preferably arthritis), cachexia (for example cancer-induced cachexia), pain, for example craniofacial pain disorders (for example migraine, headache, trigeminal neuralgia and dental pain, preferably migraine), and metabolic disorders or syndromes (for example obesity, type II diabetes, insulin resistance, dyslipidemia, hypertension, atherosclerosis and thrombosis) in a subject in need thereof, comprising administering to the subject a therapeutically effective amount of a peptide conjugate of the invention.

In various embodiments the present invention relates to a method of treating a disease or condition selected from pain or metabolic disorders.

In various embodiments the present invention relates to a method of treating a disease or condition, wherein the disease or condition is pain.

In various embodiments the present invention relates to a method of treating a disease or condition, wherein the disease or condition is migraine or headache (for example cluster headaches and post-traumatic headache).

In various embodiments the present invention relates to relates to a method of treating a disease or condition, wherein the disease or condition is migraine.

The present invention also relates to peptide conjugates for use in and uses of the peptide conjugates in antagonising a CGRP receptor.

The present invention also relates to peptide conjugates for use in and uses of the peptide conjugates in the treating a disease or condition mediated by or modulated by a CGRP receptor or characterised by excessive CGRP receptor activation.

The present invention also relates to peptide conjugates for use in and uses of the peptide conjugates in the manufacture of medicaments for antagonising a CGRP receptor and to peptide conjugates for use in and uses of the peptide conjugates in the manufacture of medicaments for treating a disease or condition mediated by or modulated by a CGRP receptor or characterised by excessive CGRP receptor activation.

A person skilled in the art will appreciate that the peptide conjugates described herein are useful for treating a variety of diseases and conditions. Examples of diseases and conditions mediated by CGRP receptors include, but are not limited to, thermal injury, circulatory shock, menopausal hot flushes, asthma, sepsis, neurogenic inflammation, inflammatory skin conditions (for example psoriasis and contact dermatitis), allergic rhinitis, joint disorders (for example arthritis and temporomandibular joint disorder, preferably arthritis), cachexia (for example cancer-induced cachexia), pain, for example craniofacial pain disorders (for example migraine, headache, trigeminal neuralgia and dental pain, preferably migraine), and metabolic disorders or syndromes (for example obesity, type II diabetes, insulin resistance, dyslipidemia, hypertension, atherosclerosis and thrombosis). For example, with respect to the treatment of metabolic disorders, for example as weight loss therapy, animal studies have shown that blockade of CGRP action can promote weight loss through increased energy expenditure. Therefore, in some embodiments the peptide conjugates described herein may be useful for weight loss therapy, for example in the treatment of metabolic disorders.

The present invention also relates to peptide conjugates for use in and uses of the peptide conjugates in the manufacture of a medicament for treating a disease or condition selected from the group consisting of thermal injury, circulatory shock, menopausal hot flushes, asthma, sepsis, neurogenic inflammation, inflammatory skin conditions (for example psoriasis and contact dermatitis), allergic rhinitis, joint disorders (for example arthritis and temporomandibular joint disorder, preferably arthritis), cachexia (for example cancer-induced cachexia), pain, for example craniofacial pain disorders (for example migraine, headache, trigeminal neuralgia and dental pain, preferably migraine), and metabolic disorders or syndromes (for example obesity, type II diabetes, insulin resistance, dyslipidemia, hypertension, atherosclerosis and thrombosis).

The present invention also relates to peptide conjugates for use in and uses of the peptide conjugates in and methods of antagonising the CGRP receptor to treat a disease or condition mediated by or modulated by the CGRP receptor or characterised by excessive CGRP receptor activation.

The present invention also relates to uses of the peptide conjugate of the invention to treat a disease or condition selected from the group consisting of thermal injury, circulatory shock, menopausal hot flushes, asthma, sepsis, neurogenic inflammation, inflammatory skin conditions (for example psoriasis and contact dermatitis), allergic rhinitis, joint disorders (for example arthritis and temporomandibular joint disorder, preferably arthritis), cachexia (for example cancer-induced cachexia), pain, for example craniofacial pain disorders (for example migraine, headache, trigeminal neuralgia and dental pain, preferably migraine), and metabolic disorders or syndromes (for example obesity, type II diabetes, insulin resistance, dyslipidemia, hypertension, atherosclerosis and thrombosis).

Thus, the present invention also relates to methods for treating such diseases or conditions comprising administering to the subject a therapeutically effective amount of a peptide conjugate of the invention.

The present invention also relates to peptide conjugates for use in and uses of the peptide conjugates in the manufacture of medicaments for treating such diseases and conditions

The present invention also relates to a method of antagonising a CGRP receptor comprising contacting a cell and a peptide conjugate according to the invention in an amount effective to antagonise the CGRP receptor.

A “subject” refers to a human or a non-human animal, preferably a vertebrate that is a mammal, preferably a human. Non-human mammals include, but are not limited to, farm animals, such as, cattle, sheep, swine, deer, and goats; sport and companion animals, such as, dogs, cats, and horses; and research animals, such as, mice, rats, rabbits, and guinea pigs. Preferably, the subject is a human.

The term “treatment”, and related terms such as “treating” and “treat”, as used herein, unless indicated otherwise, relates generally to treatment, of a human or a non-human subject, in which some desired therapeutic effect is achieved. The therapeutic effect may, for example, be inhibition, reduction, amelioration, halt, or prevention of the disease or condition.

A “therapeutically effective amount” (or “effective amount”) is an amount sufficient to effect beneficial or desired results, including clinical results. A therapeutically effective amount can be administered in one or more administrations by various routes of administration. The therapeutically effective amount to be administered to a subject depends on, for example, the purpose for administration, mode of administration, nature and dosage of any co-administered compounds, and characteristics of the subject, such as general health, other diseases, age, sex, genotype, body weight and tolerance to drugs. A person skilled in the art will be able to determine appropriate dosages having regard to these any other relevant factors.

The efficacy of a peptide conjugate can be evaluated both in vitro and in vivo. For example, the peptide conjugate can be tested in vitro or in vivo for its ability to act as an antagonist of a CGRP receptor. For in vivo studies, the peptide conjugate can be administered to an animal (e.g., a mouse), for example by injection, and its effects evaluated. For example, as described herein in the Examples, the peptide conjugates of the invention may be injected into mice and the effects on surface blood flow, which is a surrogate biological measure for migraine therapy, measured using laser Doppler imaging.

Based on the results, an appropriate dosage range and administration route can be determined.

The peptide conjugate is typically administered in the form of a pharmaceutical composition of the invention as described herein. The composition may be administered as a single dose or a multiple dose schedule.

The peptide conjugate can be used or administered as the sole therapeutic agent or in combination with one or more other additional therapeutic agents. The peptide conjugate and one or more additional therapeutic agents may be used or administered simultaneously, sequentially, or separately. The one or more additional therapeutic agents will depend on the disease or condition to be treated or other desired therapeutic benefit. The one or more additional therapeutic agents can be used in therapeutic amounts indicated or approved for the particular agent, as would be known to those skilled in the art. In some embodiments, two or more peptide conjugates of the invention are used or administered in combination.

The two or more peptide conjugates may be used or administered simultaneously, sequentially, or separately.

The present invention also relates to a method of antagonising a CGRP receptor comprising contacting a cell with a peptide conjugate of the invention in an amount effective to antagonise the receptor. The cell may be in vivo, in vitro, or ex vivo. In certain embodiments where the cell is in vivo, the cell may be contacted with the peptide conjugate by administering the peptide conjugate to a subject. Methods of antagonising or inhibiting CGRP receptors in a cell in vitro or ex vivo may be useful, for example, in a variety of diagnostic tests or laboratory research.

### Pharmaceutical Compositions

The present invention further relates to a pharmaceutical composition comprising a peptide conjugate of the invention; and a pharmaceutically acceptable carrier.

The pharmaceutical composition comprises an effective amount of the peptide conjugate.

The pharmaceutical compositions may comprise two or more peptide conjugates of the invention.

The term “pharmaceutically acceptable carrier” refers to a carrier (e.g. adjuvant or vehicle) that may be administered to a subject together with the peptide conjugate, which is generally safe, non-toxic, and neither biologically nor otherwise undesirable, including carriers suitable veterinary as well as human pharmaceutical use.

Pharmaceutically acceptable carriers that may be used in the compositions include, but are not limited to, ion exchangers, alumina, aluminum stearate, lecithin, self-emulsifying drug delivery systems (SEDDS) such as d-α-tocopherol polyethyleneglycol 1000 succinate, surfactants used in pharmaceutical dosage forms such as Tweens or other similar polymeric delivery matrices, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat. Cyclodextrins such as α-, β-, and γ-cyclodextrin, or chemically modified derivatives such as hydroxyalkylcyclodextrins, including 2- and 3-hydroxypropyl-3-cyclodextrins, or other solubilized derivatives may also be advantageously used to enhance delivery. Oil solutions or suspensions may also contain a long-chain alcohol diluent or dispersant, or carboxymethyl cellulose or similar dispersing agents, which are commonly used in the formulation of pharmaceutically acceptable dosage forms such as emulsions and or suspensions.

The compositions are formulated to allow for administration to a subject by any chosen route, including but not limited to oral or parenteral (including topical, subcutaneous, intramuscular and intravenous) administration. For example, the compositions may be formulated with an appropriate pharmaceutically acceptable carrier (including excipients, diluents, auxiliaries, and combinations thereof) selected with regard to the intended route of administration and standard pharmaceutical practice. For example, the compositions may be administered orally as a powder, liquid, tablet or capsule, or topically as an ointment, cream or lotion. Suitable formulations may contain additional agents as required, including emulsifying, antioxidant, flavouring or colouring agents, and may be adapted for immediate-, delayed-, modified-, sustained-, pulsed- or controlled-release.

The compositions may be administered via the parenteral route. Examples of parenteral dosage forms include aqueous solutions, isotonic saline or 5% glucose of the active agent, or other well-known pharmaceutically acceptable excipients. Cyclodextrins, for example, or other solubilising agents well-known to those familiar with the art, can be utilized as pharmaceutical excipients for delivery of the therapeutic agent.

Examples of dosage forms suitable for oral administration include, but are not limited to tablets, capsules, lozenges, or like forms, or any liquid forms such as syrups, aqueous solutions, emulsions and the like, capable of providing a therapeutically effective amount of the composition. Capsules can contain any standard pharmaceutically acceptable materials such as gelatin or cellulose. Tablets can be formulated in accordance with conventional procedures by compressing mixtures of the active ingredients with a solid carrier and a lubricant. Examples of solid carriers include starch and sugar bentonite.

Active ingredients can also be administered in a form of a hard shell tablet or a capsule containing a binder, e.g., lactose or mannitol, a conventional filler, and a tabletting agent.

Examples of dosage forms suitable for transdermal administration include, but are not limited, to transdermal patches, transdermal bandages, and the like.

Examples of dosage forms suitable for topical administration of the compositions include any lotion, stick, spray, ointment, paste, cream, gel, etc., whether applied directly to the skin or via an intermediary such as a pad, patch or the like.

Examples of dosage forms suitable for suppository administration of the compositions include any solid dosage form inserted into a bodily orifice particularly those inserted rectally, vaginally and urethrally.

Examples of dosage of forms suitable for injection of the compositions include delivery via bolus such as single or multiple administrations by intravenous injection, subcutaneous, subdermal, and intramuscular administration or oral administration.

Examples of dosage forms suitable for depot administration of the compositions include pellets or solid forms wherein the active(s) are entrapped in a matrix of biodegradable polymers, microemulsions, liposomes or are microencapsulated.

Examples of infusion devices for the compositions include infusion pumps for providing a desired number of doses or steady state administration, and include implantable drug pumps. Examples of implantable infusion devices for compositions include any solid form in which the active(s) are encapsulated within or dispersed throughout a biodegradable polymer or synthetic, polymer such as silicone, silicone rubber, silastic or similar polymer.

Examples of dosage forms suitable for transmucosal delivery of the compositions include depositories solutions for enemas, pessaries, tampons, creams, gels, pastes, foams, nebulised solutions, powders and similar formulations containing in addition to the active ingredients such carriers as are known in the art to be appropriate. Such dosage forms include forms suitable for inhalation or insufflation of the compositions, including compositions comprising solutions and/or suspensions in pharmaceutically acceptable, aqueous, or organic solvents, or mixture thereof and/or powders. Transmucosal administration of the compositions may utilize any mucosal membrane but commonly utilizes the nasal, buccal, vaginal and rectal tissues. Formulations suitable for nasal administration of the compositions may be administered in a liquid form, for example, nasal spray, nasal drops, or by aerosol administration by nebulizer, including aqueous or oily solutions of the polymer particles. Formulations may be prepared as aqueous solutions for example in saline, solutions employing benzyl alcohol or other suitable preservatives, absorption promoters to enhance bio-availability, fluorocarbons, and/or other solubilising or dispersing agents known in the art.

Examples of dosage forms suitable for buccal or sublingual administration of the compositions include lozenges, tablets and the like. Examples of dosage forms suitable for opthalmic administration of the compositions include inserts and/or compositions comprising solutions and/or suspensions in pharmaceutically acceptable, aqueous, or organic solvents.

Examples of formulations of compositions may be found in, for example, Sweetman, S. C. (Ed.). Martindale. The Complete Drug Reference, 33rd Edition, Pharmaceutical Press, Chicago, 2002, 2483 pp.; Aulton, M. E. (Ed.) Pharmaceutics. The Science of Dosage Form Design. Churchill Livingstone, Edinburgh, 2000, 734 pp.; and, Ansel, H. C, Allen, L. V. and Popovich, N. G. Pharmaceutical Dosage Forms and Drug Delivery Systems, 7th Ed., Lippincott 1999, 676 pp. Excipients employed in the manufacture of drug delivery systems are described in various publications known to those skilled in the art including, for example, Kibbe, E. H. Handbook of Pharmaceutical Excipients, 3rd Ed., American Pharmaceutical Association, Washington, 2000, 665 pp. The USP also provides examples of modified-release oral dosage forms, including those formulated as tablets or capsules. See, for example, The United States Pharmacopeia 23/National Formulary 18, The United States Pharmacopeial Convention, Inc., Rockville Md., 1995 (hereinafter “the USP”), which also describes specific tests to determine the drug release capabilities of extended-release and delayed-release tablets and capsules. The USP test for drug release for extended-release and delayed-release articles is based on drug dissolution from the dosage unit against elapsed test time. Descriptions of various test apparatus and procedures may be found in the USP. Further guidance concerning the analysis of extended release dosage forms has been provided by the F.D.A. (See Guidance for Industry. Extended release oral dosage forms: development, evaluation, and application of in vitro/in vivo correlations. Rockville, Md.: Center for Drug Evaluation and Research, Food and Drug Administration, 1997).

The dosage forms described herein can be in the form of physically discrete units suitable for use as unitary dosages for the subjects to be treated, each unit containing a predetermined quantity of active material calculated to produce the desired therapeutic effect.

Dosage levels of the active ingredients in the pharmaceutical compositions may be varied so as to provide an amount of the active ingredient which is effective to achieve the desired therapeutic effect for a particular patient, composition, and mode of administration, without being toxic to the patient (an effective amount).

The selected dosage level will depend upon a variety of pharmacokinetic factors including the activity of the particular compositions employed, the route of administration, the time of administration, the rate of excretion of the particular peptide conjugate being employed, other drugs, compounds and/or materials used in combination with the particular compositions employed, the age, sex, weight, condition, general health and prior medical history of the patient being treated, and like factors well known in the medical arts. Generally, the daily amount or regimen should be in the range of about 1 to about 10,000 micrograms (μg) of the CGRP peptide per kilogram (kg) of body mass, preferably about 1 to about 5000 μg per kilogram of body mass, and most preferably about 1 to about 1000 μg per kilogram of body mass.

### Kits

The present invention also provides a kit comprising a peptide conjugate of the present invention; and instructions for use.

The peptide conjugate is typically in the form of a pharmaceutical composition, and contained within a container. The instructions for use may describe the method(s) of treatment in which the peptide conjugates are administered. In various embodiments, the instructions for use describe methods of treating the diseases and conditions indicated herein.

The container may be any vessel or other sealed or sealable apparatus that can hold the pharmaceutical composition. Examples include bottles, ampules, divided or multi-chambered holders bottles, wherein each division or chamber comprises a single dose of said composition, a divided foil packet wherein each division comprises a single dose of said composition, or a dispenser that dispenses single doses of said composition. The container can be in any conventional shape or form and is made of a pharmaceutically acceptable material, for example a paper or cardboard box, a glass or plastic bottle or jar, a re-sealable bag, or a blister pack with individual doses for pressing out of the pack according to a therapeutic schedule. The container employed typically depends on the dosage form involved. More than one container can be used together in a single package for a single dosage form.

The kits may also comprise a device to administer or to measure out a unit dose of the pharmaceutical composition. The device may include, for example, an inhaler if the composition is an inhalable composition; a syringe and needle if the composition is an injectable composition; a syringe, spoon, pump, or a vessel with or without volume markings if the composition is an oral liquid composition; or any other measuring or delivery device appropriate to the dosage formulation of the composition present in the kit.

In various embodiments, the kits may comprise, for example in a separate vessel or container, one or more additional therapeutic agent, typically in the form of a pharmaceutical composition comprising the additional therapeutic agent and a pharmaceutically acceptable carrier.

The following non-limiting examples are provided to illustrate the present invention and in no way limit the scope thereof.

### EXAMPLES

### Example 1

This example describes methods for the preparation of peptide conjugates of the invention.

### 1. General Remarks

All solvents and reagents were purchased from commercial sources, and were used without further purification. Solvents for reverse phase high-performance liquid chromatography (RP-HPLC) were purchased as HPLC grade. O-(6-chlorobenzotriazol-1-yl)-N,N,N′,N′-tetramethyluronium hexafluorophosphate (HCTU), 4-[(R,S)-α-[1-(9H-fluoren-9-yl)]methoxycarbonylamino]-2,4-dimethoxy]phenoxyacetic acid (Fmoc-Rink amide linker). Fmoc-SPPS and other reactions were carried out under an air atmosphere without using anhydrous solvents. Fmoc-amino acids were purchased from GL Biochem (Shanghai, China) with the following side-chain protection: Fmoc-Asn(Trt)-OH (Trt=trityl), Fmoc-Cys(Mmt)-OH, Fmoc-Cys(tBu)-OH (tBu=tert-butyl), Fmoc-Lys(Boc)-OH (Boc=tert-butyloxycarbonyl), Fmoc-Ser(tBu)-OH, Fmoc-Cys(Trt)-OH, Fmoc-Thr(tBu)-OH, Fmoc-His(Trt)-OH, Fmoc-Arg(Pbf)-OH. Aminomethyl-ChemMatrix® was purchased from PCAS BioMatrix Inc. (Quebec, Canada). Formic acid, acetic anhydride (Ac₂O), N,N-diisopropylethylamine (DIPEA), piperidine, N-methylmorpholine (NMM), N-methylpyrrolidone (NMP), N,N′-diisopropylcarbodiimide (DIC), triisopropylsilane (TIS), 6-chloro-1-hydroxybenzotriazole (6-Cl-HOBt), methanol (MeOH), ethanol (EtOH), diethyl ether (Et₂O), chloroform (CHCl₃), deuterochloroform (CDCl₃), 2,2-dimethoxy-2-phenylacetophenone (DMPA), tert-butylthiol (tBuSH), vinyl palmitate and vinyl decanoate were purchased from Sigma-Aldrich (St. Louis, Mo.). N,N-dimethylformamide (DMF) (synthesis grade) and acetonitrile (MeCN) (HPLC grade) were purchased from Scharlau (Barcelona, Spain). Trifluoroacetic acid (TFA) was purchased from Halocarbon (River Edge, N.J.). Dichloromethane (DCM) was purchased from ECP limited (Auckland, New Zealand). Dimethyl sulfoxide (DMSO) was purchased from Romil Limited (Cambridge, United Kingdom). Ethyl acetate and petroleum ether were purchased from Burdick & Jackson® (Muskegon, Mich.). Guanidinium hydrochloride (GnHCl) was purchased from MP Biomedicals (Santa Ana, Calif.).

### 2. General Procedure for Purification and Analysis

Analytical reverse phase high-performance liquid chromatography (RP-HPLC) was performed on a Dionex UltiMate® 3000 equipped with a four-channel UV detector with either a Zorbax Eclipse Plus C18 95 Å, 2.1 mm×50 mm; 1.8 μm (0.2 mL/min), XTerra® MS-C18 110 Å 5 μm; 4.6×150 mm (1.0 mL/min), or Phenomenex Gemini C18 110 Å, 10.0 mm×250 mm; 5 μm (5.0 mL/min) as the column and using a linear gradient as specified, where solvent A was 0.1% TFA in water (H₂O) and B was 0.1% TFA in MeCN. Peptide masses were confirmed by analytical liquid-chromatography-mass spectrometry (LCMS), performed on an Agilent Technologies 1120 Compact LC connected to a HP Series 1100 MSD spectrometer using the column Agilent Zorbax 300SB-C3, 3.0 mm×150 mm; 5 μm (0.3 mL/min) using ESI in the positive mode and using a linear gradient as specified, where solvent A was 0.1% formic acid in H₂O and B was 0.1% formic acid in MeCN. Semi-preparative RP-HPLC was performed using either a Dionex UltiMate® 3000 or Waters 600E System with a Waters 2487 dual wavelength absorbance detector with the column Phenomenex Gemini C₁₈ 110 Å, 10.0 mm×250 mm; 5 μm (5 mL/min or 3 mL/min, where specified). A linear gradient of (A) 0.1% TFA/H₂O and (B) 0.1% TFA/MeCN was used with UV-Vis detection at 210 nm. Gradient systems used for semi-preparative RP-HPLC were adjusted according to the elution and peak profiles obtained from the analytical RP-HPLC chromatograms, and are specified in the experimental procedures section.

Analytical thin-layer chromatography (TLC) was carried out using Kieselgel F₂₅₄ 200 μm (Merck) silica plates. The compounds were then visualised by ultraviolet fluorescence. Column chromatography was performed using Grace Davison Discovery Sciences, Davasil LC60A 40-63 Micron Chromatographic Silica Media with the indicated eluent.

Nuclear magnetic resonance (NMR) spectra were recorded as indicated on a Bruker AVANCE 400 spectrometer operating at 400 MHz for ¹H nuclei and 100 MHz for ¹³C nuclei. All chemical shifts are reported in parts per million (ppm) on the δ scale from tetramethylsilane (TMS), and were referenced to residual solvent peaks (CDCl₃: δ=7.26 ppm for ¹H NMR, δ=77.0 ppm for ¹³C NMR). Coupling constants (J) are in Hertz (Hz). The 1H NMR shift values are reported as chemical shift δ, multiplicity (s=singlet, d=doublet, t=triplet, q=quartet, m=multiplet, dd=doublet of doublets, td=triplet of doublets, qd=quartet of doublets), coupling constant (J in Hz), relative integral and assignments. ¹³C NMR values are reported as the chemical shift δ, the degree of hybridisation and assignment.

### 3. General Procedure for Peptide Synthesis

Peptides were synthesised by automated 9-fluorenylmethoxycarbonyl solid-phase peptide synthesis (Fmoc-SPPS) using either a Tribute™ peptide synthesiser or PS3™ peptide synthesiser at room temperature, or using a Biotage® Initiator+Alstra™ microwave peptide synthesiser. To reduce aggregation of the growing peptide chain, the pseudoproline dipeptide Fmoc-Leu-Ser[Ψ^{(Me,Me)}Pro]-OH 1.6 was introduced in place of Leu¹⁶-Ser¹⁷. This pseudoproline is converted into the native Leu¹⁶-Ser¹⁷ upon resin cleavage with trifluoroacetic acid (TFA) as shown in Scheme 1 below.

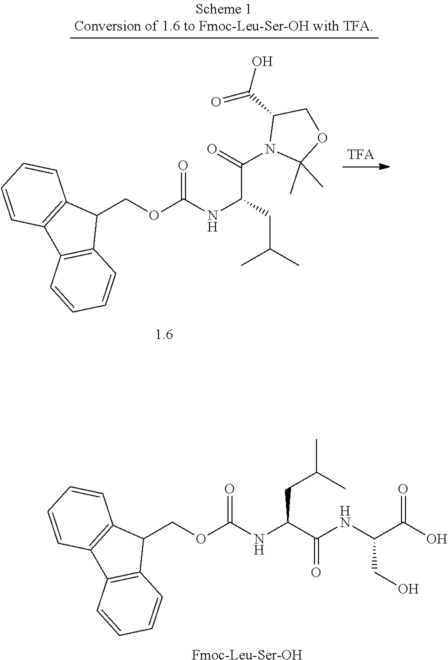 [SMILES: CC(C)C[C@H](NC(=O)OCC1c2ccccc2-c2ccccc21)C(=O)N1[C@H](C(=O)O)COC1(C)C ; "Fmoc-Leu-Ser-OH" ; CC(C)C[C@H](NC(=O)OCC1c2ccccc2-c2ccccc21)C(=O)N[C@@H](CO)C(=O)O]

### 3.1 Synthesis of Fmoc-Rink Amide-ChemMatrix® Resin

Aminomethyl-ChemMatrix® resin with an Fmoc-Rink amide linker was used as the solid support for synthesis of peptides. Fmoc-Rink amide linker (270 mg, 0.5 mmol) was coupled to Aminomethyl-ChemMatrix® resin (145 mg, 0.1 mmol) using 6-Cl-HOBt (84.8 mg, 0.5 mmol) and DIC (77.4 μL, 0.5 mmol) in DMF (2 mL) at room temperature for 2 h.

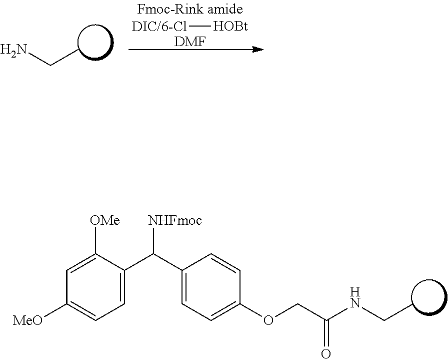

### 3.2 Automated Fmoc-SPPS

Using the PS3™ synthesiser, all amino acid couplings were performed as single coupling cycles. Protected amino acids were incorporated using Fmoc protected amino acid (Fmoc-AA-OH) (0.5 M, 0.5 mmol), HCTU (0.23 M, 0.45 mmol) and NMM (2.0 M, 1.0 mmol) for 20 min at room temperature (rt). Fmoc deprotections were carried out using 20% piperidine in DMF (2×5 min).

Using the Tribute™ peptide synthesiser, all amino acid couplings were performed as single coupling cycles. Protected amino acids were incorporated using Fmoc-AA-OH (0.5 M, 0.5 mmol), HCTU (0.23 M, 0.45 mmol) and NMM (2.0 M, 1.0 mmol) for 20 min at rt. Fmoc deprotections were carried out using 20% piperidine in DMF (2×5 min).

Using the Biotage® Initiator+Alstra™ microwave peptide synthesiser, all amino acid couplings were performed as single coupling cycles. Protected amino acids were incorporated using Fmoc-AA-OH (0.5 M, 0.5 mmol), HCTU (0.5 M, 0.45 mmol) and NMM (2.0 M, 1.0 mmol) for 5 min at 75° C. N-terminal capping was carried out where specified in the experimental procedure after every 4¹ or 5′ coupling using 20% Ac₂O and NMM (2.0 M, 0.1 mmol) in DMF (v/v) for 5 min. Fmoc deprotections were achieved using 20% piperidine in DMF (2×5 min).

Peptides were cleaved from the resin by treatment with trifluoroacetic acid/triisopropylsilane/water (TFA/TIPS/H₂) (95/2.5/2.5 v/v; 5 mL) either at room temperature for 2 h or at 30° C. for 20 min in a CEM® Discover microwave instrument. The crude peptides were precipitated and triturated with cold diethyl ether (2×30 mL), isolated (centrifugation), concentrated under a light stream of N₂ then dissolved in H₂O/MeCN (1:1, 30 mL) containing 0.1% TFA and lyophilised.

### 4. General Procedure for Solid Phase Coupling of Palmitoylated Building Block 1.1

Lipidated peptides synthesised by automated Fmoc-SPPS utilised either manual coupling of the palmitoylated building block; Fmoc-Cys(S-Pam)-OH 1.1, or an automated coupling using a Biotage® Initiator+Alstra™ microwave peptide synthesiser.

Manual coupling of the protected amino acid 1.1 was performed as a single coupling cycle using Fmoc-Cys(S-Pam)-OH 1.1 (0.03 M, 0.1 mmol), HCTU (2.3 M, 0.09 mmol) and NMM (2.0 M, 0.5 mmol) at room temperature for 1 h. Fmoc deprotection was achieved using 20% piperidine in DMF (v/v) (2×5 min). The remaining amino acids of the peptide sequence were coupled using automated Fmoc-SPPS outlined in the general procedure for peptide synthesis (see section 3 above).

For automated coupling of 1.1, the Initiator+Alstra™ microwave peptide synthesiser was used. Coupling of the protected amino acid 1.1 was performed as a single coupling cycle using Fmoc-Cys(S-Pam)-OH 1.1 (0.03 M, 0.1 mmol), HCTU (0.23 M, 0.09 mmol) and NMM (2.0 M, 0.5 mmol) at 75° C. for 20 min. Remaining amino acids of the peptide sequence were coupled using automated Fmoc-SPPS outlined in the general procedure for peptide synthesis (see section 3 above).

### 5. Synthesis of Cysteine Analogues A1-A3 and B

### 5.1 Synthesis of V8C CGRP₈₋₃₇ A1 [SEQ ID No:80]

```
        8
NH₂-CTHRLAGLLSRSGGVVKNNFVPTNVGSKAF-CONH₂
                                                                          A1
```

Automated Fmoc-SPPS using Tribute™ peptide synthesiser was used for the synthesis of V8C CGRP₈₋₃₇ A1 at a 0.05 mmol scale, followed by resin cleavage using the conditions outlined in the general procedure for peptide synthesis (see section 3 above) to afford crude A1 as a white solid (58.4 mg, 24% yield based on 63% purity by LCMS); retention time (Rₜ) 12.4 min; m/z (ESI-MS) 783.2 ([M+4H]⁴⁺ requires 783.4). LCMS was carried out using a Zorbax 300SB-C3 column (5 μm; 3.0×150 mm) using 5 to 65% B over 21 min (ca. 3% B/min) at 0.3 mL/min, rt, where A: 0.1% formic acid in H₂O, and B: 0.1% formic acid in MeCN.

A sample of the crude peptide A1 (10 mg) was purified by semi-preparative RP-HPLC using a Dionex UltiMate® 3000 on a Phenomenex Gemini C₁₈ column using a gradient of 0% B to 15% B over 15 min (ca. 1% B/min) then 15% B to 40% B over 350 min (ca. 0.1% B/min).

Fractions (2.5 mL) were collected at 0.5 min intervals and analysed by ESI-MS and RP-HPLC. Fractions identified with the correct m/z were combined and lyophilised to afford the title compound A1 as a white amorphous solid (0.98 mg, 16% yield, 93% purity); Rₜ 14.3 min; m/z (ESI-MS) 627.0 ([M+5H]⁵⁺ requires 626.1). RP-HPLC was carried out using an XTerra® MS-C18 column (5 μm; 4.6×150 mm) using 5 to 65% B over 24 min (ca. 2.5% B/min) at 1.0 mL/min, 45° C., where A: 0.1% TFA in H₂O and B: 0.1% TFA in MeCN.

### 5.2 Synthesis of K24C CGRP₈₋₃₇ A2 [SEQ ID NO:81]

```
                                        24
NH₂-VTHRLAGLLSRSGGVVCNNFVPTNVGSKAF-CONH₂
                                                                          A2
```

Automated Fmoc-SPPS using Tribute™ peptide synthesiser was used for the synthesis of K24C CGRP₈₋₃₇ A2 at a 0.05 mmol scale, followed by resin cleavage using the conditions outlined in the general procedure for peptide synthesis (see section 3 above) to afford crude A2 as a white solid (53.1 mg, 21% yield based on 62% purity by LCMS); Rₜ 13.7 min; m/z (ESI-MS) 776.0 ([M+4H]⁴⁺ requires 776.2). A sample of crude A2 (25 mg) was purified by semi-preparative RP-HPLC using a Dionex UltiMate® 3000 on a Phenomenex Gemini Cis column using a gradient of 0% B to 14% B over 7 min (ca. 2% B/min) then 14% B to 30% B over 160 min (ca. 0.1% B/min).

Fractions (2.5 mL) were collected at 0.5 min intervals and analysed by ESI-MS and RP-HPLC. Fractions identified with the correct m/z were combined and lyophilised to afford the title compound A2 as a white amorphous solid (1.8 mg, 12% yield, >95% purity); Rₜ 13.5 min; m/z (ESI-MS) 776.0 ([M+4H]⁴⁺ requires 776.2). RP-HPLC was carried out using a Zorbax 300SB-C3 column (5 μm; 3.0×150 mm) using 5 to 65% B over 21 min (ca. 3% B/min) at 0.3 mL/min, rt, where A: 0.1% formic acid in H₂O, and B: 0.1% formic acid in MeCN.

### 5.3 Synthesis of K35C CGRP₈₋₃₇ A3[SEQ ID NO:82]

```
                                                              35
NH₂-VTHRLAGLLSRSGGVVKNNFVPTNVGSCAF-CONH₂
                                                                          A3
```

Automated Fmoc-SPPS using a Tribute™ peptide synthesiser was used for the synthesis of K35C CGRP₈₋₃₇ A3 at a 0.1 mmol scale, followed by resin cleavage using the conditions outlined in the general procedure for peptide synthesis (see section 3 above) to afford crude A3 as a white solid (110 mg, 25% yield based on 70% purity by LCMS); Rₜ 13.6 min; m/z (ESI-MS) 776.0 ([M+4H]⁴⁺ requires 776.2). LCMS was carried using a Zorbax 300SB-C3 column (5 μm; 3.0×150 mm) using 5 to 65% B over 21 min (ca. 3% B/min) at 0.3 mL/min, rt, where A: 0.1% formic acid in H₂O, and B: 0.1% formic acid in MeCN.

A sample of crude A3 (25 mg) was purified by semi-preparative RP-HPLC using a Dionex UltiMate® 3000 on a Phenomenex Gemini Cis column using a gradient of 0% B to 22% B over 11 min (ca. 2% B/min) then 22% B to 35% B over 130 min (ca. 0.1% B/min).

Fractions (2.5 mL) were collected at 0.5 min intervals and analysed by ESI-MS and RP-HPLC. Fractions identified with the correct m/z were combined and lyophilised to afford the title compound A3 as a white amorphous solid (7.9 mg, 45% yield, 95% purity); Rₜ 12.2 min: m/z (ESI-MS) 776.1 ([M+4H]⁴⁺ requires 776.2). RP-HPLC was carried out using an XTerra® MS-C18 column (5 μm; 4.6×150 mm) using 5 to 65% B over 24 min (ca. 2.5% B/min) at 1.0 mL/min, 45° C., where A: 0.1% TFA in H₂O and B: 0.1% TFA in MeCN.

### 5.4 Synthesis of CGRP₇₋₃₇ B [SEQ ID NO:79]

```
        7
NH₂-CVTHRLAGLLSRSGGVVKNNFVPTNVGSKAF-CONH₂
                                                                            B
```

Automated Fmoc-SPPS using a Biotage® Initiator+Alstra™ microwave peptide synthesiser was used for the synthesis of CGRP₇₋₃₇ B on a 0.05 mmol scale, followed by resin cleavage using the conditions outlined in the general procedure for peptide synthesis (see section 3 above) to afford crude B as a white solid (49.53 mg, 10% yield based on 31% purity by LCMS); Rₜ 11.0 min; m/z (ESI-MS) 646.7 ([M+5H]⁵⁺ requires 646.8). LCMS was carried out using a Zorbax 300SB-C3 column (5 μm; 3.0×150 mm) using 5 to 65% B over 21 min (ca. 3% B/min) at 0.3 mL/min, rt, where A: 0.1% formic acid in H₂O, and B: 0.1% formic acid in MeCN.

A sample of the crude peptide B (27 mg) was purified by semi-preparative RP-HPLC using a Dionex UltiMate® 3000 on a Phenomenex Gemini C₁₈ column using a gradient of 5% B to 22% B over 17 min (ca. 1% B/min) then 22% B to 42% B over 200 min (ca. 0.1% B/min) at 45° C.

Fractions (2.5 mL) were collected at 0.5 min intervals and analysed by ESI-MS and RP-HPLC. Fractions identified with the correct m/z were combined and lyophilised to afford the title compound B as a white amorphous solid (2.85 mg, 34% yield, 97% purity); Rₜ 14.3 min; m/z (ESI-MS) 646.6 ([M+5H]⁵⁺ requires 646.8). RP-HPLC was carried out using a Zorbax Eclipse Plus-C18 column (1.8 μm; 2.1×50 mm), using 5 to 65% B over 30 min (ca. 2% B/min) at 0.2 mL/min, 45° C., where A: 0.1% TFA in H₂O and B: 0.1% TFA in MeCN.

### 6. Solution-Phase Cysteine Lipidation of Peptides and Amino Acids

### 6.1 Lipidation Conditions a

### 6.1.1 Preparation of V8C(S-Pam) CGRP₈₋₃₇ A4[SEQ ID NO:80]

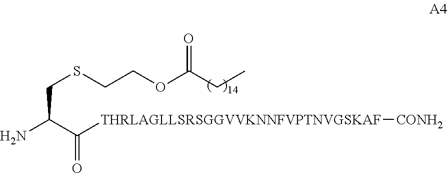

Fully-deprotected peptide A1 (10 mg, 0.003 mmol) treated with a solution of degassed N-methyl-2-pyrrolidone (NMP) (320 μL), 2,2-dimethoxy-2-phenylacetophenone (DMPA) (0.77 mg, 0.003 mmol), vinyl palmitate (28 mg, 0.01 mmol), tert-butylthiol (tBuSH) (27 μL, 0.24 mmol), triisopropylsilane (TIPS) (49 μL, 0.24 mmol) and N,N-diisopropylethylamine (DIPEA) (11 μL, 0.06 mmol). The reaction was irradiated under UV light (365 nm) for 45 min. The mixture was then diluted with GnHCl (6 M in H₂O). Analysis by LCMS using a Zorbax 300SB-C3 (5 μm; 3.0×150 mm) column (5 μm; 3.0×150 mm) and using 5 to 95% B over 34 min (ca. 3% B/min) at 0.3 mL/min, rt, (where A: 0.1% formic acid in H₂O, and B: 0.1% formic acid in MeCN) confirmed the presence of peptide A4 (9% conversion); Rₜ 17.1 min; MS: calcd. for [M+3H]³ 1138.4; found 1138.1.

### 6.1.2 Preparation of K24C(S-Pam) CGRP₈₋₃₇ A5 [SEQ ID NO:81]

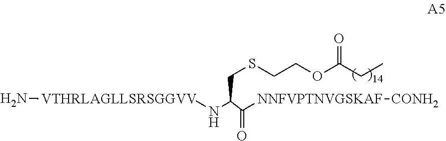

Fully-deprotected peptide A2 (10 mg, 0.003 mmol) treated with a solution of degassed NMP (320 μL), DMPA (0.77 mg, 0.003 mmol), vinyl palmitate (28 mg, 0.01 mmol), tBuSH (27 μL, 0.24 mmol), TIPS (49 μL, 0.24 mmol) and DIPEA (11 μL, 0.06 mmol). The solution was then diluted with GnHCl (6 M in H₂O). Analysis by LCMS using a Zorbax 300SB-C3 (5 μm; 3.0×150 mm) column using 5 to 95% B over 34 min (ca. 3% B/min) at 0.3 mL/min, rt, (where A: 0.1% formic acid in H₂O, and B: 0.1% formic acid in MeCN) confirmed the presence of peptide A5 (18% conversion); Rₜ 18.8 min; MS: calcd. for [M+4H]⁴⁺ 846.8; found 846.7.

### 6.1.3 Preparation of K35C(S-Pam) CGRP₈₋₃₇ A6 [SEQ ID NO:82]

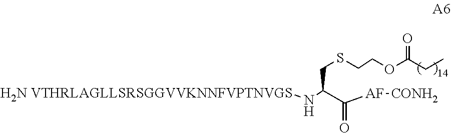

Fully-deprotected peptide A3 (10 mg, 0.003 mmol) treated with a solution of degassed NMP (320 μL), DMPA (0.77 mg, 0.03 mmol), vinyl palmitate (28 mg, 0.01 mmol), tBuSH (27 μL, 0.24 mmol), TIPS (49 μL, 0.24 mmol) and DIPEA (11 μL, 0.06 mmol). The solution was then diluted with GnHCl (6 M in H₂O). Analysis by LCMS using a Zorbax 300SB-C3 (5 μm; 3.0×150 mm) column using 5 to 95% B over 34 min (ca. 3% B/min) at 0.3 mL/min, rt, (where A: 0.1% formic acid in H₂O, and B: 0.1% formic acid in MeCN) confirmed the presence of peptide A6 (19% conversion); Rₜ 19.7 min; MS: calcd. for [M+4H]⁴⁺ 846.8; found 846.5.

### 6.2 Lipidation Conditions B

### 6.2.1 Preparation of K35C(S-Pam) CGRP₈₋₃₇ A6 [SEQ ID NO:81]

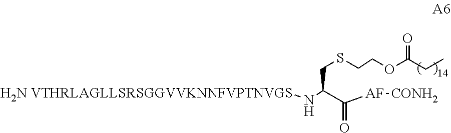

Fully-deprotected peptide A3 (10 mg, 0.003 mmol) treated with a solution of degassed NMP (320 μL), DMPA (0.77 mg, 0.003 mmol), vinyl palmitate (56 mg, 0.02 mmol), tBuSH (27 μL, 0.24 mmol), TIPS (49 μL, 0.24 mmol) and DIPEA (11 μL, 0.06 mmol). The solution was then diluted with GnHCl (6 M in H₂O). Analysis by LCMS using a Zorbax 300SB-C3 (5 μm; 3.0×150 mm) column using 5 to 95% B over 34 min (ca. 3% B/min) at 0.3 mL/min, rt, (where A: 0.1% formic acid in H₂O, and B: 0.1% formic acid in MeCN) confirmed the presence of peptide A6 (34% conversion); Rₜ 19.7 min; MS: calcd. for [M+4H]⁴⁺ 846.8; found 846.6.

Crude A6 (5 mg) was dissolved in GnHCl (6 M in H₂O) and purified by semi-preparative RP-HPLC using a Dionex UltiMate® 3000 on a Phenomenex Gemini Cis column (10×250 mm) using a gradient of 5% B to 65% B over 30 min (ca. 4% B/min).

Fractions (1.5 mL) were collected at 0.5 min intervals and analysed by ESI-MS and RP-HPLC. Fractions identified with the correct m/z were combined and lyophilised to afford the title compound A6 as a white amorphous solid (0.21 mg, 59% purity by LCMS); Rₜ16.1 min; MS: calcd. for [M+4H]⁴⁺ 864.8; found 846.7. LCMS was carried out using a Zorbax 300SB-C3 column (5 μm; 3.0×150 mm) using 5 to 95% B over 23 min (ca. 4.5% B/min) at 0.3 mL/min, rt (where A: 0.1% formic acid in H₂O, and B: 0.1% formic acid in MeCN).

### 6.3 Lipidation Conditions C

### 6.3.1 Preparation of K24C(S-Pam) CGRP₈₋₃₇ A5 [SEQ ID NO:81]

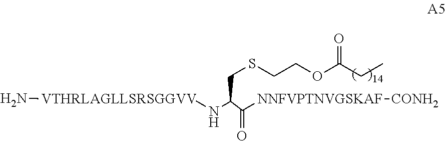

Fully-deprotected peptide A2 (10 mg, 0.003 mmol) treated with a solution of degassed NMP (310 μL), DMPA (1.54 mg, 0.006 mmol), vinyl palmitate (56 mg, 0.02 mmol), tBuSH (27 μL, 0.24 mmol), TIPS (49 μL, 0.24 mmol) and DIPEA (11 μL, 0.06 mmol). The solution was then diluted with GnHCl (6 M in H₂O). Analysis by LCMS using a Zorbax 300SB-C3 (5 μm; 3.0×150 mm) column using 5 to 95% B over 34 min (ca. 3% B/min) at 0.3 mL/min, rt (where A: 0.1% formic acid in H₂O, and B: 0.1% formic acid in MeCN) confirmed the presence of peptide A5 (18% conversion); Rₜ 18.9 min; MS: calcd. for [M+4H]⁴⁺ 846.8; found 846.7.

### 6.4 Lipidation Conditions D

### 6.4.1 Preparation of K24C(S-Pam) CGRP₈₋₃₇ A5 [SEQ ID NO:81]

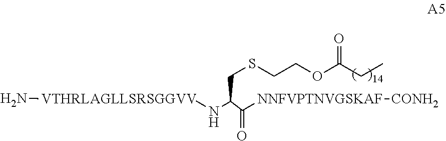

Fully-deprotected peptide A2 (5 mg, 0.0015 mmol) treated with a solution of degassed NMP (295 μL), DMPA (1.2 mg, 0.009 mmol), vinyl palmitate (59 mg, 0.42 mmol), tBuSH (14 μL, 0.12 mmol), TIPS (25 μL, 0.12 mmol), and TFA (5% v/v). The solution was then diluted with GnHCl (6 M in H₂O). Analysis by RP-HPLC using a Zorbax 300SB-C3 Zorbax Eclipse Plus-C18 (1.8 μm; 2.1×50 mm) column using 5 to 95% B over 35 min (ca. 3% B/min) at 0.2 mL/min, 45° C. (where A: 0.1% TFA in H₂O and B: 0.1% TFA in MeCN) confirmed the presence of peptide A5 (37% conversion); Rₜ 21.5 min.

### 7. Building Block Synthesis of Palmitoylated Analogues A4-A6 and B1

7.1 Synthesis of Amino Acid Building Block: (R)-2-((((9H-Fluoren-9-yl)methoxy)carb onyl)-amino-3-((2-(palmitoyloxy)ethyl)thio)propanoic acid (Fmoc-Cys(S-Pam)-OH) 1.1

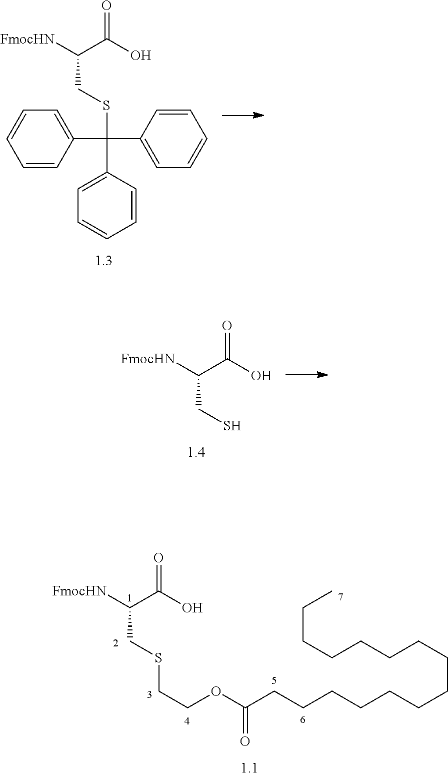

Fmoc-Cys(Trt)-OH 1.3 (2.1 g) was treated with a solution of TFA/DCM (v/v, 1:1, 15 mL) at room temperature for 3 h. The TFA solution was then concentrated under a stream of N₂, diluted with H₂O/MeCN (1:1, 30 mL) containing 0.1% TFA, and lyophilised yielding a white amorphous solid 1.4 (2060 mg, 59%); m/z (ESI-MS) 344.1 ([M+H]⁺ requires 344.1). The solid (500 mg, 1.39 mmol) was dissolved in DCM (5 mL) with vinyl palmitate 1.2 (615 mg, 2.18 mmol) and DMPA (374 mg, 1.45 mmol) and the solution was irradiated under UV light (365 nm) until TLC confirmed complete consumption of the starting material; Fmoc-Cys-OH 1.4. The solvent was evaporated in vacuo and the crude reaction mixture purified by flash column chromatography (petroleum ether/EtOAc, 3:2; followed by MeOH/DCM, 5:95), and the resulting yellow oil was lyophilised to afford the title compound 1.1 as a pale yellow solid (255 mg, 86% yield); m/z (ESI-MS) 626.4 ([M+H]⁺ requires 646.4). [α]_{D}²¹−7.4 (c 0.012 in CHCl₃) (lit. (Eur. J. Org. Chem. 2016, 2608-2616) −8.5 c 0.398, MeOH). ¹H NMR (400 MHz; CDCl₃): δ_{H}=7.76 (d, J=7.5 Hz, 2H, 2×Ar—H), 7.60 (d, J=6.6 Hz, 2H, 2×Ar—H), 7.39 (t, J=7.4 Hz, 2H, 2×Ar—H), 7.31 (m, 2H, 2×Ar—H), 5.73 (d, J=7.7 Hz, 1H, N—H), 4.66-4.65 (m, 1H, α-CH), 4.44-4.41 (m, 2H, Fmoc-CH₂), 4.24-4.20 (m 3H, Fmoc-CH and H-4), 3.14 (dd, J=13.5, J=4.5, 6.8 Hz, 1H, β-CH₂ₐ), 3.07 (dd, J=14.2, J=5.3, 1H, β-CH_{2b}), 2.78 (t, J=6.1 Hz, 2H, H-5), 2.29 (t, J=15.2, 2H, H-5), 1.58 (m, 2H, H-6), 1.31-1.24 (m, 26H), 0.88 (t, J=5.6, 3H, H-7) ppm. 13C NMR (100 MHz; CDCl₃): δ_{C}=174.9 (C, C═O), 174.0 (C, C═O), 155.1 (C, OCONH), 143.8 (CH, ArCH), 143.8 (CH, ArCH), 141.4 (CH, ArCH), 127.9 (CH, ArCH), 127.1 (CH, ArCH), 120.1 (CH, ArCH), 67.5 (CH₂, Fmoc-CH₂), 63.2 (CH₂, D-CH₂), 53.7 (CH, α-CH), 47.2 (CH, Fmoc-CH), 34.5 (CH₂, D-CH₂), 34.3 (CH₂, C-5), 32.1 (CH₂), 31.4 (CH₂, C-3), 29.7 (CH₂), 29.6 (CH₂), 29.5 (CH₂), 29.4 (CH₂), 29.3 (CH₂), 25.0 (CH₂), 22.8 (CH₂), 14.2 (CH₃, C-7) ppm.

Spectroscopic data and optical rotation were in good agreement with those previously reported in Eur. J. Org. Chem. 2016, 2608-2616.

### 7.2 Building Block Synthesis of V8C(S-Pam) CGRP₈₋₃₇ A4 [SEQ ID NO:80]

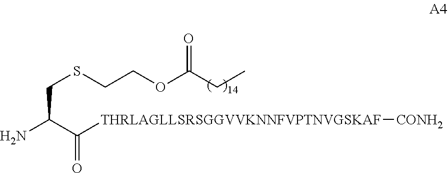

Automated Fmoc-SPPS using an Initiator+Alstra™ microwave peptide synthesiser was used for the synthesis of resin-bound CGRP₉₋₃₇ at a 0.05 mmol scale using conditions outlined in the general procedure for peptide synthesis (see section 3 above), followed by manual coupling of building block 1.1 using the conditions outlined in the general procedure for solid phase coupling of palmitoylated building block 1.1 (see section 4 above) to give V8C(S-Pam) CGRP₈₋₃₇ A4. Resin cleavage was then carried out using the conditions outlined in the general procedure for peptide synthesis (see section 3 above) to afford crude A4 as a pale pink solid (83.6 mg, 49% yield based on 59% purity by LCMS); Rₜ 17.3 min; m/z (ESI-MS) 683.3 ([M+5H]⁵⁺ requires 683.3). LCMS was carried out using a Zorbax 300SB-C3 column (5 μm; 3.0×150 mm) using 5 to 95% B over 31 min (ca. 3% B/min) at 0.3 mL/min, rt (where A: 0.1% formic acid in H₂O, and B: 0.1% formic acid in MeCN).

A sample of crude A4 (40 mg) was dissolved with 0.05% TFA in DMSO/H₂O (1:1) and purified by semi-preparative RP-HPLC using a Dionex UltiMate® 3000 on a Phenomenex Gemini C₁₈ column using a gradient of 5% B to 55% B over 50 min (ca. 1% B/min) then 55% B to 70% B over 150 min (ca. 0.1% B/min).

Fractions (1.5 mL) were collected at 0.5 min intervals and analysed by ESI-MS and RP-HPLC. Fractions identified with the correct m/z were combined and lyophilised to afford the title compound A4 as a white amorphous solid (0.64 mg, 3% yield, >95% purity); Rₜ 18.7 min; m/z (ESI-MS) 683.2 ([M+5H]⁵⁺ requires 683.3). RP-HPLC was carried out using an XTerra® MS-C18 column (5 μm; 4.6×150 mm) using 5 to 95% B over 30 min (ca. 3% B/min) at 1.0 mL/min, 45° C. (where A: 0.1% TFA in H₂O and B: 0.1% TFA in MeCN).

### 7.3 Building Block Synthesis of K24C(S-Pam) CGRP₈₋₃₇ A5 [SEQ ID NO:81]

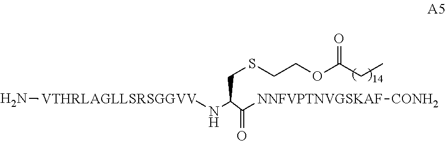

Automated Fmoc-SPPS using an Initiator+Alstra™ microwave peptide synthesiser was used for the synthesis of resin-bound CGRP₂₅₋₃₇ at a 0.05 mmol scale using conditions outlined in the general procedure for peptide synthesis (see section 3 above), and building block 1.1 was incorporated using the conditions for automated coupling outlined in the general procedure for solid phase coupling of palmitoylated building block 1.1 (see section 4 above). Elongation of the peptide sequence was continued with Ac₂O capping every 4^{th} residue as outlined in the general procedure for peptide synthesis (see section 3 above) to give K24C(S-Pam) CGRP₈₋₃₇ A5. Then resin cleavage was carried out using the conditions outlined in the general procedure for peptide synthesis (see section 3 above) to afford crude A5 as a white solid (86.9 mg, 21% yield based on 40% purity by LCMS); Rₜ 15.1 min; m/z (ESI-MS) 846.5 ([M+4H]⁴⁺ requires 846.6).

A sample (40 mg) of crude A5 was dissolved with 0.05% TFA in DMSO/H₂O (1:1) and purified by semi-preparative RP-HPLC using a Dionex UltiMate® 3000 on a Phenomenex Gemini C₁₈ column using a gradient of 5% B to 37% B over 32 min (ca. 1% B/min) then 37% B to 45% B over 360 min (ca. 0.05% B/min).

Fractions (1.5 mL) were collected at 0.5 min intervals and analysed by ESI-MS and RP-HPLC. Fractions identified with the correct m/z were combined and lyophilised to afford the title compound A5 as a white amorphous solid (1.11 mg, 7% yield, >95% purity); Rₜ 15.3 min: m/z (ESI-MS) 846.5 ([M+4H]⁴⁺ requires 846.6). LCMS was carried out using a Zorbax 300SB-C3 column (5 μm; 3.0×150 mm) using 5 to 95% B over 21 min (ca. 4.5% B/min) at 0.3 mL/min, rt (where A: 0.1% formic acid in H₂O, and B: 0.1% formic acid in MeCN).

### 7.4 Building Block Synthesis of K35C(S-Pam) CGRP₈₋₃₇ A6 [SEQ ID NO:82]

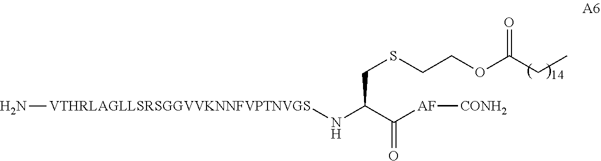

Automated Fmoc-SPPS using an Initiator+Alstra™ microwave peptide synthesiser was used for the synthesis of resin-bound CGRP₃₆₋₃₇ at a 0.05 mmol scale using conditions outlined in the general procedure for peptide synthesis (see section 3 above), and building block 1.1 was incorporated using the conditions for automated coupling outlined in the general procedure for solid phase coupling of palmitoylated building block 1.1 (see section 4 above). Elongation of the peptide sequence was continued with Ac₂O capping every 5^{th} residue as outlined in the general procedure for peptide synthesis (see section 3 above) to give K35C(S-Pam) CGRP₈₋₃₇ A6. Then resin cleavage was carried out using the conditions outlined in the general procedure for peptide synthesis (see section 3 above) to afford crude A6 as a white solid (43.3 mg, 14% yield based on 33% purity by LCMS); Rₜ 16.1 min; m/z (ESI-MS) 846.5 ([M+4H]⁴⁺ requires 846.6).

A sample (21 mg) of crude A6 was dissolved with 0.05% TFA in DMSO/H₂O (1:1) and purified by semi-preparative RP-HPLC using a Dionex UltiMate® 3000 on a Phenomenex Gemini C₁₈ column using a gradient of 5% B to 40% B over 35 min (ca. 1% B/min) then 40% B to 50% B over 200 min (ca. 0.05% B/min).

Fractions (2 mL) were collected at 0.5 min intervals and analysed by ESI-MS and RP-HPLC. Fractions identified with the correct m/z were combined and lyophilised to afford the title compound A6 as a white amorphous solid (0.60 mg, 9% yield, 95% purity); Rₜ16.0 min; m/z (ESI-MS) 846.6 ([M+4H]⁴⁺ requires 846.6). LCMS was carried out using a Zorbax 300SB-C3 (5 μm; 3.0×150 mm) using 5 to 95% B over 21 min (ca. 4.5% B/min) at 0.3 mL/min, rt (where A: 0.1% formic acid in H₂O, and B: 0.1% formic acid in MeCN).

### 7.5 Building Block Synthesis of 7C(S-Pam) CGRP₇₋₃₇ B1 [SEQ ID NO:79]

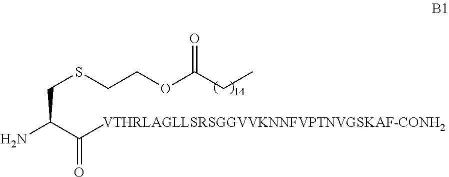

Automated Fmoc-SPPS using an Initiator+Alstra™ microwave peptide synthesiser was used for the synthesis of resin-bound CGRP₈₋₃₇ A at a 0.05 mmol scale using conditions outlined in the general procedure for peptide synthesis (see section 3 above), followed by manual coupling of building block 1.1 using the conditions outlined in the general procedure for solid phase coupling of palmitoylated building block 1.1 (see section 4 above) to give 7C(S-Pam) CGRP₇₋₃₇ B1. Resin cleavage was then carried out using the conditions outlined in the general procedure for peptide synthesis (see section 3 above) to afford crude B1 as a white solid (38.6 mg, 16% yield based on 41% purity by LCMS); Rₜ14.3 min; m/z (ESI-MS) 703.1 ([M+5H]⁵⁺ requires 703.2). LCMS was carried out using a Zorbax 300SB-C3 column (5 μm; 3.0×150 mm) using 5 to 95% B over 21 min (ca. 4.5% B/min) at 0.3 mL/min, rt (where A: 0.1% formic acid in H₂O, and B: 0.1% formic acid in MeCN).

A sample of crude B1 (21 mg) was dissolved with 0.05% TFA in DMSO/H₂O (1:1) and purified by semi-preparative RP-HPLC using a Dionex UltiMate® 3000 on a Phenomenex Gemini Cis column using a gradient of 5% B to 35% B over 30 min (ca. 1% B/min) then 35% B to 50% B over 300 min (ca. 0.05% B/min). Fractions (1.5 mL) were collected at 0.5 min intervals and analysed by ESI-MS and RP-HPLC.

Fractions identified with the correct m/z were combined and lyophilised to afford the title compound B1 as a white amorphous solid (1.08 mg, 13% yield, 95% purity); Rₜ 20.9 min; m/z (ESI-MS) 703.1 ([M+5H]⁵⁺; requires 703.2). RP-HPLC was carried out using an XTerra® MS-C18 column (5 μm; 4.6×150 mm) using 5 to 95% B over 30 min (ca. 3% B/min) at 1.0 mL/min, 45° C. (where A: 0.1% TFA in H₂O and B: 0.1% TFA in MeCN).

### 8. Solid-Phase Cysteine Lipidation of Peptides and Amino Acids

### 8.1 Synthesis of K24C(S-Dec) CGRP₈₋₃₇ A8

### 8.1.1 Synthesis of Resin-Bound K24C(Mmt) CGRP₈₋₃₇ A2 [SEQ ID NO:81]

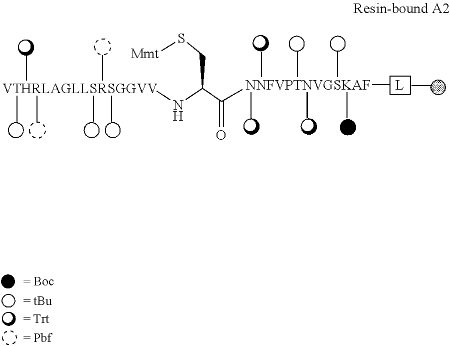

Automated Fmoc-SPPS using a Biotage® Initiator+Alstra™ peptide synthesiser was used for the synthesis of resin-bound K24C(Mmt) CGRP₈₋₃₇ A2 at a 0.05 mmol scale using conditions outlined in the general procedure for peptide synthesis (see section 3 above). A few beads of the resin-bound peptide were cleaved using the conditions outlined in the general procedure for peptide synthesis (see section 3 above) to afford crude A2 as a white solid (48% purity based on LCMS); Rₜ 11.4 min; (ESI-MS) 1034.3 ([M+3H]³⁺; requires 1034.5). LCMS was carried out using a Zorbax 300SB-C3 column (5 μm; 3.0×150 mm) using 5 to 95% B over 23 min (ca. 4.5% B/min) at 0.3 mL/min, rt (where A: 0.1% formic acid in H₂O, and B: 0.1% formic acid in MeCN).

### 8.1.2 Procedure A: Lipidation of Resin-Bound A2 to Afford K24C(S-Dec) CGRP₈₋₃₇ A8 [SEQ ID NO:81] and Bis-Decanoated Product A10

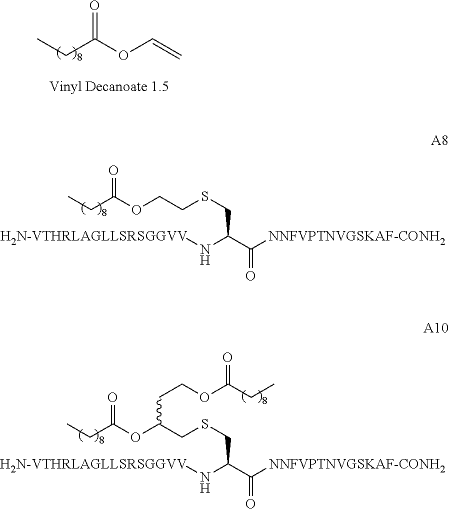

Resin-bound peptide A2 (20 mg, containing approx. 0.006 mmol peptide) was repeatedly treated with 5% TFA and 5% TIPS in DCM (v/v) for monomethoxytrityl (Mmt)-removal. The semi-deprotected, resin-bound peptide was then treated with a solution of degassed NMP (381 μL), DMPA (7.7 mg, 0.03 mmol), vinyl decanoate 1.5 (94 μL, 1.39 mmol) and TFA (25 μL, 5% v/v) and the reaction was irradiated under UV light (365 nm) for 1 h. A small amount of the resin-bound peptide was cleaved using the conditions outlined in the general procedure for peptide synthesis (see section 3 above) to afford crude A8 as a white solid. Analysis by RP-HPLC using a Zorbax Eclipse Plus-C18 (1.8 μm; 2.1×50 mm) column using 5 to 95% B over 35 min (ca. 3% B/min) at 0.2 mL/min, 45° C. (where A: 0.1% TFA in H₂O and B: 0.1% TFA in MeCN) confirmed the presence of peptide A8; Rₜ 18.3 min (35% conversion; 67% mono-S-palmitoylated product, 33% bis-decanoated product A10 at Rₜ 22.5 min; m/z (ESI-MS) 699.0 ([M+5H]⁵⁺ requires 700.9).

### 8.1.3 Procedure B: Lipidation of Resin-Bound A2 to Afford K24C(S-Dec) CGRP₈₋₃₇ A8

Resin-bound peptide A2 (20 mg, containing approx. 0.006 mmol peptide) was repeatedly treated with 2% TFA and 2% TIPS in DCM (v/v) for Mmt-removal. The semi-deprotected, resin-bound peptide was then treated with a solution of degassed NMP (220 μL), DMPA (3.08 mg, 0.012 mmol), vinyl decanoate (100 μL, 0.45 mmol), tBuSH (54 μL, 0.48 mmol), TIPS (99 μL, 0.48 mmol), and TFA (25 μL, 5% v/v) and the reaction was irradiated under UV light (365 nm) for 1 h. A small amount of the resin-bound peptide was cleaved using the conditions outlined in the general procedure for peptide synthesis (see section 3 above) to afford crude A8 as a white solid. Analysis by RP-HPLC using a Zorbax Eclipse Plus-C18 (1.8 μm; 2.1×50 mm) column using 5 to 65% B over 24 min (ca. 3% B/min) at 0.2 mL/min, 45° C. (where A: 0.1% TFA in H₂O and B: 0.1% TFA in MeCN) and ESI-MS confirmed the presence of peptide A8 (75% conversion); Rₜ 18.4 min; m/z (ESI-MS) 1100.3 ([M+3H]³⁺; requires 1100.3).

### 8.2 Synthesis of V8C(S-Pam) CGRP₈₋₃₇ A4

### 8.2.1 Synthesis of Resin-bound V8C(Mmt) CGRP₈₋₃₇ A1 [SEQ ID NO:80]

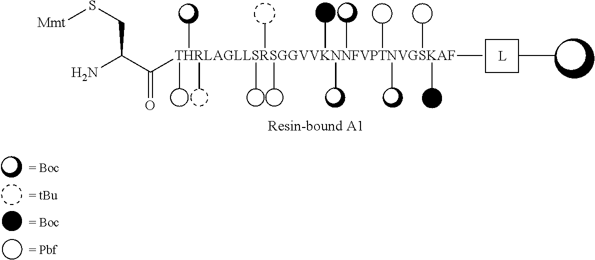

Automated Fmoc-SPPS using a PS3™ peptide synthesiser was used for the synthesis of resin-bound V8C(Mmt) CGRP₈₋₃₇ A1 at a 0.05 mmol scale using conditions outlined in the general procedure for peptide synthesis (see section 3 above). A few beads of the resin-bound peptide were cleaved using the conditions outlined in the general procedure for peptide synthesis (see section 3 above) to afford crude A1 as a white solid (84% purity based on LCMS); Rₜ 12.5 min; m/z (ESI-MS) 1044.1 ([M+3H]; requires 1044.2). LCMS was carried out using a Zorbax 300SB-C3 column (5 μm; 3.0×150 mm) using 5 to 65% B over 23 min (ca. 3% B/min) at 0.3 mL/min, 40° C. (where A: 0.1% formic acid in H₂O, and B: 0.1% formic acid in MeCN).

### 8.2.2 Procedure A: Lipidation of Resin-Bound A1 to Afford V8C(S-Pam) CGRP₈₋₃₇ A4 [SEQ ID NO:80] and Bis-Palmitoylated Product A7

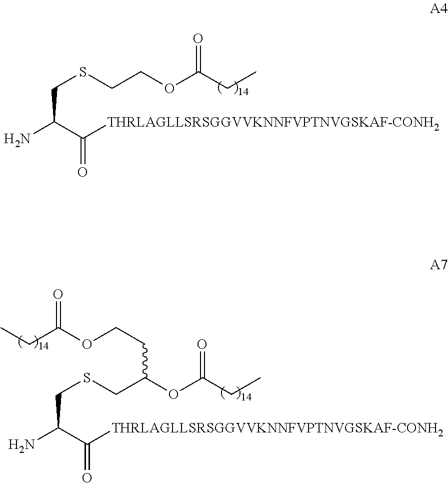

Resin-bound peptide A1 (282 mg, containing approx. 0.04 mmol peptide) was repeatedly treated with 5% TFA and 5% TIPS in DCM (v/v) for Mmt-removal. The semi-deprotected, resin-bound peptide was then treated with a solution of degassed NMP (8.1 mL), DMPA (5.1 mg, 0.02 mmol), vinyl palmitate (393 mg, 1.39 mmol), tBuSH (358 μL, 3.2 mmol), TIPS (654 μL, 3.2 mmol), and TFA (500 μL, 5% v/v) and the reaction was irradiated under UV light (365 nm) for 1 h. A small amount of the resin-bound peptide was cleaved using the conditions outlined in the general procedure for peptide synthesis (see section 3 above) to afford crude A4 as a white solid. Analysis by LCMS using a Zorbax 300SB-C3 (5 μm; 3.0×150 mm) column using 5 to 95% B over 23 min (ca. 4% B per min) at 0.3 mL/min, 40° C. (where A: 0.1% formic acid in H₂O, and B: 0.1% formic acid in MeCN) confirmed the presence of peptide A4 Rₜ 14.0 min; m/z (ESI-MS) 1138.4 ([M+3H]; requires 1138.4); (20% conversion, 86% mono-S-palmitoylated product, 14% bis-palmitoylated product A7 at Rₜ 20.3 min: m/z (ESI-MS) 739.9 ([M+5H]⁵⁺ requires 739.8)).

The reaction was then repeated upon the same peptidyl resin. A solution of degassed NMP (2.9 mL), DMPA (5.1 mg, 0.02 mmol), vinyl palmitate (786 mg, 2.8 mmol), tBuSH (358 μL, 3.2 mmol), TIPS (654 μL, 3.2 mmol), and TFA (250 μL, 5% v/v) and the reaction was irradiated under UV light (365 nm) for 1 h. A small amount of the resin-bound peptide was cleaved using the conditions outlined in the general procedure for peptide synthesis (see section 3 above) to afford crude A4 as a white solid. Analysis by LCMS using a Zorbax 300SB-C3 (5 μm; 3.0×150 mm) column using 5 to 95% B over 23 min (ca. 4% B per min) at 0.3 mL/min, 40° C. (where A: 0.1% formic acid in H₂O, and B: 0.1% formic acid in MeCN) confirmed the presence of peptide A4 Rₜ 14.0 min; m/z (ESI-MS) 1138.4 ([M+3H]³⁺; requires 1138.4); (65% conversion, 86% mono-S-palmitoylated product, 14% bis-palmitoylated product A7 at Rₜ 20.3 min; m/z (ESI-MS) 739.9 ([M+5H]⁵⁺ requires 739.8)).

The reaction was then repeated again upon the same peptidyl resin. A solution of degassed NMP (2.9 mL), DMPA (10.2 mg, 0.04 mmol), vinyl palmitate (786 mg, 2.8 mmol), tBuSH (358 μL, 3.2 mmol), TIPS (654 μL, 3.2 mmol), and TFA (250 μL, 5% v/v) and the reaction was irradiated under UV light (365 nm) for 1 h. Following removal of the reaction mixture by filtration and washing and drying of the resins, a small amount of the resin-bound peptide was cleaved using the conditions outlined in the general procedure for peptide synthesis (see section 3 above) to afford crude A4 as a white solid. Analysis by LCMS using a Zorbax 300SB-C3 (5 μm; 3.0×150 mm) column using 5 to 95% B over 23 min (ca. 4% B per min) at 0.3 mL/min, 40° C. (where A: 0.1% formic acid in H₂O, and B: 0.1% formic acid in MeCN) confirmed the presence of peptide A4 Rₜ 14.0 min; m/z (ESI-MS) 1138.4 ([M+3H]³⁺; requires 1138.4); (88% conversion, 91% mono-S-palmitoylated product, 9% bis-palmitoylated product A7 at Rₜ 20.3 min; m/z (ESI-MS) 739.9 ([M+5H]⁵⁺ requires 739.8)).

The reaction was repeated a fourth time upon the peptidyl resin. A solution of degassed DMF (2.9 mL), DMPA (10.2 mg, 0.04 mmol), vinyl palmitate (786 mg, 2.8 mmol), tBuSH (358 μL, 3.2 mmol), TIPS (654 μL, 3.2 mmol), and TFA (250 μL, 5% v/v) and the reaction was irradiated under UV light (365 nm) for 1 h. Following removal of the reaction mixture by filtration and washing and drying of the resins, the resin-bound peptide was fully cleaved using conditions outlined in the general procedure for peptide synthesis (see section 3 above) to afford crude A4 as a white solid which was then dissolved in acetonitrile/H₂O (1:1 v/v) containing TFA (0.1% v/v). Analysis by LCMS using a Zorbax 300SB-C3 (5 μm; 3.0×150 mm) column using 5 to 95% B over 23 min (ca. 4.5% B per min) at 0.3 mL/min, 40° C. (where A: 0.1% formic acid in H₂O, and B: 0.1% formic acid in MeCN) confirmed the presence of peptide A4 (91% conversion; 97% mono-S-palmitoylated product; 36.6 mg, 27% yield based on 55% purity); Rₜ 13.9 min; m/z (ESI-MS) 1138.3 ([M+3H]³⁺; requires 1138.4).

A sample of crude A4 (15 mg) was dissolved in DMSO and purified by semi-preparative RP-HPLC using a Dionex UltiMate® 3000 on a Phenomenex Gemini Cis column using a gradient of 5% B to 35% B over 30 min (ca. 1% B/min) then 35% B to 50% B over 300 min (ca. 0.05% B/min). Fractions (1.5 mL) were collected at 0.5 min intervals and analysed by ESI-MS and RP-HPLC. Fractions identified with the correct m/z were combined and lyophilised to afford the title compound A4 as a white amorphous solid (2.43 mg, 30% yield, 97% purity by LCMS); Rₜ 14.0 min; m/z (ESI-MS) 1138.3 ([M+3H]; requires 1138.4).

LCMS was carried out using a Zorbax 300SB-C3 column (5 μm; 3.0×150 mm) using 5 to 95% B over 23 min (ca. 4.5% B per min) at 0.3 mL/min, 40° C. (where A: 0.1% formic acid in H₂O, and B: 0.1% formic acid in MeCN).

### 8.2.3 Procedure B: Representative Procedure for for the Synthesis of A4 by Solid-Phase Cysteine Lipidation

Resin-bound peptide A1 (282 mg, containing approx. 0.04 mmol peptide) was repeatedly treated with 5% TFA and 5% TIPS in DCM (v/v) for Mmt-removal. The semi-protected, resin-bound peptide was then treated with a solution of degassed DMF (2.89 mL), DMPA (10.2 mg, 0.04 mmol), vinyl palmitate 1.2 (786 mg, 2.8 mmol), tBuSH (358.5 μL, 3.2 mmol), TIPS (654.0 μL, 3.2 mmol), and TFA (250 μL, 5% v/v) such that the peptide was present at a concentration of approximately 0.01 molL⁻¹. The reaction was irradiated with UV light at 365 nm with stirring for 1 h. Following removal of the reaction mixture by filtration and washing and drying of the resins, the resin-bound peptide was treated with TFA/TIPS/H₂O (95:2.5:2.5 v/v; 5 mL) at rt for 2 h. The TFA was evaporated by a flow of nitrogen, then the peptide was precipitated in ice-cold diethyl ether, isolated by centrifugation, washed twice with cold diethyl ether, and then dissolved in MeCN/H₂O (1:1 v/v) containing TFA (0.1% v/v). Analysis by LCMS using a Zorbax 300SB-C3 (5 m; 3.0×150 mm) column confirmed the presence of peptide A4 (36.6 mg, 27% yield, 55% purity by LCMS). A sample of the crude material (15 mg) was dissolved in DMSO and purified by semi-preparative RP-HPLC to afford A4 as a white amorphous solid (2.43 mg, 30% yield, 97% purity by LCMS); Rₜ 14.0 min; MS: calcd. for [M+3H]⁺ 1138.4; found 1138.3. LCMS was carried out using a Zorbax 300SB-C3 column (5 μm; 3.0×150 mm) using 5 to 95% B over 23 min (ca. 4.5% B per min) at 0.3 mL/min, 40° C. (where A: 0.1% formic acid in H₂O, and B: 0.1% formic acid in MeCN).

### 9. Solution-Phase Cysteine Lipidation of Peptides A8, A10, and C₂

### 9.1 Synthesis of R11C(S-Pam) α-CGRP₈₋₃₇ A9 [SEQ ID NO:85]

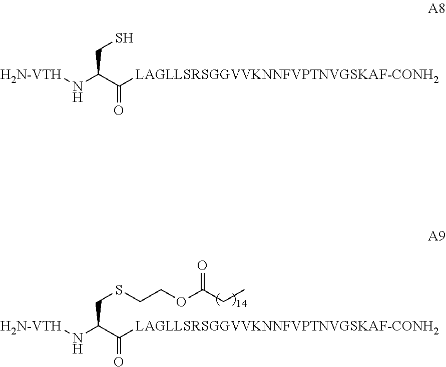

Automated Fmoc-SPPS using a Biotage® Initiator+Alstra™ microwave peptide synthesiser was used for the synthesis of R11C α-CGRP₈₋₃₇ A8 at a 0.1 mmol scale using conditions outlined in the general procedure for peptide synthesis (see section 3 above). The resin-bound peptide was then treated with TFA/TIPS/H₂O (95:2.5:2.5 v/v; 5 mL) at rt for 2 h. The TFA was evaporated by a flow of nitrogen, then the peptide was precipitated in ice-cold diethyl ether, isolated by centrifugation, washed twice with cold diethyl ether, and then dissolved in MeCN/H₂O (1:1 v/v) containing TFA (0.1% v/v) and lyophilised. The crude material A8 was used without further purification to the next step (46.1 mg, 55% crude purity by LCMS; Rₜ 12.17 min (Waters XTerra® MS C18 column (5 μm; 4.6×150 mm) at a flow rate of 1.0 m/min and a linear gradient of 5-95% B in 30 min, ca. 3% B per min); MS: calcd. for [M+3H]³⁺ 1025.19; found 1024.9. The crude peptide was then palmitoylated by a method analogous to lipidation procedure D in section 6 above and purified by semi-preparative RP-HPLC to afford R11C(S-Pam) α-CGRP₈₋₃₇ A9 as a white amorphous solid (18.57 mg, >99% purity by RP-HPLC); Rₜ 45.94 min (Waters XTerra® MS C18 column (5 μm; 4.6×150 mm) at a flow rate of 1.0 m/min and a linear gradient of 5-95% B in 90 min, ca. 1% B per min); MS: calcd. for [M+3H]³⁺ 1119.358; found 1118.9. Buffer A: H₂O containing 0.1% TFA (v/v); Buffer B: acetonitrile containing 0.1% TFA (v/v).

### 9.2 Synthesis of R11C(S-Pam), K24C(S-Pam) α-CGRP₈₋₃₇ A11 [SEQ ID NO:99]

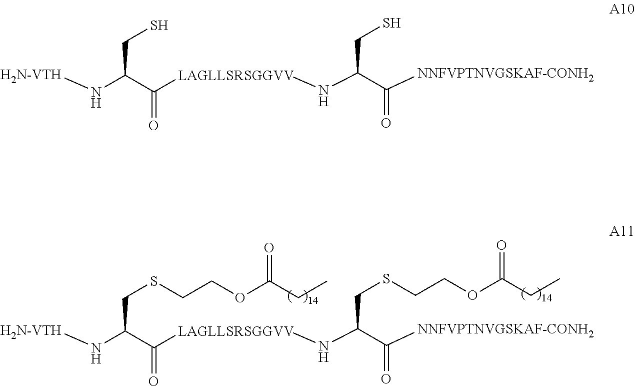

Automated Fmoc-SPPS using a Biotage® Initiator+Alstra™ microwave peptide synthesiser was used for the synthesis of R11C α-CGRP₈₋₃₇ A10 at a 0.1 mmol scale using conditions outlined in the general procedure for peptide synthesis (see section 3 above). The resin-bound peptide was then treated with TFA/TIPS/H₂O (95:2.5:2.5 v/v; 5 mL) at rt for 2 h. The TFA was evaporated by a flow of nitrogen, then the peptide was precipitated in ice-cold diethyl ether, isolated by centrifugation, washed twice with cold diethyl ether, and then dissolved in MeCN/H₂O (1:1 v/v) containing TFA (0.1% v/v) and lyophilised. The crude material A10 was used without further purification to the next step (61 mg, 70% crude purity by LCMS); Rₜ 12.65 min (Waters XTerra® MS C18 column (5 μm; 4.6×150 mm) at a flow rate of 1.0 m/min and a linear gradient of 5-95% B in 30 min, ca 3% B per min); MS: calcd. for [M+3H]³⁺ 1016.85; found 1016.4. The crude peptide was then palmitoylated by a method analogous to lipidation procedure D in section 6 above and purified by semi-preparative RP-HPLC to afford R11C(S-Pam), K24C(S-Pam) α-CGRP₈₋₃₇ A11 as a white amorphous solid (9.07 mg, >99% purity by RP-HPLC); Rₜ 70.55 min (Waters XTerra® MS C₁₈ column (5 μm; 4.6×150 mm) at a flow rate of 1.0 mL/min and a linear gradient of 5-95% B in 90 min, ca. 1% B per min); MS: calcd. for [M+3H]³⁺ 1205.174; found 1204.8. Buffer A: H₂O containing 0.1% TFA (v/v); Buffer B: acetonitrile containing 0.1% TFA (v/v).

### 9.3 Synthesis of V8C(S-Pam)O-CGRP₈₋₃₇ C3 [SEQ ID NO:88]

Automated Fmo

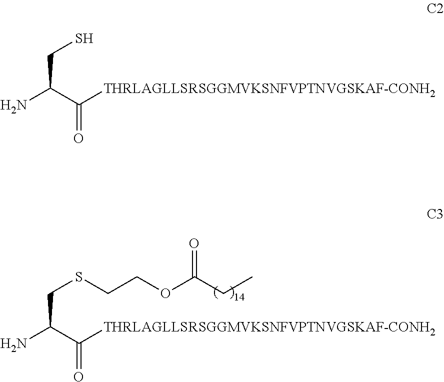

synthesiser was used for the synthesis of V8C β-CGRP₈₋₃₇ C2 at a 0.1 mmol scale using conditions outlined in the general procedure for peptide synthesis (see section 3 above). The resin-bound peptide was then treated with TFA/TIPS/H₂O (95:2.5:2.5 v/v; 5 mL) at rt for 2 h. The TFA was evaporated by a flow of nitrogen, then the peptide was precipitated in ice-cold diethyl ether, isolated by centrifugation, washed twice with cold diethyl ether, and then dissolved in MeCN/H₂O (1:1 v/v) containing TFA (0.1% v/v) and lyophilised. The crude material C₂ was used without further purification to the next step (46.95 mg, 62% crude purity by LCMS); Rₜ 11.55 min (Waters XTerra® MS C₁₈ column (5 μm; 4.6×150 mm) at a flow rate of 1.0 m/min and a linear gradient of 95% B in 30 min, ca 3% B per min); MS: calcd. for [M+3H]³⁺ 1045.89; found 1045.6. The crude peptide was then palmitoylated by a method analogous to lipidation procedure D in section 6 above and purified by semi-preparative RP-HPLC to afford V8C(S-Pam) α-CGRP₈₋₃₇ C3 as a white amorphous solid (9.15 mg, >99% purity by RP-HPLC); Rₜ 46.15 min (Waters XTerra® MS C18 column (5 μm; 4.6×150 mm) at a flow rate of 1.0 m/min and a linear gradient of 5-95% B in 90 min, ca. 1% B per min); MS: calcd. for [M+3H]³⁺ 1140.055; found 1139.8. Buffer A: H₂O containing 0.1% TFA (v/v); Buffer B: acetonitrile containing 0.1% TFA (v/v).

### Example 2

1. This example described the preparation of peptides for use in comparison with the peptide conjugates of the invention. Synthesis of native CGRP₈₋₃₇ A [SEQ ID NO:95]

NH₂-VTHRLAGLLSRSGGVVKNNFVPTNVGSKAF-CONH₂
A

Automated Fmoc-SPPS using a PS3™ peptide synthesiser was used for the synthesis of CGRP₈₋₃₇ A on a 0.1 mmol scale, followed by resin cleavage using the conditions outlined in the general procedure for peptide synthesis (see section 3 above) to afford crude A as a white solid (170 mg, 33% yield based on 61% purity by RP-HPLC); Rₜ 29.9 min; m/z (ESI-MS) 626.0 ([M+5H]⁵⁺ requires 626.1). RP-HPLC was carried out using a Phenomenex Gemini C₁₈ column (5 μm; 10.0×250 mm) using 5 to 65% B over 60 min (ca. 1% B/min) at 5.0 mL/min, 45° C. (where A: 0.1% TFA in H₂O and B: 0.1% TFA in MeCN).

The crude peptide A (170 mg) was purified by semi-preparative RP-HPLC using a Dionex UltiMate® 3000 on a Phenomenex Gemini Cis column using a gradient of 0% B to 13% B over 13 min (ca. 1% B/min) then 13% B to 50% B over 370 min (ca. 0.1% B/min).

Fractions (2.5 mL) were collected at 0.5 min intervals and analysed by ESI-MS and RP-HPLC as described in Example 1. Fractions identified with the correct m/z were combined and lyophilised to afford the title compound A as a white amorphous solid (15.9 mg, 15% yield, 93% purity); Rₜ 12.6 min; m/z (ESI-MS) 626.1 ([M+5H]⁵⁺ requires 626.1). LCMS was carried out using a Zorbax 300SB-C3 column (5 μm; 3.0×150 mm) using 5 to 65% B over 21 min (ca. 3% B/min) at 0.3 mL/min, rt (where A: 0.1% formic acid in H₂O, and B: 0.1% formic acid in MeCN).

### 9.3.1 Synthesis of β-CGRP₈₋₃₇ C1 [SEQ ID NO:31]

C1
H₂N-VTHRLAGLLSRSGGMVKSNFVPTNVGSKAF-CONH₂

Automated Fmoc-SPPS using a Biotage® Initiator+Alstra™ microwave peptide synthesiser was used for the synthesis of β-CGRP₈₋₃₇ C1 at a 0.1 mmol scale using conditions outlined in the general procedure for peptide synthesis (see section 3 above). The resin-bound peptide was then treated with TFA/TIPS/H₂O (95:2.5:2.5 v/v; 5 mL) at rt for 2 h. The TFA was evaporated by a flow of nitrogen, then the peptide was precipitated in ice-cold diethyl ether, isolated by centrifugation, washed twice with cold diethyl ether, and then dissolved in MeCN/H₂O (1:1 v/v) containing TFA (0.1% v/v) and lyophilised. The crude material was purified by semi-preparative RP-HPLC to afford C1 as a white amorphous solid (5.08 mg, >99% purity by RP-HPLC); Rₜ 25.31 min (Waters XTerra® MS C18 column (5 μm; 4.6×150 mm) and a linear gradient of 5-95% B in 90 min, ca. 1% B per min at a flow rate of 1.0 mL/min. Buffer A: H₂O containing 0.1% TFA (v/v); Buffer B: acetonitrile containing 0.1% TFA (v/v)); MS: calcd. for [M+3H]³⁺ 1044.559; found 1044.3.

### Example 3

This example describes measurement of the antagonist activity of peptide conjugates of the invention at CGRP receptors.

1. Antagonism of CGRP-Induced cAMP Accumulation in Transfected CGRP Receptor Expressing Cells

Simian vacuolating virus 40 (SV40) transformed African green monkey kidney (Cos 7) cells were transiently transfected with calcitonin receptor-like receptor (CLR) or calcitonin receptor (CTR) together with receptor activity-modifying protein 1 (RAMP1) to form CGRP or AMY₁₍ₐ₎ receptors.

Cos 7 cells were cultured in Dulbecco's Modified Eagle Medium (DMEM) supplemented with 8% heat inactivated fetal bovine serum and kept in a 37° C. humidified 95% air/5% CO₂ incubator. Cells were plated into 96 well plates at 15-20,000 cells/well in 100 μl media one day prior to transfection using polyethylenimine.

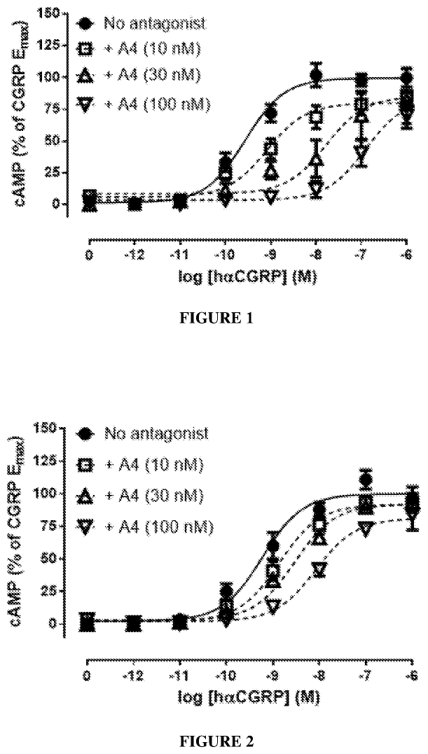

On the day of experimentation, media was replaced with 50 μl DMEM containing 1 mM isobutylmethylxanthine and 0.1% bovine serum albumin for 30 min. All peptides were diluted in assay medium and after the 30 min incubation, 25 μl antagonists were added, followed immediately by 25 μl human alpha CGRP (hαCGRP), giving a total assay volume of 100 μl. Once human alpha CGRP was added, the cells were incubated at 37° C. for 15 minutes. The media was then aspirated from the wells and reactions terminated with ice-cold absolute ethanol. Ethanol was evaporated to dryness and cAMP was quantified using a commercially-available assay (LANCE Perkin Elmer). The results are shown in FIGS. 1 and 2 and Table 1.

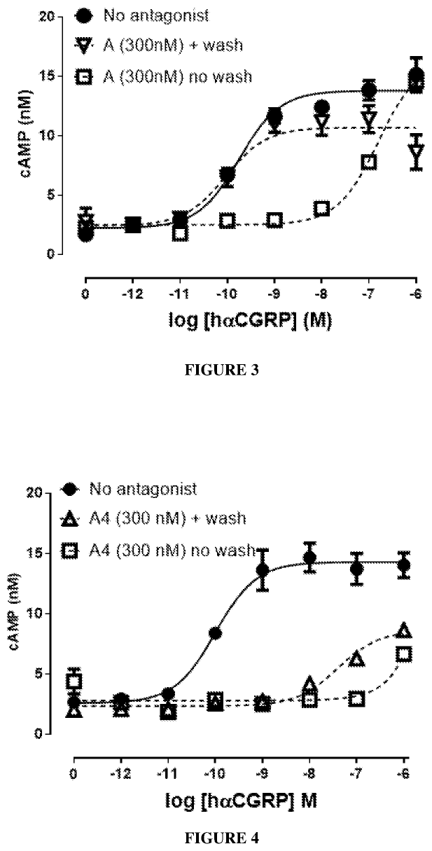

In a different specific experimental paradigm, media was replaced with DMEM containing 1 mM isobutylmethylxanthine and 0.1% bovine serum albumin for 30 min. After this period, 25 μl of antagonist or media was added for 15 minutes at 37° C. as a pre-incubation period. After pre-incubation, the antagonist or media were aspirated from some wells, washed once with phosphate-buffered saline and replaced with 75 μl new media. The other wells with antagonist were not washed. Human alpha CGRP (25 μl) was then added to a total assay volume of 100 μl. Once human alpha CGRP was added, the cells were incubated at 37° C. for 15 minutes. The media was then aspirated from the wells and reactions terminated with ice-cold absolute ethanol. Ethanol was evaporated to dryness and cAMP was quantified using a commercially-available assay (LANCE Perkin Elmer). The results are shown in FIGS. 3 and 4 and in Table 2 below.

2. Antagonism of CGRP-Induced cAMP Accumulation in SK-N-MC Cells

SK-N-MC are human neuroblastoma-derived cells that endogenously express a CGRP receptor (Choksi et al, 2002). The cells were cultured in DMEM supplemented with 8% heat inactivated fetal bovine serum and kept in a 37° C. humidified 95% air/5% CO₂ incubator. Cells were plated into 96 well plates at 15-20,000 cells/well in 100 μl media.

On the day of experimentation, media was replaced with DMEM containing 1 mM isobutylmethylxanthine and 0.1% bovine serum albumin for 30 min. After this period antagonists were added, followed by human alpha CGRP. Once human alpha CGRP was added, the cells were incubated at 37° C. for 15 minutes. The media was then aspirated from the wells and reactions terminated with ice-cold absolute ethanol. Ethanol was evaporated to dryness and cAMP was quantified using a commercially-available assay (LANCE Perkin Elmer). The results are shown in Table 1 below.

### 3. Calculation of Antagonist Potency Values

Data were analysed using Graphpad Prism 7. Antagonist pKB or pA₂ values were obtained using Global Schild analysis. Where the Schild slope was not significantly different from unity the data are expressed as pK_{B} values. Where a single concentration of antagonist was used, pA₂ values are used to describe antagonist potency.

### 4. Antagonism In Vivo of Capsaicin-Induced Ear Vasodilation in C57BL/6 Mice

On the day of experiment, 10 mM stock solutions of antagonists A and A4 in dimethylsulfoxide were diluted in saline vehicle containing a final amount of 0.1% bovine serum albumin and 3.2% dimethylsulfoxide. Capsaicin solutions were constituted at 3.03 mg/ml by dissolving capsaicin powder (at ≥95% purity) in absolute ethanol. Animal studies were conducted within a room maintained at 20 to 21 degrees Celsius.

The antagonists at a dose of 960 nmol/kg (10 ml/kg in saline containing 0.1% BSA and 3.2% dimethylsulfoxide), or saline vehicle (containing 0.1% BSA and 3.2% dimethylsulfoxide) were each administered subcutaneously to C57BL/6 mice at 10 minutes prior to anesthesia. The absolute injection volumes for A and A4 were adjusted such that the final dose administered was 960 nmol/kg. Mice were then anesthetized with a cocktail that consisted of ketamine and xylazine at 100 mg/kg and 10 mg/kg respectively (10 ml/kg), administered subcutaneously. Anesthetized mice were then placed onto a Kent Scientific heating pad set to maintain body temperature at approximately 37 degrees Celsius with the head positioned so that both ears were aligned horizontally to the Moor LDI2-HIR Laser Doppler imager.

Following administration of ketamine and xylazine to induce anesthesia (8-10 minutes), blood flow in both ears for each mouse was scanned for three minutes prior to capsaicin application to define the baseline blood flow. These studies were performed utilizing a Laser Doppler imager and MoorLDI Measurement 6.1. Following these baseline measurements, the capsaicin solution was applied topically at a volume of 20 μL per ear (10 μL on dorsal side and 10 μL on ventral side). On the contralateral ear, absolute ethanol was applied topically as a control at the same volume and procedure. Immediately, both ears were scanned with the Laser Doppler imager for 15 minutes to capture the capsaicin-induced vasodilation.

### 4.1 Calculation of Vasodilatory Activity

Mean flux values for both ears (control and capsaicin) at each timepoint (per minute) were analysed using MoorLDI Review 6.1 and Graphpad Prism 7 software. Flux mean values were normalised to the mean average of the baseline flux values. Area under the curve (AUC) of the normalised profile baseline was measured for each animal, grouped and compared between the different treatment arms. Statistical analyses were performed using One-way Anova and Tukey's Post-hoc tests.

### 5. Results

FIGS. 1 and 2 show rightward-shifts in the CGRP concentration-response curve showing antagonism of both the CGRP and the AMY₁ receptor.

The results of experiments using CGRP peptide antagonists at human CGRP or AMY₁ receptor transfected Cos-7 cells or SK-N-MC cells, which endogenously express a CGRP receptor are shown in Table 1. The data in Table 1 shows antagonist potency values (all values in Table 1 are pK_{B} except for the values for SK-N-MC and C1, C3, A6, A9, and A11 which are pA₂) for CGRP antagonists obtained from cAMP assays. Data are mean±s.e.m from n independent experiments, as indicated in parentheses. *p<0.05, **p<0.01, ***p<0.001 by one way ANOVA followed by Dunnett's multiple comparisons test, compared to CGRP₈₋₃₇. ^{#}p<0.05, ^{##}p<001, ^{###}p<0.001 by unpaired test, comparing antagonist potency between CGRP and AMY₁ receptors (for antagonists A, B, B1, A4, A5, and A6 only).

TABLE 1
Data summary of CGRP peptide antagonism at human CGRP receptors

| | | | Selectivity | |
|---|---|---|---|---|
| | | | (fold- | |
| Antag- | | | difference | |
| onist | CGRP | AMY₁ | CGRP vs. | |
| tested | (CLR/RAMP1) | (CTR/RAMP1) | AMY₁) | SK-N-MC (pA₂) |
| Telcage- | 9.06 ± 0.03 (3) | Not tested | | 9.35 ± 0.10 (3) |
| pant | (A₂: 0.87 nM) | | | (A₂: 0.45 nM) |
| A | 9.09 ± 0.16 (5) | 7.12 ± 0.13 | 93 | 9.85 ± 0.16 (3) |
| | (A₂: 0.81 nM) | (5)^{###} (A₂: 76 | | (A₂: 0.14 nM) |
| | | nM) | | |
| B | 8.31 ± 0.04 (3)* | 7.12 ± 0.12 | 15 | Not tested |
| | (A₂: 4.9 nM) | (3)^{###} (A₂: 76 | | |
| | | nM) | | |
| C1 | 8.99 ± 0.24 (2) | Not tested | — | Not tested |
| | (A₂: 1.0 nM) | | | |
| B1 | 8.78 ± 0.08 (3) | 8.66 ± 0.17 | <2 | 10.65 ± 0.25 (3) |
| | (A₂: 1.7 nM) | (3)*** | | (A₂: 0.02 nM) |
| | | (A₂: 2.2 nM) | | |
| A4 | 9.23 ± 0.21 (3) | 8.15 ± 0.24 | 12 | 10.76 ± 0.27 (3)* |
| | (A₂: 0.59 nM) | (3)***,^{#} | | (A₂: 0.02 nM) |
| | | (A₂: 7.1 nM) | | |
| A5 | 8.17 ± 0.04 | 8.75 ± 0.09 | −4 | 10.06 ± 0.18 (3) |
| | (3)** | (3)***,^{#} | | (A₂: 0.09 nM) |
| | (A₂: 6.8 nM) | (A₂: 1.8 nM) | | |
| A6 | 7.06 ± 0.29 | 5.97 ± 0.14 | 12 | Not tested |
| | (3)** | (3)***,^{###} | | |
| | (A₂: 87 nM) | (A₂: 1071 nM) | | |
| C3 | 9.68 ± 0.19 (2) | Not tested | — | Not tested |
| | (A₂: 0.21 nM) | | | |
| A9 | 7.74 ± 0.04 (2) | Not tested | — | Not tested |
| | (A₂: 18 nM) | | | |
| A11 | <7 (2) | Not tested | — | Not tested |
| | (A₂: >100 nM) | | | |

Note: A₂ is the antilog of the negative pA₂. For the purposes of defining antagonist potency the pKB equals the pA₂.

Based on Table 1 it can be concluded that lipidated CGRP peptide antagonists equipotent with antagonist A (CGRP₈₋₃₇) and with higher potency than antagonist A, and with lower potency than A have been synthesised.

Based on Table 1 it can also be concluded that lipidated CGRP peptide antagonists with lower selectivity between CGRP and AMY₁ receptors than antagonist A have been synthesised.

Based on Table 1 it can also be concluded that lipidated CGRP peptide antagonists equipotent with a small molecule CGRP receptor antagonist, Telcagepant, or with higher potency than Telcagepant have been synthesised.

FIGS. 3 and 4 show that CGRP receptor antagonism by antagonist A4 is retained following washing, compared to antagonism by antagonist A following washing.

The results of experiments using CGRP receptor antagonists at human CGRP or AMY₁ receptor transfected Cos-7 cells following washing, compared to antagonism without washing are shown in Table 2. The data in Table 2 shows antagonist potency values (all values in Table 2 are pA₂ values) for CGRP antagonists obtained from cAMP assays. Data are mean±s.e.m from n independent experiments, as indicated in parentheses. *p<0.05, ***p<0.001 by unpaired t test, comparing antagonist potency between without wash to with wash.

TABLE 2
Data summary of CGRP peptide antagonism at human CGRP
receptors with and without washing

| Antag- | | | | |
|---|---|---|---|---|
| onist | CGRP (CLR/RAMP1) | | AMY₁ (CTR/RAMP1) | |
| tested | Without wash | With wash | Without wash | With wash |
| A | 9.21 ± 0.21 (5) | <7.52 (4⁺) (A₂: | 7.23 ± 0.10 (5) | <6 (5) (A₂: |
| | (A₂: 0.62 nM) | >30 nM) | (A₂: 59 nM) | >1000 nM) |
| B1 | 9.86 ± 0.14 (5) | 9.34 ± 0.28 (5) | 9.30 ± 0.26 (4) | 8.93 ± 0.15 (4) |
| | (A₂: 0.14 nM) | (A₂: 0.46 nM) | (A₂: 0.50 nM) | (A₂: 1.2 nM) |
| A4 | 9.82 ± 0.09 (4) | 8.48 ± 0.09 | 8.98 ± 0.25 (5) | 8.54 ± 0.31 (5) |
| | (A₂: 0.15 nM) | (4)*** (A₂: 3.3 | (A₂: 1.0 nM) | (A₂: 2.9 nM) |
| | | nM) | | |
| A5 | 10.28 ± 0.12 (5) | 9.13 ± 0.14 | 9.43 ± 0.20 (5) | 8.45 ± 0.23 (5)* |
| | (A2: 0.05 nM) | (5)*** (A₂: | (A₂: 0.37 nM) | (A₂: 3.5 nM) |
| | | 0.74 nM) | | |
| A6 | 6.64 ± 0.27 (3) | <5.52 (3) (A₂: | Not tested | Not tested |
| | (A₂: 229 nM) | >3000 nM) | | |

⁺In one further experiment a pA₂ of 7.98 was reported.

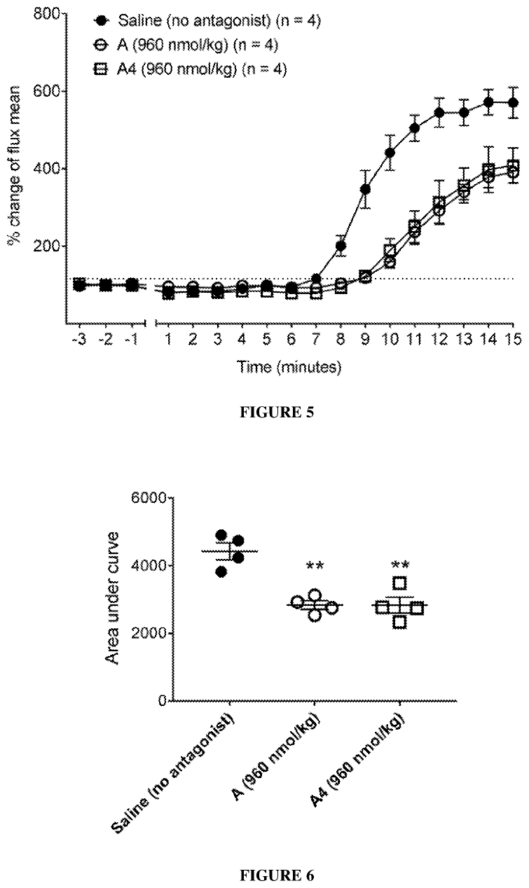

FIG. 5 shows in vivo attenuation of the capsaicin-evoked vasodilatory response in the ear of C57BL/6 mice by antagonists A and A4. Data are mean±s.e.m from n independent experiments. FIG. 6 shows that attenuation of the vasodilatory response by A and A4 compared to control is significant as analysed by AUC.

Based on FIGS. 5 and 6 it can be concluded that antagonist A4 attenuates capsaicin-evoked vasodilation in vivo and is at least equipotent with A.

Table 3 shows the fold change reduction in antagonist potency (pA₂) at human CGRP receptors with wash and the fold change of antagonists B1, A4, A5, and A6 compared to antagonist A for the data in Table 2.

TABLE 3

| | CGRP (CLR/RAMP1) | AMY₁ (CTR/RAMP1) |
|---|---|---|
| Antagonist | Fold change reduction | Fold change reduction |
| tested | in pA₂ with wash^{a} | in pA₂ with wash^{a} |
| A | >49 | >17 |
| B1 | 3.3 | 2.3 |
| A4 | 22 | 2.8 |
| A5 | 14 | 9.6 |
| A6 | >13 | Not tested |

^{a}Calculated for each antagonist by dividing the antagonist potency value (pA₂) with wash in Table 2 by the antagonist potency value (pA₂) without wash.

### INDUSTRIAL APPLICABILITY

The peptide conjugates described herein have useful CGRP receptor antagonist activity. As such, these compounds are useful for treating various diseases and conditions, such as those mediated by CGRP receptors as described herein. Such diseases and conditions include, for example, thermal injury, circulatory shock, menopausal hot flushes, asthma, sepsis, neurogenic inflammation, inflammatory skin conditions (for example psoriasis and contact dermatitis), allergic rhinitis, joint disorders (for example arthritis and temporomandibular joint disorder, preferably arthritis), cachexia (for example cancer-induced cachexia), pain, for example craniofacial pain disorders (for example migraine, headache, trigeminal neuralgia and dental pain, preferably migraine), and metabolic disorders or syndromes (for example obesity, type II diabetes, insulin resistance, dyslipidemia, hypertension, atherosclerosis and thrombosis).

The following numbered paragraphs relate to aspects and embodiments of the invention described herein.
1. A peptide conjugate comprising a calcitonin gene-related peptide (CGRP) peptide, wherein at least one amino acid of the peptide is covalently conjugated to a lipid-containing moiety, wherein the peptide conjugate is a CGRP receptor antagonist.
2. A peptide conjugate comprising a calcitonin gene-related peptide (CGRP) peptide, wherein at least one amino acid of the peptide is covalently conjugated to a lipid-containing moiety via a sulfur atom of a sulfide group, wherein the peptide conjugate is a CGRP receptor antagonist.
3. The peptide conjugate of paragraph 1 or 2, wherein the peptide conjugate has an antagonist potency value (pA₂) more than a value about 10-fold less than, 5-fold less than, 3-fold less than, 2-fold less than, 1-fold less than the antagonist potency (pA₂) of α-CGRP8-37 (SEQ ID NO:96) at a CGRP receptor or has an antagonist potency value (pA₂) more than a value equal to the antagonist potency (pA₂) of CGRP8-37 at a CGRP receptor, for example as measured by a cAMP assay as described in the Examples herein.
4. The peptide conjugate of any one of the preceding paragraphs, wherein the peptide conjugate has a half life at least 2-, 3-, 4-, 5-, 10-, 20-, 30-, 40-, or -50-fold longer than the half life of α-CGRP8-37 (SEQ ID NO:96), for example as measured in a suitable rodent model, for example a rat model.
5. The peptide conjugate of any one of the preceding paragraphs, wherein the at least one amino acid is cysteine or homocysteine.
6. The peptide conjugate of any one of the preceding paragraphs, wherein the at least one amino acid is cysteine.
7. The peptide conjugate of any one of the preceding paragraphs, wherein the peptide conjugate comprises only one amino acid conjugated to a lipid-containing moiety.
8. The peptide conjugate of any one of paragraphs 1-6, wherein the peptide conjugate comprises two or more amino acids each conjugated to a lipid-containing moiety.
9. The peptide conjugate of any one of the preceding paragraphs, wherein the lipid-containing moiety comprises one or more straight or branched aliphatic or heteroaliphatic chains each containing at least 4 or at least 6 chain-linked atoms.
10. The peptide conjugate of any one of any one of the preceding paragraphs, wherein the lipid-containing moiety comprises one or more saturated or unsaturated fatty acid esters.
11. The peptide conjugate of any one of the preceding paragraphs, wherein the lipid-containing moiety is of the formula (A):

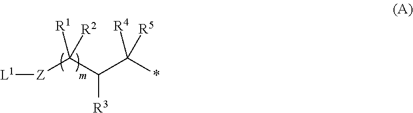

wherein
   * represents a bond to the sulfur atom of the sulfide group of the amino acid to which the lipid-containing moiety is conjugated;
   Z and Z¹ are each independently selected from the group consisting of —O—, —NR—, —S—, —S(O)—, —SO₂—, —C(O)O—, —OC(O)—, —C(O)NR—, —NRC(O)—, —C(O)S—, —SC(O)—, —OC(O)O—, —NRC(O)O—, —OC(O)NR—, and —NRC(O)NR—;
   R is hydrogen or C₁₋₆aliphatic;
   m is an integer from 0 to 4;
   n is 1 or 2;
   R¹ and R² at each instance of m are each independently hydrogen, C₁₋₆aliphatic; or R¹ is L²-Z¹—C₁₋₆alkyl;
   R³, R⁴, and R⁵ are each independently hydrogen or C₁₋₆aliphatic; or R³ is L²-Z¹—C₁₋₆ alkyl;
   L¹ and L² are each independently C₅₋₂₁aliphatic or C₄₋₂₀heteroaliphatic; provided that:
      when R³ is L²-Z¹—C₁₋₆alkyl, R¹ is not L²-Z¹—C₁₋₆alkyl; and
      when m is an integer from 2 to 4, no more than one R¹ is L²-Z¹—C₁₋₆alkyl; and
wherein any aliphatic, alkyl, or heteroaliphatic present in any of R, R¹, R², R³, R⁴, R⁵, L¹, and L² is optionally substituted with one or more independently selected optional substituents.
12. The peptide conjugate of paragraphs 11, wherein
   R is hydrogen, C₁₋₆alkyl, or C₃₋₆cycloalkyl;
   m is an integer from 0 to 4;
   n is 1 or 2;
   R¹ and R² at each instance of m are each independently hydrogen, C₁₋₆alkyl, or C₃₋₆cycloalkyl; or R¹ is L²-Z¹—C₁₋₆alkyl;
   R³, R⁴, and R⁵ are each independently hydrogen, C₁₋₆alkyl, or C₃₋₆cycloalkyl; or R³ is L²-Z¹—C₁₋₆alkyl;
   L¹ and L² are each independently C₅₋₂₁alkyl, C₅₋₂₁alkenyl, or C₄₋₂₀heteroalkyl;
   wherein any alkyl, alkenyl, cycloalkyl, or heteroalkyl present in any of R, R¹, R², R³, R⁴, R⁵, L¹, and L² is optionally substituted with one or more independently selected optional substituents.
13. The peptide conjugate of paragraph 11 or 12, wherein
   R is hydrogen or C₁₋₆alkyl;
   m is an integer from 0 to 4;
   n is 1 or 2;
   R¹ and R² at each instance of m are each independently hydrogen or C₁₋₆alkyl; or R¹ is L²-Z¹—C₁₋₆alkyl;
   R³, R⁴, and R⁵ are each independently hydrogen or C₁₋₆alkyl; or R³ is L²-Z¹—C₁₋₆alkyl;
   L and L² are each independently C₅₋₂₁alkyl, C₅₋₂₁alkenyl, or C₄₋₂₀heteroalkyl;
   wherein any alkyl, alkenyl, or heteroalkyl present in any of R, R¹, R², R³, R⁴, R⁵, L¹, and L² is optionally substituted with one or more independently selected optional substituents.
14. The peptide conjugate of any one of paragraphs 11-13, wherein Z and Z¹ are each independently selected from —C(O)O—, —C(O)NR—, and —C(O)S—, preferably —C(O)O—.
15. The peptide conjugate of any one of paragraphs 11-14, wherein the lipid-containing moiety is of the formula (I)

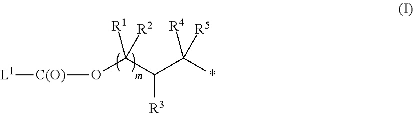

wherein
m, L¹, R¹, R², R³, R⁴, and R⁵ are as defined in any one of the preceding paragraphs; and Z¹ when present is —C(O)O—.
16. The peptide conjugate of any one of paragraphs 11-15, wherein m is an integer from 0 to 2.
17. The peptide conjugate of any one of paragraphs 11-16, wherein m is 0 or 1.
18. The peptide conjugate of any one of paragraphs 11-17, wherein m is 0.
19. The peptide conjugate of any one of paragraphs 11-18, wherein R¹ and R² at each instance of m are each independently hydrogen.
20. The peptide conjugate of any one of paragraphs 11-19, wherein R⁴ and R⁵ are each hydrogen.
21. The peptide conjugate of any one of paragraphs 11-20, wherein R³ is hydrogen or C₁₋₆alkyl.
22. The peptide conjugate of any one of paragraphs 11-20, wherein the lipid-containing moiety is of the formula (IV):

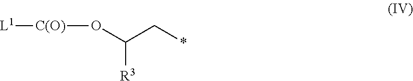

wherein
   R³ is hydrogen, L²-C(O)—OCH₂, or L²-C(O)—OCH₂CH₂; and
   L¹ and L² are each independently C₅₋₂₁alkyl, C₅₋₂₁alkenyl, or C₄₋₂₀heteroalkyl.
23. The peptide conjugate of any one of paragraphs 11-22, wherein L¹ and L² are each independently is C₅₋₂₁alkyl.
24. The peptide conjugate of any one of paragraphs 11-23, wherein L¹ and L² are each independently is C₉₋₂₁alkyl.
25. The peptide conjugate of any one of paragraphs 11-24, wherein L¹ and L² are each independently linear C₁₅alkyl.
26. The peptide conjugate of any one of paragraphs 11-20 and 22-25, wherein R³ is L²-C(O)—OCH₂CH₂.
27. The peptide conjugate of any one of paragraphs 11-25, wherein R³ is hydrogen.
28. The peptide conjugate of any one of paragraphs 11-27, wherein the one or more independently selected optional substituents are selected from halo, CN, NO₂, OH, NH₂, NHR^{x}, NR^{x}R^{y}, C₁₋₆haloalkyl, C₁₋₆haloalkoxy, C(O)NH₂, C(O)NHR^{x}, C(O)NR^{x}R^{y}, SO₂R^{x}, OR^{y}, SR^{x}, S(O)R^{x}, C(O)R^{x}, and C₁₋₆aliphatic; wherein R^{x} and R^{y} are each independently C₁₋₆aliphatic, for example C₁₋₆alkyl.
29. The peptide conjugate of any one of the preceding paragraphs, wherein the N-terminal group of the peptide is —NR^{a}R^{b}, wherein R^{a} and R^{b} are each independently hydrogen, alkyl, cycloalkyl, acyl, aryl, or arylalkyl; and/or the C-terminal group of the peptide is —CH₂OR^{c}, —C(O)OR^{c} or —C(O)NR^{c}R^{d}, wherein R^{c} and R^{d} are each independently hydrogen, alkyl, cycloalkyl, aryl, or arylalkyl.
30. The peptide conjugate of any one of the preceding paragraphs, wherein the N-terminal group of the peptide is —NH₂ or —NH(acyl), for example —NHAc; and/or the C-terminal group of the peptide is —C(O)NH₂.
31. A peptide conjugate of any one of the preceding paragraphs, wherein the peptide comprises or consists of an amino acid sequence of the formula:

[SEQ ID No. 1]
Z-Xaa⁸Xaa⁹Xaa¹⁰Xaa¹¹Leu¹²Xaa¹³Xaa¹⁴Xaa¹⁵
Leu¹⁶Xaa¹⁷Xaa¹⁸Xaa¹⁹Xaa²⁰Xaa²¹Xaa²²Xaa²³
Xaa²⁴Xaa²⁵Xaa²⁶Phe²⁷Xaa²⁸Xaa²⁹Thr³⁰Xaa³¹
Val³²Gly³³Xaa³⁴Xaa³⁵Xaa³⁶Phe³⁷

wherein:
   Z is absent or is Xaa¹Xaa²Xaa³Xaa⁴Xaa⁵Xaa⁶Xaa⁷, Xaa²Xaa³Xaa⁴Xaa⁵Xaa⁶Xaa⁷, Xaa³Xaa⁴Xaa⁵Xaa⁶Xaa⁷, Xaa⁴Xaa⁵Xaa⁶Xaa⁷, Xaa⁵Xaa⁶Xaa⁷, Xaa⁶Xaa⁷ or Xaa⁷
   wherein:
      Xaa¹ is alanine, valine, leucine, isoleucine, proline, phenylalanine, methionine, tryptophan, serine, glycine, asparagine, glutamine, threonine, tyrosine or cysteine;
      Xaa² is cysteine, serine, alanine, glycine, asparagine, glutamine, threonine, tyrosine;
      Xaa³ is aspartate, glutamate, asparagine, glutamine, glycine, serine, threonine, tyrosine or cysteine;
      Xaa⁴ is threonine, glycine, asparagine, glutamine, serine, phenylalanine, tyrosine, valine, isoleucine or cysteine;
      Xaa⁵ is alanine, valine, leucine, isoleucine, proline, phenylalanine, tyrosine methionine or tryptophan;
      Xaa⁶ is threonine, glycine, asparagine, glutamine, serine, tyrosine, phenylalanine, valine, isoleucine or cysteine;
      Xaa⁷ is cysteine, serine, alanine, glycine, asparagine, glutamine, threonine, phenylalanine or tyrosine;
   Xaa⁸ is valine, alanine, leucine, isoleucine, proline, phenylalanine, tyrosine methionine, tryptophan or threonine;
   Xaa⁹ is threonine, glycine, asparagine, glutamine, serine, tyrosine, valine, isoleucine or cysteine;
   Xaa¹⁰ is histidine, lysine, arginine, asparagine, glutamine, serine, alanine, glycine, valine, leucine or isoleucine;
   Xaa¹¹ is arginine, lysine, histidine, glutamine or asparagine;
   Xaa¹³ is alanine, valine, leucine, isoleucine, proline, phenylalanine, methionine, tryptophan, serine, glycine, asparagine, glutamine, threonine, tyrosine or cysteine;
   Xaa¹⁴ is glycine, proline, alanine, asparagine, glutamine, serine, threonine, phenylalanine, tyrosine, cysteine, glutamate or aspartate;
   Xaa¹⁵ is leucine, isoleucine, valine, alanine, methionine, phenylalanine, tyrosine, proline or tryptophan;
   Xaa¹⁷ is serine, threonine, alanine, valine, leucine, isoleucine, proline, phenylalanine, tyrosine, methionine, tryptophan, arginine, lysine, histidine, glutamine, asparagine or cysteine;
   Xaa¹⁸ is arginine, lysine, histidine, glutamine or asparagine;
   Xaa¹⁹ is serine, threonine, alanine, valine, leucine, isoleucine, proline, phenylalanine, tyrosine, methionine, tryptophan or cysteine;
   Xaa²⁰ is glycine, proline, alanine, beta alanine, asparagine, glutamine, serine, threonine, phenylalanine or tyrosine;
   Xaa²¹ is glycine, proline, alanine, beta alanine, asparagine, glutamine, serine, threonine, phenylalanine or tyrosine;
   Xaa²² is valine, alanine, leucine, isoleucine, proline, phenylalanine, tyrosine, methionine or tryptophan or threonine;
   Xaa²³ is valine, alanine, leucine, isoleucine, proline, phenylalanine, tyrosine, methionine, tryptophan or threonine;
   Xaa²⁴ is lysine, arginine, glutamine, asparagine or histidine;
   Xaa²⁵ is asparagine, glutamine, glycine, serine, threonine, tyrosine, phenylalanine, alanine, glutamate, aspartate or cysteine;
   Xaa²⁶ is asparagine, glutamine, glycine, serine, threonine, phenylalanine, tyrosine or cysteine;
   Xaa²⁸ is valine, alanine, leucine, isoleucine, proline, phenylalanine, tyrosine, methionine, tryptophan or threonine;
   Xaa²⁹ is proline, alanine, valine, leucine, isoleucine, glycine, phenylalanine, tyrosine, methionine or tryptophan;
   Xaa³¹ is asparagine, glutamine, glycine, serine, threonine, phenylalanine, tyrosine, glutamate, aspartate or cysteine;
   Xaa³⁴ is serine, threonine, alanine, valine, leucine, isoleucine, proline, phenylalanine, tyrosine, methionine, tryptophan or cysteine;
   Xaa³⁵ is lysine, arginine, glutamine, asparagine, histidine, aspartate or glutamate; and
   Xaa³⁶ is alanine, valine, leucine, isoleucine, proline, phenylalanine, tyrosine, methionine or tryptophan;
wherein one or more of Xaa1-Xaa11, Xaa13-Xaa15, Xaa17-Xaa26, Xaa28, Xaa29, Xaa31 and Xaa34-Xaa36 is or is substituted with an amino acid that is covalently conjugated to a lipid-containing moiety.
32. A peptide conjugate of paragraph 31 wherein Z is absent, or is Xaa¹Xaa²Xaa³Xaa⁴Xaa⁵Xaa⁶Xaa⁷ or Xaa⁷.
33. A peptide conjugate of paragraph 31 or 32 wherein
   a) Xaa1 is alanine, valine, leucine, isoleucine, serine, glycine, or threonine;
   b) Xaa2 is cysteine, serine or alanine;
   c) Xaa3 is aspartate, glutamate, asparagine or glutamine;
   d) Xaa4 is threonine, glycine, asparagine, glutamine or serine;
   e) Xaa5 is alanine, valine, leucine or isoleucine;
   f) Xaa6 is threonine, glycine, asparagine, glutamine or serine;
   g) Xaa7 is cysteine, serine, or alanine;
   h) Xaa8 is valine, alanine, leucine, isoleucine, phenylalanine or methionine;
   i) Xaa9 is threonine, glycine, asparagine, glutamine or serine;
   j) Xaa10 is histidine, lysine or arginine;
   k) Xaa11 is arginine, lysine or histidine;
   l) Xaa13 is alanine, valine, leucine, isoleucine, serine, glycine, or threonine;
   m) Xaa14 is glycine, proline, alanine, aspartate or glutamate;
   n) Xaa15 is leucine, isoleucine, valine, alanine, methionine or phenylalanine;
   o) Xaa17 is serine, threonine, alanine, arginine, lysine or histidine;
   p) Xaa18 is arginine, lysine or histidine;
   q) Xaa19 is serine, threonine or alanine;
   r) Xaa20 is glycine, proline or alanine;
   s) Xaa21 is glycine, proline or alanine;
   t) Xaa22 is valine, alanine, leucine, isoleucine, phenylalanine or methionine;
   u) Xaa23 is valine, alanine, leucine, isoleucine, proline, phenylalanine, methionine, tryptophan or threonine;
   v) Xaa24 is lysine, arginine or histidine;
   w) Xaa25 is asparagine, glutamine, serine, threonine, alanine;
   x) Xaa26 is asparagine, serine, glutamate or glutamine;
   y) Xaa28 is valine, alanine, leucine, isoleucine, proline, phenylalanine, methionine, tryptophan or threonine;
   z) Xaa29 is proline, alanine or glycine;
   aa) Xaa31 is asparagine, glutamine, glutamate or aspartate;
   bb) Xaa34 is serine, threonine or alanine;
   cc) Xaa35 is lysine, arginine, histidine, aspartate or glutamate;
   dd) Xaa36 is alanine, valine, leucine or isoleucine; or
   ee) any combination of any two or more of a) to dd);
   wherein one or more of Xaa1-Xaa11, Xaa13-Xaa15, Xaa17-Xaa26, Xaa28, Xaa29, Xaa31 and Xaa34-Xaa36 is or is substituted with an amino acid that is covalently conjugated to a lipid-containing moiety.
34. A peptide conjugate of any one of paragraphs 31-33 wherein
   a) Xaa1 is alanine or serine;
   b) Xaa2 is cysteine;
   c) Xaa3 is aspartate or glutamate;
   d) Xaa4 is threonine;
   e) Xaa5 is alanine;
   f) Xaa6 is threonine;
   g) Xaa7 is cysteine;
   h) Xaa8 is valine;
   i) Xaa9 is threonine;
   j) Xaa10 is histidine;
   k) Xaa11 is arginine;
   l) Xaa13 is alanine;
   m) Xaa14 is glycine or aspartate;
   n) Xaa15 is leucine;
   o) Xaa17 is serine or arginine;
   p) Xaa18 is arginine;
   q) Xaa19 is serine;
   r) Xaa20 is glycine;
   s) Xaa21 is glycine;
   t) Xaa22 is valine or methionine;
   u) Xaa23 is valine or leucine;
   v) Xaa24 is lysine;
   w) Xaa25 is asparagine or serine;
   x) Xaa26 is asparagine, serine or glutamate;
   y) Xaa28 is valine;
   z) Xaa29 is proline;
   aa) Xaa31 is asparagine or aspartate;
   bb) Xaa34 is serine;
   cc) Xaa35 is lysine or glutamate;
   dd) Xaa36 is alanine; or
   ee) any combination of any two or more of a) to dd);
   wherein one or more of Xaa1-Xaa11, Xaa13-Xaa15, Xaa17-Xaa26, Xaa28, Xaa29, Xaa31 and Xaa34-Xaa36 is or is substituted with an amino acid that is covalently conjugated to a lipid-containing moiety.
35. A peptide conjugate of any one of paragraphs 31-34 wherein the peptide comprises or consists of an amino acid sequence of the formula:

[SEQ ID No. 2]
Z-Xaa⁸Thr⁹Xaa¹⁰Xaa¹¹Leu¹²Ala¹³Xaa¹⁴Leu¹⁵Leu¹⁶Xaa¹⁷
Xaa¹⁸Xaa¹⁹Gly²⁰Xaa²¹Xaa²²Xaa²³Xaa²⁴Xaa²⁵Asn²⁶Phe²⁷
Val²⁸Pro²⁹Thr³⁰Xaa³¹Val³²Gly³³Ser³⁴Xaa³⁵Ala³⁶Phe³⁷

wherein:
   Z is absent or is Xaa¹Xaa²Xaa³Thr⁴Ala⁵Xaa⁶Xaa⁷, Xaa²Xaa³Thr⁴Ala⁵Xaa⁶Xaa⁷, Xaa³Thr⁴Ala⁵Xaa⁶Xaa⁷, Thr⁴Ala⁵Xaa⁶Xaa⁷, Ala⁵Xaa⁶Xaa⁷, Xaa⁶Xaa⁷ or Xaa⁷
   wherein:
   a) Xaa1 is alanine or serine;
   b) Xaa2 is cysteine or homocysteine;
   c) Xaa3 is aspartate or asparagine;
   d) Xaa6 is threonine, cysteine or homocysteine;
   e) Xaa7 is cysteine or homocysteine;
   f) Xaa8 is valine, cysteine or homocysteine;
   g) Xaa10 is histidine, cysteine or homocysteine,
   h) Xaa11 is arginine, cysteine or homocysteine;
   i) Xaa14 is glycine or aspartate;
   j) Xaa17 is serine, arginine, cysteine or homocysteine,
   k) Xaa18 is arginine, cysteine or homocysteine;
   l) Xaa19 is a serine, cysteine or homocysteine;
   m) Xaa21 is glycine, cysteine or homocysteine;
   n) Xaa22 is valine or methionine;
   o) Xaa23 is valine or leucine;
   p) Xaa24 is lysine, cysteine or homocysteine;
   q) Xaa25 is asparagine, serine or aspartate;
   r) Xaa31 is asparagine or aspartate; and
   s) Xaa35 is lysine, glutamate, cysteine or homocysteine;
   wherein at least one cysteine or homocysteine in the peptide is covalently conjugated to a lipid-containing moiety.
36. A peptide conjugate of any one of paragraphs 31-35 wherein one or more of Xaa6-Xaa8, Xaa10, Xaa11, Xaa17-Xaa19, Xaa21, Xaa24 and Xaa35 is or is substituted with an amino acid that is covalently conjugated to a lipid-containing moiety.
   paragraph
37. A peptide conjugate of any one of paragraphs 31-36 wherein one or more of Xaa7, Xaa8, Xaa11, Xaa24 and Xaa35 is or is substituted with an amino acid that is covalently conjugated to a lipid-containing moiety.
38. A peptide conjugate of any one of paragraphs 31-37 wherein one or more of Xaa7, Xaa8, Xaa24 and Xaa35 is or is substituted with an amino acid that is covalently conjugated to a lipid-containing moiety.
39. A peptide conjugate of any one of paragraphs 31-38 wherein 1 or 2 of Xaa6-Xaa8, Xaa10, Xaa11, Xaa17-Xaa19, Xaa21, Xaa24 and Xaa35 is or is substituted with an amino acid that is covalently conjugated to a lipid-containing moiety.
40. A peptide conjugate of any one of paragraphs 31-39 wherein 1 or 2 of Xaa⁷, Xaa⁸, Xaa¹¹, Xaa²⁴ and Xaa³⁵ is or is substituted with an amino acid that is covalently conjugated to a lipid-containing moiety.
41. A peptide conjugate of any one of paragraphs 31-40 wherein two or more of Xaa⁶-Xaa⁸, Xaa¹⁰, Xaa¹¹, Xaa¹⁷-Xaa¹⁹, Xaa²¹, Xaa²⁴ and Xaa³⁵ is or is substituted with an amino acid that is covalently conjugated to a lipid-containing moiety.
42. A peptide conjugate of any one of paragraphs 31-41 wherein two or more of Xaa⁷, Xaa⁸, Xaa¹¹, Xaa²⁴ and Xaa³⁵ is or is substituted with an amino acid that is covalently conjugated to a lipid-containing moiety.
43. A peptide conjugate of paragraph any one of paragraphs 1-30 wherein the peptide comprises or consists of
   a) the amino acid sequence of SEQ ID NO:3;
   b) 25 or more contiguous amino acids of SEQ ID NO:3;
   c) amino acids 7-37 of SEQ ID No:3;
   d) amino acids 8-37 of SEQ ID NO:3;
   e) the amino acid sequence of SEQ ID NO:4;
   f) 25 or more contiguous amino acids of SEQ ID NO:4;
   g) amino acids 7-37 of SEQ ID No:4;
   h) amino acids 8-37 of SEQ ID NO:4; or
   i) a functional variant of any one of a) to h) comprising or consisting of an amino acid sequence having at least about 60% amino acid sequence identity to the sequence defined in any one of a) to h);
   wherein one or more amino acids in the sequence is or is substituted with an amino acid covalently conjugated to a lipid-containing moiety.
44. A peptide conjugate of any of the preceding paragraphs wherein the peptide comprises or consists of an amino acid sequence selected from
   a) amino acids 2-37 of SEQ ID No:3 or SEQ ID No:4;
   b) amino acids 3-37 of SEQ ID No:3 or SEQ ID No:4;
   c) amino acids 4-37 of SEQ ID No:3 or SEQ ID No:4;
   d) amino acids 5-37 of SEQ ID No:3 or SEQ ID No:4;
   e) amino acids 6-37 of SEQ ID No:3 or SEQ ID No:4; or
   f) a functional variant of any one of a) to e) comprising or consisting of an amino acid sequence having at least about 60% amino acid sequence identity to the sequence defined in any one of a) to e);
   wherein one or more amino acids in the sequence is or is substituted with an amino acid covalently conjugated to a lipid-containing moiety.
45. A peptide conjugate of paragraph 43 (i) or paragraph 44 (f) wherein the amino acid sequence has at least about 90% sequence identity to the sequence defined in paragraph 43 a)-h) or paragraph 44 a)-e).
46. A peptide conjugate of any one of paragraphs 43 to 45 wherein the peptide comprises an amino acid covalently conjugated to a lipid-containing moiety at one or more amino acid positions corresponding to positions 1-11, 13-15, 17-26, 28, 29, 31 and 34-36 of SEQ ID No 3 or SEQ ID No:4.
47. A peptide conjugate of any one of paragraphs 43 to 46 wherein the peptide comprises an amino acid covalently conjugated to a lipid-containing moiety at one or more amino acid positions corresponding to positions 6-8, 10, 11, 17-19, 21, 24 and 35 of SEQ ID No 3 or SEQ ID NO:4.
48. A peptide conjugate of any one of paragraphs 43 to 47 wherein the peptide comprises an amino acid covalently conjugated to a lipid-containing moiety at one or more amino acid positions corresponding to positions 6-8, 10, 11, 21, 24 and 35 of SEQ ID No 3 or SEQ ID NO: 4.
49. A peptide conjugate of any one of paragraphs 43 to 48 wherein the peptide comprises an amino acid covalently conjugated to a lipid-containing moiety at one or more amino acid positions corresponding to positions 7, 8, 11, 24 and 35 of SEQ ID No 3 or SEQ ID NO: 4.
50. A peptide conjugate of any one of paragraphs 43 to 49 wherein the peptide comprises an amino acid covalently conjugated to a lipid-containing moiety at one or more amino acid positions corresponding to positions 7, 8, 24 and 35 of SEQ ID No 3 or SEQ ID NO: 4.
51. A peptide conjugate of any one of paragraphs 43 to 50 wherein the peptide comprises an amino acid covalently conjugated to a lipid-containing moiety at one or more amino acid positions corresponding to positions 7, 8, 11, 24 and 35 of SEQ ID No 3 or SEQ ID NO: 4.
52. A peptide conjugate of any one of the preceding paragraphs wherein the N-terminal amino acid of the peptide is covalently conjugated to a lipid-containing moiety.
53. A peptide conjugate of any one of the preceding paragraphs wherein the peptide comprises one or more amino acids covalently conjugated to a lipid-containing moiety in
   a) a region of the peptide comprising amino acids Xaa1-Xaa7 or a region of the peptide corresponding to amino acids 1-7 of SEQ ID No:3 or SEQ ID No:4;
   b) a region of the peptide comprising amino acids Xaa8-Xaa18 or a region of the peptide corresponding to amino acids 8-18 of SEQ ID No:3 or SEQ ID No:4;
   c) a region of the peptide comprising amino acids Xaa19-Xaa26 or a region of the peptide corresponding to amino acids 19-26 of SEQ ID No:3 or SEQ ID No:4;
   d) a region of the peptide comprising Xaa27-Xaa37 or a region of the peptide corresponding to amino acids 27-37 of SEQ ID No:3 or SEQ ID No:4; or
   e) any combination of any two or more of a) to d).
54. A peptide conjugate of any one of the preceding paragraphs wherein the peptide comprises from about 1 to about 5 amino acids covalently conjugated to a lipid-containing moiety.
55. A peptide conjugate of any one of the preceding paragraphs wherein the peptide comprises from about 1 to about 3 amino acids covalently conjugated to a lipid-containing moiety.
56. A peptide conjugate of any one of the preceding paragraphs wherein the peptide comprises 1 or 2 amino acids covalently conjugated to a lipid-containing moiety.
57. A peptide conjugate of any one of the preceding paragraphs wherein the amino acid covalently conjugated to a lipid-containing moiety is cysteine or homocysteine, and the the lipid-containing moiety is covalently attached via the sulfur atom of the sulfide group of the cysteine or homocysteine.
58. A peptide conjugate of any one of the preceding paragraphs wherein the peptide comprises or consists of an amino acid sequence selected from

a)
[SEQ ID No: 5]
AXDTATXVTHRLAGLLSRSGGVVKNNFVPTNVGSKAF;
b)
[SEQ ID No: 6]
XXDTATXVTHRLAGLLSRSGGVVKNNFVPTNVGSKAF;
c)
[SEQ ID No: 7]
AXXTATXVTHRLAGLLSRSGGVVKNNFVPTNVGSKAF;
d)
[SEQ ID No: 8]
AXDXATXVTHRLAGLLSRSGGVVKNNFVPTNVGSKAF;
e)
[SEQ ID No: 9]
AXDTXTXVTHRLAGLLSRSGGVVKNNFVPTNVGSKAF;
f)
[SEQ ID No: 10]
AXDTAXXVTHRLAGLLSRSGGVVKNNFVPTNVGSKAF;
g)
[SEQ ID No: 11]
XDTATXVTHRLAGLLSRSGGVVKNNFVPTNVGSKAF;
h)
[SEQ ID No: 12]
DTATXVTHRLAGLLSRSGGVVKNNFVPTNVGSKAF;
i)
[SEQ ID No: 13]
XTATXVTHRLAGLLSRSGGVVKNNFVPTNVGSKAF;
j)
[SEQ ID No: 14]
TATXVTHRLAGLLSRSGGVVKNNFVPTNVGSKAF;
k)
[SEQ ID No: 15]
ATXVTHRLAGLLSRSGGVVKNNFVPTNVGSKAF;
l)
[SEQ ID No: 16]
TXVTHRLAGLLSRSGGVVKNNFVPTNVGSKAF;
m)
[SEQ ID No: 17]
XVTHRLAGLLSRSGGVVKNNFVPTNVGSKAF;
n)
[SEQ ID No: 18]
XTHRLAGLLSRSGGVVKNNFVPTNVGSKAF;
o)
[SEQ ID No: 19]
VXHRLAGLLSRSGGVVKNNFVPTNVGSKAF;
p)
[SEQ ID No: 20]
VTXRLAGLLSRSGGVVKNNFVPTNVGSKAF;
q)
[SEQ ID No: 2l]
VTHXLAGLLSRSGGVVKNNFVPTNVGSKAF;
r)
[SEQ ID No: 22]
VTHRLXGLLSRSGGVVKNNFVPTNVGSKAF;
s)
[SEQ ID No: 23]
VTHRLAXLLSRSGGVVKNNFVPTNVGSKAF;
t)
[SEQ ID No: 24]
VTHRLAGXLSRSGGVVKNNFVPTNVGSKAF;
u)
[SEQ ID No: 25]
VTHRLAGLLXRSGGVVKNNFVPTNVGSKAF;
v)
[SEQ ID No: 26]
VTHRLAGLLSXSGGVVKNNFVPTNVGSKAF;
w)
[SEQ ID No: 27]
VTHRLAGLLSRXGGVVKNNFVPTNVGSKAF;
x)
[SEQ ID No: 28]
VTHRLAGLLSRSXGVVKNNFVPTNVGSKAF;
y)
[SEQ ID No: 29]
VTHRLAGLLSRSGXVVKNNFVPTNVGSKAF;
z)
[SEQ ID No: 30]
VTHRLAGLLSRSGGXVKNNFVPTNVGSKAF;
aa)
[SEQ ID No: 32]
VTHRLAGLLSRSGGVXKNNFVPTNVGSKAF;
bb)
[SEQ ID No: 33]
VTHRLAGLLSRSGGVVXNNFVPTNVGSKAE
cc)
[SEQ ID No: 34]
VTHRLAGLLSRSGGVVKXNFVPTNVGSKAF;
dd)
[SEQ ID No: 35]
VTHRLAGLLSRSGGVVKNXFVPTNVGSKAF;
ee)
[SEQ ID No: 36]
VTHRLAGLLSRSGGVVKNNFXPTNVGSKAF;
ff)
[SEQ ID No: 37]
VTHRLAGLLSRSGGVVKNNFVXTNVGSKAF;
gg)
[SEQ ID No: 38]
VTHRLAGLLSRSGGVVKNNFVPTXVGSKAF;
hh)
[SEQ ID No: 39]
VTHRLAGLLSRSGGVVKNNFVPTNVGXKAF;
ii)
[SEQ ID No: 40]
VTHRLAGLLSRSGGVVKNNFVPTNVGSXAF;
jj)
[SEQ ID No: 41]
VTHRLAGLLSRSGGVVKNNFVPTNVGSKXF;
kk)
[SEQ ID No: 42]
AXNTATXVTHRLAGLLSRSGGMVKSNFVPTNVGSKAF;
ll)
[SEQ ID No: 43]
XXNTATXVTHRLAGLLSRSGGMVKSNFVPTNVGSKAF;
mm)
[SEQ ID No: 44]
AXXTATXVTHRLAGLLSRSGGMVKSNEVPTNVGSKAF;
nn)
[SEQ ID No: 45]
AXNXATXVTHRLAGLLSRSGGMVKSNFVPTNVGSKAF;
oo)
[SEQ ID No: 46]
AXNTXTXVTHRLAGLLSRSGGMVKNFVPTNVGSKAF;
pp)
[SEQ ID No: 47]
AXNTAXXVTHRLAGLLSRSGGMVKSNFVPTNVGSKAF;
qq)
[SEQ ID No: 48]
XNTATXVTHRLAGLLSRSGGMVKSNFVPTNVGSKAF;
rr)
[SEQ ID No: 49]
NTATXVTHRLAGLLSRSGGMVKSNFVPTNVGSKAF;
ss)
[SEQ ID No: 50]
AXNXTATXVTHRLAGLLSRSGGMVKSNFVPTNVGSKAF;
tt)
[SEQ ID No: 51]
XTATXVTHRLAGLLSRSGGMVKSNFVPTNVGSKAF;
uu)
[SEQ ID No: 52]
TATXVTHRLAGLLSRSGGMVKSNFVPTNVGSKAF;
vv)
[SEQ ID No: 53]
ATXVTHRLAGLLSRSGGMVKSNFVPTNVGSKAF;
ww)
[SEQ ID No: 54]
TXVTHRLAGLLSRSGGMVKSNFVPTNVGSKAF;
xx)
[SEQ ID No: 55]
XVTHRLAGLLSRSGGMVKSNFVPTNVGSKAF;
yy)
[SEQ ID No: 56]
XTHRLAGLLSRSGGMVKSNFVPTNVGSKAF;
zz)
[SEQ ID No: 57]
VXHRLAGLLSRSGGMVKSNFVPTNVGSKAF;
aaa)
[SEQ ID No: 58]
VTXRLAGLLSRSGGMVKSNFVPTNVGSKAF;
bbb)
[SEQ ID No: 59]
VTHXLAGLLSRSGGMVKSNFVPTNVGSKAF;
ccc)
[SEQ ID No: 60]
VTHRLXGLLSRSGGMVKSNFVPTNVGSKAF;
ddd)
[SEQ ID No: 61]
VTHRLAXLLSRSGGMVKSNFVPTNVGSKAF;
eee)
[SEQ ID No: 62]
VTHRLAGXLSRSGGMVKSNFVPTNVGSKAF;
fff)
[SEQ ID No: 63]
VTHRLAGLLXRSGGMVKSNFVPTNVGSKAF;
ggg)
[SEQ ID No: 64]
VTHRLAGLLSXSGGMVKSNFVPTNVGSKAF;
hhh)
[SEQ ID No: 65]
VTHRLAGLLSRXGGMVKSNFVPTNVGSKAF;
iii)
[SEQ ID No: 66]
VTHRLAGLLSRSXGMVKSNFVPTNVGSKAF;
jjj)
[SEQ ID No: 67]
VTHRLAGLLSRSGXMVKSNFVPTNVGSKAF;
kkk)
[SEQ ID No: 68]
VTHRLAGLLSRSGGXVKSNFVPTNVGSKAF;
lll)
[SEQ ID No: 69]
VTHRLAGLLSRSGGMXKSNFVPTNVGSKAF;
mmm)
[SEQ ID No: 70]
VTHRLAGLLSRSGGMVXSNFVPTNVGSKAF;
nnn)
[SEQ ID No: 71]
VTHRLAGLLSRSGGMVKXNFVPTNVGSKAF;
ooo)
[SEQ ID No: 72]
VTHRLAGLLSRSGGMVKSXFVPTNVGSKAF;
ppp)
[SEQ ID No: 73]
VTHRLAGLLSRSGGMVKSNFXPTNVGSKAF;
qqq)
[SEQ ID No: 74]
VTHRLAGLLSRSGGMVKSNFVXTNVGSKAF;
rrr)
[SEQ ID No: 75]
VTHRLAGLLSRSGGMVKSNFVPTXVGSKAF;
sss)
[SEQ ID No: 76]
VTHRLAGLLSRSGGMVKSNFVPTNVGXKAF;
ttt)
[SEQ ID No: 77]
VTHRLAGLLSRSGGMVKSNFVPTNVGSXAF;
or
uuu)
[SEQ ID No: 78]
VTHRLAGLLSRSGGMVKSNFVPTNVGSKXF;

wherein X is cysteine or homocysteine and wherein at least one X in the peptide is covalently conjugated to a lipid-containing moiety.
59. A peptide conjugate of any one of the preceding paragraphs wherein the peptide comprises or consists of an amino acid sequence selected from

a)
[SEQ ID No: 17]
XVTHRLAGLLSRSGGVVKNNFVPTNVGSKAF;
b)
[SEQ ID No: 18]
XTHRLAGLLSRSGGVVKNNFVPTNVGSKAF;
c)
[SEQ ID No: 33]
VTHRLAGLLSRSGGVVXNNFVPTNVGSKAF;
d)
[SEQ ID No: 40]
VTHRLAGLLSRSGGVVKNNFVPTNVGSXAF;
e)
[SEQ ID No: 10]
AXDTAXXVTHRLAGLLSRSGGVVKNNFVPTNVGSKAF;
f)
[SEQ ID No: 20]
VTXRLAGLLSRSGGVVKNNFVPTNVGSKAF;
g)
[SEQ ID No: 21]
VTHXLAGLLSRSGGVVKNNFVPTNVGSKAF;
h)
[SEQ ID No: 29]
VTHRLAGLLSRSGXVVKNNFVPTNVGSKAF;
i)
[SEQ ID No: 55]
XVTHRLAGLLSRSGGMVKSNFVPTNVGSKAF;
j)
[SEQ ID No: 56]
XTHRLAGLLSRSGGMVKSNFVPTNVGSKAF;
k)
[SEQ ID No: 70]
VTHRLAGLLSRSGGMVXSNFVPTNVGSKAF;
l)
[SEQ ID No: 77]
VTHRLAGLLSRSGGMVKSNFVPTNVGSXAF;
m)
[SEQ ID No: 47]
AXNTAXXVTHRLAGLLSRSGGMVKSNFVPTNVGSKAF;
n)
[SEQ ID No: 58]
VTXRLAGLLSRSGGMVKSNFVPTNVGSKAF;
o)
[SEQ ID No: 59]
VTHXLAGLLSRSGGMVKSNFVPTNVGSKAF;
or
p)
[SEQ ID No: 67]
VTHRLAGLLSRSGXMVKSNFVPTNVGSKAF;

wherein X is cysteine or homocysteine,
and wherein at least one X in the peptide is covalently conjugated to a lipid-containing moiety.
60. A peptide conjugate of any one of the preceding paragraphs wherein the peptide comprises or consists of an amino acid sequence selected from

a)
[SEQ ID No: 79]
CVTHRLAGLLSRSGGVVKNNFVPTNVGSKAF;
b)
[SEQ ID No: 80]
CTHRLAGLLSRSGGVVKNNFVPTNVGSKAF;
c)
[SEQ ID No: 81]
VTHRLAGLLSRSGGVVCNNFVPTNVGSKAF;
d)
[SEQ ID No: 82]
VTHRLAGLLSRSGGVVKNNFVPTNVGSCAF;
e)
[SEQ ID No: 83]
ACDTACCVTHRLAGLLSRSGGVVKNNFVPTNVGSKAF;
f)
[SEQ ID No: 84]
VTCRLAGLLSRSGGVVKNNFVPTNVGSKAF;
g)
[SEQ ID No: 85]
VTHCLAGLLSRSGGVVKNNFVPTNVGSKAF;
h)
[SEQ ID No: 86]
VTHRLAGLLSRSGCVVKNNFVPTNVGSKAF;
i)
[SEQ ID No: 87]
CVTHRLAGLLSRSGGMVKSNFVPTNVGSKAF;
j)
[SEQ ID No: 88]
CTHRLAGLLSRSGGMVKSNFVPTNVGSKAF;
k)
[SEQ ID No: 89]
VTHRLAGLLSRSGGMVCSNFVPTNVGSKAF;
1)
[SEQ ID No: 90]
VTHRLAGLLSRSGGMVKSNFVPTNVGSCAF;
m)
[SEQ ID No: 91]
ACNTACCVTHRLAGLLSRSGGMVKSNFVPTNVGSKAF;
n)
[SEQ ID No: 92]
VTCRLAGLLSRSGGMVKSNFVPTNVGSKAF;
o)
[SEQ ID No: 93]
VTHCLAGLLSRSGGMVKSNFVPTNVGSKAF;
or
p)
[SEQ ID No: 94]
VTHRLAGLLSRSGCMVKSNFVPTNVGSKAF;

wherein at least one C in the peptide is covalently conjugated to a lipid-containing moiety.
61. A peptide conjugate of any one of the preceding claims wherein the peptide comprises or consists of an amino acid sequence selected from

a)
[SEQ ID No: 100]
XXTHRLAGLLSRSGGVVKNNFVPTNVGSKAF;
b)
[SEQ ID No: 101]
XVTHXLAGLLSRSGGVVKNNFVPTNVGSKAF;
c)
[SEQ ID No: 102]
XVTHRLAGLLSRSGGVVXNNFVPTNVGSKAF;
d)
[SEQ ID No: 103]
XVTHRLAGLLSRSGGVVKNNFVPTNVGSXAF;
e)
[SEQ ID No: 104]
XTHXLAGLLSRSGGVVKNNFVPTNVGSKAF;
f)
[SEQ ID No: 105]
XTHRLAGLLSRSGGVVXNNFVPTNVGSKAF;
g)
[SEQ ID No: 106]
XTHRLAGLLSRSGGVVKNNFVPTNVGSXAF;
h)
[SEQ ID No: 107]
VTHXLAGLLSRSGGVVXNNFVPTNVGSKAF;
i)
[SEQ ID No: 108]
VTHXLAGLLSRSGGVVKNNFVPTNVGSXAF;
j)
[SEQ ID No: 109]
VTHRLAGLLSRSGGVVXNNFVPTNVGSXAF;
k)
[SEQ ID No: 110]
XXTHRLAGLLSRSGGMVKSNFVPTNVGSKAF;
l)
[SEQ ID No: 111]
XVTHXLAGLLSRSGGMVKSNFVPTNVGSKAF;
m)
[SEQ ID No: 112]
XVTHRLAGLLSRSGGMVXSNFVPTNVGSKAF;
n)
[SEQ ID No: 113]
XVTHRLAGLLSRSGGMVKSNFVPTNVGSXAF;
o)
[SEQ ID No: 114]
XTHXLAGLLSRSGGMVKSNFVPTNVGSKAF;
p)
[SEQ ID No: 115]
XTHRLAGLLSRSGGMVXSNFVPTNVGSKAF;
q)
[SEQ ID No: 116]
XTHRLAGLLSRSGGMVKSNFVPTNVGSXAF;
r)
[SEQ ID No: 117]
VTHXLAGLLSRSGGMVXSNFVPTNVGSKAF;
s)
[SEQ ID No: 118]
VTHXLAGLLSRSGGMVKSNFVPTNVGSXAF;
or
t)
[SEQ ID No: 119]
VTHRLAGLLSRSGGMVXSNFVPTNVGSXAF;

wherein X is cysteine or homocysteine,
and wherein at least two X in the peptide are covalently conjugated to a lipid-containing moiety.
62. A peptide conjugate of any one of the preceding claims wherein the peptide comprises or consists of an amino acid sequence selected from

a)
[SEQ ID No: 120]
CCTHRLAGLLSRSGGVVKNNFVPTNVGSKAF;
b)
[SEQ ID No: 121]
CVTHCLAGLLSRSGGVVKNNFVPTNVGSKAF;
c)
[SEQ ID No: 122]
CVTHRLAGLLSRSGGVVCNNFVPTNVGSKAF;
d)
[SEQ ID No: 123]
CVTHRLAGLLSRSGGVVKNNFVPTNVGSCAF;
e)
[SEQ ID No: 124]
CTHCLAGLLSRSGGVVKNNFVPTNVGSKAF;
f)
[SEQ ID No: 125]
CTHRLAGLLSRSGGVVCNNFVPTNVGSKAF;
g)
[SEQ ID No: 126]
CTHRLAGLLSRSGGVVKNNFVPTNVGSCAF;
h)
[SEQ ID No: 99]
VTHCLAGLLSRSGGVVCNNFVPTNVGSKAF;
i)
[SEQ ID No: 127]
VTHCLAGLLSRSGGVVKNNFVPTNVGSCAF;
j)
[SEQ ID No: 128]
VTHRLAGLLSRSGGVVCNNFVPTNVGSCAF;
k)
[SEQ ID No: 129]
CCTHRLAGLLSRSGGMVKSNFVPTNVGSKAF;
1)
[SEQ ID No: 130]
CVTHCLAGLLSRSGGMVKSNFVPTNVGSKAF;
m)
[SEQ ID No: 131]
CVTHRLAGLLSRSGGMVCSNFVPTNVGSKAF;
n)
[SEQ ID No: 132]
CVTHRLAGLLSRSGGMVKSNFVPTNVGSCAF;
o)
[SEQ ID No: 133]
CTHCLAGLLSRSGGMVKSNFVPTNVGSKAF;
p)
[SEQ ID No: 134]
CTHRLAGLLSRSGGMVCSNFVPTNVGSKAF;
q)
[SEQ ID No: 135]
CTHRLAGLLSRSGGMVKSNFVPTNVGSCAF;
r)
[SEQ ID No: 136]
VTHCLAGLLSRSGGMVCSNFVPTNVGSKAF;
s)
[SEQ ID No: 137]
VTHCLAGLLSRSGGMVKSNFVPTNVGSCAF;
or
t)
[SEQ ID No: 138]
VTHRLAGLLSRSGGMVCSNFVPTNVGSCAF;

wherein at least two C in the peptide are covalently conjugated to a lipid-containing moiety.
63. A peptide conjugate of any one of the preceding paragraphs, wherein the peptide conjugate and α-CGRP8-37 (SEQ ID No:96) each independently have a first antagonist potency value (pA₂) at a CGRP receptor and a second antagonist potency value (pA₂) at a CGRP receptor;
   wherein the first antagonist potency value (pA₂) at a CGRP receptor is after incubating the receptor and peptide conjugate or α-CGRP8-37 (SEQ ID No:96) and not washing the receptor prior to determining the antagonist potency value;
   wherein the second antagonist potency value (pA₂) at a CGRP receptor is after incubating the receptor and peptide conjugate or α-CGRP8-37 (SEQ ID No:96) and then washing the receptor prior to determining the antagonist potency value;
   wherein the second antagonist potency value (pA₂) is less than the first antagonist potency value (pA₂); and
   the fold change reduction in antagonist potency between the first antagonist potency value (pA₂) of the peptide conjugate and the second antagonist potency value (pA₂) of the peptide conjutage is less than the fold change reduction in antagonist potency between the the first antagonist potency value (pA₂) of α-CGRP8-37 (SEQ ID No:96) and the second antagonist potency value (pA₂) of α-CGRP8-37 (SEQ ID No:96).
64. A peptide conjugate of paragraph 63, wherein the fold change reduction in antagonist potency between the first antagonist potency value (pA₂) of the peptide conjugate and the second antagonist potency value (pA₂) of the peptide conjutage is less than about 50, 45, 40, 35, 30, 25, 20, 15, 10, 9, 8, 7, 6, 5, 4, 3, or 2, wherein the antagonist potency value (pA₂) at a CGRP receptor is measured by a cAMP assay, and wherein the CGRP receptor is a CLR/RAMP1 CGRP receptor, for example as described in the Examples herein.
65. A peptide conjugate of paragraph 63 or 64, wherein the fold change reduction in antagonist potency between the first antagonist potency value (pA₂) of the peptide conjugate and the second antagonist potency value (pA₂) of the peptide conjutage is less than about 20, 15, 14, 13, 12, 11, 10, 9, 8, 7, 6, 5, 4, 3, or 2, wherein the antagonist potency value (pA₂) at a CGRP receptor is measured by a cAMP assay, and wherein the CGRP receptor is a CTR/RAMP1 AMY1 CGRP receptor, for example as described in the Examples herein.
66. A pharmaceutical composition comprising a peptide conjugate according to any one of the preceding paragraphs; and a pharmaceutically acceptable carrier.
67. A kit comprising a peptide conjugate according to any one of paragraphs 1-65; and instructions for use.
68. A method of antagonising a CGRP receptor in a subject in need thereof, comprising administering to the subject an effective amount of a peptide conjugate according to any one of paragraphs 1-65.
69. A method of treating a disease or condition mediated by or modulated by a CGRP receptor or characterised by excessive CGRP receptor activation in a subject in need thereof, comprising administering to the subject a therapeutically effective amount of a peptide conjugate according to any one of paragraphs 1-65.
70. A method of treating a disease or condition associated with or characterised by increased vasodilation in a subject in need thereof, comprising administering to the subject a therapeutically effective amount of a peptide conjugate according to any one of paragraphs 1-65.
71. A method of treating a disease or condition selected from the group consisting of thermal injury, circulatory shock, menopausal hot flushes, asthma, sepsis, neurogenic inflammation, inflammatory skin conditions (for example psoriasis and contact dermatitis), allergic rhinitis, joint disorders (for example arthritis and temporomandibular joint disorder, preferably arthritis), cachexia (for example cancer-induced cachexia), pain, for example craniofacial pain disorders (for example migraine, headache, trigeminal neuralgia and dental pain, preferably migraine), and metabolic disorders or syndromes (for example obesity, type II diabetes, insulin resistance, dyslipidemia, hypertension, atherosclerosis and thrombosis) in a subject in need thereof, comprising administering to the subject a therapeutically effective amount of a peptide conjugate according to any one of paragraphs 1-65.
72. A peptide conjugate according to any one of paragraphs 1-65 for use in antagonising a CGRP receptor.
73. A peptide conjugate according to any one of paragraphs 1-65 for use in treating a disease or condition mediated by or modulated by a CGRP receptor or characterised by excessive CGRP receptor activation.
74. A peptide conjugate according to any one of paragraphs 1-65 for use in treating a disease or condition associated with or characterised by increased vasodilation.
75. A peptide conjugate according to any one of paragraphs 1-65 for use in treating a disease or condition selected from the group consisting thermal injury, circulatory shock, menopausal hot flushes, asthma, sepsis, neurogenic inflammation, inflammatory skin conditions (for example psoriasis and contact dermatitis), allergic rhinitis, joint disorders (for example arthritis and temporomandibular joint disorder, preferably arthritis), cachexia (for example cancer-induced cachexia), pain, for example craniofacial pain disorders (for example migraine, headache, trigeminal neuralgia and dental pain, preferably migraine), and metabolic disorders or syndromes (for example obesity, type II diabetes, insulin resistance, dyslipidemia, hypertension, atherosclerosis and thrombosis).
76. Use of a peptide conjugate according to any one of paragraphs 1-65 in the manufacture of a medicament for antagonising a CGRP receptor.
77. Use of a peptide conjugate according to any one of paragraphs 1-65 in the manufacture of a medicament for treating a disease or condition mediated by or modulated by a CGRP receptor or characterised by excessive CGRP receptor activation.
78. Use of a peptide conjugate according to any one of paragraphs 1-65 in the manufacture of a medicament for treating a disease or condition associated with or characterised by increased vasodilation.
79. Use of a peptide conjugate according to any one of paragraphs 1-65 in the manufacture of a medicament for treating a disease or condition selected from the group consisting of thermal injury, circulatory shock, menopausal hot flushes, asthma, sepsis, neurogenic inflammation, inflammatory skin conditions (for example psoriasis and contact dermatitis), allergic rhinitis, joint disorders (for example arthritis and temporomandibular joint disorder, preferably arthritis), cachexia (for example cancer-induced cachexia), pain, for example craniofacial pain disorders (for example migraine, headache, trigeminal neuralgia and dental pain, preferably migraine), and metabolic disorders or syndromes (for example obesity, type II diabetes, insulin resistance, dyslipidemia, hypertension, atherosclerosis and thrombosis).
80. The method, compound for use, or use of any one of paragraphs 68, 72, and 76, wherein antagonising the CGRP receptor comprises treating a disease or condition mediated by or modulated by the CGRP receptor or characterised by excessive CGRP receptor activation.
81. The method, compound for use, or use of any one of paragraphs 69, 73, 77, and 80, wherein the disease or condition is selected from the group consisting of thermal injury, circulatory shock, menopausal hot flushes, asthma, sepsis, neurogenic inflammation, inflammatory skin conditions (for example psoriasis and contact dermatitis), allergic rhinitis, joint disorders (for example arthritis and temporomandibular joint disorder, preferably arthritis), cachexia (for example cancer-induced cachexia), pain, for example craniofacial pain disorders (for example migraine, headache, trigeminal neuralgia and dental pain, preferably migraine), and metabolic disorders or syndromes (for example obesity, type II diabetes, insulin resistance, dyslipidemia, hypertension, atherosclerosis and thrombosis).
82. The method, compound for use, or use of any one of paragraphs 69-71, 73-75, and 77-81, wherein the disease or condition is selected from pain or metabolic disorders.
83. The method, compound for use, or use according to any one of paragraphs 69-71, 73-75, and 77-82, wherein the disease or condition is pain.
84. The method, compound for use, or use according to any one of paragraphs 69-71, 73-75, and 77-83, wherein the disease or condition is migraine or headache (for example cluster headaches and post-traumatic headache).
85. The method, compound for use, or use according to any one of paragraphs 69-71, 73-75, 77-84, wherein the disease or condition is migraine.
86. A method of antagonising a CGRP receptor comprising contacting a cell and a peptide conjugate according to any one of paragraphs 1-65 in an amount effective to antagonise the CGRP receptor.
87. A method for preparing a peptide conjugate according to any one of paragraphs 1-65, the method comprising
   (A) providing an amino acid conjugate comprising an amino acid of a calcitonin gene-related peptide (CGRP) peptide, wherein the amino acid is covalently conjugated to a lipid-containing moiety via a sulfur atom of a sulfide group; and
   coupling the amino acid of the amino acid conjugate to one or more amino acids and/or one or more peptides to provide the peptide conjugate of any one of paragraphs 1-65; or
   (B) providing a peptide-conjugate comprising a peptide fragment of a calcitonin gene-related peptide (CGRP) peptide, wherein at least one amino acid of the peptide fragmentis covalently conjugated to a lipid-containing moiety via a sulfur atom of a sulfide group; and
   coupling an amino acid of the peptide conjugate to one or more amino acids and/or one or more peptides to provide the peptide conjugate of any one of paragraphs 1-65.
88. The method of paragraph 87, wherein the amino acid conjugate or the peptide conjugate comprising the peptide fragment is bound to a solid phase support; or wherein the amino acid conjugate or the peptide conjugate is coupled to an amino acid or peptide bound to a solid phase.
89. A method for preparing a peptide conjugate according to any one of paragraphs 1-65, the method comprising reacting
   a lipid-containing conjugation partner comprising a carbon carbon double bond, and
   an amino acid-comprising conjugation partner comprising at least one amino acid comprising a thiol
   under conditions effective to conjugate the lipid-containing conjugation partner to the amino acid-comprising conjugation partner.
90. The method of paragraph 89, wherein the conditions are effective to conjugate the lipid-containing conjugation partner to the amino acid-comprising conjugation partner by the hydrothiolation of the carbon-carbon double bond with the thiol.
91. The method of paragraph 89 or 90, wherein the at least one amino acid comprising the thiol is cysteine or homocysteine.
92. The method of any one of paragraphs 89-91, wherein the at least one amino acid comprising the thiol is cysteine.
93. The method of any one of paragraphs 89-92, wherein the lipid-containing conjugation partner is a compound of the formula (A-1):

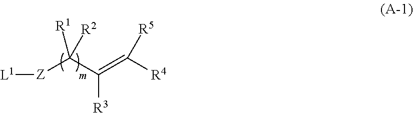

wherein
   Z and Z¹ are each independently selected from the group consisting of —O—, —NR—, —S—, —S(O)—, —SO₂—, —C(O)O—, —OC(O)—, —C(O)NR—, —NRC(O)—, —C(O)S—, —SC(O)—, —OC(O)O—, —NRC(O)O—, —OC(O)NR—, and —NRC(O)NR—;
   R is hydrogen or C₁₋₆aliphatic;
   m is an integer from 0 to 4;
   n is 1 or 2;
   R¹ and R² at each instance of m are each independently hydrogen, C₁₋₆aliphatic; or R¹ is L²-Z¹—C₁₋₆alkyl;
   R³, R⁴, and R⁵ are each independently hydrogen or C₁₋₆aliphatic; or R³ is L²-Z¹—C₁₋₆ alkyl;
   L¹ and L² are each independently C₅₋₂₁aliphatic or C₄₋₂₀heteroaliphatic; provided that:
      when R³ is L²-Z¹—C₁₋₆alkyl, R¹ is not L²-Z¹—C₁₋₆alkyl; and
      when m is an integer from 2 to 4, no more than one R¹ is L²-Z¹—C₁₋₆alkyl; and
   wherein any aliphatic, alkyl, or heteroaliphatic present in any of R, R¹, R², R³, R⁴, R⁵, L¹, and L² is optionally substituted with one or more independently selected optional substituents.
94. The method of any one of paragraphs 93, wherein the lipid-containing conjugation partner is a compound of the formula (II):

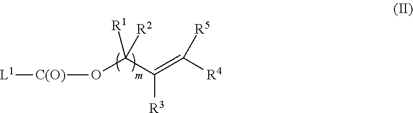

wherein
m, L¹, R¹, R², R³, R⁴, and R⁵ are as defined in paragraph 93 or in the moiety of formula (A) or (I) in any one of paragraphs 11-28; and
Z¹ when present is —C(O)O—.
95. The method of paragraph 93 or 94, wherein Z, Z¹, R, m, n, R¹, R², R³, R⁴, R⁵, L¹, and/or L² are as defined in paragraph 92 or 93 or in the moiety of formula (A) or (I) in any one of paragraphs 11-28.
96. The method of any one of paragraphs 89-95, wherein the lipid-containing conjugation partner is a vinyl ester of a fatty acid, for example vinyl palmitate.
97. The method of any one of paragraphs 89-96, wherein the method comprises
   reacting the lipid-containing conjugation partner and an amino acid-comprising conjugation partner comprising a calcitonin gene-related peptide (CGRP) peptide, wherein at least one amino acid of the peptide comprises a thiol, to provide the peptide conjugate of any one of paragraphs 1-65.
98. The method of any one of paragraphs 89-96, wherein the method comprises
   reacting the lipid-containing conjugation partner and an amino acid-comprising conjugation partner comprising a peptide fragment of a calcitonin gene-related peptide (CGRP) peptide, wherein at least one amino acid of the peptide fragment comprises a thiol, to provide a peptide conjugate; and
   coupling an amino acid of the peptide conjugate to one or more amino acids and/or one or more peptides to provide the peptide conjugate of any one of paragraphs 1-65.
99. The method of any one of paragraphs 89-96, where in the method comprises
   reacting the lipid-containing conjugation partner and an amino acid-comprising conjugation partner comprising an amino acid of a calcitonin gene-related peptide (CGRP) peptide, wherein the amino acid comprises a thiol, to provide an amino acid conjugate; and
   coupling the amino acid of the amino acid conjugate to one or more amino acids and/or one or more peptides to provide the peptide conjugate of any one of paragraphs 1-65.
100. The method of any one of paragraphs 89-99, wherein the method comprises reacting the lipid containing conjugation partner and an amino acid-comprising conjugation partner bound to a solid phase support to provide a solid phase bound amino acid conjugate or peptide conjugate.
101. The method of any one of paragraphs 87-100, wherein the method comprises coupling one or more amino acids and/or one or more peptides to a solid phase bound amino acid conjugate or peptide conjugate to provide a solid phase bound peptide conjugate.
102. The method of any one of paragraphs 88, 100, and 101, wherein the solid phase bound peptide conjugate has the amino acid sequence of the peptide conjugate of any one of paragraphs 1-65.
103. The method of any one of paragraphs 88 and 100-102, wherein the method further comprises cleaving the peptide conjugate from the solid phase.
104. The method of any one of paragraphs 87, 88, and 98-103, wherein the one or more amino acids and/or one or more peptides are coupled by SPPS.
105. The method of any one of paragraphs 97, 100, and 102-104, wherein the method comprises
   synthesising the amino acid sequence of the peptide of the amino acid-comprising conjugation partner by solid phase peptide synthesis (SPPS);
   reacting the lipid-containing conjugation partner and the solid phase bound amino-acid comprising conjugation partner to provide a solid phase bound peptide conjugate; and
   cleaving the peptide conjugate from the solid phase to provide the peptide conjugate of any one of paragraphs 1-65.
106. The method of any one of paragraphs 97 and 102-104, wherein the method comprises
   synthesising the amino acid sequence of the peptide of the amino acid-comprising conjugation partner by SPPS;
   cleaving the amino acid-comprising conjugation partner from the solid phase; and
   reacting the lipid-containing conjugation partner and the amino-acid comprising conjugation partner to provide the peptide conjugate of any one of paragraphs 1-65.
107. The method of any one of paragraphs 87, 88, 98, and 100-104, wherein the method comprises
   coupling an amino acid of the peptide conjugate comprising the peptide fragment and optionally one or more amino acids and/or one or more peptides to a solid phase bound amino acid or peptide by SPPS to provide a solid phase bound peptide conjugate having the amino acid sequence of the peptide conjugate of any one of paragraphs 1-65; and
   cleaving the peptide conjugate from the solid phase to provide the peptide conjugate of any one of paragraphs 1-65.
108. The method of any one of paragraphs 87, 88, 99, and 100-104, wherein the method comprises
   coupling the amino acid of the amino acid conjugate and optionally one or more amino acids and/or one or more peptides to a solid phase bound amino acid or peptide by SPPS to provide a solid phase bound peptide conjugate having the amino acid sequence of the peptide conjugate of any one of paragraphs 1-65; and
   cleaving the peptide conjugate from the solid phase to provide the peptide conjugate of any one of paragraphs 1-65.
109. The method of any one of paragraphs 87-108, wherein the method comprises acylating, for example acetylating, the Nα-amino group of the N-terminal amino acid of the peptide or peptide conjugate.
110. The method of any one of paragraphs 87-109, wherein the method comprises coupling coupling one or more amino acid and/or one or more peptide that reduces peptide aggregation during SPPS, for example a pseudoproline dipeptide such as Fmoc-Leu-Ser[Ψ(Me,Me)Pro]-OH.
111. The method of any one of paragraphs 89-110, wherein the method comprises
   providing a protected amino acid-comprising conjugation partner comprising at least one amino acid comprising a thiol protected with a protecting group; and
   removing the protecting group from the thiol to provide the amino acid comprising conjugation partner.
112. The method of paragraph 111, wherein the protected amino acid-comprising conjugation partner comprises one or more additional amino acids protected with one or more protecting groups different to the protecting group of the at least one amino acid comprising the thiol; and the method comprises selectively removing the protecting group from the thiol of the at least one amino acid comprising a thiol to provide the amino acid-comprising conjugation partner.
113. The method of any one of paragraphs 88 and 100-112, wherein one or more or all protecting groups are removed on cleaving the peptide from the solid phase support.
114. The method of any one of paragraphs 88 and 100-112, wherein the SPPS is Fmoc-SPPS.
115. The method of any one of paragraphs 89-114, wherein the conditions effective to conjugate the lipid-containing conjugation partner to the amino acid-comprising conjugation partner comprises the generation of one or more free radicals initiated by the thermal degradation of a thermal initiator or the photochemical degradation of a photochemical initiator.
116. The method of paragraph 115, wherein the thermal initiator is AIBN and/or the photoinitiator is DMPA.
117. The method of paragraph 115 or 116, wherein photochemical degradation of the free radical initiator comprises irradiation with ultraviolet light having a wavelength of about 365 nm.
118. The method of any one of paragraphs 89-117, wherein the reaction is carried out in a liquid medium comprising a solvent, wherein the solvent comprises NMP, DMF, DMSO, or a mixture thereof.
119. The method of any one of paragraphs 89 to 118, wherein the reaction is carried out in the presence of one or more additives that inhibit the formation of by-products and/or that improve the yield of or conversion to the conjugate.
120. The method of paragraph 119, wherein the one or more additive is an extraneous thiol, an acid, an organosilane, or a combination of any two or more thereof.
121. The method of paragraph 119 or 120, wherein the extraneous thiol is a sterically hindered thiol, for example tert-butyl mercaptan.
122. The method of paragraph 120, wherein the acid is a strong organic acid, for example TFA.
123. The method of any one of paragraphs 120, wherein the organosilane is a trialkylsilane, for example TIPS.
124. The peptide conjugate, pharmaceutical composition, kit, method, peptide conjugate for use, use, or method for preparing of any one of the preceding paragraphs, wherein the CGRP receptor is a CLR/RAMP1 CGRP receptor or a CTR/RAMP1 AMY1 CGRP receptor.
125. A peptide conjugate according to any one of paragraphs 1-65 made by a method of any one of paragraphs 87-123.

Any documents referred to herein including, but not limited to, patents, patent applications, journal articles, books, and the like, are incorporated herein by reference in their entirety. Any section headings used herein are for organizational purposes only and are not to be construed as limiting the subject matter described.

Although the invention has been described by way of example and with reference to particular embodiments, it is to be understood that modifications and/or improvements may be made without departing from the scope of the invention.

1. A peptide conjugate comprising a calcitonin gene-related peptide (CGRP) peptide, wherein at least one amino acid of the peptide is covalently conjugated to a lipid-containing moiety, wherein the peptide conjugate is a CGRP receptor antagonist.

2. A peptide conjugate comprising a calcitonin gene-related peptide (CGRP) peptide, wherein at least one amino acid of the peptide is covalently conjugated to a lipid-containing moiety via a sulfur atom of a sulfide group, wherein the peptide conjugate is a CGRP receptor antagonist.

3. The peptide conjugate of claim 1 or 2, wherein the peptide conjugate has an antagonist potency value (pA₂) more than a value about 10-fold less than, 5-fold less than, 3-fold less than, 2-fold less than, 1-fold less than the antagonist potency (pA₂) of α-CGRP8-37 (SEQ ID NO:96) at a CGRP receptor or has an antagonist potency value (pA₂) more than a value equal to the antagonist potency (pA₂) of CGRP8-37 at a CGRP receptor, for example as measured by a cAMP assay as described in the Examples herein.

4. The peptide conjugate of any one of the preceding claims, wherein the peptide conjugate has a half life at least 2-, 3-, 4-, 5-, 10-, 20-, 30-, 40-, or -50-fold longer than the half life of α-CGRP8-37 (SEQ ID NO:96), for example as measured in a suitable rodent model, for example a rat model.

5. The peptide conjugate of any one of the preceding claims, wherein the at least one amino acid is cysteine or homocysteine.

6. The peptide conjugate of any one of the preceding claims, wherein the at least one amino acid is cysteine.

7. The peptide conjugate of any one of the preceding claims, wherein the peptide conjugate comprises only one amino acid conjugated to a lipid-containing moiety.

8. The peptide conjugate of any one of claims 1-6, wherein the peptide conjugate comprises two or more amino acids each conjugated to a lipid-containing moiety.

9. The peptide conjugate of any one of the preceding claims, wherein the lipid-containing moiety comprises one or more straight or branched aliphatic or heteroaliphatic chains each containing at least 4 or at least 6 chain-linked atoms.

10. The peptide conjugate of any one of any one of the preceding claims, wherein the lipid-containing moiety comprises one or more saturated or unsaturated fatty acid esters.

11. The peptide conjugate of any one of the preceding claims, wherein the lipid-containing moiety is of the formula (A): 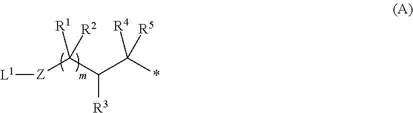
wherein
* represents a bond to the sulfur atom of the sulfide group of the amino acid to which the lipid-containing moiety is conjugated;
Z and Z¹ are each independently selected from the group consisting of —O—, —NR—, —S—, —S(O)—, —SO₂—, —C(O)O—, —OC(O)—, —C(O)NR—, —NRC(O)—, —C(O)S—, —SC(O)—, —OC(O)O—, —NRC(O)O—, —OC(O)NR—, and —NRC(O)NR—;
R is hydrogen or C₁₋₆aliphatic;
m is an integer from 0 to 4;
n is 1 or 2;
R¹ and R² at each instance of m are each independently hydrogen, C₁₋₆aliphatic; or
R¹ is L²-Z¹—C₁₋₆alkyl;
R³, R⁴, and R⁵ are each independently hydrogen or C₁₋₆aliphatic; or R³ is L²-Z¹—C₁₋₆alkyl;
L¹ and L² are each independently C₅₋₂₁aliphatic or C₄₋₂₀heteroaliphatic;
provided that:
when R³ is L²-Z¹—C₁₋₆alkyl, R¹ is not L²-Z¹—C₁₋₆alkyl; and
when m is an integer from 2 to 4, no more than one R¹ is L²-Z¹—C₁₋₆alkyl; and
wherein any aliphatic, alkyl, or heteroaliphatic present in any of R, R¹, R², R³, R⁴, R⁵, L¹, and L² is optionally substituted with one or more independently selected optional substituents.

12. The peptide conjugate of claim 11, wherein
R is hydrogen, C₁₋₆alkyl, or C₃₋₆cycloalkyl;
m is an integer from 0 to 4;
n is 1 or 2;
R¹ and R² at each instance of m are each independently hydrogen, C₁₋₆alkyl, or C₃₋₆cycloalkyl; or R¹ is L²-Z¹—C₁₋₆alkyl;
R³, R⁴, and R⁵ are each independently hydrogen, C₁₋₆alkyl, or C₃₋₆cycloalkyl; or R³ is L²-Z¹—C₁₋₆alkyl;
L¹ and L² are each independently C₅₋₂₁alkyl, C₅₋₂₁alkenyl, or C₄₋₂₀heteroalkyl;
wherein any alkyl, alkenyl, cycloalkyl, or heteroalkyl present in any of R, R¹, R², R³, R⁴, R⁵, L¹, and L² is optionally substituted with one or more independently selected optional substituents.

13. The peptide conjugate of claim 11 or 12, wherein
R is hydrogen or C₁₋₆alkyl;
m is an integer from 0 to 4;
n is 1 or 2;
R¹ and R² at each instance of m are each independently hydrogen or C₁₋₆alkyl; or
R¹ is L²-Z¹—C₁₋₆alkyl;
R³, R⁴, and R⁵ are each independently hydrogen or C₁₋₆alkyl; or R³ is L²-Z¹—C₁₋₆alkyl;
L¹ and L² are each independently C₅₋₂₁alkyl, C₅₋₂₁alkenyl, or C₄₋₂₀heteroalkyl;
wherein any alkyl, alkenyl, or heteroalkyl present in any of R, R¹, R², R³, R⁴, R⁵, L¹, and L² is optionally substituted with one or more independently selected optional substituents.

14. The peptide conjugate of any one of claims 11-13, wherein Z and Z¹ are each independently selected from —C(O)O—, —C(O)NR—, and —C(O)S—, preferably —C(O)O—.

15. The peptide conjugate of any one of claims 11-14, wherein the lipid-containing moiety is of the formula (I) 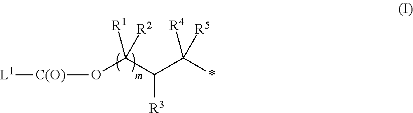
wherein
m, L¹, R¹, R², R³, R⁴, and R⁵ are as defined in any one of the preceding claims; and
Z¹ when present is —C(O)O—.

16. The peptide conjugate of any one of claims 11-15, wherein m is an integer from 0 to 2.

17. The peptide conjugate of any one of claims 11-16, wherein m is 0 or 1.

18. The peptide conjugate of any one of claims 11-17, wherein m is 0.

19. The peptide conjugate of any one of claims 11-18, wherein R¹ and R² at each instance of m are each independently hydrogen.

20. The peptide conjugate of any one of claims 11-19, wherein R⁴ and R⁵ are each hydrogen.

21. The peptide conjugate of any one of claims 11-20, wherein R³ is hydrogen or C₁₋₆alkyl.

22. The peptide conjugate of any one of claims 11-20, wherein the lipid-containing moiety is of the formula (IV): 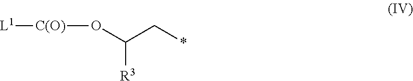
wherein
R³ is hydrogen, L²-C(O)—OCH₂, or L²-C(O)—OCH₂CH₂; and
L¹ and L² are each independently C₅₋₂₁alkyl, C₅₋₂₁alkenyl, or C₄₋₂₀heteroalkyl.

23. The peptide conjugate of any one of claims 11-22, wherein L¹ and L² are each independently is C₅₋₂₁alkyl.

24. The peptide conjugate of any one of claims 11-23, wherein L¹ and L² are each independently is C₉₋₂₁alkyl.

25. The peptide conjugate of any one of claims 11-24, wherein L¹ and L² are each independently linear C₁₅alkyl.

26. The peptide conjugate of any one of claims 11-20 and 22-25, wherein R³ is L²-C(O)—OCH₂CH₂.

27. The peptide conjugate of any one of claims 11-25, wherein R³ is hydrogen.

28. The peptide conjugate of any one of claims 11-27, wherein the one or more independently selected optional substituents are selected from halo, CN, NO₂, OH, NH₂, NHR^{x}, NR—R^{y}, C₁₋₆haloalkyl, C₁₋₆haloalkoxy, C(O)NH₂, C(O)NHR^{x}, C(O)NR^{x}R^{y}, SO₂R^{x}, OR^{y}, SR^{x}, S(O)R^{x}, C(O)R^{x}, and C₁₋₆aliphatic; wherein R^{x} and R^{y} are each independently C₁₋₆aliphatic, for example C₁₋₆alkyl.

29. The peptide conjugate of any one of the preceding claims, wherein the N-terminal group of the peptide is —NR^{a}R^{b}, wherein R^{a} and R^{b} are each independently hydrogen, alkyl, cycloalkyl, acyl, aryl, or arylalkyl; and/or the C-terminal group of the peptide is —CH₂OR^{c}, —C(O)OR^{c} or —C(O)NR^{c}R^{d}, wherein R^{c} and R^{d} are each independently hydrogen, alkyl, cycloalkyl, aryl, or arylalkyl.

30. The peptide conjugate of any one of the preceding claims, wherein the N-terminal group of the peptide is —NH2 or —NH(acyl), for example —NHAc; and/or the C-terminal group of the peptide is —C(O)NH₂.

31. A peptide conjugate of any one of the preceding claims, wherein the peptide comprises or consists of an amino acid sequence of the formula:
[SEQ ID No. 1]
Z-Xaa⁸Xaa⁹Xaa¹⁰Xaa¹¹Leu¹²Xaa¹³Xaa¹⁴Xaa¹⁵
Leu¹⁶Xaa¹⁷Xaa¹⁸Xaa¹⁹Xaa²⁰Xaa²¹Xaa²²Xaa²³
Xaa²⁴Xaa²⁵Xaa²⁶Phe²⁷Xaa²⁸Xaa²⁹Thi³⁰Xaa³¹
Val³²Gly³³Xaa³⁴Xaa³⁵Xaa³⁶Phe³⁷
wherein:
Z is absent or is Xaa¹Xaa²Xaa³Xaa⁴Xaa⁵Xaa⁶Xaa⁷, Xaa²Xaa³Xaa⁴Xaa⁵Xaa⁶Xaa⁷, Xaa³Xaa⁴Xaa⁵Xaa⁶Xaa⁷, Xaa⁴Xaa⁵Xaa⁶Xaa⁷, Xaa⁵Xaa⁶Xaa⁷, Xaa⁶Xaa⁷ or Xaa⁷
wherein:
Xaa¹ is alanine, valine, leucine, isoleucine, proline, phenylalanine, methionine, tryptophan, serine, glycine, asparagine, glutamine, threonine, tyrosine or cysteine;
Xaa² is cysteine, serine, alanine, glycine, asparagine, glutamine, threonine, tyrosine;
Xaa³ is aspartate, glutamate, asparagine, glutamine, glycine, serine, threonine, tyrosine or cysteine;
Xaa⁴ is threonine, glycine, asparagine, glutamine, serine, phenylalanine, tyrosine, valine, isoleucine or cysteine;
Xaa⁵ is alanine, valine, leucine, isoleucine, proline, phenylalanine, tyrosine methionine or tryptophan;
Xaa⁶ is threonine, glycine, asparagine, glutamine, serine, tyrosine, phenylalanine, valine, isoleucine or cysteine;
Xaa⁷ is cysteine, serine, alanine, glycine, asparagine, glutamine, threonine, phenylalanine or tyrosine;
Xaa⁸ is valine, alanine, leucine, isoleucine, proline, phenylalanine, tyrosine methionine, tryptophan or threonine;
Xaa⁹ is threonine, glycine, asparagine, glutamine, serine, tyrosine, valine, isoleucine or cysteine;
Xaa¹⁰ is histidine, lysine, arginine, asparagine, glutamine, serine, alanine, glycine, valine, leucine or isoleucine;
Xaa¹¹ is arginine, lysine, histidine, glutamine or asparagine;
Xaa¹³ is alanine, valine, leucine, isoleucine, proline, phenylalanine, methionine, tryptophan, serine, glycine, asparagine, glutamine, threonine, tyrosine or cysteine;
Xaa¹⁴ is glycine, proline, alanine, asparagine, glutamine, serine, threonine, phenylalanine, tyrosine, cysteine, glutamate or aspartate;
Xaa¹⁵ is leucine, isoleucine, valine, alanine, methionine, phenylalanine, tyrosine, proline or tryptophan;
Xaa¹⁷ is serine, threonine, alanine, valine, leucine, isoleucine, proline, phenylalanine, tyrosine, methionine, tryptophan, arginine, lysine, histidine, glutamine, asparagine or cysteine;
Xaa¹⁸ is arginine, lysine, histidine, glutamine or asparagine;
Xaa¹⁹ is serine, threonine, alanine, valine, leucine, isoleucine, proline, phenylalanine, tyrosine, methionine, tryptophan or cysteine;
Xaa²⁰ is glycine, proline, alanine, beta alanine, asparagine, glutamine, serine, threonine, phenylalanine or tyrosine;
Xaa²¹ is glycine, proline, alanine, beta alanine, asparagine, glutamine, serine, threonine, phenylalanine or tyrosine;
Xaa²² is valine, alanine, leucine, isoleucine, proline, phenylalanine, tyrosine, methionine or tryptophan or threonine;
Xaa²³ is valine, alanine, leucine, isoleucine, proline, phenylalanine, tyrosine, methionine, tryptophan or threonine;
Xaa²⁴ is lysine, arginine, glutamine, asparagine or histidine;
Xaa²⁵ is asparagine, glutamine, glycine, serine, threonine, tyrosine, phenylalanine, alanine, glutamate, aspartate or cysteine;
Xaa²⁶ is asparagine, glutamine, glycine, serine, threonine, phenylalanine, tyrosine or cysteine;
Xaa²⁸ is valine, alanine, leucine, isoleucine, proline, phenylalanine, tyrosine, methionine, tryptophan or threonine;
Xaa²⁹ is proline, alanine, valine, leucine, isoleucine, glycine, phenylalanine, tyrosine, methionine or tryptophan;
Xaa³¹ is asparagine, glutamine, glycine, serine, threonine, phenylalanine, tyrosine, glutamate, aspartate or cysteine;
Xaa³⁴ is serine, threonine, alanine, valine, leucine, isoleucine, proline, phenylalanine, tyrosine, methionine, tryptophan or cysteine;
Xaa³⁵ is lysine, arginine, glutamine, asparagine, histidine, aspartate or glutamate; and
Xaa³⁶ is alanine, valine, leucine, isoleucine, proline, phenylalanine, tyrosine, methionine or tryptophan;
wherein one or more of Xaa1-Xaa11, Xaa13-Xaa15, Xaa17-Xaa26, Xaa28, Xaa29, Xaa31 and Xaa34-Xaa36 is or is substituted with an amino acid that is covalently conjugated to a lipid-containing moiety.

32. A peptide conjugate of claim 31 wherein Z is absent, or is Xaa1Xaa2Xaa3Xaa4Xaa5Xaa6Xaa7 or Xaa7.

33. A peptide conjugate of claim 31 or 32 wherein
a) Xaa1 is alanine, valine, leucine, isoleucine, serine, glycine, or threonine;
b) Xaa2 is cysteine, serine or alanine;
c) Xaa3 is aspartate, glutamate, asparagine or glutamine;
d) Xaa4 is threonine, glycine, asparagine, glutamine or serine;
e) Xaa5 is alanine, valine, leucine or isoleucine;
f) Xaa6 is threonine, glycine, asparagine, glutamine or serine;
g) Xaa7 is cysteine, serine, or alanine;
h) Xaa8 is valine, alanine, leucine, isoleucine, phenylalanine or methionine;
i) Xaa9 is threonine, glycine, asparagine, glutamine or serine;
j) Xaa10 is histidine, lysine or arginine;
k) Xaa11 is arginine, lysine or histidine;
l) Xaa13 is alanine, valine, leucine, isoleucine, serine, glycine, or threonine;
m) Xaa14 is glycine, proline, alanine, aspartate or glutamate;
n) Xaa15 is leucine, isoleucine, valine, alanine, methionine or phenylalanine;
o) Xaa17 is serine, threonine, alanine, arginine, lysine or histidine;
p) Xaa18 is arginine, lysine or histidine;
q) Xaa19 is serine, threonine or alanine;
r) Xaa20 is glycine, proline or alanine;
s) Xaa21 is glycine, proline or alanine;
t) Xaa22 is valine, alanine, leucine, isoleucine, phenylalanine or methionine;
u) Xaa23 is valine, alanine, leucine, isoleucine, proline, phenylalanine, methionine, tryptophan or threonine;
v) Xaa24 is lysine, arginine or histidine;
w) Xaa25 is asparagine, glutamine, serine, threonine, alanine;
x) Xaa26 is asparagine, serine, glutamate or glutamine;
y) Xaa28 is valine, alanine, leucine, isoleucine, proline, phenylalanine, methionine, tryptophan or threonine;
z) Xaa29 is proline, alanine or glycine;
aa) Xaa31 is asparagine, glutamine, glutamate or aspartate;
bb) Xaa34 is serine, threonine or alanine;
cc) Xaa35 is lysine, arginine, histidine, aspartate or glutamate;
dd) Xaa36 is alanine, valine, leucine or isoleucine; or
ee) any combination of any two or more of a) to dd);
wherein one or more of Xaa1-Xaa11, Xaa13-Xaa15, Xaa17-Xaa26, Xaa28, Xaa29, Xaa31 and Xaa34-Xaa36 is or is substituted with an amino acid that is covalently conjugated to a lipid-containing moiety.

34. A peptide conjugate of any one of claims 31-33 wherein
a) Xaa1 is alanine or serine;
b) Xaa2 is cysteine;
c) Xaa3 is aspartate or glutamate;
d) Xaa4 is threonine;
e) Xaa5 is alanine;
f) Xaa6 is threonine;
g) Xaa7 is cysteine;
h) Xaa8 is valine;
i) Xaa9 is threonine;
j) Xaa10 is histidine;
k) Xaa11 is arginine;
l) Xaa13 is alanine;
m) Xaa14 is glycine or aspartate;
n) Xaa15 is leucine;
o) Xaa17 is serine or arginine;
p) Xaa18 is arginine;
q) Xaa19 is serine;
r) Xaa20 is glycine;
s) Xaa21 is glycine;
t) Xaa22 is valine or methionine;
u) Xaa23 is valine or leucine;
v) Xaa24 is lysine;
w) Xaa25 is asparagine or serine;
x) Xaa26 is asparagine, serine or glutamate;
y) Xaa28 is valine;
z) Xaa29 is proline;
aa) Xaa31 is asparagine or aspartate;
bb) Xaa34 is serine;
cc) Xaa35 is lysine or glutamate;
dd) Xaa36 is alanine; or
ee) any combination of any two or more of a) to dd);
wherein one or more of Xaa1-Xaa11, Xaa13-Xaa15, Xaa17-Xaa26, Xaa28, Xaa29, Xaa31 and Xaa34-Xaa36 is or is substituted with an amino acid that is covalently conjugated to a lipid-containing moiety.

35. A peptide conjugate of any one of claims 31-34 wherein the peptide comprises or consists of an amino acid sequence of the formula:
[SEQ ID No. 2]
Z-Xaa⁸Thr⁹Xaa¹⁰Xaa¹¹Leu¹²Ala¹³Xaa¹⁴Leu¹⁵
Leu¹⁶Xaa¹⁷Xaa¹⁸Xaa¹⁹Gly²⁰Xaa²¹Xaa²²Xaa²³
Xaa²⁴Xaa²⁵Asn²⁶Phc²⁷Val²⁸Pro²⁹Thr³⁰Xaa³¹
Val³²Gly³³Scr³⁴Xaa³⁵Ala³⁶Phc³⁷
wherein:
Z is absent or is Xaa¹Xaa²Xaa³Thr⁴Ala⁵Xaa⁶Xaa⁷, Xaa²Xaa³Thr⁴Ala⁵Xaa⁶Xaa⁷, Xaa³Thr⁴Ala⁵Xaa⁶Xaa⁷, Thr⁴Ala⁵Xaa⁶Xaa⁷, Ala⁵Xaa⁶Xaa⁷, Xaa⁶Xaa⁷ or Xaa⁷
wherein:
a) Xaa1 is alanine or serine;
b) Xaa2 is cysteine or homocysteine;
c) Xaa3 is aspartate or asparagine;
d) Xaa6 is threonine, cysteine or homocysteine;
e) Xaa7 is cysteine or homocysteine;
f) Xaa8 is valine, cysteine or homocysteine;
g) Xaa10 is histidine, cysteine or homocysteine,
h) Xaa11 is arginine, cysteine or homocysteine;
i) Xaa14 is glycine or aspartate;
j) Xaa17 is serine, arginine, cysteine or homocysteine,
k) Xaa18 is arginine, cysteine or homocysteine;
l) Xaa19 is a serine, cysteine or homocysteine;
m) Xaa21 is glycine, cysteine or homocysteine;
n) Xaa22 is valine or methionine;
o) Xaa23 is valine or leucine;
p) Xaa24 is lysine, cysteine or homocysteine;
q) Xaa25 is asparagine, serine or aspartate;
r) Xaa31 is asparagine or aspartate; and
s) Xaa35 is lysine, glutamate, cysteine or homocysteine;
wherein at least one cysteine or homocysteine in the peptide is covalently conjugated to a lipid-containing moiety.

36. A peptide conjugate of any one of claims 31-35 wherein one or more of Xaa6-Xaa8, Xaa10, Xaa11, Xaa17-Xaa19, Xaa21, Xaa24 and Xaa35 is or is substituted with an amino acid that is covalently conjugated to a lipid-containing moiety.

37. A peptide conjugate of any one of claims 31-36 wherein one or more of Xaa7, Xaa8, Xaa11, Xaa24 and Xaa35 is or is substituted with an amino acid that is covalently conjugated to a lipid-containing moiety.

38. A peptide conjugate of any one of claims 31-37 wherein one or more of Xaa7, Xaa8, Xaa24 and Xaa35 is or is substituted with an amino acid that is covalently conjugated to a lipid-containing moiety.

39. A peptide conjugate of any one of claims 31-38 wherein 1 or 2 of Xaa6-Xaa8, Xaa10, Xaa11, Xaa17-Xaa19, Xaa21, Xaa24 and Xaa35 is or is substituted with an amino acid that is covalently conjugated to a lipid-containing moiety.

40. A peptide conjugate of any one of claims 31-39 wherein 1 or 2 of Xaa7, Xaa8, Xaa11, Xaa24 and Xaa35 is or is substituted with an amino acid that is covalently conjugated to a lipid-containing moiety.

41. A peptide conjugate of any one of claims 31-40 wherein two or more of Xaa6-Xaa8, Xaa10, Xaa11, Xaa17-Xaa19, Xaa21, Xaa24 and Xaa35 is or is substituted with an amino acid that is covalently conjugated to a lipid-containing moiety.

42. A peptide conjugate of any one of claims 31-41 wherein two or more of Xaa7, Xaa8, Xaa11, Xaa24 and Xaa35 is or is substituted with an amino acid that is covalently conjugated to a lipid-containing moiety.

43. A peptide conjugate of any one of claims 1-30 wherein the peptide comprises or consists of
a) the amino acid sequence of SEQ ID NO:3;
b) 25 or more contiguous amino acids of SEQ ID NO:3;
c) amino acids 7-37 of SEQ ID No:3;
d) amino acids 8-37 of SEQ ID NO:3;
e) the amino acid sequence of SEQ ID NO:4;
f) 25 or more contiguous amino acids of SEQ ID NO:4;
g) amino acids 7-37 of SEQ ID No:4;
h) amino acids 8-37 of SEQ ID NO:4; or
i) a functional variant of any one of a) to h) comprising or consisting of an amino acid sequence having at least about 60% amino acid sequence identity to the sequence defined in any one of a) to h);
wherein one or more amino acids in the sequence is or is substituted with an amino acid covalently conjugated to a lipid-containing moiety.

44. A peptide conjugate of any of the preceding claims wherein the peptide comprises or consists of an amino acid sequence selected from
a) amino acids 2-37 of SEQ ID No:3 or SEQ ID No:4;
b) amino acids 3-37 of SEQ ID No:3 or SEQ ID No:4;
c) amino acids 4-37 of SEQ ID No:3 or SEQ ID No:4;
d) amino acids 5-37 of SEQ ID No:3 or SEQ ID No:4;
e) amino acids 6-37 of SEQ ID No:3 or SEQ ID No:4; or
f) a functional variant of any one of a) to e) comprising or consisting of an amino acid sequence having at least about 60% amino acid sequence identity to the sequence defined in any one of a) to e);
wherein one or more amino acids in the sequence is or is substituted with an amino acid covalently conjugated to a lipid-containing moiety.

45. A peptide conjugate of claim 43 (i) or claim 44 (f) wherein the amino acid sequence has at least about 90% sequence identity to the sequence defined in claim 43 a)-h) or claim 44 a)-e).

46. A peptide conjugate of any one of claims 43 to 45 wherein the peptide comprises an amino acid covalently conjugated to a lipid-containing moiety at one or more amino acid positions corresponding to positions 1-11, 13-15, 17-26, 28, 29, 31 and 34-36 of SEQ ID No 3 or SEQ ID No:4.

47. A peptide conjugate of any one of claims 43 to 46 wherein the peptide comprises an amino acid covalently conjugated to a lipid-containing moiety at one or more amino acid positions corresponding to positions 6-8, 10, 11, 17-19, 21, 24 and 35 of SEQ ID No 3 or SEQ ID NO:4.

48. A peptide conjugate of any one of claims 43 to 47 wherein the peptide comprises an amino acid covalently conjugated to a lipid-containing moiety at one or more amino acid positions corresponding to positions 6-8, 10, 11, 21, 24 and 35 of SEQ ID No 3 or SEQ ID NO: 4.

49. A peptide conjugate of any one of claims 43 to 48 wherein the peptide comprises an amino acid covalently conjugated to a lipid-containing moiety at one or more amino acid positions corresponding to positions 7, 8, 11, 24 and 35 of SEQ ID No 3 or SEQ ID NO: 4.

50. A peptide conjugate of any one of claims 43 to 49 wherein the peptide comprises an amino acid covalently conjugated to a lipid-containing moiety at one or more amino acid positions corresponding to positions 7, 8, 24 and 35 of SEQ ID No 3 or SEQ ID NO: 4.

51. A peptide conjugate of any one of the preceding claims wherein the N-terminal amino acid of the peptide is covalently conjugated to a lipid-containing moiety.

52. A peptide conjugate of any one of the preceding claims wherein the peptide comprises one or more amino acids covalently conjugated to a lipid-containing moiety in
a) a region of the peptide comprising amino acids Xaa1-Xaa7 or a region of the peptide corresponding to amino acids 1-7 of SEQ ID No:3 or SEQ ID No:4;
b) a region of the peptide comprising amino acids Xaa8-Xaa18 or a region of the peptide corresponding to amino acids 8-18 of SEQ ID No:3 or SEQ ID No:4;
c) a region of the peptide comprising amino acids Xaa19-Xaa26 or a region of the peptide corresponding to amino acids 19-26 of SEQ ID No:3 or SEQ ID No:4;
d) a region of the peptide comprising Xaa27-Xaa37 or a region of the peptide corresponding to amino acids 27-37 of SEQ ID No:3 or SEQ ID No:4; or
e) any combination of any two or more of a) to d).

53. A peptide conjugate of any one of the preceding claims wherein the peptide comprises from about 1 to about 5 amino acids covalently conjugated to a lipid-containing moiety.

54. A peptide conjugate of any one of the preceding claims wherein the peptide comprises from about 1 to about 3 amino acids covalently conjugated to a lipid-containing moiety.

55. A peptide conjugate of any one of the preceding claims wherein the peptide comprises 1 or 2 amino acids covalently conjugated to a lipid-containing moiety.

56. A peptide conjugate of any one of the preceding claims wherein the amino acid covalently conjugated to a lipid-containing moiety is cysteine or homocysteine, and the the lipid-containing moiety is covalently attached via the sulfur atom of the sulfide group of the cysteine or homocysteine.

57. A peptide conjugate of any one of the preceding claims wherein the peptide comprises or consists of an amino acid sequence selected from
a)
[SEQ ID No: 5]
AXDTATXVTHRLAGLLSRSGGVVKNNFVPTNVGSKAF;
b)
[SEQ ID No: 6]
XXDTATXVTHRLAGLLSRSGGVVKNNFVPTNVGSKAF;
c)
[SEQ ID No: 7]
AXXTATXVTHRLAGLLSRSGGVVKNNFVPTNVGSKAF;
d)
[SEQ ID No: 8]
AXDXATXVTHRLAGLLSRSGGVVKNNFVPTNVGSKAF;
e)
[SEQ ID No: 9]
AXDTXTXVTHRLAGLLSRSGGVVKNNFVPTNVGSKAF;
f)
[SEQ ID No: 10]
AXDTAXXVTHRLAGLLSRSGGVVKNNFVPTNVGSKAF;
g)
[SEQ ID No: 11]
XDTATXVTHRLAGLLSRSGGVVKNNFVPTNVGSKAF;
h)
[SEQ ID No: 12]
DTATXVTHRLAGLLSRSGGVVKNNFVPTNVGSKAF;
i)
[SEQ ID No: 13]
XTATXVTHRLAGLLSRSGGVVKNNFVPTNVGSKAF;
j)
[SEQ ID No: 14]
TATXVTHRLAGLLSRSGGVVKNNFVPTNVGSKAF;
k)
[SEQ ID No: 15]
ATXVTHRLAGLLSRSGGVVKNNFVPTNVGSKAF;
l)
[SEQ ID No: 16]
TXVTHRLAGLLSRSGGVVKNNFVPTNVGSKAF;
m)
[SEQ ID No: 17]
XVTHRLAGLLSRSGGVVKNNFVPTNVGSKAF;
n)
[SEQ ID No: 18]
XTHRLAGLLSRSGGVVKNNFVPTNVGSKAF;
o)
[SEQ ID No: 19]
VXHRLAGLLSRSGGVVKNNFVPTNVGSKAF;
p)
[SEQ ID No: 20]
VTXRLAGLLSRSGGVVKNNFVPTNVGSKAF;
q)
[SEQ ID No: 21]
VTHXLAGLLSRSGGVVKNNFVPTNVGSKAF;
r)
[SEQ ID No: 22]
VTHRLXGLLSRSGGVVKNNFVPTNVGSKAF;
s)
[SEQ ID No: 23]
VTHRLAXLLSRSGGVVKNNFVPTNVGSKAF;
t)
[SEQ ID No: 24]
VTHRLAGXLSRSGGVVKNNFVPTNVGSKAF;
u)
[SEQ ID No: 25]
VTHRLAGLLXRSGGVVKNNFVPTNVGSKAF;
v)
[SEQ ID No: 26]
VTHRLAGLLSXSGGVVKNNFVPTNVGSKAF;
w)
[SEQ ID No: 27]
VTHRLAGLLSRXGGVVKNNFVPTNVGSKAF;
x)
[SEQ ID No: 28]
VTHRLAGLLSRSXGVVKNNFVPTNVGSKAF;
y)
[SEQ ID No: 29]
VTHRLAGLLSRSGXVVKNNFVPTNVGSKAF;
z)
[SEQ ID No: 30]
VTHRLAGLLSRSGGXVKNNFVPTNVGSKAF;
aa)
[SEQ ID No: 32]
VTHRLAGLLSRSGGVXKNNFVPTNVGSKAF;
bb)
[SEQ ID No: 33]
VTHRLAGLLSRSGGVVXNNFVPTNVGSKAF;
cc)
[SEQ ID No: 34]
VTHRLAGLLSRSGGVVKXNFVPTNVGSKAF;
dd)
[SEQ ID No: 35]
VTHRLAGLLSRSGGVVKNXFVPTNVGSKAF;
ee)
[SEQ ID No: 36]
VTHRLAGLLSRSGGVVKNNFXPTNVGSKAF;
ff)
[SEQ ID No: 37]
VTHRLAGLLSRSGGVVKNNFVXTNVGSKAF;
gg)
[SEQ ID No: 38]
VTHRLAGLLSRSGGVVKNNFVPTXVGSKAF;
hh)
[SEQ ID No: 39]
VTHRLAGLLSRSGGVVKNNFVPTNVGXKAF;
ii)
[SEQ ID No: 40]
VTHRLAGLLSRSGGVVKNNFVPTNVGSXAF;
jj)
[SEQ ID No: 41]
VTHRLAGLLSRSGGVVKNNFVPTNVGSKXF;
kk)
[SEQ ID No: 42]
AXNTATXVTHRLAGLLSRSGGMVKSNFVPTNVGSKAF;
ll)
[SEQ ID No: 43]
XXNTATXVTHRLAGLLSRSGGMVKSNFVPTNVGSKAF;
mm)
[SEQ ID No: 44]
AXXTATXVTHRLAGLLSRSGGMVKSNFVPTNVGSKAF;
nn)
[SEQ ID No: 45]
AXNXATXVTHRLAGLLSRSGGMVKSNFVPTNVGSKAF;
oo)
[SEQ ID No: 46]
AXNTXTXVTHRLAGLLSRSGGMVKSNFVPTNVGSKAF;
pp)
[SEQ ID No: 47]
AXNTAXXVTHRLAGLLSRSGGMVKSNFVPTNVGSKAF;
qq)
[SEQ ID No: 48]
XNTATXVTHRLAGLLSRSGGMVKSNFVPTNVGSKAF;
rr)
[SEQ ID No: 49]
NTATXVTHRLAGLLSRSGGMVKSNFVPTNVGSKAF;
ss)
[SEQ ID No: 50]
AXNXTATXVTHRLAGLLSRSGGMVKSNFVPTNVGSKAF;
tt)
[SEQ ID No: 51]
XTATXVTHRLAGLLSRSGGMVKSNFVPTNVGSKAF;
uu)
[SEQ ID No: 52]
TATXVTHRLAGLLSRSGGMVKSNFVPTNVGSKAF;
vv)
[SEQ ID No: 53]
ATXVTHRLAGLLSRSGGMVKSNFVPTNVGSKAF;
ww)
[SEQ ID No: 54]
TXVTHRLAGLLSRSGGMVKSNFVPTNVGSKAF;
xx)
[SEQ ID No: 55]
XVTHRLAGLLSRSGGMVKSNFVPTNVGSKAF;
yy)
[SEQ ID No: 56]
XTHRLAGLLSRSGGMVKSNFVPTNVGSKAF;
zz)
[SEQ ID No: 57]
VXHRLAGLLSRSGGMVKSNFVPTNVGSKAF;
aaa)
[SEQ ID No: 58]
VTXRLAGLLSRSGGMVKSNFVPTNVGSKAF;
bbb)
[SEQ ID No: 59]
VTHXLAGLLSRSGGMVKSNFVPTNVGSKAF;
ccc)
[SEQ ID No: 60]
VTHRLXGLLSRSGGMVKSNFVPTNVGSKAF;
ddd)
[SEQ ID No: 61]
VTHRLAXLLSRSGGMVKSNFVPTNVGSKAF;
eee)
[SEQ ID No: 62]
VTHRLAGXLSRSGGMVKSNFVPTNVGSKAF;
fff)
[SEQ ID No: 63]
VTHRLAGLLXRSGGMVKSNFVPTNVGSKAF;
ggg)
[SEQ ID No: 64]
VTHRLAGLLSXSGGMVKSNFVPTNVGSKAF;
hhh)
[SEQ ID No: 65]
VTHRLAGLLSRXGGMVKSNFVPTNVGSKAF;
iii)
[SEQ ID No: 66]
VTHRLAGLLSRSXGMVKSNFVPTNVGSKAF;
jjj)
[SEQ ID No: 67]
VTHRLAGLLSRSGXMVKSNFVPTNVGSKAF;
kkk)
[SEQ ID No: 68]
VTHRLAGLLSRSGGXVKSNFVPTNVGSKAF;
lll)
[SEQ ID No: 69]
VTHRLAGLLSRSGGMXKSNFVPTNVGSKAF;
mmm)
[SEQ ID No: 70]
VTHRLAGLLSRSGGMVXSNFVPTNVGSKAF;
nnn)
[SEQ ID No: 71]
VTHRLAGLLSRSGGMVKXNFVPTNVGSKAF;
ooo)
[SEQ ID No: 72]
VTHRLAGLLSRSGGMVKSXFVPTNVGSKAF;
ppp)
[SEQ ID No: 73]
VTHRLAGLLSRSGGMVKSNFXPTNVGSKAF;
qqq)
[SEQ ID No: 74]
VTHRLAGLLSRSGGMVKSNFVXTNVGSKAF;
rrr)
[SEQ ID No: 75]
VTHRLAGLLSRSGGMVKSNFVPTXVGSKAF;
sss)
[SEQ ID No: 76]
VTHRLAGLLSRSGGMVKSNFVPTNVGXKAF;
ttt)
[SEQ ID No: 77]
VTHRLAGLLSRSGGMVKSNFVPTNVGSXAF;
or
uuu)
[SEQ ID No: 78]
VTHRLAGLLSRSGGMVKSNFVPTNVGSKXF;
wherein X is cysteine or homocysteine and wherein at least one X in the peptide is covalently conjugated to a lipid-containing moiety.

58. A peptide conjugate of any one of the preceding claims wherein the peptide comprises or consists of an amino acid sequence selected from
a)
[SEQ ID No: 17]
XVTHRLAGLLSRSGGVVKNNFVPTNVGSKAF;
b)
[SEQ ID No: 18]
XTHRLAGLLSRSGGVVKNNFVPTNVGSKAF;
c)
[SEQ ID No: 33]
VTHRLAGLLSRSGGVVXNNFVPTNVGSKAF;
d)
[SEQ ID No: 40]
VTHRLAGLLSRSGGVVKNNFVPTNVGSXAF;
e)
[SEQ ID No: 10]
AXDTAXXVTHRLAGLLSRSGGVVKNNFVPTNVGSKAF;
f)
[SEQ ID No: 20]
VTXRLAGLLSRSGGVVKNNFVPTNVGSKAF;
g)
[SEQ ID No: 21]
VTHXLAGLLSRSGGVVKNNFVPTNVGSKAF;
h)
[SEQ ID No: 29]
VTHRLAGLLSRSGXVVKNNFVPTNVGSKAF;
i)
[SEQ ID No: 55]
XVTHRLAGLLSRSGGMVKSNFVPTNVGSKAF;
j)
[SEQ ID No: 56]
XTHRLAGLLSRSGGMVKSNFVPTNVGSKAF;
k)
[SEQ ID No: 70]
VTHRLAGLLSRSGGMVXSNFVPTNVGSKAF;
l)
[SEQ ID No: 77]
VTHRLAGLLSRSGGMVKSNFVPTNVGSXAF;
m)
[SEQ ID No: 47]
AXNTAXXVTHRLAGLLSRSGGMVKSNFVPTNVGSKAF;
n)
[SEQ ID No: 58]
VTXRLAGLLSRSGGMVKSNFVPTNVGSKAF;
o)
[SEQ ID No: 59
VTHXLAGLLSRSGGMVKSNFVPTNVGSKAF;
or
p)
[SEQ ID No: 67]
VTHRLAGLLSRSGXMVKSNFVPTNVGSKAF;
wherein X is cysteine or homocysteine,
and wherein at least one X in the peptide is covalently conjugated to a lipid-containing moiety.

59. A peptide conjugate of any one of the preceding claims wherein the peptide comprises or consists of an amino acid sequence selected from
a)
[SEQ ID No: 79]
CVTHRLAGLLSRSGGVVKNNFVPTNVGSKAF;
b)
[SEQ ID No: 80]
CTHRLAGLLSRSGGVVKNNFVPTNVGSKAF;
c)
[SEQ ID No: 81]
VTHRLAGLLSRSGGVVCNNFVPTNVGSKAF;
d)
[SEQ ID No: 82]
VTHRLAGLLSRSGGVVKNNFVPTNVGSCAF;
e)
[SEQ ID No: 83]
ACDTACCVTHRLAGLLSRSGGVVKNNFVPTNVGSKAF;
f)
[SEQ ID No: 84]
VTCRLAGLLSRSGGVVKNNFVPTNVGSKAF;
g)
[SEQ ID No: 85]
VTHCLAGLLSRSGGVVKNNFVPTNVGSKAF;
h)
[SEQ ID No: 86]
VTHRLAGLLSRSGCVVKNNFVPTNVGSKAF;
i)
[SEQ ID No: 87]
CVTHRLAGLLSRSGGMVKSNFVPTNVGSKAF;
j)
[SEQ ID No: 88]
CTHRLAGLLSRSGGMVKSNFVPTNVGSKAF;
k)
[SEQ ID No: 89]
VTHRLAGLLSRSGGMVCSNFVPTNVGSKAF;
1)
[SEQ ID No: 90]
VTHRLAGLLSRSGGMVKSNFVPTNVGSCAF;
m)
[SEQ ID No: 91]
ACNTACCVTHRLAGLLSRSGGMVKSNFVPTNVGSKAF;
n)
[SEQ ID No: 92]
VTCRLAGLLSRSGGMVKSNFVPTNVGSKAF;
o)
[SEQ ID No: 93]
VTHCLAGLLSRSGGMVKSNFVPTNVGSKAF;
or
p)
[SEQ ID No: 94]
VTHRLAGLLSRSGCMVKSNFVPTNVGSKAF;
wherein at least one C in the peptide is covalently conjugated to a lipid-containing moiety.

60. A peptide conjugate of any one of the preceding claims wherein the peptide comprises or consists of an amino acid sequence selected from
a)
[SEQ ID No: 100]
XXTHRLAGLLSRSGGVVKNNFVPTNVGSKAF;
b)
[SEQ ID No: 101]
XVTHXLAGLLSRSGGVVKNNFVPTNVGSKAF;
c)
[SEQ ID No: 102]
XVTHRLAGLLSRSGGVVXNNFVPTNVGSKAF;
d)
[SEQ ID No: 103]
XVTHRLAGLLSRSGGVVKNNFVPTNVGSXAF;
e)
[SEQ ID No: 104]
XTHXLAGLLSRSGGVVKNNFVPTNVGSKAF;
f)
[SEQ ID No: 105]
XTHRLAGLLSRSGGVVXNNFVPTNVGSKAF;
g)
[SEQ ID No: 106]
XTHRLAGLLSRSGGVVKNNFVPTNVGSXAF;
h)
[SEQ ID No: 107]
VTHXLAGLLSRSGGVVXNNFVPTNVGSKAF;
i)
[SEQ ID No: 108]
VTHXLAGLLSRSGGVVKNNFVPTNVGSXAF;
j)
[SEQ ID No: 109]
VTHRLAGLLSRSGGVVXNNFVPTNVGSXAF;
k)
[SEQ ID No: 110]
XXTHRLAGLLSRSGGMVKSNFVPTNVGSKAF;
1)
[SEQ ID No: 111]
XVTHXLAGLLSRSGGMVKSNFVPTNVGSKAF;
m)
[SEQ ID No: 112]
XVTHRLAGLLSRSGGMVXSNFVPTNVGSKAF;
n)
[SEQ ID No: 113]
XVTHRLAGLLSRSGGMVKSNFVPTNVGSXAF;
o)
[SEQ ID No: 114]
XTHXLAGLLSRSGGMVKSNFVPTNVGSKAF;
p)
[SEQ ID No: 115]
XTHRLAGLLSRSGGMVXSNFVPTNVGSKAF;
q)
[SEQ ID No: 116]
XTHRLAGLLSRSGGMVKSNFVPTNVGSXAF;
r)
[SEQ ID No: 117]
VTHXLAGLLSRSGGMVXSNFVPTNVGSKAF;
s)
[SEQ ID No: 118]
VTHXLAGLLSRSGGMVKSNFVPTNVGSXAF;
or
t)
[SEQ ID No: 119]
VTHRLAGLLSRSGGMVXSNFVPTNVGSXAF;
wherein X is cysteine or homocysteine,
and wherein at least two X in the peptide are covalently conjugated to a lipid-containing moiety.

61. A peptide conjugate of any one of the preceding claims wherein the peptide comprises or consists of an amino acid sequence selected from
a)
[SEQ ID No: 120]
CCTHRLAGLLSRSGGVVKNNFVPTNVGSKAF;
b)
[SEQ ID No: 121]
CVTHCLAGLLSRSGGVVKNNFVPTNVGSKAF;
c)
[SEQ ID No: 122]
CVTHRLAGLLSRSGGVVCNNFVPTNVGSKAF;
d)
[SEQ ID No: 123]
CVTHRLAGLLSRSGGVVKNNFVPTNVGSCAF;
e)
[SEQ ID No: 124]
CTHCLAGLLSRSGGVVKNNFVPTNVGSKAF;
f)
[SEQ ID No: 125]
CTHRLAGLLSRSGGVVCNNFVPTNVGSKAF;
g)
[SEQ ID No: 126]
CTHRLAGLLSRSGGVVKNNFVPTNVGSCAF;
h)
[SEQ ID No: 99]
VTHCLAGLLSRSGGVVCNNFVPTNVGSKAF;
i)
[SEQ ID No: 127]
VTHCLAGLLSRSGGVVKNNFVPTNVGSCAF;
j)
[SEQ ID No: 128]
VTHRLAGLLSRSGGVVCNNFVPTNVGSCAF;
k)
[SEQ ID No: 129]
CCTHRLAGLLSRSGGMVKSNFVPTNVGSKAF;
l)
[SEQ ID No: 130]
CVTHCLAGLLSRSGGMVKSNFVPTNVGSKAF;
m)
[SEQ ID No: 131]
CVTHRLAGLLSRSGGMVCSNFVPTNVGSKAF;
n)
[SEQ ID No: 132]
CVTHRLAGLLSRSGGMVKSNFVPTNVGSCAF;
o)
[SEQ ID No: 133]
CTHCLAGLLSRSGGMVKSNFVPTNVGSKAF;
p)
[SEQ ID No: 134]
CTHRLAGLLSRSGGMVCSNFVPTNVGSKAF;
q)
[SEQ ID No: 135]
CTHRLAGLLSRSGGMVKSNFVPTNVGSCAF;
r)
[SEQ ID No: 136]
VTHCLAGLLSRSGGMVCSNFVPTNVGSKAF;
s)
[SEQ ID No: 137]
VTHCLAGLLSRSGGMVKSNFVPTNVGSCAF;
or
t)
[SEQ ID No: 138]
VTHRLAGLLSRSGGMVCSNFVPTNVGSCAF;
wherein at least two C in the peptide are covalently conjugated to a lipid-containing moiety.

62. A peptide conjugate of any one of the preceding claims, wherein the peptide conjugate and α-CGRP8-37 (SEQ ID No:96) each independently have a first antagonist potency value (pA₂) at a CGRP receptor and a second antagonist potency value (pA₂) at a CGRP receptor;
wherein the first antagonist potency value (pA₂) at a CGRP receptor is after incubating the receptor and peptide conjugate or α-CGRP8-37 (SEQ ID No:96) and not washing the receptor prior to determining the antagonist potency value;
wherein the second antagonist potency value (pA₂) at a CGRP receptor is after incubating the receptor and peptide conjugate or α-CGRP8-37 (SEQ ID No:96) and then washing the receptor prior to determining the antagonist potency value;
wherein the second antagonist potency value (pA₂) is less than the first antagonist potency value (pA₂); and
the fold change reduction in antagonist potency between the first antagonist potency value (pA₂) of the peptide conjugate and the second antagonist potency value (pA₂) of the peptide conjutage is less than the fold change reduction in antagonist potency between the the first antagonist potency value (pA₂) of α-CGRP8-37 (SEQ ID No:96) and the second antagonist potency value (pA₂) of α-CGRP8-37 (SEQ ID No:96).

63. A peptide conjugate of claim 62, wherein the fold change reduction in antagonist potency between the first antagonist potency value (pA₂) of the peptide conjugate and the second antagonist potency value (pA₂) of the peptide conjutage is less than about 50, 45, 40, 35, 30, 25, 20, 15, 10, 9, 8, 7, 6, 5, 4, 3, or 2, wherein the antagonist potency value (pA₂) at a CGRP receptor is measured by a cAMP assay, and wherein the CGRP receptor is a CLR/RAMP1 CGRP receptor, for example as described in the Examples herein.

64. A peptide conjugate of claim 62 or 63, wherein the fold change reduction in antagonist potency between the first antagonist potency value (pA₂) of the peptide conjugate and the second antagonist potency value (pA₂) of the peptide conjutage is less than about 20, 15, 14, 13, 12, 11, 10, 9, 8, 7, 6, 5, 4, 3, or 2, wherein the antagonist potency value (pA₂) at a CGRP receptor is measured by a cAMP assay, and wherein the CGRP receptor is a CTR/RAMP1 AMY1 CGRP receptor, for example as described in the Examples herein.

65. A pharmaceutical composition comprising a peptide conjugate according to any one of the preceding claims; and a pharmaceutically acceptable carrier.

66. A method of antagonising a CGRP receptor in a subject in need thereof, comprising administering to the subject an effective amount of a peptide conjugate according to any one of claims 1-64.

67. A method of treating a disease or condition mediated by or modulated by a CGRP receptor or characterised by excessive CGRP receptor activation in a subject in need thereof, comprising administering to the subject a therapeutically effective amount of a peptide conjugate according to any one of claims 1-64.

68. A method of treating a disease or condition associated with or characterised by increased vasodilation in a subject in need thereof, comprising administering to the subject a therapeutically effective amount of a peptide conjugate according to any one of claims 1-64.

69. A method of treating a disease or condition selected from the group consisting of thermal injury, circulatory shock, menopausal hot flushes, asthma, sepsis, neurogenic inflammation, inflammatory skin conditions (for example psoriasis and contact dermatitis), allergic rhinitis, joint disorders (for example arthritis and temporomandibular joint disorder, preferably arthritis), cachexia (for example cancer-induced cachexia), pain, for example craniofacial pain disorders (for example migraine, headache, trigeminal neuralgia and dental pain, preferably migraine), and metabolic disorders or syndromes (for example obesity, type II diabetes, insulin resistance, dyslipidemia, hypertension, atherosclerosis and thrombosis) in a subject in need thereof, comprising administering to the subject a therapeutically effective amount of a peptide conjugate according to any one of claims 1-64.

70. The method according to any one of claims 67-69, wherein the disease or condition is migraine or headache (for example cluster headaches and post-traumatic headache).

71. A method for preparing a peptide conjugate according to any one of claims 1-64, the method comprising
(A) providing an amino acid conjugate comprising an amino acid of a calcitonin gene-related peptide (CGRP) peptide, wherein the amino acid is covalently conjugated to a lipid-containing moiety via a sulfur atom of a sulfide group; and
coupling the amino acid of the amino acid conjugate to one or more amino acids and/or one or more peptides to provide the peptide conjugate of any one of claims 1-64; or
(B) providing a peptide-conjugate comprising a peptide fragment of a calcitonin gene-related peptide (CGRP) peptide, wherein at least one amino acid of the peptide fragmentis covalently conjugated to a lipid-containing moiety via a sulfur atom of a sulfide group; and
coupling an amino acid of the peptide conjugate to one or more amino acids and/or one or more peptides to provide the peptide conjugate of any one of claims 1-64.

72. A method for preparing a peptide conjugate according to any one of claims 1-64, the method comprising reacting
a lipid-containing conjugation partner comprising a carbon carbon double bond, and
an amino acid-comprising conjugation partner comprising at least one amino acid comprising a thiol
under conditions effective to conjugate the lipid-containing conjugation partner to the amino acid-comprising conjugation partner.

73. The peptide conjugate, pharmaceutical composition, or method of any one of the preceding claims, wherein the CGRP receptor is a CLR/RAMP1 CGRP receptor or a CTR/RAMP1 AMY1 CGRP receptor.


SEQUENCE LISTING (138 sequences)

SEQ ID NO: 1 - protein, 37 aa, Artificial
  Synthetic peptide sequence
  MISC_FEATURE (1)..(1): Xaa1 is absent or is alanine, valine, leucine, isoleucine, proline, phenylalanine, methionine, tryptophan, serine, glycine, asparagine, glutamine, threonine, tyrosine or cysteine
  MISC_FEATURE (2)..(2): Xaa2 is absent or is cysteine, serine, alanine, glycine, asparagine, glutamine, threonine, tyrosine
  MISC_FEATURE (3)..(3): Xaa3 is absent or is aspartate, glutamate, asparagine, glutamine, glycine, serine, threonine, tyrosine or cysteine
  MISC_FEATURE (4)..(4): Xaa4 is absent or is threonine, glycine, asparagine, glutamine, serine, phenylalanine, tyrosine, valine isoleucine or cysteine
  MISC_FEATURE (5)..(5): Xaa5 is absent or is alanine, valine, leucine, isoleucine, proline, phenylalanine, tyrosine methionine or tryptophan
  MISC_FEATURE (6)..(6): Xaa6 is absent or is threonine, glycine, asparagine, glutamine, serine, tyrosine, phenylalanine, valine, isoleucine, cysteine or homocysteine, selenocysteine or homoselenocysteine
  MISC_FEATURE (7)..(7): Xaa7 is absent or is cysteine, serine, alanine, glycine, asparagine, glutamine, threonine, phenylalanine or tyrosine
  MISC_FEATURE (8)..(8): Xaa8 is valine, alanine, leucine, isoleucine, proline, phenylalanine, tyrosine methionine, tryptophan or threonine
  MISC_FEATURE (9)..(9): Xaa9 is threonine, glycine, asparagine, glutamine, serine, tyrosine, valine, isoleucine or cysteine
  MISC_FEATURE (10)..(10): Xaa10 is histidine, lysine, arginine, asparagine, glutamine, serine, alanine, glycine, valine, leucine or isoleucine
  MISC_FEATURE (11)..(11): Xaa11 is arginine, lysine, histidine, glutamine or asparagine
  MISC_FEATURE (13)..(13): Xaa13 is alanine, valine, leucine, isoleucine, proline, phenylalanine, methionine, tryptophan, serine, glycine, asparagine, glutamine, threonine, tyrosine or cysteine
  MISC_FEATURE (14)..(14): Xaa14 is glycine, proline, alanine, asparagine, glutamine, serine, threonine, phenylalanine, tyrosine, cysteine, glutamate or aspartate
  MISC_FEATURE (15)..(15): Xaa15 is leucine, isoleucine, valine, alanine, methionine, phenylalanine, tyrosine, proline or tryptophan
  MISC_FEATURE (17)..(17): Xaa17 is serine, threonine, alanine, valine, leucine, isoleucine, proline, phenylalanine, tyrosine, methionine, tryptophan, arginine, lysine, histidine, glutamine, asparagine or cysteine
  MISC_FEATURE (18)..(18): Xaa18 is arginine, lysine, histidine, glutamine or asparagine
  MISC_FEATURE (19)..(19): Xaa19 is serine, threonine, alanine, valine, leucine, isoleucine, proline, phenylalanine, tyrosine, methionine, tryptophan or cysteine
  MISC_FEATURE (20)..(20): Xaa20 is glycine, proline, alanine, beta alanine, asparagine, glutamine, serine, threonine, phenylalanine or tyrosine
  MISC_FEATURE (21)..(21): Xaa21 is glycine, proline, alanine, beta alanine, asparagine, glutamine, serine, threonine, phenylalanine or tyrosine
  MISC_FEATURE (22)..(22): Xaa22 is valine, alanine, leucine, isoleucine, proline, phenylalanine, tyrosine, methionine or tryptophan or threonine
  MISC_FEATURE (23)..(23): Xaa23 is valine, alanine, leucine, isoleucine, proline, phenylalanine, tyrosine, methionine, tryptophan or threonine
  MISC_FEATURE (24)..(24): Xaa24 is lysine, arginine, glutamine, asparagine or histidine
  MISC_FEATURE (25)..(25): Xaa25 is asparagine, glutamine, glycine, serine, threonine, tyrosine, phenylalanine, alanine, glutamate, aspartate or cysteine
  MISC_FEATURE (26)..(26): Xaa26 is asparagine, glutamine, glycine, serine, threonine, phenylalanine, tyrosine or cysteine
  MISC_FEATURE (28)..(28): Xaa28 is valine, alanine, leucine, isoleucine, proline, phenylalanine, tyrosine, methionine, tryptophan or threonine
  MISC_FEATURE (29)..(29): Xaa29 is proline, alanine, valine, leucine, isoleucine, glycine, phenylalanine, tyrosine, methionine or tryptophan
  MISC_FEATURE (31)..(31): Xaa31 is asparagine, glutamine, glycine, serine, threonine, phenylalanine, tyrosine, glutamate, aspartate or cysteine
  MISC_FEATURE (34)..(34): Xaa34 is serine, threonine, alanine, valine, leucine, isoleucine, proline, phenylalanine, tyrosine, methionine, tryptophan or cysteine
  MISC_FEATURE (35)..(35): Xaa35 is lysine, arginine, glutamine, asparagine, histidine, aspartate or glutamate
  MISC_FEATURE (36)..(36): Xaa36 is alanine, valine, leucine, isoleucine, proline, phenylalanine, tyrosine, methionine or tryptophan
XXXXXXXXXXXLXXXLXXXXXXXXXXFXXTXVGXXXF

SEQ ID NO: 2 - protein, 37 aa, Artificial
  Synthetic peptide sequence
  MISC_FEATURE (1)..(1): Xaa1 is absent or is alanine or serine
  MISC_FEATURE (2)..(2): Xaa2 is absent or is cysteine or homocysteine
  MISC_FEATURE (3)..(3): Xaa3 is absent or is aspartate or asparagine
  MISC_FEATURE (4)..(4): Xaa4 is absent or is threonine
  MISC_FEATURE (5)..(5): Xaa5 is absent or is alanine
  MISC_FEATURE (6)..(6): Xaa6 is absent or is threonine, cysteine or homocysteine
  MISC_FEATURE (7)..(7): Xaa7 is absent or is cysteine or homocysteine
  MISC_FEATURE (8)..(8): Xaa8 is valine, cysteine or homocysteine
  MISC_FEATURE (10)..(10): Xaa10 is histidine, cysteine or homocysteine
  MISC_FEATURE (11)..(11): Xaa11 is arginine, cysteine or homocysteine
  MISC_FEATURE (14)..(14): Xaa14 is glycine or aspartate
  MISC_FEATURE (17)..(17): Xaa17 is serine, arginine, cysteine or homocysteine
  MISC_FEATURE (18)..(18): Xaa18 is arginine, cysteine or homocysteine
  MISC_FEATURE (19)..(19): Xaa19 is a serine, cysteine or homocysteine
  MISC_FEATURE (21)..(21): Xaa21 is glycine, cysteine or homocysteine
  MISC_FEATURE (22)..(22): Xaa22 is valine or methionine
  MISC_FEATURE (23)..(23): Xaa23 is valine or leucine
  MISC_FEATURE (24)..(24): Xaa24 is lysine, cysteine or homocysteine
  MISC_FEATURE (25)..(25): Xaa25 is asparagine, serine or aspartate
  MISC_FEATURE (31)..(31): Xaa31 is asparagine or aspartate
  MISC_FEATURE (35)..(35): Xaa35 is lysine, glutamate, cysteine or homocysteine
XXXXXXXXTXXLAXLLXXXGXXXXXNFVPTXVGSXAF

SEQ ID NO: 3 - protein, 37 aa, Homo sapiens
ACDTATCVTHRLAGLLSRSGGVVKNNFVPTNVGSKAF

SEQ ID NO: 4 - protein, 37 aa, Homo sapiens
ACNTATCVTHRLAGLLSRSGGMVKSNFVPTNVGSKAF

SEQ ID NO: 5 - protein, 37 aa, Artificial
  Synthetic peptide sequence
  MISC_FEATURE (2)..(2): Xaa2 is cysteine or homocysteine
  MISC_FEATURE (7)..(7): Xaa7 is cysteine or homocysteine
AXDTATXVTHRLAGLLSRSGGVVKNNFVPTNVGSKAF

SEQ ID NO: 6 - protein, 37 aa, Artificial
  Synthetic peptide sequence
  MISC_FEATURE (1)..(1): Xaa1 is cysteine or homocysteine
  MISC_FEATURE (2)..(2): Xaa2 is cysteine or homocysteine
  MISC_FEATURE (7)..(7): Xaa7 is cysteine or homocysteine
XXDTATXVTHRLAGLLSRSGGVVKNNFVPTNVGSKAF

SEQ ID NO: 7 - protein, 37 aa, Artificial
  Synthetic peptide sequence
  MISC_FEATURE (2)..(2): Xaa2 is cysteine or homocysteine
  MISC_FEATURE (3)..(3): Xaa3 is cysteine or homocysteine
  MISC_FEATURE (7)..(7): Xaa7 is cysteine or homocysteine
AXXTATXVTHRLAGLLSRSGGVVKNNFVPTNVGSKAF

SEQ ID NO: 8 - protein, 37 aa, Artificial
  Synthetic peptide sequence
  MISC_FEATURE (2)..(2): Xaa2 is cysteine or homocysteine
  MISC_FEATURE (4)..(4): Xaa4 is cysteine or homocysteine
  MISC_FEATURE (7)..(7): Xaa7 is cysteine or homocysteine
AXDXATXVTHRLAGLLSRSGGVVKNNFVPTNVGSKAF

SEQ ID NO: 9 - protein, 37 aa, Artificial
  Synthetic peptide sequence
  MISC_FEATURE (2)..(2): Xaa2 is cysteine or homocysteine
  MISC_FEATURE (5)..(5): Xaa5 is cysteine or homocysteine
  MISC_FEATURE (7)..(7): Xaa7 is cysteine or homocysteine
AXDTXTXVTHRLAGLLSRSGGVVKNNFVPTNVGSKAF

SEQ ID NO: 10 - protein, 37 aa, Artificial
  Synthetic peptide sequence
  MISC_FEATURE (2)..(2): Xaa2 is cysteine or homocysteine
  MISC_FEATURE (6)..(6): Xaa6 is cysteine or homocysteine
  MISC_FEATURE (7)..(7): Xaa7 is cysteine or homocysteine
AXDTAXXVTHRLAGLLSRSGGVVKNNFVPTNVGSKAF

SEQ ID NO: 11 - protein, 36 aa, Artificial
  Synthetic peptide sequence
  MISC_FEATURE (1)..(1): Xaa1 is cysteine or homocysteine
  MISC_FEATURE (6)..(6): Xaa6 is cysteine or homocysteine
XDTATXVTHRLAGLLSRSGGVVKNNFVPTNVGSKAF

SEQ ID NO: 12 - protein, 35 aa, Artificial
  Synthetic peptide sequence
  MISC_FEATURE (5)..(5): Xaa5 is cysteine or homocysteine
DTATXVTHRLAGLLSRSGGVVKNNFVPTNVGSKAF

SEQ ID NO: 13 - protein, 35 aa, Artificial
  Synthetic peptide sequence
  MISC_FEATURE (1)..(1): Xaa1 is cysteine or homocysteine
  MISC_FEATURE (5)..(5): Xaa5 is cysteine or homocysteine
XTATXVTHRLAGLLSRSGGVVKNNFVPTNVGSKAF

SEQ ID NO: 14 - protein, 34 aa, Artificial
  Synthetic peptide sequence
  MISC_FEATURE (4)..(4): Xaa4 is cysteine or homocysteine
TATXVTHRLAGLLSRSGGVVKNNFVPTNVGSKAF

SEQ ID NO: 15 - protein, 33 aa, Artificial
  Synthetic peptide sequence
  MISC_FEATURE (3)..(3): Xaa3 is cysteine or homocysteine
ATXVTHRLAGLLSRSGGVVKNNFVPTNVGSKAF

SEQ ID NO: 16 - protein, 32 aa, Artificial
  Synthetic peptide sequence
  MISC_FEATURE (2)..(2): Xaa2 is cysteine or homocysteine
TXVTHRLAGLLSRSGGVVKNNFVPTNVGSKAF

SEQ ID NO: 17 - protein, 31 aa, Artificial
  Synthetic peptide sequence
  MISC_FEATURE (1)..(1): Xaa1 is cysteine or homocysteine
XVTHRLAGLLSRSGGVVKNNFVPTNVGSKAF

SEQ ID NO: 18 - protein, 30 aa, Artificial
  Synthetic peptide sequence
  MISC_FEATURE (1)..(1): Xaa1 is cysteine or homocysteine
XTHRLAGLLSRSGGVVKNNFVPTNVGSKAF

SEQ ID NO: 19 - protein, 30 aa, Artificial
  Synthetic peptide sequence
  MISC_FEATURE (2)..(2): Xaa2 is cysteine or homocysteine
VXHRLAGLLSRSGGVVKNNFVPTNVGSKAF

SEQ ID NO: 20 - protein, 30 aa, Artificial
  Synthetic peptide sequence
  MISC_FEATURE (3)..(3): Xaa3 is cysteine or homocysteine
VTXRLAGLLSRSGGVVKNNFVPTNVGSKAF

SEQ ID NO: 21 - protein, 30 aa, Artificial
  Synthetic peptide sequence
  MISC_FEATURE (4)..(4): X is cysteine or homocysteine
VTHXLAGLLSRSGGVVKNNFVPTNVGSKAF

SEQ ID NO: 22 - protein, 30 aa, Artificial
  Synthetic peptide sequence
  MISC_FEATURE (6)..(6): Xaa6 is cysteine or homocysteine
VTHRLXGLLSRSGGVVKNNFVPTNVGSKAF

SEQ ID NO: 23 - protein, 30 aa, Artificial
  Synthetic peptide sequence
  MISC_FEATURE (7)..(7): Xaa7 is cysteine or homocysteine
VTHRLAXLLSRSGGVVKNNFVPTNVGSKAF

SEQ ID NO: 24 - protein, 30 aa, Artificial
  Synthetic peptide sequence
  MISC_FEATURE (8)..(8): Xaa8 is cysteine or homocysteine
VTHRLAGXLSRSGGVVKNNFVPTNVGSKAF

SEQ ID NO: 25 - protein, 30 aa, Artificial
  Synthetic peptide sequence
  MISC_FEATURE (10)..(10): Xaa10 is cysteine or homocysteine
VTHRLAGLLXRSGGVVKNNFVPTNVGSKAF

SEQ ID NO: 26 - protein, 30 aa, Artificial
  Synthetic peptide sequence
  MISC_FEATURE (11)..(11): Xaa11 is cysteine or homocysteine
VTHRLAGLLSXSGGVVKNNFVPTNVGSKAF

SEQ ID NO: 27 - protein, 30 aa, Artificial
  Synthetic peptide sequence
  MISC_FEATURE (12)..(12): Xaa12 is cysteine or homocysteine
VTHRLAGLLSRXGGVVKNNFVPTNVGSKAF

SEQ ID NO: 28 - protein, 30 aa, Artificial
  Synthetic peptide sequence
  MISC_FEATURE (13)..(13): Xaa13 is cysteine or homocysteine
VTHRLAGLLSRSXGVVKNNFVPTNVGSKAF

SEQ ID NO: 29 - protein, 30 aa, Artificial
  Synthetic peptide sequence
  MISC_FEATURE (14)..(14): Xaa14 is cysteine or homocysteine
VTHRLAGLLSRSGXVVKNNFVPTNVGSKAF

SEQ ID NO: 30 - protein, 30 aa, Artificial
  Synthetic peptide sequence
  MISC_FEATURE (15)..(15): Xaa15 is cysteine or homocysteine
VTHRLAGLLSRSGGXVKNNFVPTNVGSKAF

SEQ ID NO: 31 - protein, 30 aa, Artificial
  Synthetic peptide sequence
VTHRLAGLLSRSGGMVKSNFVPTNVGSKAF

SEQ ID NO: 32 - protein, 30 aa, Artificial
  Synthetic peptide sequence
  MISC_FEATURE (16)..(16): Xaa16 is cysteine or homocysteine
VTHRLAGLLSRSGGVXKNNFVPTNVGSKAF

SEQ ID NO: 33 - protein, 30 aa, Artificial
  Synthetic peptide sequence
  MISC_FEATURE (17)..(17): Xaa17 is cysteine or homocysteine
VTHRLAGLLSRSGGVVXNNFVPTNVGSKAF

SEQ ID NO: 34 - protein, 30 aa, Artificial
  Synthetic peptide sequence
  MISC_FEATURE (18)..(18): Xaa18 is cysteine or homocysteine
VTHRLAGLLSRSGGVVKXNFVPTNVGSKAF

SEQ ID NO: 35 - protein, 30 aa, Artificial
  Synthetic peptide sequence
  MISC_FEATURE (19)..(19): Xaa19 is cysteine or homocysteine
VTHRLAGLLSRSGGVVKNXFVPTNVGSKAF

SEQ ID NO: 36 - protein, 30 aa, Artificial
  Synthetic peptide sequence
  MISC_FEATURE (21)..(21): Xaa21 is cysteine or homocysteine
VTHRLAGLLSRSGGVVKNNFXPTNVGSKAF

SEQ ID NO: 37 - protein, 30 aa, Artificial
  Synthetic peptide sequence
  MISC_FEATURE (22)..(22): Xaa22 is cysteine or homocysteine
VTHRLAGLLSRSGGVVKNNFVXTNVGSKAF

SEQ ID NO: 38 - protein, 30 aa, Artificial
  Synthetic peptide sequence
  MISC_FEATURE (24)..(24): Xaa24 is cysteine or homocysteine
VTHRLAGLLSRSGGVVKNNFVPTXVGSKAF

SEQ ID NO: 39 - protein, 30 aa, Artificial
  Synthetic peptide sequence
  MISC_FEATURE (27)..(27): Xaa27 is cysteine or homocysteine
VTHRLAGLLSRSGGVVKNNFVPTNVGXKAF

SEQ ID NO: 40 - protein, 30 aa, Artificial
  Synthetic peptide sequence
  MISC_FEATURE (28)..(28): Xaa28 is cysteine or homocysteine
VTHRLAGLLSRSGGVVKNNFVPTNVGSXAF

SEQ ID NO: 41 - protein, 30 aa, Artificial
  Synthetic peptide sequence
  MISC_FEATURE (29)..(29): Xaa29 is cysteine or homocysteine
VTHRLAGLLSRSGGVVKNNFVPTNVGSKXF

SEQ ID NO: 42 - protein, 37 aa, Artificial
  Synthetic peptide sequence
  MISC_FEATURE (2)..(2): Xaa2 is cysteine or homocysteine
  MISC_FEATURE (7)..(7): Xaa7 is cysteine or homocysteine
AXNTATXVTHRLAGLLSRSGGMVKSNFVPTNVGSKAF

SEQ ID NO: 43 - protein, 37 aa, Artificial
  Synthetic peptide sequence
  MISC_FEATURE (1)..(1): Xaa1 is cysteine or homocysteine
  MISC_FEATURE (2)..(2): Xaa2 is cysteine or homocysteine
  MISC_FEATURE (7)..(7): Xaa7 is cysteine or homocysteine
XXNTATXVTHRLAGLLSRSGGMVKSNFVPTNVGSKAF

SEQ ID NO: 44 - protein, 37 aa, Artificial
  Synthetic peptide sequence
  MISC_FEATURE (2)..(2): Xaa2 is cysteine or homocysteine
  MISC_FEATURE (3)..(3): Xaa3 is cysteine or homocysteine
  MISC_FEATURE (7)..(7): Xaa7 is cysteine or homocysteine
AXXTATXVTHRLAGLLSRSGGMVKSNFVPTNVGSKAF

SEQ ID NO: 45 - protein, 37 aa, Artificial
  Synthetic peptide sequence
  MISC_FEATURE (2)..(2): Xaa2 is cysteine or homocysteine
  MISC_FEATURE (4)..(4): Xaa4 is cysteine or homocysteine
  MISC_FEATURE (7)..(7): Xaa7 is cysteine or homocysteine
AXNXATXVTHRLAGLLSRSGGMVKSNFVPTNVGSKAF

SEQ ID NO: 46 - protein, 37 aa, Artificial
  Synthetic peptide sequence
  MISC_FEATURE (2)..(2): Xaa2 is cysteine or homocysteine
  MISC_FEATURE (5)..(5): Xaa5 is cysteine or homocysteine
  MISC_FEATURE (7)..(7): Xaa7 is cysteine or homocysteine
AXNTXTXVTHRLAGLLSRSGGMVKSNFVPTNVGSKAF

SEQ ID NO: 47 - protein, 37 aa, Artificial
  Synthetic peptide sequence
  MISC_FEATURE (2)..(2): Xaa2 is cysteine or homocysteine
  MISC_FEATURE (6)..(6): Xaa6 is cysteine or homocysteine
  MISC_FEATURE (7)..(7): Xaa7 is cysteine or homocysteine
AXNTAXXVTHRLAGLLSRSGGMVKSNFVPTNVGSKAF

SEQ ID NO: 48 - protein, 36 aa, Artificial
  Synthetic peptide sequence
  MISC_FEATURE (1)..(1): Xaa1 is cysteine or homocysteine
  MISC_FEATURE (6)..(6): Xaa6 is cysteine or homocysteine
XNTATXVTHRLAGLLSRSGGMVKSNFVPTNVGSKAF

SEQ ID NO: 49 - protein, 35 aa, Artificial
  Synthetic peptide sequence
  MISC_FEATURE (5)..(5): Xaa5 is cysteine or homocysteine
NTATXVTHRLAGLLSRSGGMVKSNFVPTNVGSKAF

SEQ ID NO: 50 - protein, 38 aa, Artificial
  Synthetic peptide sequence
  MISC_FEATURE (2)..(2): Xaa2 is cysteine or homocysteine
  MISC_FEATURE (4)..(4): Xaa4 is cysteine or homocysteine
  MISC_FEATURE (8)..(8): Xaa8 is cysteine or homocysteine
AXNXTATXVTHRLAGLLSRSGGMVKSNFVPTNVGSKAF

SEQ ID NO: 51 - protein, 35 aa, Artificial
  Synthetic peptide sequence
  MISC_FEATURE (1)..(1): Xaa1 is cysteine or homocysteine
  MISC_FEATURE (5)..(5): Xaa5 is cysteine or homocysteine
XTATXVTHRLAGLLSRSGGMVKSNFVPTNVGSKAF

SEQ ID NO: 52 - protein, 34 aa, Artificial
  Synthetic peptide sequence
  MISC_FEATURE (4)..(4): Xaa4 is cysteine or homocysteine
TATXVTHRLAGLLSRSGGMVKSNFVPTNVGSKAF

SEQ ID NO: 53 - protein, 33 aa, Artificial
  Synthetic peptide sequence
  MISC_FEATURE (3)..(3): Xaa3 is cysteine or homocysteine
ATXVTHRLAGLLSRSGGMVKSNFVPTNVGSKAF

SEQ ID NO: 54 - protein, 32 aa, Artificial
  Synthetic peptide sequence
  MISC_FEATURE (2)..(2): Xaa2 is cysteine or homocysteine
TXVTHRLAGLLSRSGGMVKSNFVPTNVGSKAF

SEQ ID NO: 55 - protein, 31 aa, Artificial
  Synthetic peptide sequence
  MISC_FEATURE (1)..(1): Xaa1 is cysteine or homocysteine
XVTHRLAGLLSRSGGMVKSNFVPTNVGSKAF

SEQ ID NO: 56 - protein, 30 aa, Artificial
  Synthetic peptide sequence
  MISC_FEATURE (1)..(1): Xaa1 is cysteine or homocysteine
XTHRLAGLLSRSGGMVKSNFVPTNVGSKAF

SEQ ID NO: 57 - protein, 30 aa, Artificial
  Synthetic peptide sequence
  MISC_FEATURE (2)..(2): Xaa2 is cysteine or homocysteine
VXHRLAGLLSRSGGMVKSNFVPTNVGSKAF

SEQ ID NO: 58 - protein, 30 aa, Artificial
  Synthetic peptide sequence
  MISC_FEATURE (3)..(3): Xaa3 is cysteine or homocysteine
VTXRLAGLLSRSGGMVKSNFVPTNVGSKAF

SEQ ID NO: 59 - protein, 30 aa, Artificial
  Synthetic peptide sequence
  MISC_FEATURE (4)..(4): Xaa4 is cysteine or homocysteine
VTHXLAGLLSRSGGMVKSNFVPTNVGSKAF

SEQ ID NO: 60 - protein, 30 aa, Artificial
  Synthetic peptide sequence
  MISC_FEATURE (6)..(6): Xaa6 is cysteine or homocysteine
VTHRLXGLLSRSGGMVKSNFVPTNVGSKAF

SEQ ID NO: 61 - protein, 30 aa, Artificial
  Synthetic peptide sequence
  MISC_FEATURE (7)..(7): Xaa7 is cysteine or homocysteine
VTHRLAXLLSRSGGMVKSNFVPTNVGSKAF

SEQ ID NO: 62 - protein, 30 aa, Artificial
  Synthetic peptide sequence
  MISC_FEATURE (8)..(8): Xaa8 is cysteine or homocysteine
VTHRLAGXLSRSGGMVKSNFVPTNVGSKAF

SEQ ID NO: 63 - protein, 30 aa, Artificial
  Synthetic peptide sequence
  MISC_FEATURE (10)..(10): Xaa10 is cysteine or homocysteine
VTHRLAGLLXRSGGMVKSNFVPTNVGSKAF

SEQ ID NO: 64 - protein, 30 aa, Artificial
  Synthetic peptide sequence
  MISC_FEATURE (11)..(11): Xaa11 is cysteine or homocysteine
VTHRLAGLLSXSGGMVKSNFVPTNVGSKAF

SEQ ID NO: 65 - protein, 30 aa, Artificial
  Synthetic peptide sequence
  MISC_FEATURE (12)..(12): Xaa12 is cysteine or homocysteine
VTHRLAGLLSRXGGMVKSNFVPTNVGSKAF

SEQ ID NO: 66 - protein, 30 aa, Artificial
  Synthetic peptide sequence
  MISC_FEATURE (13)..(13): Xaa13 is cysteine or homocysteine
VTHRLAGLLSRSXGMVKSNFVPTNVGSKAF

SEQ ID NO: 67 - protein, 30 aa, Artificial
  Synthetic peptide sequence
  MISC_FEATURE (14)..(14): Xaa14 is cysteine or homocysteine
VTHRLAGLLSRSGXMVKSNFVPTNVGSKAF

SEQ ID NO: 68 - protein, 30 aa, Artificial
  Synthetic peptide sequence
  MISC_FEATURE (15)..(15): Xaa15 is cysteine or homocysteine
VTHRLAGLLSRSGGXVKSNFVPTNVGSKAF

SEQ ID NO: 69 - protein, 30 aa, Artificial
  Synthetic peptide sequence
  MISC_FEATURE (16)..(16): Xaa16 is cysteine or homocysteine
VTHRLAGLLSRSGGMXKSNFVPTNVGSKAF

SEQ ID NO: 70 - protein, 30 aa, Artificial
  Synthetic peptide sequence
  MISC_FEATURE (17)..(17): Xaa17 is cysteine or homocysteine
VTHRLAGLLSRSGGMVXSNFVPTNVGSKAF

SEQ ID NO: 71 - protein, 30 aa, Artificial
  Synthetic peptide sequence
  MISC_FEATURE (18)..(18): Xaa18 is cysteine or homocysteine
VTHRLAGLLSRSGGMVKXNFVPTNVGSKAF

SEQ ID NO: 72 - protein, 30 aa, Artificial
  Synthetic peptide sequence
  MISC_FEATURE (19)..(19): Xaa19 is cysteine or homocysteine
VTHRLAGLLSRSGGMVKSXFVPTNVGSKAF

SEQ ID NO: 73 - protein, 30 aa, Artificial
  Synthetic peptide sequence
  MISC_FEATURE (21)..(21): Xaa21 is cysteine or homocysteine
VTHRLAGLLSRSGGMVKSNFXPTNVGSKAF

SEQ ID NO: 74 - protein, 30 aa, Artificial
  Synthetic peptide sequence
  MISC_FEATURE (22)..(22): Xaa22 is cysteine or homocysteine
VTHRLAGLLSRSGGMVKSNFVXTNVGSKAF

SEQ ID NO: 75 - protein, 30 aa, Artificial
  Synthetic peptide sequence
  MISC_FEATURE (24)..(24): Xaa24 is cysteine or homocysteine
VTHRLAGLLSRSGGMVKSNFVPTXVGSKAF

SEQ ID NO: 76 - protein, 30 aa, Artificial
  Synthetic peptide sequence
  MISC_FEATURE (27)..(27): Xaa27 is cysteine or homocysteine
VTHRLAGLLSRSGGMVKSNFVPTNVGXKAF

SEQ ID NO: 77 - protein, 30 aa, Artificial
  Synthetic peptide sequence
  MISC_FEATURE (28)..(28): Xaa28 is cysteine or homocysteine
VTHRLAGLLSRSGGMVKSNFVPTNVGSXAF

SEQ ID NO: 78 - protein, 30 aa, Artificial
  Synthetic peptide sequence
  MISC_FEATURE (29)..(29): Xaa29 is cysteine or homocysteine
VTHRLAGLLSRSGGMVKSNFVPTNVGSKXF

SEQ ID NO: 79 - protein, 31 aa, Artificial
  Synthetic peptide sequence
CVTHRLAGLLSRSGGVVKNNFVPTNVGSKAF

SEQ ID NO: 80 - protein, 30 aa, Artificial
  Synthetic peptide sequence
CTHRLAGLLSRSGGVVKNNFVPTNVGSKAF

SEQ ID NO: 81 - protein, 30 aa, Artificial
  Synthetic peptide sequence
VTHRLAGLLSRSGGVVCNNFVPTNVGSKAF

SEQ ID NO: 82 - protein, 30 aa, Artificial
  Synthetic peptide sequence
VTHRLAGLLSRSGGVVKNNFVPTNVGSCAF

SEQ ID NO: 83 - protein, 37 aa, Artificial
  Synthetic peptide sequence
ACDTACCVTHRLAGLLSRSGGVVKNNFVPTNVGSKAF

SEQ ID NO: 84 - protein, 30 aa, Artificial
  Synthetic peptide sequence
VTCRLAGLLSRSGGVVKNNFVPTNVGSKAF

SEQ ID NO: 85 - protein, 30 aa, Artificial
  Synthetic peptide sequence
VTHCLAGLLSRSGGVVKNNFVPTNVGSKAF

SEQ ID NO: 86 - protein, 30 aa, Artificial
  Synthetic peptide sequence
VTHRLAGLLSRSGCVVKNNFVPTNVGSKAF

SEQ ID NO: 87 - protein, 31 aa, Artificial
  Synthetic peptide sequence
CVTHRLAGLLSRSGGMVKSNFVPTNVGSKAF

SEQ ID NO: 88 - protein, 30 aa, Artificial
  Synthetic peptide sequence
CTHRLAGLLSRSGGMVKSNFVPTNVGSKAF

SEQ ID NO: 89 - protein, 30 aa, Artificial
  Synthetic peptide sequence
VTHRLAGLLSRSGGMVCSNFVPTNVGSKAF

SEQ ID NO: 90 - protein, 30 aa, Artificial
  Synthetic peptide sequence
VTHRLAGLLSRSGGMVKSNFVPTNVGSCAF

SEQ ID NO: 91 - protein, 37 aa, Artificial
  Synthetic peptide sequence
ACNTACCVTHRLAGLLSRSGGMVKSNFVPTNVGSKAF

SEQ ID NO: 92 - protein, 30 aa, Artificial
  Synthetic peptide sequence
VTCRLAGLLSRSGGMVKSNFVPTNVGSKAF

SEQ ID NO: 93 - protein, 30 aa, Artificial
  Synthetic peptide sequence
VTHCLAGLLSRSGGMVKSNFVPTNVGSKAF

SEQ ID NO: 94 - protein, 30 aa, Artificial
  Synthetic peptide sequence
VTHRLAGLLSRSGCMVKSNFVPTNVGSKAF

SEQ ID NO: 95 - protein, 31 aa, Homo sapiens; identical to SEQ ID NO: 79

SEQ ID NO: 96 - protein, 30 aa, Homo sapiens
VTHRLAGLLSRSGGVVKNNFVPTNVGSKAF

SEQ ID NO: 97 - protein, 31 aa, Homo sapiens; identical to SEQ ID NO: 87

SEQ ID NO: 98 - protein, 30 aa, Homo sapiens; identical to SEQ ID NO: 31

SEQ ID NO: 99 - protein, 30 aa, Artificial
  Synthetic peptide sequence
VTHCLAGLLSRSGGVVCNNFVPTNVGSKAF

SEQ ID NO: 100 - protein, 31 aa, Artificial
  Synthetic peptide sequence
  MISC_FEATURE (1)..(1): Xaa1 is cysteine or homocysteine
  MISC_FEATURE (2)..(2): Xaa2 is cysteine or homocysteine
XXTHRLAGLLSRSGGVVKNNFVPTNVGSKAF

SEQ ID NO: 101 - protein, 31 aa, Artificial
  Synthetic peptide sequence
  MISC_FEATURE (1)..(1): Xaa1 is cysteine or homocysteine
  MISC_FEATURE (5)..(5): Xaa5 is cysteine or homocysteine
XVTHXLAGLLSRSGGVVKNNFVPTNVGSKAF

SEQ ID NO: 102 - protein, 31 aa, Artificial
  Synthetic peptide sequence
  MISC_FEATURE (1)..(1): Xaa1 is cysteine or homocysteine
  MISC_FEATURE (18)..(18): Xaa18 is cysteine or homocysteine
XVTHRLAGLLSRSGGVVXNNFVPTNVGSKAF

SEQ ID NO: 103 - protein, 31 aa, Artificial
  Synthetic peptide sequence
  MISC_FEATURE (1)..(1): Xaa1 is cysteine or homocysteine
  MISC_FEATURE (29)..(29): Xaa29 is cysteine or homocysteine
XVTHRLAGLLSRSGGVVKNNFVPTNVGSXAF

SEQ ID NO: 104 - protein, 30 aa, Artificial
  Synthetic peptide sequence
  MISC_FEATURE (1)..(1): Xaa1 is cysteine or homocysteine
  MISC_FEATURE (4)..(4): Xaa4 is cysteine or homocysteine
XTHXLAGLLSRSGGVVKNNFVPTNVGSKAF

SEQ ID NO: 105 - protein, 30 aa, Artificial
  Synthetic peptide sequence
  MISC_FEATURE (1)..(1): Xaa1 is cysteine or homocysteine
  MISC_FEATURE (17)..(17): Xaa17 is cysteine or homocysteine
XTHRLAGLLSRSGGVVXNNFVPTNVGSKAF

SEQ ID NO: 106 - protein, 30 aa, Artificial
  Synthetic peptide sequence
  MISC_FEATURE (1)..(1): Xaa1 is cysteine or homocysteine
  MISC_FEATURE (28)..(28): Xaa28  is cysteine or homocysteine
XTHRLAGLLSRSGGVVKNNFVPTNVGSXAF

SEQ ID NO: 107 - protein, 30 aa, Artificial
  Synthetic petpide sequence
  MISC_FEATURE (4)..(4): Xaa4 is cysteine or homocysteine
  MISC_FEATURE (17)..(17): Xaa17 is cysteine or homocysteine
VTHXLAGLLSRSGGVVXNNFVPTNVGSKAF

SEQ ID NO: 108 - protein, 30 aa, Artificial
  Synthetic peptide sequence
  MISC_FEATURE (4)..(4): Xaa4 is cysteine or homocysteine
  MISC_FEATURE (28)..(28): Xaa28 is cysteine or homocysteine
VTHXLAGLLSRSGGVVKNNFVPTNVGSXAF

SEQ ID NO: 109 - protein, 30 aa, Artificial
  Synthetic peptide sequence
  MISC_FEATURE (17)..(17): Xaa17 is cysteine or homocysteine
  MISC_FEATURE (28)..(28): Xaa28 is cysteine or homocysteine
VTHRLAGLLSRSGGVVXNNFVPTNVGSXAF

SEQ ID NO: 110 - protein, 31 aa, Artificial
  Synthetic peptide sequence
  MISC_FEATURE (1)..(1): Xaa1 is cysteine or homocysteine
  MISC_FEATURE (2)..(2): Xaa2 is cysteine or homocysteine
XXTHRLAGLLSRSGGMVKSNFVPTNVGSKAF

SEQ ID NO: 111 - protein, 31 aa, Artificial
  Synthetic peptide sequence
  MISC_FEATURE (1)..(1): Xaa1 is cysteine or homocysteine
  MISC_FEATURE (5)..(5): Xaa5 is cysteine or homocysteine
XVTHXLAGLLSRSGGMVKSNFVPTNVGSKAF

SEQ ID NO: 112 - protein, 31 aa, Artificial
  Synthetic peptide sequence
  MISC_FEATURE (1)..(1): Xaa1 is cysteine or homocysteine
  MISC_FEATURE (18)..(18): Xaa18 is cysteine or homocysteine
XVTHRLAGLLSRSGGMVXSNFVPTNVGSKAF

SEQ ID NO: 113 - protein, 31 aa, Artificial
  Synthetic peptide sequence
  MISC_FEATURE (1)..(1): Xaa1 is cysteine or homocysteine
  MISC_FEATURE (29)..(29): Xaa19 is cysteine or homocysteine
XVTHRLAGLLSRSGGMVKSNFVPTNVGSXAF

SEQ ID NO: 114 - protein, 30 aa, Artificial
  Synthetic peptide sequence
  MISC_FEATURE (1)..(1): Xaa1 is cysteine or homocysteine
  MISC_FEATURE (4)..(4): Xaa4 is cysteine or homocysteine
XTHXLAGLLSRSGGMVKSNFVPTNVGSKAF

SEQ ID NO: 115 - protein, 30 aa, Artificial
  Synthetic peptide sequence
  MISC_FEATURE (1)..(1): Xaa1 is cysteine or homocysteine
  MISC_FEATURE (17)..(17): Xaa17 is cysteine or homocysteine
XTHRLAGLLSRSGGMVXSNFVPTNVGSKAF

SEQ ID NO: 116 - protein, 30 aa, Artificial
  Synthetic peptide sequence
  MISC_FEATURE (1)..(1): Xaa1 is cysteine or homocysteine
  MISC_FEATURE (28)..(28): Xaa28 is cysteine or homocysteine
XTHRLAGLLSRSGGMVKSNFVPTNVGSXAF

SEQ ID NO: 117 - protein, 30 aa, Artificial
  Synthetic peptide sequence
  MISC_FEATURE (4)..(4): Xaa4 is cysteine or homocysteine
  MISC_FEATURE (17)..(17): Xaa17 is cysteine or homocysteine
VTHXLAGLLSRSGGMVXSNFVPTNVGSKAF

SEQ ID NO: 118 - protein, 30 aa, Artificial
  Synthetic peptide sequence
  MISC_FEATURE (4)..(4): Xaa4 is cysteine or homocysteine
  MISC_FEATURE (28)..(28): Xaa28 is cysteine or homocysteine
VTHXLAGLLSRSGGMVKSNFVPTNVGSXAF

SEQ ID NO: 119 - protein, 30 aa, Artificial
  Synthetic peptide sequence
  MISC_FEATURE (17)..(17): Xaa17 is cysteine or homocysteine
  MISC_FEATURE (28)..(28): Xaa28 is cysteine or homocysteine
VTHRLAGLLSRSGGMVXSNFVPTNVGSXAF

SEQ ID NO: 120 - protein, 31 aa, Artificial
  Synthetic peptide sequence
CCTHRLAGLLSRSGGVVKNNFVPTNVGSKAF

SEQ ID NO: 121 - protein, 31 aa, Artificial
  Synthetic peptide sequence
CVTHCLAGLLSRSGGVVKNNFVPTNVGSKAF

SEQ ID NO: 122 - protein, 31 aa, Artificial
  Synthetic peptide sequence
CVTHRLAGLLSRSGGVVCNNFVPTNVGSKAF

SEQ ID NO: 123 - protein, 31 aa, Artificial
  Synthetic peptide sequence
CVTHRLAGLLSRSGGVVKNNFVPTNVGSCAF

SEQ ID NO: 124 - protein, 30 aa, Artificial
  Synthetic peptide sequence
CTHCLAGLLSRSGGVVKNNFVPTNVGSKAF

SEQ ID NO: 125 - protein, 30 aa, Artificial
  Synthetic peptide sequence
CTHRLAGLLSRSGGVVCNNFVPTNVGSKAF

SEQ ID NO: 126 - protein, 30 aa, Artificial
  Synthetic peptide sequence
CTHRLAGLLSRSGGVVKNNFVPTNVGSCAF

SEQ ID NO: 127 - protein, 30 aa, Artificial
  Synthetic peptide sequence
VTHCLAGLLSRSGGVVKNNFVPTNVGSCAF

SEQ ID NO: 128 - protein, 30 aa, Artificial
  Synthetic peptide sequence
VTHRLAGLLSRSGGVVCNNFVPTNVGSCAF

SEQ ID NO: 129 - protein, 31 aa, Artificial
  Synthetic peptide sequence
CCTHRLAGLLSRSGGMVKSNFVPTNVGSKAF

SEQ ID NO: 130 - protein, 31 aa, Artificial
  Synthetic peptide sequence
CVTHCLAGLLSRSGGMVKSNFVPTNVGSKAF

SEQ ID NO: 131 - protein, 31 aa, Artificial
  Synthetic peptide sequence
CVTHRLAGLLSRSGGMVCSNFVPTNVGSKAF

SEQ ID NO: 132 - protein, 31 aa, Artificial
  Synthetic peptide sequence
CVTHRLAGLLSRSGGMVKSNFVPTNVGSCAF

SEQ ID NO: 133 - protein, 30 aa, Artificial
  Synthetic peptide sequence
CTHCLAGLLSRSGGMVKSNFVPTNVGSKAF

SEQ ID NO: 134 - protein, 30 aa, Artificial
  Synthetic peptide sequence
CTHRLAGLLSRSGGMVCSNFVPTNVGSKAF

SEQ ID NO: 135 - protein, 30 aa, Artificial
  Synthetic peptide sequence
CTHRLAGLLSRSGGMVKSNFVPTNVGSCAF

SEQ ID NO: 136 - protein, 30 aa, Artificial
  Synthetic peptide sequence
VTHCLAGLLSRSGGMVCSNFVPTNVGSKAF

SEQ ID NO: 137 - protein, 30 aa, Artificial
  Synthetic peptide sequence
VTHCLAGLLSRSGGMVKSNFVPTNVGSCAF

SEQ ID NO: 138 - protein, 30 aa, Artificial
  Synthetic peptide sequence
VTHRLAGLLSRSGGMVCSNFVPTNVGSCAF